(19) United States
(12) Patent Application Publication
(10) Pub. No.: US 2024/0024520 A1
(43) Pub. Date: Jan. 25, 2024

(54) AUTONOMOUS KNOB DOMAIN PEPTIDES

(71) Applicant: UCB BIOPHARMA SRL, Brussels (BE)

(72) Inventors: Alastair David Griffiths LAWSON, Slough, Berkshire (GB); Alexander MACPHERSON, Slough, Berkshire (GB); Anthony SCOTT-TUCKER, Slough, Berkshire (GB); Anastasios SPILIOTOPOULOS, Slough, Berkshire (GB)

(21) Appl. No.: 17/907,116

(22) PCT Filed: Mar. 26, 2021
(86) PCT No.: PCT/EP2021/057946

§ 371 Date: Sep. 23, 2022

(30) Foreign Application Priority Data
Mar. 27, 2020 (GB) 2004462.4
May 29, 2020 (GB) 2008095.8

(51) Int. Cl. A61K 51/10; C07K 16/18; C12N 15/10

(52) U.S. Cl. CPC A61K 51/1096; C07K 16/18; C12N 15/1037; C07K 2317/20; C07K 2317/24

(57) ABSTRACT

The present disclosure relates to isolated fragments of antibodies, in particular to isolated knob domains of bovine ultralong CDR-H3 or portions thereof which bind to an antigen of interest, and formulations comprising the same. The disclosure further relates to the use of the isolated antibody fragments and formulations in therapy. The present disclosure also extends to methods of preparing said isolated antibody fragments.

### FIELD OF THE INVENTION

The present disclosure relates to isolated fragments of antibodies, in particular to isolated knob domains of bovine ultralong CDR-H3 or portions thereof which bind to an antigen of interest, and formulations comprising the same. The disclosure further relates to the use of the isolated antibody fragments and formulations in therapy. The present disclosure also extends to methods of preparing said isolated antibody fragments.

### BACKGROUND OF THE INVENTION

The high specificity and affinity of antibodies makes them ideal diagnostic and therapeutic agents. Standard full-length monoclonal antibodies have a size of 150 kDa. Advances in the field of recombinant antibody technology have resulted in the production of antibody fragments, such as Fv, Fab, Fab′ and F(ab′)₂ fragments. These smaller molecules retain the antigen binding activity of whole antibodies and can also exhibit altered biodistribution, tissue penetration and pharmacokinetic properties in comparison to whole immunoglobulin molecules. Indeed, antibody fragments are proving to be versatile therapeutic agents. To date, the smallest autonomous, naturally occurring, functional antibody fragment reported has been the VHH fragment derived from camelids (Hamers-Casterman, C. et al. Naturally occurring antibodies devoid of light chains. Nature 363, 446-448 (1993)) and the VNAR (Variable New antigen Receptor) fragment from sharks (Greenberg, A. S. et al. A new antigen receptor gene family that undergoes rearrangement and extensive somatic diversification in sharks. Nature 374, 168-173 (1995)), resulting in heavy chain variable region fragments, of some 12-15 kDa.

Whilst such fragments appear to exhibit a number of advantages over whole immunoglobulins, they also suffer from an increased rate of clearance from serum since they lack the Fc domain that imparts a long lifetime in vivo. In addition, the availability of recombinant production methods and systems is limiting on the antibody production and may present technical challenges, e.g. in terms of DNA engineering, productivity in cells, etc. . . . . Further miniaturising antibody fragments may allow them to be produced without recombinant antibody expression. The smallest fragments may be amenable to chemical synthesis and remove entirely the need for DNA or cells.

Therefore, there is a need to provide new therapeutics, including new antibody derived therapeutics, having improved properties useful in therapy, notably having improved pharmacokinetic properties (e.g. biodistribution, bioavailability, cell and tissue penetration, clearance) and/or improved biological function (e.g. specificity, binding affinity, neutralisation, cell cytotoxicity) and/or the capacity to be manufactured by chemical synthesis, independent of cells or cellular machinery.

There is also a need to identify new methods of producing highly diverse and specific antibody derived therapeutics.

Some bovine antibodies have been characterized by unusually long CDR-H3 (so called “bovine ultralong CDR-H3”) with lengths of up to 69 residues, which participate to the high diversity of the antibody repertoire. The bovine ultralong CDR-H3 have been characterized by a very unusual tridimensional structure comprising a “stalk domain” and a “knob domain”, for example by Stanfield et al. (Stanfield, R. L., Wilson, I. A. & Smider, V. V. Conservation and diversity in the ultralong third heavy-chain complementarity-determining region of bovine antibodies. Sci Immunol 1, (2016) (hereinafter “Stanfield et al. (supra)”). For example, WO2013/106485 describes humanized antibodies comprising an ultralong CDR-H3, in particular wherein a heterologous polypeptide is inserted into or replaces at least a portion of the knob domain of the ultralong CDR-H3. So far, bovine ultralong CDR-H3 have only been characterized when associated with additional domains of the whole antibody structure, notably when integrated as part of a Fab fragment as described in Stanfield et al. mentioned above.

### SUMMARY OF THE INVENTION

The present inventors show for the first time that bovine antibody knob domains are capable of binding antigen autonomously, with high affinity, in the absence of the ultralong CDR-H3 stalk region, neighbouring CDRs or Fab infrastructure.

In particular, the invention provides isolated knob domains of bovine ultralong CDR-H3 and portions thereof, which surprisingly retain functionality and are able to bind their antigen of interest outside of the full-length bovine antibody scaffold, i.e. when expressed on its own.

The present invention provides improved antibody fragments, notably bovine antibody fragments having a high specificity for their antigen of interest, and having improved properties, notably pharmacokinetic properties (e.g. biodistribution, bioavailability, cell and tissue penetration, clearance) and/or biological properties (e.g. specificity, binding affinity, neutralisation, cell cytotoxicity) useful in therapy.

Advantageously, the antibody fragments as disclosed herein effectively bridge a molecular weight gap between camelid-derived VHH antibody domains and chemical macrocycles, with potential for therapeutic utility. In addition, by virtue of their low molecular weight, the invention provides an antibody fragment which can be manufactured by chemical synthesis, without the requirement for a cellular machinery. Hence, the invention also provides peptides encoding antibody fragments according to the invention, which are not isolated from bovine but are produced synthetically.

In addition, the invention provides a new antibody format which may be useful in multiple applications, based on the diversity of the antigens, diseases and disorders than may be targeted. Advantageously, the new antibody format may lead to the discovery of new epitopes on an antigen of interest, as well as new biological pathways and new mechanisms of action associated thereto.

Thus, in one aspect, there is provided an isolated antibody fragment, wherein the fragment is a knob domain of a bovine ultralong CDR-H3 or portion thereof which binds to an antigen of interest.

In one embodiment, the isolated antibody fragment is the knob domain of the bovine ultralong CDR-H3. In one embodiment, the isolated antibody fragment comprises at least two, or at least four, or at least six, or at least eight, or at least ten cysteine residues. In one embodiment, the isolated antibody fragment comprises at least one, or at least two, or at least three, or at least four, or a at least five disulphide bonds. In one embodiment, the isolated antibody fragment comprises a (Z₁) X₁ C X₂ motif at its N-terminal extremity, wherein:
a. Z₁ is present or absent, and when Z₁ is present, Z₁ represents 1 amino acid or 2, 3, 4, or 5 independently selected amino acids; and,
b. X₁ is any amino acid residue, preferably selected from the list consisting of Serine, Threonine, Asparagine, Alanine, Glycine, Proline, Histidine, Lysine, Valine, Arginine, Isoleucine, Leucine, Phenylalanine and Aspartic acid; and,
c. C is cysteine; and,
d. X₂ is an amino acid selected from the list consisting of Proline, Arginine, Histidine, Lysine, Glycine and Serine.

In one embodiment, the isolated antibody fragment comprises a (AB)n and/or (BA)n motif, wherein A is any amino acid residue, B is an aromatic amino acid selected from the group consisting of: tyrosine (Y), phenylalanine (F), tryptophan (W), and histidine (H), and wherein n is 1, 2, 3, or 4.

In one embodiment, the isolated antibody fragment is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length or more, and is up to 55 amino acids in length. In one embodiment, the isolated antibody fragment is between 5 and 55, or between 15 and 50, or between 20 and 45 or between 25 and 40 amino acids in length.

In one embodiment, the isolated antibody fragment comprises a sequence which is a variant of a naturally occurring sequence.

In one embodiment, the isolated antibody fragment according to the invention further comprises a bridging moiety between two amino acids. In one embodiment, the bridging moiety comprises a feature selected from the group consisting of a disulphide bond, an amide bond (lactam), a thioether bond, an aromatic ring, an unsaturated aliphatic hydrocarbon chain, a saturated aliphatic hydrocarbon chain and a triazole ring.

In one embodiment, the isolated antibody fragment is fully bovine. In one embodiment, the isolated antibody fragment is chimeric. In one embodiment, the isolated antibody fragment is synthetic.

In one embodiment, the isolated antibody fragment binds to the component C5 of the Complement, i.e. the antigen of interest is C5. In one embodiment, the isolated bovine antibody fragment has a sequence selected from the list consisting of SEQ ID NO: 157 to SEQ ID NO: 310, SEQ ID NO: 313, SEQ ID NO: 315, SEQ ID NO: 317, SEQ ID NO: 318, SEQ ID NO: 320, SEQ ID NO: 322, SEQ ID NO: 324, SEQ ID NO: 326 to SEQ ID NO: 331, SEQ ID NO: 334, SEQ ID NO: 336, SEQ ID NO: 339, SEQ ID NO: 341 to SEQ ID NO: 350, SEQ ID NO: 352, and SEQ ID NO: 572 to SEQ ID NO: 609, or any one of the same with at least 95%, 96%, 97%, 98% or 99% similarity or identity.

In one embodiment, the isolated antibody fragment binds human serum albumin, i.e. the antigen of interest is human serum albumin. In one embodiment, the isolated antibody fragment has the sequence SEQ ID NO: 510.

In one embodiment, there is provided a polypeptide comprising at least one isolated antibody fragment according to the invention. In one embodiment, there is provided a polypeptide comprising at least two isolated antibody fragments according to the invention, wherein the antibody fragments are linked together, optionally via a linker, for example a cleavable linker. In one embodiment, the at least two isolated antibody fragments bind to a same antigen. In another embodiment, the at least two isolated antibody fragments bind to different antigens. In one embodiment, the polypeptide comprises at least one bridging moiety between two amino acids.

In one embodiment, the isolated antibody fragment or polypeptide according to the invention, is fused to one or more effector molecules, optionally via a linker, for example a cleavable linker. In one embodiment, the effector molecule is an antibody. In one embodiment, the effector molecule is a full IgG. In another embodiment, the effector molecule is selected from the list consisting of a Fab, a VHH, a VH, a VL, a scFv, and a dsscFv. In one embodiment, the effector molecule comprises an albumin binding domain. In one embodiment, the effector molecule is albumin or a protein comprising an albumin binding domain. In one embodiment, the albumin binding domain comprises SEQ ID NO: 435 for CDR-H1, SEQ ID NO: 436 for CDR-H2, SEQ ID NO: 437 for CDR-H3, SEQ ID NO: 430 for CDR-L1, SEQ ID NO: 431 for CDR-L2 and SEQ ID NO: 432 for CDR-L3; or a heavy chain variable domain selected from SEQ ID NO: 434 and SEQ ID NO: 444 and a light chain variable domain selected from SEQ ID NO: 429 and SEQ ID NO: 443.

In another embodiment, the invention also provides pharmaceutical compositions comprising an isolated antibody fragment or a polypeptide according to the present invention, in combination with one or more of a pharmaceutically acceptable excipient.

In another embodiment, the invention also provides an isolated antibody fragment or a polypeptide according to the present invention, for use in therapy.

In another embodiment, the invention also provides a polynucleotide encoding an isolated antibody fragment or a polypeptide according to the invention. In another embodiment, the invention provides a vector comprising a polynucleotide according to the invention. In another embodiment, the invention provides a host cell comprising a polynucleotide or vector of the invention. In another embodiment, the invention provides a process for producing an isolated antibody fragment or polypeptide according to the invention, said process comprising expressing an isolated antibody fragment or a polypeptide of the invention, from a host cell of the invention.

In another aspect, the invention provides methods of producing an isolated antibody fragment or a polypeptide as defined in the present disclosure, said method comprising a step of chemical synthesis. In one embodiment, the chemical synthesis comprises a step of incorporating a coupling reagent with a radioisotope. In one embodiment, the radioisotope is an alpha emitting radioisotope, preferably Astatine 211.

The present disclosure also provides new methods of discovering therapeutic antibody fragments and polypeptides derived therefrom, comprising immunising a bovine with an antigen of interest.

Thus, in one aspect, there is provided a method of producing an isolated antibody fragment or polypeptide of the invention, said method comprising:
a) immunising a bovine with an immunogenic composition, and;
b) isolating antigen-specific memory B-cells, and;
c) sequencing the cDNA of CDR-H3 or portions thereof, and;
d) expressing or synthesising the knob domain of the ultralong CDR-H3 or portion thereof,
wherein the immunogenic composition comprises an antigen of interest or immunogenic portions thereof, or DNA encoding the same.

In one embodiment, the method further comprises a step of screening, for example for binding to said antigen of interest, wherein, optionally, the screening step is preceded by a step of reformatting the ultralong CDR-H3 or the knob domain of the ultralong CDR-H3 or portion thereof into a screening format. In one embodiment, the step of reformatting the ultralong CDR-H3 or the knob domain of the ultralong CDR-H3 or portion thereof into a screening format comprises fusing the ultralong CDR-H3 or the knob domain of the ultralong CDR-H3 or portion thereof, to a carrier, optionally via a linker, for example a cleavable linker. In one embodiment, the carrier is an Fc polypeptide. In one embodiment, the Fc polypeptide is a scFc.

In another aspect, there is provided a library comprising at least one isolated antibody fragment of the invention. In one embodiment, the library is a synthetic library. In one embodiment, the library is a phage library. In one embodiment, the phage library is a naive library. In one embodiment, the phage library is an immune library. In one embodiment, the library is prepared from cattle.

In another aspect, the invention provides a phage display library, comprising a plurality of recombinant phages; each of the plurality of recombinant phages comprising an M13-derived expression vector, wherein the M13-derived expression vector comprises a polynucleotide sequence encoding an isolated antibody fragment of the invention, optionally displayed within the full sequence of ultralong CDR-H3. In one embodiment, the isolated antibody fragment optionally displayed within the full sequence of ultralong CDR-H3, is fused to the sequence encoding the pIII coat protein of the M13 phage, directly or via a spacer.

The invention also provides a method for generating a phage display library of ultralong CDR-H3 sequences, said method comprising:
a) immunising a bovine with an immunogenic composition, and;
b) isolating total RNA from PBMC or secondary lymphoid organ, and;
c) amplifying the cDNA sequences of the ultralong CDR-H3, and;
d) fusing the sequences obtained in c) to the sequence coding for the pIII protein of a M13 phage within a phagemid vector, and;
e) transforming host bacteria with the phagemid vector obtained at step d) in combination with a helper phage co-infection, and;
f) culturing the bacteria obtained at step e), and;
g) recovering the phages from the culture medium of the bacteria, wherein the immunogenic composition comprises an antigen of interest or immunogenic portions thereof, or DNA encoding the same.

In one embodiment, step c) comprises:
a) a primary PCR with primers flanking CDR-H3, annealing to the conserved framework 3 and framework 4 of the VH, to amplify all CDR-H3 sequences, and
b) a second round of PCR using stalk primers to specifically amplify ultralong sequences from the primary PCR.

In one embodiment, the primers used at step a) comprise or consist of SEQ ID NO:446 and SEQ ID NO: 447 and/or the primers used at step b) are selected from the group consisting of SEQ ID NO:482 to SEQ ID NO:494.

The invention also provides a method for producing an isolated antibody fragment of the invention which binds to an antigen of interest, said method comprising:
a) generating a phage display library of ultralong CDR-H3 sequences, and,
b) enriching the phage display library against the antigen of interest to produce an enriched population of phage which bind the antigen of interest; and,
c) sequencing an ultralong CDR-H3 from the enriched population of phage obtained in step b); and,
d) expressing or synthesising an isolated antibody fragment derived from the ultralong CDR-H3 obtained in step c).

The invention also provides a method for producing an isolated antibody fragment of the invention which binds to an antigen of interest, said method comprising:
a) generating a phage display library of isolated antibody fragments of the invention; and,
b) enriching the phage display library against the antigen of interest to produce an enriched population of phage which bind the antigen of interest; and,
c) sequencing an isolated antibody fragment from the enriched population of phage obtained in step b); and,
d) expressing or synthesising an isolated antibody fragment obtained in step c).

It will be understood that the ability to harness the diversity of the bovine immune repertoire, to the ability to chemically synthesise the output, bridges chemistry and biology in a new way, and offers significant advantages over current procedures in immunotherapy.

### DETAILED DESCRIPTION OF THE INVENTION

### Isolated Antibody Fragments

In one aspect, the present disclosure provides an isolated antibody fragment, wherein the fragment is the knob domain of a bovine ultralong CDR-H3 or a portion thereof which binds to an antigen of interest.

An “isolated” antibody fragment is one which has been separated (e.g. by purification means) from a component of its natural environment. In the context of the present disclosure, an “isolated” antibody fragment may be obtained from bovine, and optionally engineered to produce any variant according to the invention, or may be produced recombinantly or synthetically, for example by chemical synthesis. The term “knob domain peptide” may be used to refer to an isolated antibody fragment as described in the present disclosure.

Antibody fragments for use in the context of the present disclosure encompass whole knob domains of bovine ultralong CDR-H3 and any portion thereof, notably any functionally active portion thereof (i.e., any portion of a knob domain of a bovine ultralong CDR-H3 that contains an antigen binding domain that specifically binds an antigen of interest).

Whole antibodies also known as “immunoglobulins (Ig)” generally relate to intact or full-length antibodies i.e. comprising the elements of two heavy chains and two light chains, inter-connected by disulphide bonds, which assemble to define a characteristic Y-shaped three-dimensional structure. Classical natural whole antibodies are monospecific in that they bind one antigen type, and bivalent in that they have two independent antigen binding domains. The terms “intact antibody”, “full-length antibody” and “whole antibody” are used interchangeably to refer to a monospecific bivalent antibody having a structure similar to a native antibody structure, including an Fc region as defined herein.

Each light chain is comprised of a light chain variable region (abbreviated herein as V_{L}) and a light chain constant region (C_{L}). Each heavy chain is comprised of a heavy variable region (abbreviated herein as V_{H}) and a heavy chain constant region (CH) constituted of three constant domains C_{H1}, C_{H2} and C_{H3}, or four constant domains C_{H1}, C_{H2}, C_{H3} and C_{H4}, depending on the Ig class. The “class” of an Ig or antibody refers to the type of constant region and includes IgA, IgD, IgE, IgG and IgM and several of them can be further divided into subclasses, e.g. IgG1, IgG2, IgG3, IgG4. The constant regions of the antibodies may mediate the binding of the immunoglobulin to host tissues or factors, including various cells of the immune system (e.g., effector cells) and the first component (C1q) of the classical complement system.

The term “constant domain(s)”, “constant region”, as used herein are used interchangeably to refer to the domain(s) of an antibody which is outside the variable regions. The constant domains are identical in all antibodies of the same isotype but are different from one isotype to another. Typically, the constant region of a heavy chain is formed, from N to C terminal, by CH1-hinge-CH2-CH3-optionnaly CH4, comprising three or four constant domains.

“Fc”, “Fc fragment”, “Fc region” are used interchangeably to refer to the C-terminal region of an antibody comprising the constant region of an antibody excluding the first constant region domain. Thus, Fc refers to the last two constant domains, C_{H2} and C_{H3}, of IgA, IgD, and IgG, or the last three constant domains of IgE and IgM, and the flexible hinge N-terminal to these domains.

The V_{H} and V_{L} regions of a whole antibody can be further subdivided into regions of hypervariability (or “hypervariable regions”) determining the recognition of the antigen, termed complementarity determining regions (CDR), interspersed with regions that are more structurally conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The CDRs and the FR together form a variable region. By convention, the CDRs in the heavy chain variable region of an antibody or antigen-binding fragment thereof are referred as CDR-H1, CDR-H2 and CDR-H3 and in the light chain variable region as CDR-L1, CDR-L2 and CDR-L3. They are numbered sequentially in the direction from the N-terminus to the C-terminus of each chain.

CDRs are conventionally numbered according to a system devised by Kabat et al. This system is set forth in Kabat et al., 1991, in Sequences of Proteins of Immunological Interest, US Department of Health and Human Services, NIH, USA (hereafter “Kabat et al. (supra)”). This numbering system is used in the present specification except where otherwise indicated. The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 93-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus, unless indicated otherwise ‘CDR-H1’ as employed herein is intended to refer to residues 26 to 35, as described by a combination of the Kabat numbering system and Chothia's topological loop definition. The CDRs of the light chain variable domain are located at residues 24-34 (CDR-L1), residues 50-56 (CDR-L2) and residues 89-97 (CDR-L3) according to the Kabat numbering system. Based on the alignment of sequences of different members of the immunoglobulin family, numbering schemes have been proposed and are for example described in Kabat et al., 1991, and Dondelinger et al., Frontiers in Immunology, Vol 9, article 2278 (2018).

Different species exhibit a diversity of CDR-H3 lengths. Some bovine antibodies have been characterized by unusually long CDR-H3 (so called “bovine ultralong CDR-H3”) with lengths of up to 69 residues, representing 1-15% of the bovine repertoire, whereas more conventional bovine antibodies have CDR-H3 of around 23 residues. Camelid single chain antibodies have up to 24 residues and shark IgNAR antibodies have up to 27 residues. The CDR-H3 are too long to be accommodated by any of these numbering schemes, but alternative systems have been used, as the one discussed in Stanfield et al. (supra).

“Bovine CDR-H3” as used herein encompasses all CDR-H3 found in bovines, including bovine regular CDR-H3 and bovine ultralong CDR-H3.

The term “Bovine ultralong CDR-H3” refers to the subset of CDR-H3 which has the features of characterized ultralong CDR-H3 as defined hereinafter, notably comprising a duplication of the IGHVI-7 gene segment. The ultralong CDR-H3 has been found in bovine IgG of all classes.

Bovine ultralong CDR-H3 have been characterized by a very unusual tridimensional structure comprising a “stalk domain” and a “knob domain”. The stalk domain is composed of two antiparallel β strands (each strand generally corresponding to about 12 residues). The knob domain is a disulfide rich domain which comprises a loop motif and sits atop of the stalk, which serves as a bridge to link the knob domain with the main bovine antibody scaffold.

The CDR-H3 is derived from DNA rearrangement of variable (V), diversity (D), and joining (J) gene segments. The ultralong CDR-H3 are encoded by the VH_{BUL} (Bovine Ultra Long), D_{H}2, and J_{H}1 gene segments, and their length is due to an unusually long D_{H}2 segment. Ultralong CDR-H3 have been characterized by a duplication of the IGHVI-7 gene segment.

The isolated antibody fragment of the present disclosure does not comprise the stalk domain of the bovine ultralong CDR-H3.

The “stalk domain” of bovine ultralong CDR-H3 has been characterised by its structure notably. The skilled person will appreciate that the definition of a “stalk domain” may rely on crystal structure analysis and/or sequencing information, notably as he will understand that the stalk domain position and structure may vary slightly from one ultralong CDR-H3 to another, e.g. in terms of size. The term “stalk domain” will be generally appreciated by the skilled person to correspond to the antiparallel β strands that bridge the knob domain with the main bovine antibody scaffold. The length of the stalk β strands can differ, notably from long β strands (12 or more residues) to shorter β strands.

The skilled person will appreciate that the definition of a knob domain may rely on crystal structure analysis and/or sequencing information, notably as he will understand that the knob domain position and structure may vary slightly from one ultralong CDR-H3 to another, e.g. in terms of size, cysteine content, disulphide bond content. In particular, the sequence of ultralong CDR-H3 can be determined by well-known sequencing methods, and the skilled person will be able to identify the minimal sequence which define a knob domain, based for example on a comparative analysis, with well characterised ultralong CDR-H3 as well as stalk and knob domains thereof, e.g. by alignment with well-known and/or standard nucleic and/or amino acid sequences, and/or based on crystal structure analysis.

As mentioned above, the ultralong CDR-H3 are too long to be accommodated by any of existing numbering scheme, but alternative systems have been used, as the one discussed in Stanfield et al. (supra). Structural analysis has also been provided for example by Wang et al. (Wang, F. et al. Reshaping antibody diversity. Cell 153, 1379-1393 (2013)).

The conserved Cysteine at position 92 (Kabat) and the conserved Tryptophan at position 103 (Kabat) respectively defines the start and the end of the CDR-H3, as illustrated in FIG. 14. The germline encoded VH_{BUL} D_{H}2 J_{H}1 has the following sequence:

CTTVHQ [symbol] [symbol] YVDA
WGQGLLVTVSS

(VH_{BUL}; followed by D_{H}2 gene region in bold; followed by J_{H}1 gene region underlined; The sequence coding the CDR-H3 is in italic, between positions 92 and 103 according to Kabat)

Kabat numbering system may be used for heavy-chain residues 1 to 100 and 101 to 228 but residues between 100 and 101 (corresponding to residues encoded by D_{H}2 and J_{H}1 genes) do not accommodate to the Kabat numbering system and may be numbered differently, for example sequentially with a D identifier, as described in Stanfield et al. (supra), with the conserved Cysteine residue at the start of D_{H}2 being “D2”, followed by D3, D4 etc. . . . ). For illustration purposes, FIG. 14 indicates identifiers D2, D10, D20, D30 and D40 within the D_{H}2 segment.

Following Cys H92, the common motif TTVHQ (positions 93-97 in the germline VH_{BUL}, according to Kabat) starts the ascending strand of the β-stalk region of the CDR-H3. The length between the end of the VH_{BUL} and the “CPD” conserved motif in D_{H}2 is variable due to differences in junctional diversity formed through V-D recombination. In Stanfield, those junctional residues are referred as “a,b,c” following H100 residue, depending on the length (for example, as illustrated in FIG. 14, the bovine CDR-H3 BLV1H12 comprises 3 residues following H100, referred as a, b and c).

The D_{H}2 region has been characterised to encode the knob domain and part of the descending strand of the stalk region. D_{H}2 begins with a conserved Cysteine which is part of a conserved “CPD” motif in the germline sequence, which characterises the beginning of the knob domain. The knob domain terminates at the beginning of the descending strand of the β-stalk region. The descending strand of the β-stalk region has been characterised by alternating aromatic-aliphatic residues in some ultralong CDR-H3. The descending strand of the β-stalk region ends with the residues encoded by the genetic J region, followed by residue H101, H102 according to Kabat.

In the context of the present disclosure, the minimal sequence that may define a knob domain corresponds to the portion of the ultralong CDR-H3 encapsulated by disulphide bonds, more particularly the minimal knob domain sequence starts from the first cysteine residue of an ultralong CDR-H3 and ends with the last cysteine residue of the ultralong CDR-H3. Therefore, a minimal knob domain typically comprises at least two cysteines. In one embodiment, the knob domain sequence starts from one residue preceding the first cysteine residue of an ultralong CDR-H3 and ends after the residue subsequent to the last cysteine residue of the ultralong CDR-H3. Additional amino acids may be present in the N-terminal extremity and/or in the C-terminal extremity of the knob domain sequence, preferably up to 5 additional amino acids may be present in the N-terminal and/or in the C-terminal extremity.

As an example, the sequence of the bovine ultralong CDR-H3 BLV1H12 is in italic in the following sequence comprising VH_{BUL}, D_{H2} (underlined), J_{H1} (Cys92 and Trp103 Kabat are in bold):

CTSVHQ ETKKYQ SCPDGYRERSDCSNRPACGTSDCCRVSVFGNCLTT
LPVSYSYTYNYEWHVDVWGQGLLVTVSS

The knob domain of this sequence may therefore be defined as the following sequence: SCPDGYRERSDCSNRPACGTSDCCRVSVFGNCL

(i.e. from one residue preceding the first cysteine to the residue subsequent to the last cysteine residue of the ultralong CDR-H3; cysteine residues in bold):

Another example is provided below with the K149 ultralong CDR-H3 (SEQ ID NO:1 of the present patent application):

TSVLQSTKPQKSCPDGFSYRSWDDFCCPMVGRCLAPRNTYTTEFTIEA

The knob domain that may be defined according to the present application for this sequence is in bold, starting from one residue preceding the first cysteine residue of the ultralong CDR-H3 and ending after the residue subsequent to the last cysteine residue of the ultralong CDR-H3.

In one embodiment, the isolated antibody fragment consists of the knob domain of a bovine ultralong CDR-H3, i.e. is a full-length knob domain, notably comprised between the ascending stalk and the descending stalk of the ultralong CDR-H3.

In one embodiment, the isolated antibody fragment comprises or consists of a portion of the knob domain of a bovine ultralong CDR-H3 which binds to an antigen of interest.

In one embodiment, the isolated antibody fragment comprises at least two, or at least four, or at least six, or at least eight, or at least ten cysteine residues. In one embodiment, the isolated antibody fragment comprises at least two cysteine residues. In one embodiment, the isolated antibody fragment comprises at least four cysteine residues. In one embodiment, the isolated antibody fragment comprises at least six cysteine residues. In one embodiment, the isolated antibody fragment comprises at least eight cysteine residues. In one embodiment, the isolated antibody fragment comprises at least ten cysteine residues.

In one embodiment, the isolated antibody fragment comprises two, three, four, five, six, seven, eight, nine, ten, eleven, twelve, thirteen or fourteen cysteine residues. In one embodiment, the isolated antibody fragment comprises between two cysteine residues and ten cysteine residues. In one embodiment, the isolated antibody fragment comprises between four cysteine residues and eight cysteine residues.

Two cysteine residues may bridge together to form a disulphide bond within the knob domain.

In one embodiment, the isolated antibody fragment comprises at least one, or at least two, or at least three, or at least four, or a at least five disulphide bonds. In one embodiment, the isolated antibody fragment comprises one, two, three, four, five, six, or seven disulphide bonds. In one embodiment, the isolated antibody fragment comprises between one disulphide bond and five disulphide bonds. In one embodiment, the isolated antibody fragment comprises between two disulphide bonds and four disulphide bonds.

It will be appreciated that an increased content in cysteine residues will increase the possibility to form disulphide bonds within the isolated antibody fragment. Such disulphide bonds contribute to form a loop motif within the isolated antibody fragment, which may be advantageous to increase the stability, and/or rigidity and/or binding specificity and/or binding affinity of the isolated antibody fragment.

In one embodiment, the isolated antibody fragment comprises a (Z₁) X₁ C X₂ motif at its N-terminal extremity, wherein:
a. Z₁ is present or absent, and when Z₁ is present, Z₁ represents 1 amino acid or 2, 3, 4, or 5 independently selected amino acids; and,
b. X₁ is any amino acid residue; and,
c. C is cysteine; and,
d. X₂ is an amino acid selected from the list consisting of Proline, Arginine,
Histidine, Lysine, Glycine and Serine.

Z₁ as defined in the present invention represents any amino acid or any sequence of 2, 3, 4, or independently selected amino acids that may be the same or different. In one embodiment, Z₁ is 1 amino acid. In another embodiment, Z₁ is 2 amino acids, which may be the same or different. In another embodiment, Z₁ is 3 amino acids, which may be the same or different. In another embodiment, Z₁ is 4 amino acids, which may be the same or different. In another embodiment, Z₁ is 5 amino acids, which may be the same or different.

In one embodiment, X₁ is selected from the list consisting of Serine, Threonine, Asparagine, Alanine, Glycine, Proline, Histidine, Lysine, Valine, Arginine, Isoleucine, Leucine, Phenylalanine and Aspartic acid. Thus, in one aspect, the invention provides an isolated antibody fragment, wherein the knob domain or portion thereof comprises a (Z₁) X₁ C X₂ motif at its N-terminal extremity, wherein:
a. Z₁ is present or absent, and when Z₁ is present, Z₁ represents 1 amino acid or 2, 3, 4, or 5 independently selected amino acids; and,
b. X₁ is any amino acid residue, preferably selected from the list consisting of Serine, Threonine, Asparagine, Alanine, Glycine, Proline, Histidine, Lysine, Valine, Arginine, Isoleucine, Leucine, Phenylalanine and Aspartic acid; and,
c. C is cysteine; and,
d. X₂ is an amino acid selected from the list consisting of Proline, Arginine, Histidine, Lysine, Glycine and Serine.

In one embodiment, the isolated antibody fragment comprises a (Z₁)X₁ C X₂ motif at its N-terminal extremity, wherein C is cysteine; and X₁ is selected in the list consisting of Serine (S), Threonine (T), Asparagine (N), Alanine (A), Glycine (G), Proline (P), Histidine (H), Lysine (K), Valine (V), Arginine (R), Isoleucine (I), Leucine (L), Phenylalanine (F) and Aspartic acid (D), and X₂ is selected from the list consisting of Proline (P), Arginine (R), Histidine (H), Lysine (K), Glycine (G) and Serine (S), and wherein Z₁ is present or absent, and when Z₁ is present, Z₁ represents 1 amino acid or 2, 3, 4, or 5 independently selected amino acids.

In one embodiment, the N-terminal extremity of the isolated antibody fragment comprises a motif which comprises 3 amino acid residues, corresponding to a X₁C X₂ motif, selected in the list consisting of SCP, TCP, NCP, ACP, GCP, PCR, HCP, SCR, KCP, VCP, TCH, RCP, ICP, ICR, HCR, LCR, SCK, SCG, NCP, TCS, DCP and FCR.

Preferably, the N-terminal extremity of the isolated antibody fragment is initiated by a motif selected in the list consisting of (Z₁)SCP, (Z₁)TCP, (Z₁)NCP, (Z₁)ACP, (Z₁)GCP, (Z₁)HCP, (Z₁)KCP, (Z₁)VCP, (Z₁)RCP, (Z₁)ICP, (Z₁)DCP, wherein Z₁ is present or absent, and when Z₁ is present, Z₁ represents 1 amino acid or 2, 3, 4, or 5 independently selected amino acids.

In one embodiment, the isolated antibody fragment comprises a (AB)n and/or (BA)n motif, wherein A is any amino acid residue, B is an aromatic amino acid selected from the group consisting of: tyrosine (Y), phenylalanine (F), tryptophan (W), and histidine (H), and wherein n is 1, 2, 3 or 4.

In one embodiment, A is an aliphatic amino acid residue. An aliphatic amino acid is an amino acid containing an aliphatic side chain functional group. Aliphatic amino acid residues include Alanine, isoleucine, leucine, proline, and valine.

In one embodiment, the isolated antibody fragment comprises a motif of 2-8 amino acids which is rich in aromatic and/or aliphatic amino acids. In one embodiment, the knob domain comprises a motif of 2-8 amino acids which comprises at least 2, or at least 3 or at least 4, or at least 5 amino acids selected from the group consisting of: tyrosine (Y), phenylalanine (F), tryptophan (W), and histidine (H).

In one embodiment, the isolated antibody fragment is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length or more. In one embodiment, the isolated antibody fragment is up to 50 amino acids in length or up to 55 amino acids in length. In one embodiment, the isolated antibody fragment is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length or more, and is up to 55 amino acids in length. In one embodiment, the isolated antibody fragment is a portion of a knob domain of a bovine ultralong CDR-H3 which is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids in length. In one embodiment, the isolated antibody fragment is between 5 and 55, or between 15 and 50, or between 20 and 45, or between 25 and 40 amino acids in length. In one embodiment, the isolated antibody fragment is a knob domain of a bovine ultralong CDR-H3 which is between 5 and 55, or between 15 and 50, or between 20 and 45, or between 25 and 40 amino acids in length.

In one embodiment, the knob domain of the ultralong CDR-H3, when expressed on its own, binds to an antigen of interest with a binding affinity which is at least 50%, 55%, 60%, 65%, 70%, 75%, 80%, 85%, 90% or 95% of that of the ultralong CDR-H3 which comprises said knob domain or portion thereof, e.g. when the knob domain of the ultralong CDR-H3 is expressed or synthesised as part of the entire ultralong CDR-H3.

As mentioned above, the isolated antibody fragment may be produced synthetically, for example by chemical synthesis.

In one aspect, the invention provides a peptide which binds an antigen of interest comprising or consisting of the sequence of formula (I):

(Z₁)(X₁)C X₂(Y)ₙ₁(C)ₙ₂(Y)ₙ₃(C)ₙ₄(Y)ₙ₅(C)ₙ₆(Y)ₙ₇(C)ₙ₈(Y)ₙ₉(C)ₙ₁₀(Y)ₙ₁₁(C)ₙ₁₂(Y)ₙ₁₃(C)ₙ₁₄(Y)ₙ₁₅(C)ₙ₁₆(Y)ₙ₁₇C(X₃)(Z₂) (I)

wherein:
C represents one cysteine residue; and,
Z₁ is present or absent, and when Z₁ is present, Z₁ represents 1 amino acid or 2, 3, 4, or 5 independently selected amino acids; and,
X₁ is present or absent, and when X₁ is present, X₁ is any amino acid residue, preferably selected from the list consisting of Serine, Threonine, Asparagine, Alanine, Glycine, Proline, Histidine, Lysine, Valine, Arginine, Isoleucine, Leucine, Phenylalanine and Aspartic acid; and,
X₂ is selected from the list consisting of Proline, Arginine, Histidine, Lysine, Glycine and Serine; and,
Z₂ is present or absent, and when Z₂ is present, Z₂ represents 1 amino acid or 2, 3, 4, or 5 independently selected amino acids; and,
n2, n4, n6, n8, n10, n12, n14 and n16 are independently 0 or 1; and,
Y represents any amino acid or any sequence of amino acids that may be the same or different; and,
n1, n3, n5, n7, n9, n11, n13, n15 and n17 represent the number of amino acids in Y, and are independently selected from 0 to 22, preferably from 1 to 15; and,
at least one of n1, n3, n5, n7, n9, n11, n13, n15 and n17 is not equal to 0; and,
X₃ is present or absent, and when X₃ is present, X₃ represents any amino acid, preferably selected from the list consisting of Leucine, Serine, Glycine, Threonine, Tryptophan, Asparagine, Tyrosine, Arginine, Isoleucine, aspartic acid, Histidine, Glutamic acid, Valine, Lysine, Proline; and,
wherein the peptide is up to 55 amino acids in length.

Z₁ represents any amino acid or any sequence of 2, 3, 4, or 5 independently selected amino acids that may be the same or different. In one embodiment, Z₁ is 1 amino acid. In another embodiment, Z₁ is 2 amino acids, which may be the same or different. In another embodiment, Z₁ is 3 amino acids, which may be the same or different. In another embodiment, Z₁ is 4 amino acids, which may be the same or different. In another embodiment, Z₁ is 5 amino acids, which may be the same or different.

Z₂ represents any amino acid or any sequence of 2, 3, 4, or 5 independently selected amino acids that may be the same or different. In one embodiment, Z₂ is 1 amino acid. In another embodiment, Z₂ is 2 amino acids, which may be the same or different. In another embodiment, Z₂ is 3 amino acids, which may be the same or different. In another embodiment, Z₂ is 4 amino acids, which may be the same or different. In another embodiment, Z₂ is 5 amino acids, which may be the same or different.

Z₁ and Z₂ may comprise any amino acid as long as the properties of the peptide otherwise defined is retained, e.g. binding capability to an antigen of interest.

In one embodiment, the peptide which binds an antigen of interest comprising or consisting of the sequence of formula (I) is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length. In one embodiment, the peptide which binds an antigen of interest comprising or consisting of the sequence of formula (I) is between 5 and 55, or between 15 and 50, or between 20 and 45, or between 25 and 40 amino acids in length.

Brackets are generally used for optional residues or sequences. For example, (C) generally indicates an optional Cysteine residue, in the context of the present disclosure.

In one embodiment, the peptide comprises 2 Cysteine residues. Therefore, in one particular aspect, the invention provides a peptide which binds an antigen of interest comprising or consisting of the sequence of formula (II):

(Z₁)(X₁)C X₂(Y)ₙ₁C(X₃)(Z₂) (II)

wherein Z₁, X₁, C, X₂, Y, ₙ₁, X₃, and Z₂ are defined as above, and wherein the peptide is up to amino acids in length.

In such embodiment, n₁ may be comprised between 1 and 20 amino acids. In one embodiment, n1 is 1, 2, 3, 4, 5, 6, 7, 8, 9, 10, 11, 12, 13, 14, 15, 16, 17, 18, 19, or 20.

In one embodiment, the peptide which binds an antigen of interest comprising or consisting of the sequence of formula (II) is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length. In one embodiment, the peptide which binds an antigen of interest comprising or consisting of the sequence of formula (II) is between 5 and or between 15 and 50, or between 20 and 45, or between 25 and 40 amino acids in length.

In another embodiment, the peptide comprises 4 Cysteine residues. Therefore, in one particular aspect, the invention provides a peptide which binds an antigen of interest comprising or consisting of the sequence of formula (III):

(Z₁)(X₁)C X₂(Y)ₙ₁C(Y)ₙ₃C(Y)ₙ₅C(X₃)(Z₂) (III)

wherein Z₁, X₁, C, X₂, Y, n1, n3, n5, X₃, and Z₂ are defined as above, and wherein the peptide is up to 55 amino acids in length.

In one embodiment, n1 is comprised between 3 and 15 and/or n3 is comprised between 4 and 12 and/or n5 is comprised between 1 and 14. In one embodiment, n1 is 3, 5, 7, 8, 10, 11, 14, or 15. In one embodiment, n3 is 4, 5, 6, 8, 10, 11 or 12. In one embodiment, n5 is 3, 4, 5, 6, 7, 9, 10, 11, or 14.

In another embodiment, n1 and/or n3 and/or n5 is equal to 0 and two or three Cysteine residues are contiguous.

In one embodiment, the peptide has the sequence of formula (IIIa):

(Z₁)(X₁)C X₂C C(Y)ₙ₅C(X₃)(Z₂) (IIIa)

wherein Z₁, X₁, C, X₂, Y, ₙ₅, X₃, and Z₂ are defined as above and wherein the peptide is up to amino acids in length.

In one embodiment, the peptide has the sequence of formula (IIIb):

(Z₁)(X₁)C X₂(Y)ₙ₁C C(Y)ₙ₅C(X₃)(Z₂) (IIIb)

wherein Z₁, X₁, C, X₂, Y, ₙ₁, ₙ₅, X₃, and Z₂ are defined as above and wherein the peptide is up to 55 amino acids in length.

In one embodiment, the peptide has the sequence of formula (IIIc):

(Z₁)(X₁)C X₂(Y)ₙ₁C(Y)ₙ₃C C(X₃)(Z₂) (IIIc)

wherein Z₁, X₁, C, X₂, Y, ₙ₁, ₙ₃, X₃, and Z₂ are defined as above and wherein the peptide is up to 55 amino acids in length.

In one embodiment, the peptide which binds an antigen of interest comprising or consisting of the sequence of formula (III), (IIIa), (Mb), or (IIIc) is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length. In one embodiment, the peptide which binds an antigen of interest comprising or consisting of the sequence of formula (III), (IIIa), (IIIb), or (IIIc) is between 5 and 55, or between 15 and 50, or between and 45, or between 25 and 40 amino acids in length.

In another embodiment, the peptide comprises 6 Cysteine residues. Therefore, in one particular aspect, the invention provides a peptide which binds an antigen of interest comprising or consisting of the sequence of formula (IV):

(Z₁)(X₁)C X₂(Y)ₙ₁C(Y)ₙ₃C(Y)ₙ₅C(Y)ₙ₇C C(X₃)(Z₂) (IV)

wherein Z₁, X₁, C, X₂, Y, n₁, n₃, n₅, n₇, n₉, X₃, and Z₂ are defined as above, and wherein the peptide is up to 55 amino acids in length.

In one embodiment, n₁=2 to 9 and/or n₃=1 to 10 and/or n₅=2 to 9 and/or n₇=1 to 15 and/or n₉=1 to 14. In one embodiment, n₁=2, 3, 4, 5, 6, 7, 8 or 9. In one embodiment, n₃=1, 2, 3, 4, 6, 7, 8, 9 or 10. In one embodiment, n₅=2, 3, 4, 5, 6, 7, 8, or 9. In one embodiment, n₇=1, 2, 3, 4, 5, 6, 7, 8, 9, 10, 11, 12, 13, 14, or 15. In one embodiment, n₉=1, 2, 3, 4, 5, 6, 7, 8, 9, 10, 11, 12, 13, or 14.

In one embodiment, n₁=0 and/or n₃=0 and/or n₅=0 and/or n₇=0 and/or n₉=0 and two or three Cysteine residues are contiguous. In one embodiment, the peptide has the sequence of formula (IVa):

(Z₁)(X₁)C X₂C C(Y)ₙ₅C(Y)ₙ₇C C(X₃)(Z₂) (IVa)

wherein Z₁, X₁, C, X₂, Y, n₅, n₇, n₉, X₃, and Z₂ are defined as above, and wherein the peptide is up to 55 amino acids in length.

In one embodiment, the peptide has the sequence of formula (IVb):

(Z₁)(X₁)C X₂(Y)ₙ₁C C(Y)ₙ₅C(Y)ₙ₇C(Y)ₙ₉C(X₃)(Z₂) (IVb)

wherein Z₁, X₁, C, X₂, Y, n₁, n₅, n₇, n₉, X₃, and Z₂ are defined as above, and wherein the peptide is up to 55 amino acids in length.

In one embodiment, the peptide has the sequence of formula (IVc):

(Z₁)(X₁)C X₂(Y)ₙ₁C(Y)ₙ₃C C(Y)ₙ₇C C(X₃)(Z₂) (IVc)

wherein Z₁, X₁, C, X₂, Y, n₁, n₃, n₇, n₉, X₃, and Z₂ are defined as above, and wherein the peptide is up to 55 amino acids in length.

In one embodiment, the peptide has the sequence of formula (IVd):

(Z₁)(X₁)C X₂(Y)ₙ₁C(Y)ₙ₃C(Y)ₙ₅C C(Y)ₙ₉C(X₃)(Z₂) (IVd)

wherein Z₁, X₁, C, X₂, Y, n₁, n₃, n₅, n₉, X₃, and Z₂ are defined as above, and wherein the peptide is up to 55 amino acids in length.

In one embodiment, the peptide has the sequence of formula (IVe):

(Z₁)(X₁)C X₂(Y)ₙ₁C(Y)ₙ₃C(Y)ₙ₅C(Y)ₙ₇C C(X₃)(Z₂) (IVe)

wherein Z₁, X₁, C, X₂, Y, n₁, n₃, n₅, n₇, X₃, and Z₂ are defined as above, and wherein the peptide is up to 55 amino acids in length.

In one embodiment, the peptide has the sequence of formula (IVf):

(Z₁)(X₁)C X₂(Y)ₙ₁C C C(Y)ₙ₇C C(X₃)(Z₂) (IVf)

wherein Z₁, X₁, C, X₂, Y, n₁, n₇, n₉, X₃, and Z₂ are defined as above, and wherein the peptide is up to 55 amino acids in length.

In one embodiment, the peptide has the sequence of formula (IVg):

(Z₁)(X₁)C X₂(Y)ₙ₁C(Y)ₙ₃C C C C(Y)ₙ₉C(X₃)(Z₂) (IVg)

wherein Z₁, X₁, C, X₂, Y, n₁, n₃, n₉, X₃, and Z₂ are defined as above, and wherein the peptide is up to 55 amino acids in length.

In one embodiment, the peptide has the sequence of formula (IVh):

(Z₁)(X₁)C X₂(Y)ₙ₁C(Y)ₙ₃C(Y)ₙ₅C C C(X₃)(Z₂) (IVh)

wherein Z₁, X₁, C, X₂, Y, n₁, n₃, n₅, X₃, and Z₂ are defined as above, and wherein the peptide is up to 55 amino acids in length.

In one embodiment, the peptide which binds an antigen of interest comprising or consisting of the sequence of formula (IV), (IVa), (IVb), (IVc), (IVd), (IVe), (IVf), (IVg), or (IVh), is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length. In one embodiment, the peptide which binds an antigen of interest comprising or consisting of the sequence of formula (IV), (IVa), (IVb), (IVc), (IVd), (IVe), (IVf), (IVg), or (IVh), is between 5 and 55, or between 15 and 50, or between 20 and 45, or between 25 and 40 amino acids in length.

In another embodiment, the peptide comprises 8 Cysteine residues. Therefore, in one particular aspect, the invention provides a peptide which binds an antigen of interest comprising or consisting of the sequence of formula (V):

(Z₁)(X₁)C X₂(Y)ₙ₁C(Y)ₙ₃C(Y)ₙ₅C(Y)ₙ₇C(Y)ₙ₉C(Y)ₙ₁₁C(Y)ₙ₁₃C(X₃)(Z₂) (V)

wherein Z₁, X₁, C, X₂, Y, n₁, n₃, n₅, n₇, n₉, n₁₁, n₁₃, X₃, and Z₂ are defined as above, and wherein the peptide is up to 55 amino acids in length.

In one embodiment, n₁=0 and/or n₃=0 and/or n₅=0 and/or n₇=0 and/or n₉=0 and/or n₁₁=0, and/or n₁₃=0 and two or three Cysteine residues are contiguous.

In one embodiment, the peptide which binds an antigen of interest comprising or consisting of the sequence of formula (V) is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length. In one embodiment, the peptide which binds an antigen of interest comprising or consisting of the sequence of formula (V) is between 5 and or between 15 and 50, or between 20 and 45, or between 25 and 40 amino acids in length.

In another embodiment, the peptide comprises 10 Cysteine residues. Therefore, in one particular aspect, the invention provides a peptide which binds an antigen of interest comprising or consisting of the sequence of formula (VI):

(Z₁)(X₁)C X₂(Y)ₙ₁C(Y)ₙ₃C(Y)ₙ₅C(Y)ₙ₇C(Y)ₙ₉C(Y)ₙ₁₁C(Y)ₙ₁₃C(Y)ₙ₁₅C(Y)ₙ₁₇C(X₃)(Z₂) (VI)

wherein Z₁, X₁, C, X₂, Y, n₁, n₃, n₅, n₇, n₉, n₁₁, n₁₃, n₁₅, n₁₇, X₃, and Z₂ are defined as above, and wherein the peptide is up to 55 amino acids in length.

In one embodiment, n₁=0 and/or n₃=0 and/or n₅=0 and/or n₇=0 and/or n₉=0 and/or n₁₁=0, and/or n₁₃=0 and/or n₁₅=0 and/or n₁₇=0 and two or three Cysteine residues are contiguous.

In one embodiment, the peptide which binds an antigen of interest comprising or consisting of the sequence of formula (VI) is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length. In one embodiment, the peptide which binds an antigen of interest comprising or consisting of the sequence of formula (VI) is between 5 and 55, or between 15 and 50, or between 20 and 45, or between 25 and 40 amino acids in length.

Preferably, the isolated antibody fragment of the present invention specifically binds to an antigen of interest, i.e. comprises a specific binding domain to an antigen of interest. “Specifically,” as employed herein is intended to refer to a binding domain that only recognises the antigen to which it is specific or a binding domain that has significantly higher binding affinity to the antigen to which is specific compared to affinity to antigens to which it is non-specific, for example 5, 6, 7, 8, 9, 10 times higher binding affinity.

Preferably, the isolated antibody fragment of the present invention has a specific binding affinity (as measured by its dissociation constant K_{D}) for its cognate antigen of 10⁻⁵ M or less, M or less, 10⁻⁶ M or less, 10⁻⁸ M or less, 10⁻⁹ M or less, 10⁻¹⁰ M or less, or 10-11 M or less. In one embodiment, the isolated antibody fragment of the present invention has a specific binding affinity (as measured by its dissociation constant K_{D}) for its cognate antigen between 1. 10⁻⁷ M and 1. 10⁻⁸ M, or between 1. 10⁻⁸ M and 1. 10⁻⁹ M, or between 1. 10⁻⁹ M and 1. 10⁻¹⁰ M.

Affinity can be measured by known techniques such as surface plasmon resonance techniques including Biacore™. Affinity may be measured at room temperature, 25° C. or 37° C. Affinity may be measured at physiological pH, i.e. at about pH 7.4. In one embodiment, the affinity values as described above are measured using Biacore, notably Biacore 8K, at pH 7.4.

It will be appreciated that the affinity of antibodies fragments provided by the present invention may be altered using any suitable method known in the art.

Isolated Antibody Fragment Variants

In some aspects, the isolated antibody fragment comprises a sequence which is a variant of a naturally occurring sequence of a knob domain of a bovine ultralong CDR-H3.

In other words, the present disclosure provides variants of isolated antibody fragments as described above, which comprise non-naturally occurring sequences, i.e. which have been further engineered, for example to improve at least one pharmacokinetic and/or biological function. In such aspects, the isolated antibody fragment comprising a naturally occurring sequence may be referred as “parent”.

The present invention also includes antibody fragments, i.e. knob domains of bovine ultralong CDR-H3 or portions thereof, which comprise sequences which are at least 90%, 91%, 92%, 93%, 94%, 95%, 96%, 97%, 98% or 99% similar or identical to a sequence given herein. “Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated by methods well known, for example the BLAST™ software available from NCBI.

In one embodiment, antibody fragments of the present disclosure are processed to provide improved affinity for a target antigen or antigens. Such variants can be obtained by a number of affinity maturation protocols including mutating the CDRs, chain shuffling, use of mutator strains of E. coli, DNA shuffling, phage display and sexual PCR. Vaughan et al (Nature Biotechnology, 16, 535-539, 1998) discusses these methods of affinity maturation. Another method useful in the context of the present disclosure to improve binding of the isolated antibody fragment at a binding site on the antigen of interest is a method as described in WO2014/198951. Improved affinity as employed herein in this context refers to an improvement over the starting isolated antibody fragment. Affinity can be measured as described above.

In one embodiment, the isolated antibody fragment is a variant of a parent bovine antibody fragment which has an affinity which is at least 50%, 60%, 65%, 70%, 75%, 80%, 85%, 90%, or 95% higher than the affinity of the parent bovine antibody fragment, as measured for example by Biacore.

“Truncated variants” when referring to antibody fragments are those with one or more amino acids in the native or starting amino acid sequence removed from either terminus of the polypeptide.

In some embodiments, the isolated antibody fragment is a variant which has been engineered to comprise a disulfide bond which is in a non-naturally occurring position. This may be engineered into the molecule by introducing cysteine(s) into the amino acid chain at the position or positions required. This non-natural disulfide bond is in addition to or as an alternative to the natural disulfide bond(s) which may be present in the parent isolated antibody fragment. The cysteine(s) in natural positions can be replaced by an amino acid such as serine which is incapable on forming a disulfide bridge. Introduction of engineered cysteines can be performed using any method known in the art. These methods include, but are not limited to, PCR extension overlap mutagenesis, site-directed mutagenesis or cassette mutagenesis (see, generally, Sambrook et al., Molecular Cloning, A Laboratory Manual, Cold Spring Harbour Laboratory Press, Cold Spring Harbour, N Y, 1989; Ausubel et al., Current Protocols in Molecular Biology, Greene Publishing & Wiley-Interscience, N Y, 1993). Site-directed mutagenesis kits are commercially available, e.g. QuikChange® Site-Directed Mutagenesis kit (e.g. Stratagene, La Jolla, CA). Cassette mutagenesis can be performed based on Wells et al., 1985, Gene, 34:315-323. Alternatively, mutants can be made by total gene synthesis by annealing, ligation and PCR amplification and cloning of overlapping oligonucleotides.

In one aspect, it may be useful to decrease or remove the cysteine residues and/or disulfide bonds in an isolated antibody fragment of the disclosure, e.g. to lower the risk of immunogenicity, i.e. of side reactions occurring during or after the administration to a patient. In such aspect, one or all of the cysteine(s) in natural positions can be replaced by an amino acid such as serine which is incapable on forming a disulfide bridge. It will be appreciated that alternative bridging moieties may be used to stabilise and/or form a cyclised isolated antibody fragment in the absence of cysteine residues. In one embodiment, the isolated antibody fragment is a variant which has been engineered to remove the cysteine residues and which comprises at least one bridging moiety as defined in the present disclosure. In one embodiment, the isolated antibody fragment is a variant which has been engineered to contain only one, or only two, or only three, or only four, cysteine residues, and/or to contain only one or only two disulphide bonds and which optionally further comprises at least one bridging moiety as defined in the present disclosure.

Additional modifications include hydroxylation of proline and lysine, phosphorylation of hydroxyl groups of tyrosinyl, seryl or threonyl residues, methylation of the alpha-amino groups of lysine, arginine, and histidine side chains (Creighton, T. E., Proteins: Structure and Molecular Properties, W.H. Freeman and Co., San Francisco, 1983, pp. 79-86).

The isolated antibody fragment of the invention may be cyclised. Cyclisation may be advantageous to confer more resistance to proteolysis, resulting notably in an improved stability.

Therefore, in one embodiment, the isolated antibody fragment of the present disclosure further comprises a bridging moiety between two amino acids.

Cyclised antibody fragments include any antibody fragments that have as part of their structure one or more cyclic features such as a loop, bridging moiety, and/or an internal linkage. As used herein, the term “bridging moiety” refers to one or more components of a bridge formed between two adjacent or non-adjacent amino acids, unnatural amino acids or non-amino acids in an isolated antibody fragment. Bridging moieties may be of any size or composition.

In one embodiment, a bridging moiety may be between the amino acid residue in N-terminal position and the amino acid residue in C-terminal position such as to create a head-to-tail cyclisation. In one embodiment, a bridging moiety may be between amino acids which are not in terminal position.

In one embodiment, the isolated antibody fragment comprises only one bridging moiety between two amino acids. In another embodiment, the isolated antibody fragment comprises more than one bridging moiety between two amino acids, e.g. two, or three, or five bridging moieties, each one being between two amino acids.

In one embodiment, the bridging moiety comprises a feature selected from the group consisting of a disulphide bond, an amide bond (lactam), a thioether bond, an aromatic ring, an unsaturated aliphatic hydrocarbon chain, a saturated aliphatic hydrocarbon chain and a triazole ring.

In one embodiment, the disulphide bond is formed between two naturally occurring cysteine residues. In another embodiment, the disulphide bond is formed between cysteine residues, with at least one cysteine residue being engineered, as described above.

In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof. Bridging moieties may comprise one or more features including, but not limited to an amide bond (lactam), disulfide bond, thioether bond, aromatic ring, triazole ring, and hydrocarbon chain. In some embodiments, bridging moieties comprise an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue. In some cases, bridging moieties may comprise bonds formed between residues that may include, but are not limited to (S)-2-amino-5-azidopentanoic acid, (S)-2-aminohept-6-enoic acid, (S)-2-aminopent-4-ynoic acid and (S)-2-aminopent-4-enoic acid. Bridging moieties may be formed through cyclisation reactions using olefin metathesis. In some cases, such bridging moieties may be formed between (S)-2-aminopent-4-enoic acid and (S)-2-aminohept-6-enoic acid residues. In some embodiments, the bridging moiety comprises a disulfide bond formed between two thiol containing residues. In some embodiments, the bridging moiety comprises one or more thioether bonds. Such thioether bonds, may include those found in cyclo-thioalkyl compounds. These bonds are formed during a chemical cyclization reaction between chloro acetic acid, N-terminal modified groups and cysteine residues. In some cases, bridging moieties comprise one or more triazole ring. Such triazole rings may include, but are not limited to those formed by cyclization reaction between (S)-2-amino-5-azidopentanoic acid and (S)-2-aminopent-4-ynoic acid. In some embodiments, bridging moieties comprise non-protein or non-polypeptide based moieties, including, but not limited to cyclic rings (including, but not limited to aromatic ring structures (e.g. xylyls)). Such bridging moieties may be introduced by reaction with reagents containing multiple reactive halides, including, but not limited to poly(bromomethyl)benzenes, poly(bromomethyl)pyridines, poly(bromomethyl)alkylbenzenes and/or (E)-1,4-dibromobut-2-ene.

In one embodiment, the antibody fragment of the invention is fully bovine. In such embodiment, each and every residue is derived from a bovine germline sequence. In some embodiments, each and every residue is derived from a bovine germline sequence which can have undergone affinity maturation for an antigen.

In one embodiment, the isolated antibody fragment of the invention is chimeric.

The term “chimeric” refers to an antibody fragment comprising at least two portions, one being derived from a particular source or species, such as bovine, while the other portion is derived from a different source or species, such as human. In one embodiment, the antibody fragment is human/bovine chimeric. In one embodiment, the antibody fragment comprises at least one residue derived from a human sequence.

In one embodiment, the isolated antibody fragment of the invention is synthetic. The term “synthetic” refers to an isolated antibody fragment that has been produced de novo by synthesis, notably by chemical synthesis as described in the present disclosure.

Antigens of Interest

Antigens of interest may be any medically relevant protein such as those proteins upregulated during disease or infection, for example receptors and/or their corresponding ligands. Particular examples of antigens include cell surface receptors such as T cell or B cell signalling receptors, co-stimulatory molecules, checkpoint inhibitors, natural killer cell receptors, Immunoglobulin receptors, TNFR family receptors, B7 family receptors, adhesion molecules, integrins, cytokine/chemokine receptors, GPCRs, growth factor receptors, kinase receptors, tissue-specific antigens, cancer antigens, pathogen recognition receptors, complement receptors, hormone receptors or soluble molecules such as cytokines, chemokines, leukotrienes, growth factors, hormones or enzymes or ion channels, epitopes, fragments and post translationally modified forms thereof.

In one embodiment, an antigen of interest bound by the isolated antibody fragment provides the ability to recruit effector functions, such as complement pathway activation and/or effector cell recruitment.

The recruitment of effector function may be direct in that effector function is associated with a cell, said cell bearing a recruitment molecule on its surface. Indirect recruitment may occur when binding of an antigen to an antigen binding domain in the isolated antibody fragment according to the present disclosure to a recruitment polypeptide causes release of, for example, a factor which in turn may directly or indirectly recruit effector function, or may be via activation of a signalling pathway. Examples include IL2, IL6, IL8, IFNγ, histamine, C1q, opsonin and other members of the classical and alternative complement activation cascades, such as C2, C4, C3-convertase, and C5 to C9.

As used herein, “a recruitment polypeptide” includes a FcγR such as FcγRI, FcγRII and FcγRIII, a complement pathway protein such as, but without limitation, C1q and C3, a CD marker protein (Cluster of Differentiation marker) or a fragment thereof which retains the ability to recruit cell-mediated effector function either directly or indirectly. A recruitment polypeptide also includes immunoglobulin molecules such as IgG1, IgG2, IgG3, IgG4, IgE and IgA which possess effector function.

In one embodiment an antigen binding domain in the isolated antibody fragment according to the present disclosure has specificity for a complement pathway protein, with C5 being particularly preferred.

Further, isolated antibody fragments of the present disclosure may be used to chelate radionuclides by virtue of a single domain antibody which binds to a nuclide chelator protein. Such fusion proteins are of use in imaging or radionuclide targeting approaches to therapy. In one embodiment, an antigen binding domain within an isolated antibody fragment according to the disclosure has specificity for a serum carrier protein, a circulating immunoglobulin molecule, or CD35/CR1, for example for providing an extended half-life to the isolated antibody fragment with specificity for said antigen of interest by binding to said serum carrier protein, circulating immunoglobulin molecule or CD35/CR1.

As used herein, “serum carrier proteins” include thyroxine-binding protein, transthyretin, α1-acid glycoprotein, transferrin, fibrinogen and albumin, or a fragment of any thereof.

As used herein, a “circulating immunoglobulin molecule” includes IgG1, IgG2, IgG3, IgG4, sIgA, IgM and IgD, or a fragment of any thereof.

CD35/CR1 is a protein present on red blood cells which have a half-life of 36 days (normal range of 28 to 47 days; Lanaro et al., 1971, Cancer, 28(3):658-661).

In one embodiment, the antigen of interest for which the isolated antibody fragment has specificity is a serum carrier protein, such as a human serum carrier, such as human serum albumin.

Isolated Antibody Fragments which Bind to C5

In one aspect, the present invention provides an isolated antibody fragment of the invention which binds to the component C5 of the Complement.

In one embodiment, the isolated antibody fragment of the invention specifically binds to the component C5 of the Complement.

The complement system is a component of the innate immune response and includes about 20 circulating complement component proteins, including C5. Activation occurs by way of a pathway of proteolytic cleavage initiated by pathogen recognition and leading to pathogen destruction. Three such pathways are known in the complement system and are referred to as the classical pathway, the lectin pathway, and the alternative pathway. Complement component C5 is cleaved by either C5-convertase complex into C5a and C5b. C5a, much like C3a, diffuses into the circulation and promotes inflammation, acting as a chemoattractant for inflammatory cells. C5b remains attached to the cell surface where it triggers the formation of the MAC through interactions with C6, C7, C8 and C9. The MAC is a hydrophilic pore that spans the membrane and promotes the free flow of fluid into and out of the cell, thereby destroying it.

In one embodiment, the isolated antibody fragment is the knob domain of a bovine ultralong CDR-H3, wherein the bovine ultralong CDR-H3 has a sequence selected from the list consisting of SEQ ID NO: 1 to SEQ ID NO: 154. In one embodiment, the isolated antibody fragment is the knob domain of a bovine ultralong CDR-H3, wherein the bovine ultralong CDR-H3 has a sequence which is a variant of any one of SEQ ID NO: 1 to SEQ ID NO: 154 with at least 95, 96, 97, 98 or 99% similarity or identity.

In one embodiment, the isolated antibody fragment comprises or consists of a truncated variant of any one of the sequences SEQ ID NO:1 to SEQ ID NO: 154.

In one embodiment, the isolated antibody fragment corresponds to the knob domain of a bovine ultralong CDR-H3 or portion thereof which binds to C5 and has a sequence selected from the list consisting of SEQ ID NO: 157 to SEQ ID NO: 310, SEQ ID NO: 313. SEQ ID NO: 315, SEQ ID NO: 317, SEQ ID NO: 318, SEQ ID NO: 320, SEQ ID NO: 322, SEQ ID NO: 324, SEQ ID NO: 326 to SEQ ID NO: 331, SEQ ID NO: 334, SEQ ID NO: 336, SEQ ID NO: 339, SEQ ID NO: 341 to SEQ ID NO: 350, and SEQ ID NO: 352. In one embodiment, the isolated antibody fragment has the sequence SEQ ID NO: 450. In one embodiment, the isolated antibody fragment corresponds to the knob domain of a bovine ultralong CDR-H3 or portion thereof which binds to C5 and comprises a sequence of any one of SEQ ID NO: 157 to SEQ ID NO: 310, SEQ ID NO: 313. SEQ ID NO: 315, SEQ ID NO: 317, SEQ ID NO: 318, SEQ ID NO: 320, SEQ ID NO: 322, SEQ ID NO: 324, SEQ ID NO: 326 to SEQ ID NO: 331, SEQ ID NO: 334, SEQ ID NO: 336, SEQ ID NO: 339, SEQ ID NO: 341 to SEQ ID NO: 350, SEQ ID NO: 352, and SEQ ID NO: 450 or any one of the same with at least 95%, 96%, 97%, 98% or 99% similarity or identity.

In one embodiment, the isolated antibody fragment is the knob domain of a bovine ultralong CDR-H3, wherein the bovine ultralong CDR-H3 has a sequence of any one of SEQ ID NO: 521 to SEQ ID NO: 571. In one embodiment, the isolated antibody fragment is the knob domain of a bovine ultralong CDR-H3, wherein the bovine ultralong CDR-H3 has a sequence which is a variant of any one of SEQ ID NO: 521 to SEQ ID NO: 571 with at least 95, 96, 97, 98 or 99% similarity or identity.

In one embodiment, the isolated antibody fragment comprises or consists of a truncated variant of any one of the sequences SEQ ID NO:521 to SEQ ID NO: 571.

In one embodiment, the isolated antibody fragment corresponds to the knob domain of a bovine ultralong CDR-H3 or portion thereof which binds to C5, in particular to human C5, and comprises a sequence of any one of SEQ ID NO: 572 to SEQ ID NO: 609, or any one of the same with at least 95%, 96%, 97%, 98% or 99% similarity or identity. In one embodiment, the isolated antibody fragment comprises or consists of a truncated variant of any one of the sequences SEQ ID NO: 572 to SEQ ID NO: 609 which binds to C5.

In one embodiment, the isolated antibody fragment corresponds to the knob domain of a bovine ultralong CDR-H3 or portion thereof which binds to human C5 and rat C5, and comprises a sequence of any one of SEQ ID NO: 572 to SEQ ID NO: 578, or a sequence of any one of SEQ ID NO: 594 to SEQ ID NO: 599, or any one of the same with at least 95%, 96%, 97%, 98% or 99% similarity or identity.

In one embodiment, the isolated antibody fragment of the present invention is species cross-reactive. This represents a technical advantage in the context of the development of a therapeutic as it enables testing in in vivo models that generate reliable and reproducible results, predictive of the situation in humans for the same molecule. In one embodiment, the antibody fragment of the present invention binds human C5, and at least one of rabbit C5, murine C5, rat C5 or cynomolgus C5. In one embodiment, the antibody fragment of the present invention binds human C5, rabbit C5 and murine C5, and optionally rat C5 and/or cynomolgus C5. In one embodiment, the antibody fragment of the present invention binds human C5, rabbit C5, murine C5, rat C5 and cynomolgus C5.

Isolated Antibody Fragments which Bind to Albumin

In one embodiment, the present disclosure provides an isolated antibody fragment of the invention which binds to human serum albumin, i.e. the antibody fragment as defined herein comprises an albumin binding domain. In one embodiment, the isolated antibody fragment specifically binds to human serum albumin. In such embodiments, the isolated antibody fragment may have an extended serum half-life. In addition, where the isolated antibody fragment of the invention is fused to an effector molecule, it may be useful to extend the serum half-life of said effector molecule.

In one embodiment, the isolated antibody fragment of the present invention binds cynomolgus serum albumin, murine serum albumin and/or rat serum albumin.

In one embodiment, the isolated antibody fragment is the knob domain of a bovine ultralong CDR-H3, wherein the bovine ultralong CDR-H3 has a sequence of any one of SEQ ID NO: 497 to SEQ ID NO: 508. In one embodiment, the isolated antibody fragment is the knob domain of a bovine ultralong CDR-H3, wherein the bovine ultralong CDR-H3 has a sequence which is a variant of any one of SEQ ID NO: 497 to SEQ ID NO: 508 with at least 95, 96, 97, 98 or 99% similarity or identity.

In one embodiment, the isolated antibody fragment comprises or consists of a truncated variant of any one of the sequences SEQ ID NO:497 to SEQ ID NO: 508.

In one embodiment, the isolated bovine antibody fragment of the present invention has a sequence of any one of SEQ ID NO: 509 to SEQ ID NO: 520 or any one of the same with at least 95%, 96%, 97%, 98% or 99% similarity or identity. In one embodiment, the isolated antibody fragment comprises or consists of a truncated variant of any one of the sequences SEQ ID NO: 509 to SEQ ID NO: 520 which binds to serum albumin. In one embodiment, the isolated bovine antibody fragment of the present invention specifically binds murine serum albumin and comprises or has the sequence SEQ ID NO: 509. In one embodiment, the isolated bovine antibody fragment of the present invention specifically binds human serum albumin and comprises or has the sequence SEQ ID NO: 510. In one embodiment, the isolated bovine antibody fragment of the present invention binds to murine and rat albumin. In one embodiment, the isolated bovine antibody fragment of the present invention binds to murine and rat albumin and comprises or has the sequence of any one of SEQ ID NO: 511 to SEQ ID NO: 520.

Isolated Antibody Fragment Fusion Proteins

In some embodiments, the isolated antibody fragment of the invention is fused to one or more effector molecules, optionally via a linker, for example a cleavable linker.

In the context of the present disclosure, the terms “Fused to”, “inserted into”, and “conjugated to” may be used interchangeably. Thus, antibody fragment fusion proteins encompass molecules comprising an isolated antibody fragment of the invention inserted into an exogenous protein, e.g. a second antibody.

Antibody fragment fusion proteins also encompass isolated antibody fragments conjugated to an effector molecule, e.g. by chemical conjugation.

In one embodiment, the isolated antibody fragment of the invention is genetically fused to one or more effector molecules, optionally via a linker. In one embodiment, the isolated antibody fragment of the invention is genetically fused to one or more effector molecules directly, i.e. without a linker. In another embodiment, the isolated antibody fragment of the invention is genetically fused to one or more effector molecules via a linker. In one embodiment, the isolated antibody fragment of the invention is genetically fused to one or more effector molecules directly, and additionally genetically fused to one or more effector molecules via a linker.

In one embodiment, the linker is a peptide linker. The term “peptide linker” as used herein refers to a peptide comprised of amino acids. A range of suitable peptide linkers will be known to the person of skill in the art. In one embodiment, the linker is a flexible linker. In one embodiment, the linker is selected from a sequence comprised in the list consisting of SEQ ID NO: 361 to SEQ ID NO: 427.

TABLE 1
Flexible linker sequences

| SEQ ID NO: | SEQUENCE |
|---|---|
| 361 | (S)GGGGTGGGGS |
| 362 | SGGGGSGGGGTGGGGS |
| 363 | SGGGGSE |
| 364 | DKTHTS |
| 365 | (S)GGGGS |
| 366 | (S)GGGGSGGGGS |
| 367 | (S)GGGGSGGGGSGGGGS |
| 368 | (S)GGGGSGGGGSGGGGSGGGGS |
| 369 | (S)GGGGSGGGGSGGGGSGGGGSGGGGS |
| 370 | AAAGSG-GASAS |
| 371 | AAAGSG-XGGGS-GASAS |
| 372 | AAAGSG-XGGGSXGGGS-GASAS |
| 373 | AAAGSG-XGGGSXGGGSXGGGS-GASAS |
| 374 | AAAGSG-XGGGSXGGGSXGGGSXGGGS-GASAS |
| 375 | AAAGSG-XS-GASAS |
| 376 | PGGNRGTTTTRRPATTTGSSPGPTQSHY |
| 377 | ATTTGSSPGPT |
| 378 | ATTTGS |
| — | GS |
| 379 | EPSGPISTINSPPSKESHKSP |
| 380 | GTVAAPSVFIFPPSD |
| 381 | GGGGIAPSMVGGGGS |
| 382 | GGGGKVEGAGGGGGS |
| 383 | GGGGSMKSHDGGGGS |
| 384 | GGGGNLITIVGGGGS |
| 385 | GGGGVVPSLPGGGGS |
| 386 | GGEKSIPGGGGS |
| 387 | RPLSYRPPFPFGFPSVRP |
| 388 | YPRSIYIRRRHPSPSLTT |
| 389 | TPSHLSHILPSFGLPTFN |
| 390 | RPVSPFTFPRLSNSWLPA |
| 391 | SPAAHFPRSIPRPGPIRT |
| 392 | APGPSAPSHRSLPSRAFG |
| 393 | PRNSIHFLHPLLVAPLGA |
| 394 | MPSLSGVLQVRYLSPPDL |
| 395 | SPQYPSPLTLTLPPHPSL |
| 396 | NPSLNPPSYLHRAPSRIS |
| 397 | LPWRTSLLPSLPLRRRP |
| 398 | PPLFAKGPVGLLSRSFPP |
| 399 | VPPAPVVSLRSAHARPPY |
| 400 | LRPTPPRVRSYTCCPTP- |
| 401 | PNVAHVLPLLTVPWDNLR |
| 402 | CNPLLPLCARSPAVRTFP |

(S) is optional in sequences 361 and 365 to 369.

TABLE 2
Hinge linker sequences

| SEQ ID NO: | SEQUENCE |
|---|---|
| 403 | DKTHTCAA |
| 404 | DKTHTCPPCPA |
| 405 | DKTHTCPPCPATCPPCPA |
| 406 | DKTHTCPPCPATCPPCPATCPPCPA |
| 407 | DKTHTCPPCPAGKPTLYNSLVMSDTAGTCY |
| 408 | DKTHTCPPCPAGKPTHVNVSVVMAEVDGTCY |
| 409 | DKTHTCCVECPPCPA |
| 410 | DKTHTCPRCPEPKSCDTPPPCPRCPA |
| 411 | DKTHTCPSCPA |

Examples of rigid linkers include the peptide sequences GAPAPAAPAPA (SEQ ID NO:412), PPPP (SEQ ID NO: 413) and PPP.

In one embodiment the peptide linker is an albumin binding peptide.

Examples of albumin binding peptides are provided in WO2007/106120 and include:

TABLE 3
Albumin binding peptides

| SEQ ID NO: | SEQUENCE |
|---|---|
| 414 | DLCLRDWGCLW |
| 415 | DICLPRWGCLW |
| 416 | MEDICLPRWGCLWGD |
| 417 | QRLMEDICLPRWGCLWEDDE |
| 418 | QGLIGDICLPRWGCLWGRSV |
| 419 | QGLIGDICLPRWGCLWGRSVK |
| 420 | EDICLPRWGCLWEDD |
| 421 | RLMEDICLPRWGCLWEDD |
| 422 | MEDICLPRWGCLWEDD |
| 423 | MEDICLPRWGCLWED |
| 424 | RLMEDICLARWGCLWEDD |
| 425 | EVRSFCTRWPAEKSCKPLRG |
| 426 | RAPESFVCYWETICFERSEQ |
| 427 | EMCYFPGICWM |

Effector Molecules

The term “effector molecule” as used herein includes, for example, biologically active proteins, for example enzymes, polypeptides, peptides, other antibody or antibody fragments, synthetic or naturally occurring polymers, nucleic acids and fragments thereof e.g. DNA, RNA and fragments thereof, radionuclides, particularly radioiodides, radioisotopes, chelated metals, nanoparticles and reporter groups such as fluorescent compounds or compounds which may be detected by NMR or ESR spectroscopy.

Particular radioisotopes of interest are alpha emitting radioisotopes, in particular short-lived alpha-emitting isotopes such as Astatine isotopes. In one embodiment, the effector molecule is Astatine 211. Astatine 211 may be advantageously used for targeted alpha-particle therapy (TAT) in particular in cancer treatment, with a potential to deliver radiation in a highly localised and toxic manner, while having advantageously having a low half-life of 7.2 hours. Thus, in one aspect, the present disclosure provides an isolated antibody fragment conjugated to Astatine 211. Radiochemical methodologies using coupling agents have been described.

In one embodiment, the isolated antibody fragment comprises a chemical cage for halogen capture. The ability to chemically synthesise the isolated antibody fragment of the invention enables the incorporation of coupling reagents into the synthesis itself, eliminating the need for conjugation of drugs or radioisotopes onto biologically produced antibodies, where controlling the substitution ratio can be difficult, and can easily result in high values, risking solubility and activity of the antibody, and low values, risking inefficient product.

An example would be the incorporation of a boron cage, such as decaborate, directly into the isolated antibody fragment synthesis, so that the product could be readily labelled with astatine-211 in the clinic, immediately prior to administration. This would simplify current labelling conditions, which involve two steps, the first of which is the coupling a bifunctional linker to a biologically produced antibody, usually employing succinimide chemistry to target amines or maleimide groups to target sulphydryl groups on the antibody, followed by labelling with the radioisotope. Astatine-211 emits alpha particles, and is being trialed in immunotherapy, where the high energy and short path length are attractive in targeted cell killing. Its half-life is only 7.2 hours, so simplification and shortening of the labelling procedure would be beneficial to enable the optimum dose to be administered to patients.

Thus, in one embodiment, the isolated antibody fragment or polypeptide as defined in the present disclosure is produced by chemical synthesis which comprises a step of incorporating a coupling reagent with a radioisotope. In one embodiment, the radioisotope is an alpha emitting radioisotope. In one embodiment, the radioisotope is Astatine 211.

Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions.

In another embodiment the effector molecule may increase the half-life of the isolated antibody fragment in vivo, and/or reduce immunogenicity of the isolated antibody fragment and/or enhance the delivery of an isolated antibody fragment across an epithelial barrier to the immune system. Examples of suitable effector molecules of this type include Fc fragments, polymers, albumin, albumin binding proteins or albumin binding compounds such as those described in WO05/117984. In one embodiment, the effector molecule is palmitic acid. Palmitic acid has the advantageous property to bind albumin and improve interaction with cells. In one embodiment, the effector molecule is an activated form of palmitic acid such as palmitoyl.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, for example from 5000 to 40000 Da such as from 20000 to 40000 Da. Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da. In one embodiment antibodies for use in the present disclosure are attached to poly(ethyleneglycol) (PEG) moieties. In one particular example, the PEG molecules may be attached through any available amino acid side-chain or terminal amino acid functional group located in the isolated antibody fragment, for example any free amino, imino, thiol, hydroxyl or carboxyl group. Such amino acids may occur naturally in the isolated antibody fragment or may be engineered into the fragment using recombinant DNA methods. Suitably PEG molecules are covalently linked through a thiol group of at least one cysteine residue located in the isolated antibody fragment.

According to the present disclosure, the isolated antibody fragment may be modified by the addition of one or more conjugate groups.

As used herein, a “conjugate” refers to any molecule or moiety appended to another molecule. In the present invention, conjugates may be polypeptide (amino acid) based or not. Conjugates may comprise lipids, small molecules, RNA, DNA, polypeptides, polymers, or combinations thereof. Functionally, conjugates may serve as targeting molecules or may serve as payload to be delivered to a cell, organ or tissue. Conjugates are typically covalent modifications introduced by reacting targeted amino acid residues or the termini of the polypeptide with an organic derivatizing agent that is capable of reacting with selected side-chains or terminal residues. Such modifications are within the ordinary skill in the art and are performed without undue experimentation.

The conjugation process may involve PEGylation, lipidation, albumination, biotinylation, desthiobiotinylation, the addition of other polypeptide tails, or grafting onto antibody Fc domains, CDR regions of intact antibodies, or antibody domains produced by any number of means. The conjugate may include anchors including cholesterol oleate moiety, cholesteryl laurate moiety, an a-tocopherol moiety, a phytol moiety, an oleate moiety, or an unsaturated cholesterol-ester moiety or a lipophilic compound selected from acetanilides, anilides, aminoquinolines, benzhydryl compounds, benzodiazepines, benzofurans, cannabinoids, cyclic polypeptides, dibenzazepines, digitalis glycosides, ergot alkaloids, flavonoids, imidazoles, quinolines, macrolides, naphthalenes, opiates (such as, but not limited to, morphinans or other psychoactive drugs), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids. In one embodiment, the conjugation process involves palmitoylation. Palmitoylation can be employed to improve the pharmacokinetics of an isolated antibody fragment or polypeptide as described in the present disclosure.

In one embodiment, the effector molecule is albumin. In one embodiment, the effector molecule is human serum albumin. In one embodiment, the effector molecule is rat serum albumin. In one embodiment, the isolated antibody fragment is fused to the N- and/or C-terminal extremity of albumin. In one embodiment, the isolated antibody fragment is inserted into albumin. In such embodiment, the isolated antibody fragment is preferably inserted at a position distal to the albumin interaction site with FcRn. In one embodiment, the isolated antibody fragment is inserted into human serum albumin. Residues on albumin, distal to the interaction with FcRn, may be selected as sites for inserting the isolated antibody fragment of the invention, for example Alanine 59, Alanine 171, Alanine 364, Aspartic acid 562 on human serum albumin. In one embodiment, the isolated antibody fragment is inserted into albumin, optionally via one or more, for example two, linker(s). For example, the isolated antibody fragment may be inserted into albumin via two linkers, one linker at the N-terminal extremity of the isolated antibody fragment and the other linker at the C-terminal extremity of the isolated antibody fragment. A suitable linker may be a flexible linker as described herein. In one embodiment, the linker or at least one of the linkers is SGGGS.

In one embodiment, the invention provides a human serum albumin-knob domain fusion protein (i.e. a fusion protein comprising an isolated antibody fragment of the invention and human serum albumin) which comprises or has a sequence selected in the list consisting of SEQ ID NO: 452, SEQ ID NO: 454, SEQ ID NO: 456, SEQ ID NO: 458, SEQ ID NO: 460, SEQ ID NO: 462, SEQ ID NO: 464, and SEQ ID NO: 466.

In one embodiment, the effector molecule is a Fc fragment or any derivative thereof which may increase the half-life of the isolated antibody fragment in vivo. Examples of derivatives of Fc fragments include Fc variants, multimers of Fc fragments, Fc polypeptide such as scFc.

In one embodiment, the effector molecule is an Fc fragment. In one embodiment, the effector molecule is a Fc fragment of a human IgG1. The human IgG1 heavy chain Fc region is defined herein to comprise residues C226 to its carboxyl-terminus, wherein the numbering is according to the EU index as in Kabat. In the context of human IgG1, the lower hinge refers to positions 226-236, the CH2 domain refers to positions 237-340 and the CH3 domain refers to positions 341-447 according to the EU index as in Kabat. The corresponding Fc region of other immunoglobulins can be identified by sequence alignments.

In one embodiment, the isolated antibody fragment according to the invention is fused to an Fc fragment. In one embodiment, the isolated antibody fragment is fused to the N- and/or C-terminal extremity of an Fc fragment. In one embodiment, the isolated antibody fragment according to the invention is inserted into an Fc fragment. In such embodiment, the isolated antibody fragment is preferably inserted at a position distal to the Fc interaction site with FcRn. In one embodiment, the isolated antibody fragment according to the invention is inserted into an Fc fragment of a human IgG1. Residues on the Fc, distal to the interaction with FcRn, may be selected as sites for inserting the isolated antibody fragment of the invention, for example Alanine 327, Glycine 341, Asparagine 384, Glycine 402 on the IgG1 Fc fragment. In one embodiment, the isolated antibody fragment is inserted into an Fc fragment of a human IgG1, optionally via one or more, for example two, linker(s). For example, the isolated antibody fragment may be inserted into the IgG1 Fc fragment via two linkers, one linker at the N-terminal extremity of the isolated antibody fragment and the other linker at the C-terminal extremity of the isolated antibody fragment. A suitable linker may be a flexible linker as described herein. In one embodiment, the linker or at least one of the linkers has the sequence SEQ ID NO: 365.

In one embodiment, the invention provides a human IgG1 Fc-knob domain fusion protein (i.e. a fusion protein comprising an isolated antibody fragment of the invention and human IgG1 Fc fragment) which comprises or has a sequence selected in the list consisting of SEQ ID NO: 471 to SEQ ID NO: 474.

In one embodiment, the effector molecule is an antibody.

The antibody for use as an effector molecule in the context of the present disclosure includes whole antibodies as defined above and functionally active fragments thereof (i.e., molecules that contain an antigen binding domain that specifically binds an antigen, also termed antigen-binding fragments). The antibody may be (or derived from) monoclonal, multi-valent, multi-specific, bispecific, fully human, humanised, bovine or chimeric.

The constant region domains of the antibody, if present, may be selected having regard to the proposed function of the antibody, and in particular the effector functions which may be required. For example, the constant region domains may be human IgG1, IgG2 or IgG4 domains. In particular, human IgG constant region domains may be used, especially of the IgG1 isotype when the antibody molecule is intended for therapeutic uses and antibody effector functions are required. Alternatively, IgG2 and IgG4 isotypes may be used when the antibody is intended for therapeutic purposes and antibody effector functions are not required. It will be appreciated that sequence variants of these constant region domains may also be used. For example, IgG4 molecules in which the serine at position 241 (numbered according to the Kabat numbering system) has been changed to proline as described in Angal et al. (Angal et al., 1993. A single amino acid substitution abolishes the heterogeneity of chimeric mouse/human (IgG4) antibody as observed during SDS-PAGE analysis Mol Immunol 30, 105-108) and termed IgG4P herein, may be used.

The human IgG1 heavy chain Fc region is defined herein to comprise residues C226 to its carboxyl-terminus, wherein the numbering is according to the EU index as in Kabat. In the context of human IgG1, the lower hinge refers to positions 226-236, the CH2 domain refers to positions 237-340 and the CH3 domain refers to positions 341-447 according to the EU index as in Kabat. The corresponding Fc region of other immunoglobulins can be identified by sequence alignments.

In one embodiment, the effector molecule is a full IgG. In one embodiment, the effector molecule is a full IgG1. In one embodiment, the effector molecule is a full IgG4.

In another embodiment, the effector molecule is an antigen-binding fragment of an antibody.

Antigen-binding fragments of antibodies generally comprise at least one variable light (VL) or variable heavy (VH) domain and include: single chain antibodies (e.g. a full length heavy chain or light chain), Fab, modified Fab, Fab′, modified Fab′, F(ab′)2, Fv, Fab-Fv, Fab-dsFv, single domain antibodies (sdAb, e.g. VH or VL or VHH), scFv, dsscFv, Bis-scFv, diabodies, tribodies, triabodies, tetrabodies and epitope-binding fragments of any of the above (see for example Holliger and Hudson, 2005, Nature Biotech. 23(9):1126-1136; Adair and Lawson, 2005, Drug Design Reviews-Online 2(3), 209-217). The methods for creating and manufacturing these antibody binding fragments are well known in the art (see for example Verma et al., 1998, Journal of Immunological Methods, 216, 165-181). For example, antibody binding fragments may be obtained from any whole antibody, especially a whole monoclonal antibody, using any suitable enzymatic cleavage and/or digestion techniques, e.g. treatment with pepsin. Alternatively, the antibody starting material may be prepared by the use of recombinant DNA techniques involving the manipulation and re-expression of DNA encoding antibody variable and/or constant regions. Standard molecular biology techniques may be used to modify, add or delete amino acids or domains as desired. Any alterations to the variable or constant regions are still encompassed by the terms ‘variable’ and ‘constant’ regions as used herein. The antibody fragment starting material may be obtained from any species including, for example, mouse, rat, rabbit, hamster, camel, llama, goat or human. Parts of the antibody fragment may be obtained from more than one species; for example, the antibody fragments may be chimeric. In one example, the constant regions are from one species and the variable regions from another. The antibody fragment starting material may also be modified. In another example, the variable region of the antibody fragment has been created using recombinant DNA engineering techniques. Such engineered versions include those created for example from natural antibody variable regions by insertions, deletions or changes in or to the amino acid sequences of the natural antibodies. Particular examples of this type include those engineered variable region domains containing at least one CDR and, optionally, one or more framework amino acids from one antibody and the remainder of the variable region domain from a second antibody.

Antigen-binding fragments of antibodies include single chain antibodies (e.g. scFv and dsscfv), Fab, Fab′, F(ab′)2, Fv, single domain antibodies or nanobodies (e.g. V_{H} or V_{L}, or V_{HH} or V_{NAR}). Other antibody fragments for use in the present invention include the Fab and Fab′ fragments described in International patent applications WO2011/117648, WO2005/003169, WO2005/003170 and WO2005/003171. The methods for creating and manufacturing these antibody fragments are well known in the art (see for example Verma et al., 1998, Journal of Immunological Methods, 216, 165-181).

The term “Fab fragment” as used herein refers to an antibody fragment comprising a light chain fragment comprising a VL (variable light) domain and a constant domain of a light chain (CL), and a VH (variable heavy) domain and a first constant domain (CH1) of a heavy chain.

A typical “Fab′ fragment” comprises a heavy and a light chain pair in which the heavy chain comprises a variable region VH, a constant domain CH1 and a natural or modified hinge region and the light chain comprises a variable region VL and a constant domain CL. Dimers of a Fab′ according to the present disclosure create a F(ab′)2 where, for example, dimerization may be through the hinge.

The term “single domain antibody” as used herein refers to an antibody fragment consisting of a single monomeric variable antibody domain. Examples of single domain antibodies include V_{H} or V_{L} or V_{H}H or V-NAR.

The “Fv” refers to two variable domains, for example co-operative variable domains, such as a cognate pair or affinity matured variable domains, i.e. a VH and VL pair.

“Single chain variable fragment” or “scFv” as employed herein refers to a single chain variable fragment comprising or consisting of a heavy chain variable domain (V_{H}) and a light chain variable domain (V_{L}) which is stabilised by a peptide linker between the V_{H} and V_{L} variable domains. The V_{H} and V_{L} variable domains may be in any suitable orientation, for example the C-terminus of V_{H} may be linked to the N-terminus of V_{L} or the C-terminus of V_{L} may be linked to the N-terminus of V_{H}.

“Disulphide-stabilised single chain variable fragment” or “dsscFv” as employed herein refers to a single chain variable fragment which is stabilised by a peptide linker between the V_{H} and V_{L} variable domain and also includes an inter-domain disulphide bond between V_{H} and V_{L}.

In one embodiment, the effector molecule is a multispecific antibody. Multispecific antibody as employed herein refers to an antibody which has at least two binding domains, i-e two or more binding domains, for example two or three binding domains, wherein the at least two binding domains independently bind two different antigens or two different epitopes on the same antigen. Multispecific antibodies encompass monovalent and multivalent, e.g. bivalent, trivalent, tetravalent multi-specific antibodies. In one embodiment, the effector molecule is a bispecific antibody. Bispecific antibody as employed herein refers to an antibody with two antigen specificities. In one embodiment, the effector molecule is a trispecific antibody. Trispecific antibody as employed herein refers to an antibody with three antigen specificities.

A variety of multispecific antibody formats have been generated and may be used in the present invention, for example bispecific IgG, appended IgG, multispecific (e.g. bispecific) antibody fragments, multispecific (e.g. bispecific) fusion proteins, and multispecific (e.g. bispecific) antibody conjugates, as described for example in Spiess et al. (Spiess et al., Alternative molecular formats and therapeutic applications for bispecific antibodies. Mol Immunol. 67(2015):95-106.)

Preferred multispecific antibodies for use as an effector molecule in the present invention include appended IgG and appended Fab, wherein a whole IgG or a Fab fragment, respectively, is engineered by appending at least one additional antigen-binding domain (e.g. two, three or four additional antigen-binding domains), for example a single domain antibody (such as VH or VL, or VHH), a scFv, a dsscFv, a dsFv to the N- and/or C-terminus of the heavy and/or light chain of said IgG or Fab, for example as described in WO2009/040562, WO2010035012, WO2011/030107, WO2011/061492, WO2011/061246 and WO2011/086091 all incorporated herein by reference. In particular, the Fab-Fv format was first disclosed in WO2009/040562 and the disulphide stabilized version thereof, the Fab-dsFv, first disclosed in WO2010/035012. A single linker Fab-dsFv, wherein the dsFv is connected to the Fab via a single linker between either the VL or VH domain of the Fv, and the C terminal of the LC or HC of the Fab, was first disclosed in WO2014/096390, incorporated herein by reference. An appended IgG comprising a full-length IgG1 engineered by appending a dsFv to the C-terminus of the heavy or light chain of the IgG, was first disclosed in WO2015/197789, incorporated herein by reference.

Another preferred antibody for use as an effector molecule in the present invention comprises a Fab linked to two scFvs or dsscFvs, each scFv or dsscFv binding the same or a different target (e.g., one scFv or dsscFv binding a therapeutic target and one scFv or dsscFv that increases half-life by binding, for instance, albumin). Such antibody fragments are described in International Patent Application Publication No WO2015/197772, which is hereby incorporated by reference in its entirety. Another preferred antibody for use as an effector molecule in the present invention fragment comprises a Fab linked to only one scFv or dsscFv, as described for example in WO2013/068571 incorporated herein by reference, and Dave et al., Mabs, 8(7) 1319-1335 (2016).

In one embodiment, the effector molecule is selected from the list consisting of a Fab, a single domain antibody (a VHH, or a VH, or a VL), a scFv, and a dsscFv.

In one embodiment, the effector molecule is a VHH, i.e. the invention provides a fusion protein between a VHH and an isolated antibody fragment of the invention. An isolated antibody fragment of the invention may be inserted into the framework turns of a VHH antibody, at the opposing end to the CDRs, to make a single chain bi-specific antibody. In one embodiment, the VHH is the hC3nb1 VHH, which binds C3 and C3b. In one embodiment, the VHH comprises or has the sequence SEQ ID NO: 351. In one embodiment, a hC3nb1 VHH-knob fusion protein comprises or has a sequence selected from the list consisting of SEQ ID NO: 353 to SEQ ID NO: 357. In another embodiment, the invention provides a hC3nb1 VHH-ultralong CDR-H3 fusion protein which comprises or has a sequence SEQ ID NO: 359 or SEQ ID NO: 360.

In another embodiment, the effector molecule is a Fab, i.e. the invention provides a fusion protein between a Fab and an isolated antibody fragment of the invention. In one embodiment, the isolated antibody fragment is inserted into the CDR-H3 of a Fab. In one embodiment, the Fab comprises a heavy chain having the sequence SEQ ID NO: 311, which is paired with the light chain of SEQ ID NO: 325. In one embodiment, the Fab-knob domain fusion protein has a sequence selected from the list consisting of SEQ ID NO: 312, SEQ ID NO: 314, SEQ ID NO: 316, SEQ ID NO: 319, SEQ ID NO: 321 and SEQ ID NO: 323.

In one embodiment, the antibody, i.e. the effector molecule comprises an albumin binding domain.

In one embodiment, the effector molecule is albumin or a protein comprising an albumin binding domain.

“Albumin binding domain” as employed herein refers to a portion of a protein that interacts specifically with serum albumin. In particular in the context of the antibody used as effector molecule, it refers to a portion of the antibody, which comprises a part or the whole of one or more variable domains, for example a pair of variable domains VH and VL, that interact specifically with albumin. An albumin binding domain may comprise a single domain antibody. As such, an albumin binding domain according to the present disclosure may refer to the VH, VL, or pair of VH/VL which binds to albumin.

In one embodiment, the antibody, comprising an albumin binding domain, comprises a light and/or heavy chain sequence; and/or a light and/or heavy chain variable domain sequence; and/or at least one of CDR-L1, CDR-L2, and CDR-L3 sequence; and/or at least one of CDR-H1, CDR-H2, and CDR-H3 selected from below (CDRs in bold):

CA645 Fab Light Chain (gL5):
(SEQ ID NO: 428)
DIQMTQSPSSVSASVGDRVTITC [symbol] WYQQKPGKAPKLLIY [symbol] G
VPSRFSGSGSGTDFTLTISSLQPEDFATYYC [symbol] FGGGTKVEIKRTVAAPS
VFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
VL domain (gL5):
(SEQ ID NO: 429)
DIQMTQSPSSVSASVGDRVTITCQSSPSVWSNFLSWYQQKPGKAPKLLIYEASKLTS
GVPSRFSGSGSGTDFTLTISSLQPEDFATYYCGGGYSSISDTTFGGGTKVEIK
CDR-L1:
(SEQ ID NO: 430)
QSSPSVWSNFLS
CDR-L2:
(SEQ ID NO: 431)
EASKLTS
CDR-L3:
(SEQ ID NO: 432)
GGGYSSISDTT
CA645 Fab Heavy Chain (gH5):
(SEQ ID NO: 433)
EVQLLESGGGLVQPGGSLRLSCAVS [symbol] WVRQAPGKGLEWIG [symbol] [symbol]
RFTISRDNSKNTVYLQMNSLRAEDTAVYYCAR [symbol] WGQ
GTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSC
VH domain (gH5):
(SEQ ID NO: 434)
EVQLLESGGGLVQPGGSLRLSCAVSGIDLSNYAINWVRQAPGKGLEWIGIIWASGT
TFYATWAKGRFTISRDNSKNTVYLQMNSLRAEDTAVYYCARTVPGYSTAPYFDL
WGQGTLVTVSS
CDR-H1:
(SEQ ID NO: 435)
GIDLSNYAIN
CDR-H2:
(SEQ ID NO: 436)
IIWASGTTFYATWAKG
CDR-H3:
(SEQ ID NO: 437)
TVPGYSTAPYFDL

Additional VH and VL sequences useful in the context of the present disclosure are listed below:

CA645 VH domain (gH1):
(SEQ ID NO: 438)
EVQLLESGGGLVQPGGSLRLSCAVS [symbol] WVRQAPGKGLEWIG [symbol] [symbol]
RFTISRDSTTVYLQMNSLRAEDTAVYYCARTVPGYSTAPYFDLWGQGT
LVTVSS
CA645 VH domain (gH37):
(SEQ ID NO: 439)
EVQLLESGGGLVQPGGSLRLSCAVS [symbol] WVRQAPGKGLEWIG [symbol] [symbol]
FTISRDNSKNTVYLQMNSLRAEDTAVYYCARTVPGYSTAPYFDLWGQ
GTLVTVSS
CA645 VH domain (gH47):
(SEQ ID NO: 440)
EVQLLESGGGLVQPGGSLRLSCAVS [symbol] WVRQAPGKGLEWIG [symbol] [symbol]
RFTISRDNSKNTVYLQMNSLRAEDAVYYCARTVPGYSAAPYFDLWGQ
GTLVTVSS
CA645 VL domain (gL1):
(SEQ ID NO: 441)
DIVMTQSPSSVSASVGDRVTITC [symbol] WYQQKPGKAPKLLIY [symbol]
GVPSRFKGSGSGTDFTLTISSLQPEDFATYYC [symbol] FGGGTKVEIK
CA645 VL domain (gL4):
(SEQ ID NO: 442)
DIQMTQSPSSVSASVGDRVTITC [symbol] WYQQKPGKAPKLLIY [symbol]
GVPSRFSGSGSGTDFTLTISSLQPEDFATYYC [symbol] FGGGTKVEIK

In some embodiments, the albumin binding domain comprises variants of the VL and VH domains which bind human serum albumin as described above (SEQ ID NO: 429, SEQ ID NO: 434 and SEQ ID NO: 442 respectively) that comprise an additional cysteine residue such that a disulphide bond may be formed between the VL and VH domains. The additional cysteine-containing variants may have the following sequences (wherein the additional cysteine residues are underlined):

CA645-Cys VL domain (gL5):
(SEQ ID NO: 443)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGCGTKVEIK
CA645-Cys VH domain (gH5):
(SEQ ID NO: 444)
EVQLLESGGGLVQPGGSLRLSCAVSGIDLSNYAINWVRQAPGKCLEWIG
IIWASGTTFYATWAKGRFTISRDNSKNTVYLQMNSLRAEDTAVYYCART
VPGYSTAPYFDLWGQGTLVTVSS
CA645-Cys VL (gL4):
(SEQ ID NO: 445)
DIQMTQSPSSVSASVGDRVTITCQSSPSVWSNFLSWYQQKPGKAPKLLI
YEASKLTSGVPSRFSGSGSGTDFTLTISSLQPEDFATYYCGGGYSSISD
TTFGCGTKVEIKRT

In some embodiments, the VH framework of the albumin binding domain is human (for example VH3, such as VH3 1-3 3-23), and comprises for example 1, 2, 3, 4, 5 or 6 amino acid substitutions, such as amino acids which are donor residues. In such embodiments, the VH may have a sequence shown in SEQ ID NO: 434, SEQ ID NO: 438, SEQ ID NO: 439, SEQ ID NO: 440, SEQ ID NO: 444 or a variant of any one of the same with at least 95, 96, 97, 98 or 99% similarity of identity.

In some embodiments, the VL framework of the albumin binding domain is human (for example Vκ1, such as 2-1-(1) L5), and comprises for example 1, 2, 3, 4, 5 or 6 amino acid substitutions, such as amino acids which are donor residues. In such embodiments, the VL may have a sequence shown in SEQ ID NO: 429, SEQ ID NO: 441, SEQ ID NO: 442, SEQ ID NO: 443, SEQ ID NO: 445, or a variant of any one of the same with at least 95, 96, 97, 98 or 99% similarity of identity.

In some embodiments, the albumin binding domain comprises VH and VL sequences selected from the combinations SEQ ID NO: 434 and SEQ ID NO: 429, or SEQ ID NO: 444 and SEQ ID NO: 443 or a variant or variants of any of the same with at least 95, 96, 97, 98 or 99% similarity or identity.

In some embodiments, the VH and VL sequences of the albumin binding domain are SEQ ID NO: 434 and SEQ ID NO: 429, respectively. In some embodiments, the VH and VL sequences of the albumin binding domain are SEQ ID NO: 444 and SEQ ID NO: 443, respectively.

In one embodiment, the albumin binding domain comprises SEQ ID NO: 435 for CDR-H1, SEQ ID NO: 436 for CDR-H2, SEQ ID NO: 437 for CDR-H3, SEQ ID NO: 430 for CDR-L1, SEQ ID NO: 431 for CDR-L2 and SEQ ID NO: 432 for CDR-L3; or a heavy chain variable domain selected from SEQ ID NO: 434 and SEQ ID NO: 444 and a light chain variable domain selected from SEQ ID NO: 429 and SEQ ID NO: 443.

In one embodiment, the effector molecule is a Fab which binds human serum albumin, i.e. the Fab comprises an albumin binding domain. Thus, in one aspect, the invention provides a Fab which binds serum albumin, wherein an isolated antibody fragment according to the invention is inserted into its framework, e.g. the framework 3 region (FW3) of the V domain, notably the VH domain as described in WO2020/011868 (published on Jan. 16, 2020). As explained, in addition to three CDR loops, antibody light and heavy chains, both conventional and single-chain camelid VHH, have a fourth loop which is formed by framework 3. The Kabat numbering system defines framework 3 as positions 66-94 in a heavy chain and positions 57-88 in a light chain.

Thus, in one aspect, the invention also provides a bispecific antibody format, in particular stable and capable of simultaneously binding two antigens. Advantageously, a CA645 Fab-isolated antibody fragment fusion protein (which may be also termed CA645 Fab-knob fusion protein) as described herein may simultaneously bind C5 and albumin, which may confer an increased serum half-life to the isolated antibody fragment.

In one embodiment, the invention provides an isolated antibody fragment of the invention, inserted into the FW3 of the VH of a 645 Fab. In one embodiment, the 645Fab comprises a heavy chain having the sequence SEQ ID NO: 334, which is paired with a light chain of SEQ ID NO:329. In one embodiment, the invention provides a CA645 Fab-knob fusion protein comprising a sequence selected in the list consisting of SEQ ID NO: 332, SEQ ID NO: 333, SEQ ID NO: 335, SEQ ID NO: 337, SEQ ID NO: 338 and SEQ ID NO: 340.

Polypeptides Comprising Isolated Antibody Fragments

In one aspect, the present disclosure provides a polypeptide comprising at least one isolated antibody fragment according to the invention.

In one aspect, the present disclosure provides a polypeptide comprising at least two isolated antibody fragments according to the invention, wherein the isolated antibody fragments are linked together, optionally via a linker, for example a cleavable linker.

In one embodiment, the at least two isolated antibody fragments bind to a same antigen including binding to the same epitope on the antigen or binding to different epitopes on the antigen.

In another embodiment, the at least two isolated antibody fragments bind to different antigens.

The polypeptide may be monospecific, multi-specific, multi-valent, bispecific.

“Monospecific polypeptide” as employed herein refers to a polypeptide comprising at least two isolated antibody fragments of the present disclosure, wherein the polypeptide binds to only one antigen of interest.

“Multi-specific polypeptide” as employed herein refers to a polypeptide comprising at least two isolated antibody fragments of the present disclosure, wherein the polypeptide comprises at least two antigen binding domains, i-e two or more antigen binding domains, for example two or three antigen binding domains, wherein the at least two antigen binding domains independently bind two different antigens or two different epitopes on the same antigen. Multi-specific polypeptides may be monovalent for each specificity (antigen). Multi-specific polypeptides described herein encompass monovalent and multivalent, e.g. bivalent, trivalent, tetravalent multi-specific polypeptides, as well as multi-specific polypeptides having different valences for different epitopes (e.g, a multi-specific polypeptide which is monovalent for a first antigen specificity and bivalent for a second antigen specificity which is different from the first one).

In one embodiment, the polypeptide is monospecific and bivalent. In another embodiment, the polypeptide is bispecific.

“Bispecific polypeptide” as employed herein refers to a polypeptide with two antigen specificities. In one embodiment, the polypeptide comprises two antigen binding domains wherein one binding domain binds ANTIGEN 1 and the other binding domain binds ANTIGEN 2, i-e each binding domain is monovalent for each antigen. In one embodiment, the antibody is a tetravalent bispecific polypeptide, i-e the polypeptide comprises four antigen binding domains, wherein for example two binding domains bind ANTIGEN 1 and the other two binding domains bind ANTIGEN 2. In one embodiment, the polypeptide is a trivalent bispecific polypeptide.

It will be appreciated that a polypeptide of the invention comprising at least two isolated antibody fragments may be produced for example synthetically or recombinantly and may comprise bovine or chimeric or synthetic isolated antibody fragments or a combination thereof. For example, a polypeptide according to the invention may comprise two isolated antibody fragments, both being synthetic or one being synthetic and the other one being bovine. In one embodiment, the polypeptide according to the invention comprises only synthetic isolated antibody fragments.

In one aspect, the polypeptide comprising at least two isolated antibody fragments, is cyclised. In some embodiments, the polypeptide comprising at least two isolated antibody fragments, comprises at least one bridging moiety between two amino acids.

When the polypeptide is cyclic and does not have end-amino acids, it may be referred to as a macrocycle.

The definitions of bridging moiety described above in connection with cyclised antibody fragments also apply to the cyclised polypeptides of the present disclosure.

In particular, in one embodiment, the bridging moiety comprises a feature selected from the group consisting of a disulphide bond, an amide bond (lactam), a thioether bond, an aromatic ring, an unsaturated aliphatic hydrocarbon chain, a saturated aliphatic hydrocarbon chain and a triazole ring.

Methods of Production

The isolated antibody fragment or polypeptide of the invention may be produced by any suitable method, such as recombinant expression and/or chemical synthesis.

In one aspect, the present disclosure also provides methods of producing an isolated antibody fragment of the invention or a polypeptide of the invention, said method comprising a step of chemical synthesis.

Chemical synthesis approaches have been described, such as solid phase polypeptide synthesis (see e.g., Coin, I et al. (2007); Nature Protocols 2(12):3247-56).

In one embodiment, the isolated antibody fragment of the invention is produced by solid phase polypeptide synthesis.

In one embodiment, the isolated antibody fragment of the invention is produced using standard solid-phase Fmoc/tBu methods. Such methods are for example described in Atherton and Sheppard 1989, Fluorenylmethoxycarbonylpolyamide solid phase peptide synthesis: general principles and development. In Solid Phase Peptide Synthesis: A Practical Approach. IRL Press, Eynsham, Oxford, pp. 25-37); and Merrifield R. B. “Solid Phase Peptide Synthesis. I. The Synthesis of a Tetrapeptide”. J. Am. Chem. Soc. 85 (14): 2149-2154 (1963).

The synthesis is typically performed in a sequential manner in the C to N direction on robotic synthesisers. The synthesis may be started upon appropriate polystyrene supports with the first amino acid attached via a linkage to the support. An example of synthesis protocol is described in the Example section of the present disclosure. It will be appreciated by the skilled person that other protocols can be used, e.g. using different reagents, protective groups, other experimental conditions, and the skilled person will be able to adapt the protocol depending on the nature of the desired peptide and synthetic strategy.

The chemical synthesis of the isolated antibody fragments of the invention advantageously comprises the formation of disulphide bonds between two cysteine residues, which lead to the cyclisation of the isolated antibody fragments. Cyclic peptides can be produced by forming a disulphide bond between two cysteine residues, or by head-to-tail or side chain cyclisation, forming an amide bond. Using special groups, it is possible to cyclise between two specific cysteines in a peptide, thus it is possible to have more than one disulphide in a peptide. Different methods are available and comprise a site-directed method as described in the Example section of the present disclosure. The choice of the protecting groups to be used in an orthogonal protection strategy may vary. Cyclisation between two cysteine residues may alternatively be achieved by using thermodynamic controlled air oxidation to obtain the minimum energy form of the disulphides in the sequence, employing a mixture of reduced and oxidised glutathione, for example as described in the Examples.

In one aspect, a boron cage, such as decaborate, is directly incorporated into the isolated antibody fragment during chemical synthesis. The isolated antibody fragment may thus be labelled easily, just before administration, with a radioisotope, e.g. with astatine-211. Therefore, in one aspect the invention provides a method of producing an isolated antibody fragment or a polypeptide as defined in the present disclosure, said method comprising a step of chemical synthesis, and wherein the chemical synthesis comprises a step of incorporating a coupling reagent with a radioisotope. In one embodiment, the radioisotope is an alpha emitting radioisotope. In one embodiment, the radioisotope is Astatine 211.

The present disclosure also provides a polynucleotide encoding the isolated antibody fragment or polypeptide of the present invention. The polynucleotide (i.e. DNA sequence) of the present invention may comprise synthetic DNA, for instance produced by chemical processing, cDNA, genomic DNA or any combination thereof.

It will be appreciated that in the context of a polypeptide comprising at least two isolated antibody fragments of the invention, the DNA may be synthetic, and includes in a single DNA sequence, the sequence coding for the at least two isolated antibody fragments. Alternatively, the polypeptide comprising at least two isolated antibody fragments of the invention may use two separate non-synthetic or synthetic DNA sequences, each one coding one of the at least two isolated antibody fragments, which will then be conjugated or linked together after expression.

DNA sequences which encode an isolated antibody fragment of the present invention can be obtained by methods well known to those skilled in the art.

The present invention also relates to a cloning or expression vector comprising one or more polynucleotides or DNA sequences of the present invention. Accordingly, provided is a cloning or expression vector comprising one or more polynucleotides encoding an isolated antibody fragment or polypeptide of the present invention. In the context of a polypeptide comprising at least two isolated antibody fragments of the invention, the cloning or expression vector comprises at least two polynucleotides, encoding the at least two isolated antibody fragments of the present invention, respectively and suitable signal sequences. General methods by which the vectors may be constructed, transfection methods and culture methods are well known to those skilled in the art.

Also provided is a host cell comprising one or more cloning or expression vectors comprising one or more polynucleotides encoding an isolated antibody fragment of the present invention, or one or more vectors comprising the same. Any suitable host cell/vector system may be used for expression of CDR-H3 polynucleotide sequences encoding the isolated antibody fragment or polypeptide according to the present disclosure. Bacterial, for example E. coli, and other microbial systems may be used or eukaryotic, for example mammalian, host cell expression systems may also be used. Suitable mammalian host cells include CHO, myeloma or hybridoma cells. Suitable types of Chinese Hamster Ovary (CHO cells) for use in the present invention may include CHO and CHO-K1 cells including dhfr-CHO cells, such as CHO-DG44 cells and CHO-DXB11 cells, which may be used with a DHFR selectable marker or CHOK1-SV cells which may be used with a glutamine synthetase selectable marker. Other cell types of use in expressing antibodies include lymphocytic cell lines, e.g., NSO myeloma cells and SP2 cells, COS cells.

In one aspect, there is provided a process for producing an isolated antibody fragment or polypeptide of the invention, said process comprising expressing an isolated antibody fragment or polypeptide of the invention, from a host cell as defined in the present disclosure.

In one aspect, there is provided a method of producing an isolated antibody fragment or polypeptide as described in the present disclosure, said method comprising:
a) immunising a bovine with an immunogenic composition, and;
b) isolating antigen-specific memory B-cells, and;
c) sequencing the cDNA of CDR-H3 or portions thereof, and;
d) expressing or synthesising the knob domain of the ultralong CDR-H3 or portion thereof,
   wherein the immunogenic composition comprises an antigen of interest or immunogenic portions thereof, or DNA encoding the same.

Step a)

An “immunogenic composition” refers to a composition which is able to generate an immune response in bovine administered with said composition. An immunogenic composition typically allows the expression of an immunogenic antigen of interest in the administered bovine, against which bovine antibodies may be raised as part of the immune response.

“Protein immunisation” refers to the technique of administration of an immunogenic protein comprising an antigen of interest, or immunogenic portion of said protein, comprising said antigen of interest or immunogenic portion thereof.

In one embodiment, the immunogenic composition comprises a full-length protein. In another embodiment, the immunogenic composition comprises an immunogenic portion of a protein.

“DNA immunisation” refers to the technique of direct administration into the cells of the bovine of a genetically engineered nucleic acid molecule encoding a full-length protein or an immunogenic portion thereof comprising an antigen of interest (also referred to as nucleic acid vaccine or DNA vaccine herein) to produce an immunological response in said cells, against said antigen of interest. DNA immunisation uses the host cellular machinery for expressing peptide(s) corresponding to the administered nucleic acid molecule and/or achieving the expected effect, in particular antigen expression at the cellular level, and furthermore immunotherapeutic effect(s) at the cellular level or within the host organism.

“Cell immunisation” refers to the technique of administration of cells naturally expressing or transfected with an immunogenic protein comprising an antigen of interest, or immunogenic portion of said protein, comprising said antigen of interest or immunogenic portion thereof. In one embodiment, the immunisation at step a) is performed using cell immunisation with fibroblasts transfected with an immunogenic protein comprising an antigen of interest, or immunogenic portion of said protein, comprising said antigen of interest or immunogenic portion thereof.

By “Immunogenic portion”, it is meant a portion of the protein or antigen of interest which retains the capacity of inducing an immune response in the bovine animal administered with said portion of the protein or antigen of interest or DNA encoding the same, in order to enable the production of antibody fragments of the invention as disclosed herein.

In one embodiment, the immunisation step a) may be performed using protein immunisation, DNA immunisation, or cell immunisation or any combination thereof.

The immunisation step a) may be performed using a prime-boost immunisation protocol implying a first administration (prime immunisation or prime administration) of the immunogenic composition, and then at least one further administration (boost immunisation or boost administration) that is separated in time from the first administration within the course of the immunisation protocol. Boost immunisations encompass one, two, three or more administrations.

In one embodiment, the immunisation step a) is performed using a prime-boost immunisation protocol comprising a prime immunisation with an antigen of interest in presence of a first adjuvant, then at least one boost immunisation with said antigen of interest in presence of a second adjuvant.

In one embodiment, the immunogenic composition is administered by sub-cutaneous injection, for example into the shoulder. In one embodiment, the antigen of interest is the component C5 of the Complement.

“Adjuvant” refers to an immune stimulator. Adjuvants are substances well known in the art. Traditional adjuvants, which act as immune stimulators or antigen delivery systems, or both, encompass, for example, Alum, polysaccharides, liposomes, nanoparticles based on biodegradable polymers, lipopolysaccharides. For example, the adjuvant may be a Freund's adjuvant, a Montanide adjuvant, or a Fama adjuvant.

Step b)

Methods for isolating antigen-specific memory B-cells are well known and generally comprise isolating B-cells from PBMC (Peripheral Blood Mononuclear Cells), or from secondary lymphoid organs, i.e. from lymphoid node, or the spleen. In one embodiment, isolating antigen-specific memory B-cells is performed 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the immunisation step a). In one embodiment, isolating antigen-specific memory B-cells is performed 24 hours, 36 hours, 48 hours, 60 hours, 72 hours, 84 hours, 96 hours after the immunisation step a). In one embodiment, step b) comprises sorting of the antigen-specific B cells by flow cytometry.

Step c)

Step c) generally comprises a first step of obtaining cDNA from the memory B-cells obtained at step b), using methods well known in the art, for example methods comprising a RT-PCR performed on the lysate of memory B-cells. Methods for sequencing cDNA are well known in the art. Step c) comprises sequencing the cDNA of CDR-H3 or portions thereof to identify ultralong CDR-H3. The analysis of the sequences at step c) allows the identification of ultralong CDR-H3 as compared with standard CDR-H3, as disclosed herein based on the size of the CDR-H3 sequence and/or using alternative methods such as sequence alignments with well-known and/or standard nucleic or amino acid sequences of ultralong CDR-H3. The knob domain may then be defined as described in the present disclosure and its sequence isolated.

For example, a method for amplifying the cDNA of CDR-H3 as described in the Examples may be used. The method may comprise a first step of RT-PCR performed on the lysate of memory B-cells isolated at step b). The method may comprise a primary polymerase chain reaction (PCR) with primers flanking CDR-H3, annealing to the conserved framework 3 and framework 4 of the VH, to amplify all CDR-H3 sequences, irrespective of their length or amino acid sequence. The method may additionally comprise a second round of PCR to barcode the CDR-H3 sequences for ion torrent sequencing, as described in the Examples.

In one embodiment, the method for sequencing the cDNA of CDR-H3 or portions thereof comprises:
1) a primary PCR with primers flanking CDR-H3, annealing to the conserved framework 3 and framework 4 of the VH, to amplify all CDR-H3 sequences, and
2) a second round of PCR to barcode the CDR-H3 sequences for ion torrent sequencing.

In one embodiment, the primers used at step 1) comprise or consist of SEQ ID NO:446 and SEQ ID NO: 447. In one embodiment, the primers used at step 2) comprise or consist of SEQ ID NO:448 and SEQ ID NO: 449).

Step d)

Step d) may be performed according to well-known methods to express polypeptides, notably by using cloning, expression vectors, and host cells as described above.

Step d) may alternatively comprise the chemical synthesis of the knob domain of the ultralong CDR-H3 or portion thereof, which may be performed according to well-known methods including as described above.

In one embodiment, the method of producing an isolated antibody fragment of the invention further comprises a step of screening, for example for binding to an antigen of interest.

Optionally, the screening step is preceded by a step of reformatting the ultralong CDR-H3 or the knob domain of the ultralong CDR-H3 or portion thereof into a screening format.

In one embodiment, the step of reformatting the ultralong CDR-H3 or the knob domain of the ultralong CDR-H3 or portion thereof into a screening format comprises fusing the ultralong CDR-H3 or the knob domain of the ultralong CDR-H3 or portion thereof, to a carrier, optionally via a linker, for example a cleavable linker.

It will be appreciated that the screening step may be performed before or after step d). For example, the isolated antibody fragments of the invention (i.e. the knob domain of the ultralong CDR-H3 or portion thereof) may be expressed in a host cell according to step d), then recovered and screened in vitro for binding to the antigen of interest, optionally following a step of reformatting the knob domain of the ultralong CDR-H3 or portion thereof into a screening format as described in the present disclosure.

Alternatively, the knob domain of the ultralong CDR-H3 may be expressed or synthesised as part of the entire ultralong CDR-H3 after step c) and screened for binding to the antigen of interest before step d) optionally after a step of reformatting the ultralong CDR-H3 into a screening format as described herein. In such alternative, the knob domains or portions thereof comprised in the ultralong CDR-H3 which have been found to specifically bind to the antigen of interest may be expressed or synthesised at step d).

In one embodiment, the carrier is an Fc polypeptide. An “Fc polypeptide” as used herein is a polypeptide comprising a Fc fragment. In one embodiment, the Fc polypeptide is a scFc. “Single-chain Fc polypeptide” or “scFc” as employed herein refers to a single chain polypeptide comprising two CH2 domains and two CH3 domains characterized in that said CH2 and CH3 domains form a functional Fc domain within the chain. The functional Fc domain in the single-chain polypeptides of the present invention is not formed by dimerisation of two chains i.e. the two CH2 domains and two CH3 domains are present in a single chain and form a functional Fc domain within the single chain. The term ‘functional’ as used herein refers to the ability of the Fc domain formed within the single chain polypeptide to provide one or more effector functions usually associated with Fc domains although it will be appreciated that other functions may be engineered into such domains.

In one embodiment, the carrier is a scFc and comprises the sequence SEQ ID NO: 155. In one embodiment, the carrier is a scFc and the fusion protein comprises a linker, wherein the linker comprises a TEV protease cleavage site and a Gly-Ser linker. In one embodiment, the carrier is a scFc and the fusion protein comprises the sequence SEQ ID NO:156.

Additional scFc sequences and variants useful in the context of the present disclosure have been described in WO2008/012543.

In one aspect, the invention provides a method of producing an isolated antibody fragment as described in the present disclosure, said method comprising:
a) immunising a bovine with an immunogenic composition, and;
b) isolating total RNA from PBMC or secondary lymphoid organ, and;
c) amplifying the cDNA of the ultralong CDR-H3, and;
d) sequencing an ultralong CDR-H3 or portion thereof; and,
e) expressing or synthesising the knob domain of the ultralong CDR-H3 or portion thereof,
   wherein the immunogenic composition comprises an antigen of interest or immunogenic portions thereof, or DNA encoding the same.

Step a) is as described above.

Step b) Methods for isolating total RNA from PBMC or secondary lymphoid organ are well known in the art.

It will be appreciated that step c) generally comprises a first step of obtaining cDNA from the total RNA obtained at step b), using RT-PCR. Advantageously, at step c) a method for amplifying directly the cDNA of ultralong CDR-H3 and discriminate from standard CDR-H3 may be used. The method may comprise a primary polymerase chain reaction (PCR) with primers flanking CDR-H3, annealing to the conserved framework 3 and framework 4 of the VH, to amplify all CDR-H3 sequences, irrespective of their length or amino acid sequence. The method may additionally comprise a second round of PCR with stalk primers to specifically amplify ultralong sequences from the primary PCR. This method is advantageous as it allows to directly clone a sequence of ultralong CDR-H3 of interest into expression vectors.

In one embodiment, the method for amplifying the cDNA of CDR-H3 comprises:
1) a primary PCR using primers flanking CDR-H3, annealing to the conserved framework 3 and framework 4 of the VH, to amplify all CDR-H3 sequences, and
2) a second round of PCR using stalk primers to specifically amplify ultralong sequences from the primary PCR.

In one embodiment, the primers used at step 1) comprise or consist of SEQ ID NO: 446 and SEQ ID NO: 447. In one embodiment, the primers used at step 2) are selected from the group consisting of SEQ ID NO:482 to SEQ ID NO:494. It will be appreciated that the primers used at step 2) comprise one ascending primer and one descending primer, i.e. the primers may comprise one ascending primer of any one of SEQ ID NO: 482 to SED ID NO: 488, and one descending primer of any one of SEQ ID NO: 489 to SEQ ID NO:494.

Step d) comprises sequencing the cDNA of CDR-H3 or portion thereof in order to identify the knob domain peptide of the ultralong CDR-H3 or portions thereof. Step d) may be performed according to methods well known in the art such as direct nucleotide sequencing.

Step e) is as described above.

In one embodiment, the method of producing an isolated antibody fragment of the invention further comprises a step of screening, for example for binding to an antigen of interest. Optionally, the screening step is preceded by a step of reformatting the ultralong CDR-H3 or the knob domain of the ultralong CDR-H3 or portion thereof into a screening format as described herein. The screening step may be performed before or after step e). For example, the isolated antibody fragments of the invention (i.e. the knob domain of the ultralong CDR-H3 or portion thereof) may be expressed in a host cell according to step e), then recovered and screened in vitro for binding to the antigen of interest, optionally following a step of reformatting the knob domain of the ultralong CDR-H3 or portion thereof into a screening format as described in the present disclosure.

Alternatively, the knob domain of the ultralong CDR-H3 may be expressed or synthesised as part of the entire ultralong CDR-H3 after step d) and screened for binding to the antigen of interest before step e) optionally after a step of reformatting the ultralong CDR-H3 into a screening format as described herein. In such alternative, the knob domains or portions thereof comprised in the ultralong CDR-H3 which have been found to specifically bind to the antigen of interest may be expressed or synthesised at step e).

In another aspect, the invention provides a method of producing an isolated antibody fragment as described in the present disclosure, said method comprising:
a) immunising a bovine with an immunogenic composition, and;
b) isolating antigen-specific memory B-cells, and;
c) amplifying the cDNA of ultralong CDR-H3, and;
d) sequencing the ultralong CDR-H3; and,
e) expressing or synthesising the knob domain of the ultralong CDR-H3 or portion thereof,
   wherein the immunogenic composition comprises an antigen of interest of immunogenic portions thereof, or DNA encoding the same.

Steps a) to e) are as described above.

In one embodiment, the method of producing an isolated antibody fragment as described in the present disclosure, further comprises a step of screening, for example for binding to an antigen of interest, which is as described above, and notably may be performed before or after step e).

Antibody Fragment Libraries

In one aspect, the disclosure provides immune libraries and methods for generating immune libraries, comprising a diversity of isolated antibody fragments of the invention, notably knob domains of bovine ultralong CDR-H3 or portions thereof, or a diversity of DNA or RNA sequences coding the same.

Advantageously, the invention provides new methods of discovering therapeutic antibody fragments and polypeptides derived therefrom, comprising immunising bovine with an antigen of interest as described above. It may be possible to generate extensive immune libraries of isolated bovine antibody fragments, notably knob domains of bovine ultralong CDR-H3 and portions thereof and to screen and select for those knob domains that have a desired effect, for example for their binding and/or binding affinity to an antigen of interest, in particular by display technologies.

Immune repertoires of antibody fragments are generated by using the genetic information coding for bovine antibody fragments of the present disclosure, which can be derived from B cells isolated from bovine administered with an antigen of interest. Immune libraries may be screened using display technologies of the bovine antibody fragments, e.g. using in vitro display technologies (such as phage display, bacterial display, yeast display, ribosome display, mRNA display). Mammalian cell display may be advantageous for the display of disulfide rich proteins such as bovine ultralong CDR-H3 or fragment thereof, e.g. as described in Crook, Z. R. et al. Publisher Correction: Mammalian display screening of diverse cystine-dense peptides for difficult to drug targets. Nat Commun 9, 1072, (2018).

In one embodiment, the invention provides libraries of isolated antibody fragments of the invention expressed at the surface of mammalian cells as fusion proteins, such as Fc polypeptides fusion proteins. In one embodiment, the invention provides libraries of knob domains of bovine ultralong CDR-H3.

In one embodiment, the invention provides immune libraries or naive libraries of isolated antibody fragments of the invention, prepared from animals which have not been administered an immunogen. In one embodiment, the invention provides phage display libraries of isolated antibody fragments of the invention. In such embodiment, the isolated antibody fragments of the invention may be expressed directly at the surface of phages using any suitable method.

In one aspect, the invention provides libraries of ultralong CDR-H3 sequences, i-e libraries of isolated antibody fragments of the invention, when expressed as part of the full sequence of CDR-H3 (i.e. comprising the knob and stalk domains). In one embodiment, the libraries are naïve libraries. In one embodiment, the naive libraries are prepared from cattle. In another embodiment, the libraries are immune libraries. In one embodiment, the libraries are prepared from immunised cattle. In one specific aspect, the disclosure provides a phage display library of isolated antibody fragments of the invention, optionally displayed within the full sequences of CDR-H3. In one embodiment, the phage display library is a M13 phage display library. In one embodiment, the isolated antibody fragments of the invention, optionally displayed within the full sequences of CDR-H3, are fused directly to the pIII coat protein of the M13 phage. In one embodiment, the isolated antibody fragments of the invention, optionally displayed within the full sequences of CDR-H3, are fused to the pIII coat protein of the M13 phage via a linker (or “spacer”). A suitable linker may be a linker which allows to separate the cysteine-rich domain from the cysteines of the pIII, notably to ensure that the pIII and the knob domain peptide fold independently and correctly. Methods for producing a phage display library are well known. Phagemid vectors have for example been described in Hoogenboom H R at al. (Hoogenboom H R, Multi-subunit proteins on the surface of filamentous phage: methodologies for displaying antibody (Fab) heavy and light chains. Nucleic Acids Res. 1991; 19(15):4133-4137). In one embodiment, the phage display library of isolated antibody fragments of the invention comprises CDR-H3 of sequence SEQ ID NO: 477 and/or SEQ ID NO: 104 and/or SEQ ID NO: 13 and/or SEQ ID NO: 1.

In one aspect, the disclosure provides a phage display library, comprising a plurality of recombinant phages; each of the plurality of recombinant phages comprising an M13-derived expression vector, wherein the M13-derived expression vector comprises a polynucleotide sequence encoding an isolated antibody fragment as disclosed in the present disclosure, optionally displayed within the full sequence of ultralong CDR-H3. In one embodiment, the isolated antibody fragment optionally displayed within the full sequence of ultralong CDR-H3, is fused to the sequence encoding the pIII coat protein of the M13 phage, directly or via a spacer.

In one aspect, the disclosure provides methods for generating phage display libraries of ultralong CDR-H3 sequences, i-e libraries of isolated antibody fragments of the invention, displayed within the full sequence of CDR-H3. For example, a method as described in Example 12 may be used, wherein the full sequences of the ultralong CDR-H3 are fused directly to the pIII coat protein of the M13 phage.

In one aspect, there is provided a method for generating an immune phage display library of ultralong CDR-H3 sequences, said method comprising:
a) immunising a bovine with an immunogenic composition, and;
b) isolating total RNA from PBMC or secondary lymphoid organ, and;
c) amplifying the sequences of the ultralong CDR-H3, and;
d) fusing the sequences obtained in c) to the sequence coding for the pIII protein of a M13 phage within a phagemid vector, and;
e) transforming host bacteria with the phagemid vector obtained at step d) in combination with a helper phage co-infection, and;
f) culturing the bacteria obtained at step e), and;
g) recovering the phages from the culture medium of the bacteria,
   wherein the immunogenic composition comprises an antigen of interest or immunogenic portions thereof, or DNA encoding the same.

Steps a) to g) are methods well known in the art. For example, septs a) to g) may be performed as described in Example 12. In particular, for step c), a method for amplifying the cDNA of CDR-H3 as described in Example 12 may be used. The method may comprise a primary PCR with primers flanking CDR-H3, annealing to the conserved framework 3 and framework 4 of the VH, to amplify all CDR-H3 sequences, irrespective of their length or amino acid sequence. The method may additionally comprise a second round of PCR with stalk primers to specifically amplify ultralong sequences from the primary PCR.

In one embodiment, the method for amplifying the cDNA of CDR-H3 comprises:
1) a primary PCR using primers flanking CDR-H3, annealing to the conserved framework 3 and framework 4 of the VH, to amplify all CDR-H3 sequences, and
2) a second round of PCR using stalk primers to specifically amplify ultralong sequences from the primary PCR.

In one embodiment, the primers used at step 1) comprise or consist of SEQ ID NO:446 and SEQ ID NO: 447). In one embodiment, the primers used at step 2) are selected from the group consisting of SEQ ID NO:482 to SEQ ID NO:494. It will be appreciated that the primers used at step 2) comprise one ascending primer and one descending primer, i.e. the primers may comprise one ascending primer of any one of SEQ ID NO: 482 to SED ID NO: 488, and one descending primer of any one of SEQ ID NO: 489 to SEQ ID NO:494.

In one aspect, the invention provides a method for producing an isolated antibody fragment of the invention which binds to an antigen of interest, said method comprising:
a) generating a phage display library of ultralong CDR-H3; and,
b) enriching the phage display library against the antigen of interest to produce an enriched population of phage which bind the antigen of interest; and,
c) sequencing an ultralong CDR-H3 from the enriched population of phage obtained in step b); and,
d) expressing or synthesising an isolated antibody fragment (i.e. the knob domain of the ultralong CDR-H3 or portion thereof) derived from the ultralong CDR-H3 obtained in step c).

Steps a) to d) are methods well known in the art. For example, septs a) to d) may be performed as described in Example 12. For example, enriching the phage display library against the antigen of interest at step b) may be performed by panning the library obtained at step a) against the antigen of interest. Enriched sub-libraries can be further screened by monoclonal Phage screening ELISA as described in the Examples.

At step c), the sequence of the ultralong CDR-H3 sequences may be amplified using PCR using appropriate primers, for example sequencing primers annealing to the phagemid vector. In one embodiment, the primers used comprise or consist of SEQ ID NO: 495 and/or SEQ ID NO:496.

In another aspect, the invention provides a method for producing an isolated antibody fragment of the invention which binds to an antigen of interest, said method comprising:
a) generating a phage display library of isolated antibody fragments of the invention; and,
b) enriching the phage display library against the antigen of interest to produce an enriched population of phage which bind the antigen of interest; and,
c) sequencing an isolated antibody fragment from the enriched population of phage obtained in step b); and,
d) expressing or synthesising an isolated antibody fragment (i.e. the knob domain of the ultralong CDR-H3 or portion thereof) obtained in step c).

Steps a) may be performed according to methods as disclosed in the present disclosure.

Steps b) to d) are as disclosed above.

Advantageously, the disclosure provides a route to circumvent cell sorting and deep sequencing for the discovery of bovine antibody fragments, whereby libraries of CDR-H3 sequences can be cloned and screened for binding to an antigen, or a panel of antigens, using in vitro display technologies.

A library will generally contain at least 10² members, more preferably at least 10⁶ members, and more preferably at least 10¹ members (e.g., any of the mRNA-polypeptide complexes). In some embodiments, the library will include at least 10¹² members or at least 10¹⁴ members. In general, the members will differ from each other; however, it is expected there will be some degree of redundancy in any library.

In another aspect, the disclosure provides synthetic libraries and methods for generating synthetic libraries, comprising a diversity of isolated antibody fragments of the invention, or a diversity of DNA or RNA sequences coding the same.

Synthetic libraries may comprise isolated antibody fragments expressed at the surface of cells. Synthetic libraries may be screened using display technologies, e.g. using in vitro display technologies (such as phage display, bacterial display, yeast display, ribosome display, mRNA display). Mammalian cell display may also be used.

In one aspect, the disclosure provides synthetic libraries and methods for generating synthetic libraries, comprising isolated antibody fragments of the invention fused to (or inserted into) a suitable scaffold, preferably a protein scaffold. A suitable protein scaffold may be another antibody fragment, for example an antigen-binding fragment of an antibody, such as a VHH, a VH, a VL, a Fab, a scFv, and a dsscFv, or any other suitable infrastructure. For example, isolated antibody fragments of the invention may be inserted into a VH or a VL domain, more particularly into a framework 3 region of a VH or VL for example as described in WO2020/011868 incorporated herein by reference.

Synthetic libraries may be screened using display technologies, e.g. using in vitro display technologies (such as phage display, bacterial display, yeast display, ribosome display, mRNA display) wherein each isolated antibody fragment of the invention is expressed as part of a fusion protein with a suitable protein scaffold such as an antigen-binding fragment.

In one embodiment, the fusion protein consists of an isolated antibody fragment of the invention fused to a suitable protein scaffold such as an antigen-binding fragment, optionally via one or more, for example two, linker(s). In another embodiment, the fusion protein comprises an isolated antibody fragment of the invention, optionally displayed within the full sequences of CDR-H3 or portion thereof, fused to a suitable protein scaffold such as an antigen-binding fragment, optionally via one or more, for example two, linker(s).

In one aspect, the disclosure provides a synthetic phage display library of isolated antibody fragments of the invention, wherein each of the isolated antibody fragments is displayed as part of a fusion protein with an antigen-binding fragment of an antibody. In one embodiment, the fusion protein comprises an isolated antibody fragment of the invention optionally displayed within the full sequence of CDR-H3 or portion thereof. In one embodiment, the antigen-binding fragment of an antibody is a VHH. Therefore, the invention provides a phage display library comprising isolated antibody fragments of the invention, optionally displayed within the full sequences of CDR-H3 or portion thereof, expressed at the surface of phages, as VHH fusion proteins. In one embodiment, each isolated antibody fragment of the invention, optionally displayed within the full sequence of CDR-H3 or portion thereof, is inserted into a VHH, for example in the non-binding VH framework 3 loop, optionally via one or more linker. A suitable linker may improve the independent and correct folding of the isolated antibody fragment or full sequence of CDR-H3, and the VHH. In one embodiment, the VHH phage display library is a VHH M13 phage display library. In one embodiment, the VHH fusion proteins are fused directly to the pIII coat protein of the M13 phage. In one embodiment, the VHH fusion proteins are fused to the pIII coat protein of the M13 phage via a linker. In one embodiment, the VHH comprises or has the sequence SEQ ID NO: 351. In one embodiment, the phage display library of isolated antibody fragments of the invention comprises VHH fusion proteins of sequence SEQ ID NO: 476 and/or SEQ ID NO: 478 and/or SEQ ID NO: 479 and/or SEQ ID NO: 480.

Methods for producing a Phage display library are well known. The method described in the present disclosure to produce Phage-VHH libraries wherein CDR-H3 are inserted into VHH may be used as an example.

Pharmaceutical Compositions and Medical Uses

In one aspect, the invention provides a pharmaceutical composition comprising an isolated antibody fragment or polypeptide as defined in the present disclosure, in combination with one or more of a pharmaceutically acceptable excipient.

The term “pharmaceutically acceptable excipient” as used herein refers to a pharmaceutically acceptable formulation carrier, solution or additive to enhance the desired characteristics of the compositions of the present disclosure. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes. This may include production and sterilization by filtration of the buffered solvent solution used for the formulation, aseptic suspension of the isolated antibody fragment in the sterile buffered solvent solution, and dispensing of the formulation into sterile receptacles by methods familiar to those of ordinary skill in the art.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

The isolated antibody fragments or polypeptides of the disclosure can be delivered dispersed in a solvent, e.g., in the form of a solution or a suspension. It can be suspended in an appropriate physiological solution, e.g., physiological saline, a pharmacologically acceptable solvent or a buffered solution.

A thorough discussion of pharmaceutically acceptable carriers is available in Remington's Pharmaceutical Sciences (Mack Publishing Company, N.J. 1991).

The pharmaceutical compositions suitably comprise a therapeutically effective amount of the isolated antibody fragments or polypeptides of the invention. The term “therapeutically effective amount” as used herein refers to an amount of a therapeutic agent needed to treat, ameliorate or prevent a targeted disease or condition, or to exhibit a detectable therapeutic or preventative effect. For any antibody fragment, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans. The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician.

Compositions may be administered individually to a patient or may be administered in combination (e.g. simultaneously, sequentially or separately) with other agents, drugs or hormones. Agents as employed herein refers to an entity which when administered has a physiological affect. Drug as employed herein refers to a chemical entity which at a therapeutic dose has an appropriate physiological affect.

The dose at which the isolated antibody fragment or polypeptide of the present disclosure is administered depends on the nature of the condition to be treated, the extent of the inflammation present and on whether the isolated antibody fragment is being used prophylactically or to treat an existing condition.

The frequency of dose will depend on the half-life of the isolated antibody fragment or polypeptide and the duration of its effect. If the isolated antibody fragment or polypeptide has a short half-life (e.g. 2 to 10 hours) it may be necessary to give one or more doses per day. Alternatively, if the isolated antibody fragment or polypeptide has a long half-life (e.g. 2 to 15 days) it may only be necessary to give a dosage once per day, once per week or even once every 1 or 2 months.

The pharmaceutical compositions of this invention may be administered by any number of routes including, but not limited to, oral, intravenous, intramuscular, intra-arterial, intramedullary, intrathecal, intraventricular, transdermal, transcutaneous, subcutaneous, intraperitoneal, intranasal, enteral, topical, sublingual, intravaginal or rectal routes. Hyposprays may also be used to administer the pharmaceutical compositions of the invention.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the isolated antibody fragment may be in dry form, for reconstitution before use with an appropriate sterile liquid.

Direct delivery of the compositions will generally be accomplished by injection, subcutaneously, intraperitoneally, intravenously or intramuscularly, or delivered to the interstitial space of a tissue. The compositions can also be administered into a specific tissue of interest. Dosage treatment may be a single dose schedule or a multiple dose schedule.

In one embodiment the formulation is provided as a formulation for topical administrations including inhalation.

Suitable inhalable preparations include inhalable powders, metering aerosols containing propellant gases or inhalable solutions free from propellant gases (such as nebulisable solutions or suspensions). Inhalable powders according to the disclosure containing the active substance may consist solely of the abovementioned active substances or of a mixture of the above-mentioned active substances with physiologically acceptable excipient. The propellent gases which can be used to prepare the inhalable aerosols are known in the art. Suitable propellent gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellent gases may be used on their own or in mixtures thereof.

The propellent-gas-containing inhalable aerosols may also contain other ingredients such as cosolvents, stabilisers, surface-active agents (surfactants), antioxidants, lubricants and means for adjusting the pH. All these ingredients are known in the art. The propellant-gas-containing inhalable aerosols according to the invention may contain up to 5% by weight of active substance. Aerosols according to the invention contain, for example, 0.002 to 5% by weight, to 3% by weight, 0.015 to 2% by weight, 0.1 to 2% by weight, 0.5 to 2% by weight or to 1% by weight of active.

Alternatively, topical administrations to the lung may also be by administration of a liquid solution or suspension formulation, for example employing a device such as a nebulizer, for example, a nebulizer connected to a compressor (e.g., the Pan LC-Jet Plus® nebulizer connected to a Pari Master® compressor manufactured by Pari Respiratory Equipment, Inc., Richmond, Va.).

In one embodiment the formulation is provided as discrete ampoules containing a unit dose for delivery by nebulisation.

In one embodiment the isolated antibody fragment or polypeptide is supplied in lyophilised form, for reconstitutions or alternatively as a suspension formulation.

The isolated antibody fragment or polypeptide of the present disclosure can be delivered dispersed in a solvent, e.g., in the form of a solution or a suspension. It can be suspended in an appropriate physiological solution, e.g., physiological saline, a pharmacologically acceptable solvent or a buffered solution. Buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. As mentioned supra a suspension can made, for example, from lyophilised isolated antibody fragment or polypeptide.

Nebulisable formulation according to the present disclosure may be provided, for example, as single dose units (e.g., sealed plastic containers or vials) packed in foil envelopes. Each vial contains a unit dose in a volume, e.g., 2 ml, of solvent/solution buffer.

The isolated antibody fragments or polypeptides of the present disclosure are thought to be suitable for delivery via nebulisation.

The present invention also provides a process for preparation of a pharmaceutical or diagnostic composition comprising adding and mixing the isolated antibody fragment or polypeptide of the present invention together with one or more of a pharmaceutically acceptable excipient, diluent or carrier.

The present invention also provides for methods and compositions for the delivery of the isolated antibody fragments as described herein by gene therapy, particularly by adeno-associated virus (AAV) vector.

Hence, the present invention provides for a pharmaceutical composition comprising a viral vector having a viral capsid and an artificial genome comprising an expression cassette flanked by inverted terminal repeats (ITRs) wherein the expression cassette comprises a transgene comprising a polynucleotide sequence encoding the isolated antibody as described herein. The ITRs sequences may be used for packaging the artificial genome comprising the polynucleotide sequences encoding the isolated antibody fragment or polypeptide as described herein into the virion of the viral vector.

The transgene in the expression cassette is operably linked to expression control elements such as promoters that will control expression of the transgene in human cells.

The viral vector is preferably AAV based viral vectors. A variety of AAV capsids have been described in the art. Methods of generating AAV vectors have also been described extensively in the literature (e.g., WO 2003/042397; WO 2005/033321, WO 2006/110689; U.S. Pat. No. 7,588,772 B2). The source of AAV capsids may be selected from an AAV which targets a desired tissue. For example, suitable AAV may include, e.g., AAV9 (U.S. Pat. No. 7,906,111; US 2011-0236353-A1), rh10 (WO 2003/042397) and/or hu37 (U.S. Pat. No. 7,906,111B2; US20110236353). However, other AAV, including, e.g., AAV1, AAV2, AAV-TT, AAV3, AAV4, AAV5, AAV6, AAV7, AAV8 and AAV.PHP.B (or variants thereof) and others may also be selected.

Methods for generating and isolating AAV viral vectors suitable for delivery to a subject are known in the art (U.S. Pat. No. 7,790,449B2; U.S. Pat. No. 7,282,199B2; WO 2003/042397; WO 2005/033321, WO 2006/110689; and U.S. Pat. No. 7,588,772 B2).

In one aspect, the invention provides an isolated antibody fragment or a polypeptide as defined in the present disclosure, for use in therapy.

Isolated antibody fragments and polypeptides of the invention are useful in the treatment of diseases or disorders including inflammatory diseases and disorders, immune diseases and disorders, complement-related diseases and disorders, autoimmune diseases, vascular indications, neurological diseases and disorders, kidney-related indication, ocular diseases.

In some embodiments, isolated antibody fragments, polypeptides, and pharmaceutical compositions thereof according to the present invention may be useful in the treatment of diseases, disorders and/or conditions where C5 cleavage leads to progression of the disease, disorder and/or condition. Such diseases, disorders and/or conditions may include, but are not limited to immune and autoimmune, neurological, cardiovascular, pulmonary, and ocular diseases, disorders and/or conditions.

Immune and autoimmune diseases and/or disorders may include, but are not limited to Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Acute antibody-mediated rejection following organ transplantation, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Autoimmune urticaria, Axonal and neuronal neuropathies, Bacterial sepsis and septic shock, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Diabetes Type I, Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia (including atypical hemolytic uremic syndrome and plasma therapy-resistant atypical hemolytic-uremic syndrome), Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Insulin-dependent diabetes (type1), Interstitial cystitis, Juvenile arthritis, Juvenile diabetes, Kawasaki syndrome, Lambert-Eaton syndrome, Large vessel vasculopathy, Leukocytoclastic vasculitis, Lichen planus, Lichen sclerosus, Ligneous conjunctivitis, Linear IgA disease (LAD), Lupus (SLE), Lyme disease, Meniere's disease, Microscopic polyangiitis, Mixed connective tissue disease (MCTD), Mooren's ulcer, Mucha-Habermann disease, Multiple endocrine neoplasia syndromes, Multiple sclerosis, Multifocal motor neuropathy, Myositis, Myasthenia gravis, Narcolepsy, Neuromyelitis optica (Devic's), Neutropenia, Ocular cicatricial pemphigoid, Optic neuritis, Osteoarthritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome. Polyarteritis nodosa. Type I, II, and III autoimmune polyglandular syndromes, Polyendocrinopathies, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic Pulmonary fibrosis, Pyoderma gangrenosum, Pure red cell aplasia, Raynauds phenomenon, Reactive arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Shiga-Toxin producing Escherichia Coli Hemolytic-Uremic Syndrome (STEC-HUS), Sjogren's syndrome, Small vessel vasculopathy, Sperm and testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Tubular autoimmune disorder, Ulcerative colitis, Undifferentiated connective tissue disease (UCTD), Uveitis, Vesiculobullous dermatosis, Vasculitis, Vitiligo and Wegener's granulomatosis (also known as Granulomatosis with Polyangiitis (GPA)).

Neurological diseases, disorders and/or conditions may include, but are not limited to Alzheimer's disease, Parkinson's disease, Lewy body dementia and Multiple sclerosis.

Cardiovascular diseases, disorders and/or conditions may include, but are not limited to atherosclerosis, myocardial infarction, stroke, vasculitis, trauma (surgery), and conditions arising from cardiovascular intervention (including, but not limited to cardiac bypass surgery, arterial grafting and angioplasty).

Pulmonary diseases, disorders and/or conditions may include, but are not limited to asthma, pulmonary fibrosis, chronic obstructive pulmonary disease (COPD) and adult respiratory distress syndrome.

Ocular related applications include, but are not limited to: Age-related macular degeneration, allergic and giant papillary conjunctivitis, Behcet's disease, choroidal inflammation, complications related to intraocular surgery, corneal transplant rejection, corneal ulcers, cytomegalovirus retinitis, dry eye syndrome, endophthalmitis, Fuch's disease, Glaucoma, immune complex vasculitis, inflammatory conjunctivitis, ischemic retinal disease, keratitis, macular edema, ocular parasitic infestation/migration, retinitis pigmentosa, scleritis, Stargardt disease, subretinal fibrosis, uveitis, vitreo-retinal inflammation, and Vogt-Koyanagi-Harada disease.

In one embodiment, the invention provides an isolated antibody fragment or polypeptide or pharmaceutical composition as defined in the present disclosure for use in the prevention and/or the treatment of a pathological disease, disorder or condition selected from the group consisting of infections (viral, bacterial, fungal and parasitic), endotoxic shock associated with infection, arthritis such as rheumatoid arthritis, asthma such as severe asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases such as myocardial infarction as well as atherosclerosis, intravascular coagulation, bone resorption, osteoporosis, osteoarthritis, periodontitis and hypochlorhydia.

Isolated Antibody Fragments to Enable Small Molecule Drug Discovery

In another aspect, the present invention relates to an improved method for identifying compounds of therapeutic interest employing antibody-protein target interactions to help present and/or hold the protein in a conformation that exposes or presents a binding site that has the potential to modify the protein function and which may be occluded in the “natural” conformation. Such method has been described in WO2014/001557 published on Jan. 3, 2014, and Lawson, A. Nat Rev Drug Discovery 11, 519-525, (2012).

Thus, isolated antibody fragments of the present disclosure may be employed as a tool to facilitate chemical drug discovery.

Thus, in one aspect there is provided a method of identifying compounds capable of binding to a functional conformational state of a protein of interest or protein fragment thereof, said method comprising the steps of:
(a) Binding a function-modifying isolated antibody fragment of the invention to the target protein of interest or a fragment thereof to provide an antibody-constrained protein or fragment, wherein the isolated antibody fragment has binding kinetics with the protein or fragment which are such that it has a low dissociation rate constant,
(b) Providing a test compound which has a low molecular weight,
(c) Evaluating whether the test compound of step b) binds the antibody constrained protein or fragment, and
(d) Select a compound from step c) based on the ability to bind to the protein or fragment thereof.

In one embodiment, the method comprises a further step of evaluating binding of an analogue of a compound selected in step d) for binding to an antibody constrained protein or fragment prepared in step a).

In one embodiment, the method further comprises the step of performing synthetic chemical methods to modify or elaborate a first test compound selected in step d).

In one embodiment, the method comprises a further step of generating three-dimensional structural information, for example employing X-ray crystallography between step c) and step d), or following step d) to gain structural information on binding of a test compound.

In one embodiment, the antibody-constrained protein or fragment is used to generate three-dimensional structural information, for example employing X-ray crystallography in the presence of a bound compound identified in step c) and optionally comprises the further step of performing computation modelling based on the three-dimensional structural information obtained therefrom.

3D Structural Representation

In one embodiment a three-dimensional structural representation of at least one such isolated antibody fragment in complex with the target protein is subsequently generated in order to obtain information about where the isolated antibody fragment is binding the target protein, the functional conformational state of the target protein and which amino acid residues and hence atoms on the target protein and the isolated antibody fragment are in contact with each other or interact with each other. This may be done prior to step a) or step b) if desired.

The functional conformational state revealed by the structural analysis may be previously known or unknown. In one example the functional conformational state revealed by the antibody-target protein structural analysis is new. In one example the structural analysis of the antibody-target protein reveals previously occluded structural features that are not available in the unconstrained protein. In one example these previously occluded structural features may be suitable targets for small molecule binding.

In some embodiments, the invention provides the use of an isolated antibody fragment of the invention to identify functional binding sites for small molecules on proteins and/or to define biologically relevant conformations; conformations in which proteins are either binding or signalling incompetent, which stabilise the complex, or which induce signalling.

Any suitable method known in the art can be used to generate the three dimensional structural representation of an antibody:target protein complex. Examples of such methods include X-ray crystallography, NMR (Nuclear Magnetic Resonance) spectroscopy and hydrogen deuterium mass spectrometry in solution. Preferably X-ray crystallography is used. As set out herein above, the target protein may be the mature protein or a suitable fragment or derivative thereof.

Chemical Compound Screening

In the method of the present invention, candidate compounds, compound fragments or isolated antibody fragments may each be tested for their effect on the biological activity of the target protein. For example, the isolated antibody fragments and compounds identified which bind to the target protein may be introduced via standard screening formats into biological assays to determine the inhibitory or stimulatory activity of the compounds or isolated antibody fragment, or alternatively or in addition, binding assays to determine binding or blocking, such as ELISA, BIAcore, Protein X-ray crystallography and NMR-based screening may be appropriate, alternatively or additionally the ability of the isolated antibody fragment or the compound to induce structural alterations may be identified using for example FRET based assays as described in WO2014/001557.

Thus, in some embodiments, the invention provides the use of an isolated antibody fragment of the invention to screen in vitro for new chemical matter at specific functional sites on a protein by techniques such as Förster resonance energy transfer/fluorescence resonance energy transfer (FRET).

In the method of the present invention, the ability of test compound fragments to bind the isolated antibody fragment constrained protein is determined. In one example the compound selected in step (d) of the method does not bind the unconstrained target protein. In one example the compound selected in step (d) of the method does not bind the isolated antibody fragment in the absence of target protein. In one example the compound selected in step (d) of the method does not bind the unconstrained target protein or the isolated antibody fragment alone. In one example of the method of the present invention step c) further comprises evaluating whether the test compound of step b) binds the protein or fragment in the absence of isolated antibody fragment and step (d) further comprises selecting a compound from step c) based on the ability of the test compound to only bind the isolated antibody fragment—constrained protein or fragment and not the unconstrained protein or fragment.

Typically, in later screening stages following further elaboration of the compound fragments identified by the method and once the potency of the chemical compound has reached an appropriate level the target protein binding is sufficient to allow the compound to bind the target protein in the absence of the isolated antibody fragment.

“Comprising” in the context of the present specification is intended to mean including. Where technically appropriate, embodiments of the invention may be combined. Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present disclosure is further described by way of illustration only in the following examples, which refer to the accompanying Figures, in which:

### BRIEF DESCRIPTION OF THE FIGURES

FIG. 1: SPR single-cycle kinetics of PGT121 Fab-knob domain fusion proteins binding to C5. Vertical axis: RU (Refractive Index Unit); horizontal axis: time in seconds.

FIG. 2: Chromatogram showing purification of the K57 knob domain peptide from the 645 Fab and TEV protease proteins, by hydrophobic interaction chromatography. Vertical axis: AU (Arbitrary Unit); horizontal axis; time in minutes.

FIG. 3: SPR single-cycle kinetics of knob domain binding to C5 proteolytically cleaved from the 645 Fab. Vertical axis: RU (Refractive Index Unit); horizontal axis: time in seconds.

FIG. 4: Sensorgrams from SPR single-cycle kinetics showing the K8 and K92 knob domain peptides binding to mouse and rabbit C5. Vertical axis: RU (Refractive Index Unit); horizontal axis: time in seconds.

FIG. 5: Complement Activation ELISAs. Vertical axis: inhibition of Complement activation in %; horizontal axis: concentration of knob domain peptide in nM. C5b n/e: C5b neoepitope.

FIG. 5A: K8 inhibition of Classical Pathway; FIG. 5B: K8 inhibition of Alternative Pathway;

FIG. 5C: K57 inhibition of Classical Pathway; FIG. 5D: K57 inhibition of Alternative Pathway;

FIG. 5E: K92 inhibition of Classical Pathway; FIG. 5F: K92 inhibition of Alternative Pathway;

FIG. 5G: K149 inhibition of Classical Pathway; FIG. 5H: K149 inhibition of Alternative Pathway.

FIG. 6: Example curves from the Alternative and Classical pathway bacterial killing assays. Vertical axis: Survival of Escherichia Coli in %; horizontal axis: concentration in micromolar of the knob domain peptide. FIG. 6A: K8, K57 and K92 assessed in the Classical Pathway bacterial killing assay; FIG. 6B: K57, K92 and K8 assessed in the Alternative Pathway bacterial killing assay.

FIG. 7: Binding of synthetic knob domain peptides to C5 by single-cycle kinetics. Vertical axis: RU (Refractive Index Unit); horizontal axis: time in seconds.

FIG. 8: Vertical axis: inhibition of Complement activation in %; horizontal axis: concentration of knob domain peptide in nM. FIG. 8A: example curves for chemically derived knob domain peptides in the Classical Pathway C5b neoepitope ELISA. FIG. 8B: example curves for chemically derived knob domain peptides in the Alternative Pathway C5b neoepitope ELISA.

FIG. 9: Crystal structure of the K8 peptide in complex with C5. The K8 peptide is shown with a mesh surface.

FIG. 10: The K8 peptide interacts with the MG8 domain of C5. The MG8 domain of C5 is shown in isolation with the K8 peptide. Certain important K8 residues which participate in H-bond or salt bridge interactions are shown.

FIG. 11: The K8 peptide interacts with the MG8 domain of C5. The MG8 domain of C5 is shown in isolation with the K8 peptide. Certain important C5 residues which participate in H-bond or salt bridge interactions are shown.

FIG. 12: The disulphide bond arrangements for the K8 peptide.

FIG. 13: Vertical axis: inhibition of Complement activation in %; horizontal axis: concentration of knob domain peptide in nM. FIG. 13A: example curves for hC3nb1-K57 constructs in the Alternative Pathway C5b neoepitope ELISA. FIG. 13B: example curves for hC3nb1-K57 in the Classical pathway C5b neoepitope ELISA.

FIG. 14: Bovine ultralong CDR-H3 sequence features and numbering system: sequence alignment of BLV1H12 with the germline encoded VH_{BUL}, D_{H2}, J_{H1} segments. The Cysteine residues are in bold, the conserved Cysteine at position 92 Kabat in the VH_{BUL} segment (H92), the conserved Tryptophan at position 103 Kabat in the J_{H1} segment (H103), and the conserved Cysteine at the start of D_{H2}, are in bold and in a rectangle.

FIG. 15: Two different views of the crystal structure of the Human serum albumin, neonatal Fc receptor (FcRn), human IgG1 Fc (single chain only) ternary complex (PDB accession code: 4N0F). The human serum albumin residues, distal to the interface with FcRn, which have been selected as insertion sites for the K57 and K92 knob domain peptides are highlighted (Alanine 59, Alanine 171, Alanine 364, Aspartic acid 562).

FIG. 16: Crystal structure of the human serum albumin, neonatal Fc receptor (FcRn), human IgG1 Fc (single chain, CH2 and CH3 domains only) ternary complex (PDB accession code: 4N0F). Residues on the Fc, distal to the interaction with FcRn, were selected as sites for engineering of the K149 knob domain peptide (Alanine 327, Glycine 341, Asparagine 384, Glycine 402).

FIG. 17: Phage display of ultralong CDR-H3 as assessed by ELISA. Horizontal axis: Optical Density OD 630 nm; Vertical axis; biotinylated C3, biotinylated C5, C5, C3, anti-myc used to assess binding. The constructs used were hC3nb1-K149 CDR-H3, hC3nb1-K92 CDR-H3, hC3nb1-K57 CDR-H3, hC3nb1-K8 CDR-H3, hC3nb1 (without any insertion).

FIG. 18: Complement Inhibition ELISA data for CP and AP driven assays for K8_{chem}FE, K57_{chem}FE, and K92_{chem}FE. Vertical axis: inhibition of Complement activation in %; horizontal axis: Log concentration of knob domain peptide in nM. FIG. 18A: CP ELISA. FIG. 18B: AP ELISA.

FIG. 19: Haemolysis assays specific for either alternative (AP) or classical pathway (CP) activation for K8_{chem}FE, K8_{chem}FEcyclic, K57_{chem}FE, and K92_{chem}FE, RA101295-14 and His-SOBI002. Vertical axis: Inhibition of haemolysis (in %); horizontal axis: Log concentration of knob domain peptide in nM. FIG. 19A: CP Haemolysis assay. FIG. 19B: AP Haemolysis assay.

FIG. 20: Plasma stability. Vertical axis: concentration of knob domain in plasma (in ng/mL); horizontal axis: time (hours). FIG. 20A: K57_{chem}FE. FIG. 20B: K57_{chem}FE-Palmitoyl. FIG. 20C: K8_{chem}FE.

FIG. 21: In vivo pharmacokinetics following intravenous dosing to Sprague Dawley rats for K8_{chem}FE, K57_{chem}FE and K57_{chem}FE-Palmitoyl. Vertical axis: concentration of knob domain (in ng/mL); horizontal axis: time (hours).

FIG. 22: Crystal structure of the K92 peptide in complex with C5. FIG. 22A: K92 peptide in complex with C5. FIG. 22B: cysteine arrangements for the K92 peptide. FIG. 22C: position of mutations of K92.

### EXAMPLES

### Example 1: Generation of Bovine Antibody Fragments from Immunisation of Cows with the C5 Component of the Complement and Biological Activity

As described below, cows were immunised with C5, immune material was then isolated and cell sorting of antigen-specific memory B-cells was performed from a draining lymph node, taken proximal to the site of immunisation. Flow cytometry was used to identify memory B-cells which were double-positive for two fluorescently labelled populations of C5, and a polyclonal mixture of antigen enriched B-cells was collected.

1. Immunisation of Holstein Friesians with Complement C5 and Isolation of Antigen-Specific Memory B-Cells

Two adult Friesian cows were immunised with Complement C5 (recombinant full-length C5 protein, obtained from example from CompTech). Three sub-cutaneous injections were made into the shoulder at one-month intervals, with 1.25 mg of C5, mixed 1:1 (v/v) with Adjuvant Fama (GERBU Biotechnik). A fourth injection into the shoulder was performed three weeks later with 1.25 mg of C5, emulsified 1:1 with Freund's complete adjuvant (Sigma). A final injection into the shoulder was performed a month later, with 1.25 mg of C5, emulsified 1:1 with Montanide (Seppic). Serum bleeds was taken ten days after each injection to check the serum antibody titre.

Harvesting of Immune Material

500 mL samples of whole blood were taken, post-immunisation with C5. PBMCs were isolated using Leucosep tubes (Griener Bio-one), as per the manufacturer's instructions. In addition, a draining lymph node from the neck, adjacent to the site of immunisation, and a portion of spleen were collected. The tissues were homogenised using a gentle MACS tissue dissociator (Miltenyi), passed through a 40 μm cell strainer, and collected in RPMI 10% Foetal calf serum. Cells were frozen in foetal calf serum, 10% DMSO.

Sorting of Antigen-Specific Memory B-Cells by Flow Cytometry

A sample of draining lymph node was thawed at 37° C. and re-suspended in warm RPMI, 10% FCS (v/v), 1 mM EDTA. The cells were centrifuged for 5 minutes at 400 g and the supernatant removed. The cell pellet was disrupted and resuspended in Assay Buffer (AB) comprising PBS, 1 mM EDTA, 1% BSA (w/v), 25 mM Hepes, at room temperature. The cells were centrifuged as before and resuspended in 2 mL ice-cold AB containing 2 μg/mL each of C5-AF488 (Alexa Fluor 488 fluorochrome) and C5-AF647 (Alexa Fluor 647 fluorochrome), and incubated for minutes on ice. The cells were then centrifuged, the supernatant removed, and the pellet washed in ice cold AB. An aliquot was taken for counting. The cells were centrifuged again, the supernatant removed, and the cells resuspended to 5×10⁶/mL in ice-cold AB before filtering through a 40 μm mesh. DAPI was added at a final concentration of 1 μg/mL just before acquisition on a BD Biosciences FACSAria III (San Jose) cell sorter. Cells were identified by forward and side scatters and DAPI positive dead cells were removed from the analysis. Single cells were identified by pulse processing of height and area scatter parameters. Cells positive for both C5-AF488 and C5-AF647 were then identified and sorted into 1.5 mL eppendorfs containing 1 mL PBS, 20% FCS, 25 mM Hepes kept at 4° C.

The AF488 was excited by a 488 nm laser and collected through a 530/30 BP filter and the AF647 was excited by a 640 nm laser and collected through a 660/20 BP filter. DAPI was excited by a 407 nm laser and collected through a 450/40 BP filter.

2. Deep Sequencing of C5 Enriched CDR-H3 Library Revealed Ultralong CDR-H3 Clonotypes.

Similar to camelid VHH1 discovery from heavy chain only antibodies, there is no requirement to pair heavy (HC) and light (LC) chains, since the extended CDR-H3 knob domain contains the antigen-binding components; consequently, sequencing of polyclonal libraries of antigen-enriched CDR-H3 was used as a rapid approach to discover knob domains.

The antigen enriched pool of memory B-cells were lysed, and RT-PCR was performed directly on the lysate. A primary polymerase chain reaction (PCR) with primers flanking CDR-H3, annealing to the conserved framework 3 and framework 4 of the VH, was used to amplify all CDR-H3 sequences, irrespective of their length or amino acid sequence. A second round of PCR was used to barcode the CDR-H3 sequences for ion torrent sequencing.

Methods:

RT PCR on B-Cell Lysate

The C5 specific memory B-cells from the FACS were pelleted by centrifugation at 10,000 g in a 4° C. centrifuge. The cells were resuspended and lysed with 120 μL of an ice-cold solution of NP-40 detergent (0.5% v/v) and RNasin (Promega) at 1 U/μL. An RT PCR mix was prepared using Super Script IV vilo Master Mix (Invitrogen), comprising 32 μL of cell lysate and 8 of Master Mix. The reaction mix was incubated at 25° C. for 10 minutes, 50° C. for a further 10 minutes and, finally, heated to 85° C. for 5 minutes.

Primary PCR

A primary PCR was used to specifically amplify IgG CDR3 cDNA sequences. The forward primer anneals to the conserved framework 3 sequence of the variable domain of the heavy chain, VH, and the reverse primer sequence anneals to the conserved framework 4 VH sequence. The PCR product when read from 5′ to 3′ therefore encodes the CDR3 sequence irrespective of length, amino acid sequence, or composition of V-, D-, J-gene segments. The PCR mix was prepared using a Hot-start KOD master mix kit (Merck Millipore), as per the manufacturer's instructions. The primers used were 5′-GGACTCGGCCACMTAYTACTG-3′ (SEQ ID NO: 446) and 5′-GCTCGAGACGGTGAYCAG-3′ (SEQ ID NO: 447) and 2 μL of cDNA template was used per 50 μL PCR. The reaction mix was heated for 2 minutes at 96° C. and then subjected to thirty cycles of: 96° C. for 30 seconds, 55° C. for 30 seconds and 68° C. for 60 seconds. Finally, the mix was heated at 68° C. for 5 minutes.

Gel Purification

The PCR product contained a polyclonal mixture of CDR-H3 sequences, comprising regular and ultralong CDR-H3, which were visualised on an analytical gel. An excision was taken with spanning approximately 250-500 bp, based on the marker. A QiaQUICK gel extraction kit (Qiagen) was used to extract the DNA from the excised portion of the gel, as per the manufacturer's instructions.

Barcoding PCR and Gel Purification

A secondary PCR was performed to barcode sequences for ion torrent sequencing. The primers were as used before but with the addition of adaptor (italics) and barcoding sequence (bold):

(SEQ ID NO: 448)
5′CCATCTCATCCCTGCGTGTCTCCGACTCAGTAAGGAGAACGGACTCG
GCCACMTAYTACTG-3′
and
(SEQ ID NO: 449)
5′-CCTCTCTATGGGCAGTCGGTGATGCTCGAGACGGTGAYCAG-3′.

The secondary PCR was performed using a KOD Master Mix kit, as described for the primary PCR. The secondary PCR product was gel purified and concentrated. Finally, the samples were purified using Beckman coulter AMP magnetic beads, as per the manufacturer's instructions.

Ion Torrent Deep Sequencing of CDR3 Libraries

The purified DNA sample was diluted to 20 ng/μL (1 μg total) and stored at −20° C. Deep sequencing of all samples was performed using an Ion Torrent PGM technology commercial service by MACROGEN on a 318 chip.

Briefly, approximately 1.1 Gb of data was obtained per chip, which translates to 6613415 raw reads of a 170 bp mean length. The first processing step consisted of conversion of the FASTQ to FASTA followed by demultiplexing of the later to sort the sequences according to their barcodes and to generate individual FASTA files. Each FASTA file was then translated in all three reading frames and concatenated into one file containing all frames. Using Perl scripts, only sequences of interest, flanked between conserved FR3 and FR4 protein sequences (DSATYY and LL[V,I]TVSS motifs were used), were kept in a single file. Ultimately, all sequences were exported to excel files with frequencies: the number of copies of each sequence found. The number of meaningful reads, after processing, for each sample was found to be 680000 and 530000 for barcodes 1 and 2 respectively.

Results:

Deep sequencing of the CDR-H3 library revealed ultralong CDR-H3 sequences at 4.3 percent of the total sequences. Ultralong CDR-H3 were easily identified by their length (>90 bp) and by a characteristic duplication of the IGHV1-7 gene segment, which has been reported as a universal feature of ultralong CDR-H3. After filtering, 3559 unique CDR-H3 sequences were obtained from the single draining lymph node sample. Of these, 154 were ultralong CDR-H3, the complete list is shown in Table 4.

TABLE 4
Ultralong CDR-H3 sequences derived from cows immunised with C5

| K | SEQ | |
|---|---|---|
| reference | ID | |
| number | NO: | Full Sequence |
| 149 | 1 | TSVLQSTKPQKSCPDGFSYRSWDDFCCPMVGRCLAPRNTY |
| | | TTEFTIEA |
| 152 | 2 | TSVLQSTKPQKSCPDGFSYRSWDDFLLSYGWECLAPRNTY |
| | | TTEFTIEA |
| 147 | 3 | VTVHQQTKRTCPRGYEYVSCWWGATCTYGGRCSGSRDD |
| | | GSLTYEFHVDA |
| 148 | 4 | VTVHQQTKRTCPRGYEYVSCWWGATCTYGGRCSAVGD |
| | | DGSLTYEFHVDA |
| 142 | 5 | TTVHQEPKKSCPEGYTYVWGCDDDSGGVGYGCAPNGAS |
| | | SCSFTYTYEFHIDA |
| 143 | 6 | TTVHQETKKSCPEGYTYVWGCDDDSGGVGYGCAPNGAS |
| | | SCSFTYTYEFHIDA |
| 132 | 7 | TTVHQRTLHNRNCPDGYGYQRHCTVGEDCTERCCDNYG |
| | | LCTSYTDTYTYEFNVNA |
| 133 | 8 | TTVHQRTLKNRNCPAGYGYQRHCTVGEDCTDSCCDRYG |
| | | LCTTSTETYTYEFNVDA |
| 140 | 9 | TAVHQRTKRTCPEGLVYNSDQSRCCAADSGVCWEYWRG |
| | | ERVTRGFTYEWYVEA |
| 141 | 10 | TAVHQRTKRTCPEGLIYNSDQSRCCAADSGVCWEYWRGE |
| | | RVTRGFTYEWYVEA |
| 126 | 11 | TTVHQQTHKKRSCPANHSVRDMCSYGPDDCGRSCCTDGI |
| | | YVRRGSCSSAYEFHVDA |
| 129 | 12 | TTVHQQTHKKRSCPENHSVRDMCSYGPDDCGRSCCTDGI |
| | | YVRRGSCSSAYEFHVDA |
| 92 | 13 | SIVHQKAHTSVTCPEGWSECGVAIYGYECGRWGCGHFLN |
| | | SGPNISPYVSTHKYEWYVDA |
| 93 | 14 | SIVHQKTQTSEGCPEGWSECGVGTYGYDCGRWGCGHYL |
| | | NTGPLISGYVTTNKYEWHVEA |
| 94 | 15 | STVHQKAHTSVACPEGWSECGVAIYGYDCGRWGCGHFL |
| | | NSGPNISPYVTTDAYEWYVDA |
| 95 | 16 | SIVHQRTQTSKGCPEGWNDCGGNTYGYDCGRWGCGHYL |
| | | NSGPRISAYQTTYNYEWYVDA |
| 97 | 17 | TIVHQKTQTREGCPEGWNECGEAIYGYDCGRWGCGHFL |
| | | NTGPRISGYVTTYSYEWFVDT |
| 98 | 18 | SIVHQKTQTSKGCPEGWNDCGVNIYGYDCGRWGCGHFL |
| | | NSGPRISAYQTTYNYEWYVDA |
| 99 | 19 | SIVHQRTQTRTGCPEGWNDCGRNTYGYDCGRWGCGHFL |
| | | NSGPRISDYLTTYNYEWYVDA |
| 100 | 20 | SIVHQKAHTSVTCPEGWSECGVAIYGYECGRWGCGHFLN |
| | | SGPNISPYVTTDAYEWYVDA |
| 101 | 21 | STVHQKAHTSVACPEGWSECGVAIYGYDCGRWGCGHFL |
| | | NSGPNISPYVSTHKYEWYVDA |
| 102 | 22 | STVHQKAHTSVACPEGWSECGVAIYGYDCGRWGCGHFL |
| | | NSGSKYQSYVTTDAYEWYVDA |
| 103 | 23 | TTVHQKAHTSVACPEGWSECGVAIYGYDCGRWGCGHFL |
| | | NSGPNISPYVTTDAYEWYVDA |
| 104 | 24 | SIVHQKAHTSVACPEGWSECGVAIYGYDCGRWGCGHFLN |
| | | SGPNISPYVTTDAYEWYVDA |
| 107 | 25 | SIVHQKAHTSVTCPEGWSECGVAIYGYDCGRWGCGHFLN |
| | | SGPNISPYVTTDAYEWYVDA |
| 108 | 26 | SIVHQKTQTSEGCPEGWSECGVGTYGYDCGRWGCGHYL |
| | | NTGPLISGYVTTNKYEWHVDA |
| 109 | 27 | STVHQKAHTSVACPEGWSECGVAIYGYECGRWGCGHFL |
| | | NSGPNISPYVSTHKYEWYVDA |
| 110 | 28 | SIVHQRTQTSKGCPEGWNDCGGNTYGYDCGRWGCGHFL |
| | | NSGPNISPYVTTDAYEWYVDA |
| 115 | 29 | TTIQQLTERTCPEGSMLGSECNSHWSCEGCDCAKHCTW |
| | | GGRCVDCSPYMSTHEWHIET |
| 119 | 30 | TTIQQSTERTCPEGSMLGSECNSHWSCEACDCARHCTW |
| | | GGRCVDCSPYMSTYEWHIET |
| 27 | 31 | TSVYQKTDTIRHPCRDDSSYACVCRWTRGCSGTDCSGCT |
| | | PDSDIDYGCDTIACNYTYQLYVDA |
| 32 | 32 | TTVYQKTDTKKHPCRDDSSYACVCRWTRGCSGTDCSGC |
| | | TPDSDIDYGCDTIACNYTYQLYVDT |
| 44 | 33 | TTVVPENRHKKHPCRDDSSYACVCRWTRGCSGTDCSGC |
| | | TPDSDIDYGCDTIACNYTYQLYVDT |
| 52 | 34 | TTVHQHSNNKKTCPDGTSSHSACILGTGGCCLDQYYRRG |
| | | ICGRVDACYEYSSSVNYEWYVDA |
| 54 | 35 | TTVHQHTNNKKTCPDGSSSHSACKLGTGGCCLDGYYRR |
| | | GICGRVDACYEYSSSVNYEWYVDA |
| 83 | 36 | ATVHQRTERSCPDGSSDAESGVCSGCCRGWDCCSFEVD |
| | | WVGCKGCTAYTYRTVYEHHVDA |
| 85 | 37 | ATVHQRTERSCPDGSSDAESGVCSGCCRGWDCCSFEVD |
| | | WVGCKGCTAYTYRTIHEHHVDA |
| 87 | 38 | ATVHQRTERSCPDGSSDAESGVCSGCCRGWDCCSFEVD |
| | | WVGCKGCTAYTYRSIYEHHVDA |
| 89 | 39 | VTVHQRAERTCPDGSSDAESGVCSGCCGGWDCCSFKVD |
| | | WVGCKECTAYPYNTRYEHHVDA |
| 13 | 40 | TTVHQQTKTKKNPCRDVASPVCVCRWAEGCSGTDCSECT |
| | | PDPDRDYGTCEIIACTHTYELHVDA |
| 14 | 41 | TTVHQKTKTKKNPCRDVTSPVCVCRWAEGCSGTDCSDCT |
| | | PDPDRDYGTCELIACTHAYELHVDA |
| 20 | 42 | TTVHQKTKTKKNPCRDVTSPVCVCRWAEGCSGTDCSDCT |
| | | PDPDRDYGTCEIIACTHTYELHVDA |
| 29 | 43 | TTVIQKTATKQSCPDDYRDGGECCIYGRCSAEDCSVTGW |
| | | EYYGSTLCRVPYITTHAYQWHVDA |
| 34 | 44 | TTVIQKTATKQSCPDDYRDGGECCIYGRCSAEDCSVTGW |
| | | EYYGSTLCRVPYITTHSYQWHVDA |
| 37 | 45 | TTVIQKTATKQSCPDDYRDGGECCIYERCSAEDCSVTGW |
| | | EYYGSTLCRVPYITTLAYQWHVDA |
| 46 | 46 | TTVIQKTATKQSCPDDYRDGGECCIYGRCSAEDCSVTGW |
| | | EYYGSTLCRVPYITTLCLPVARRA |
| 59 | 47 | TTVHQETRRNCPDGYSEINACGDRYKASGGLCCGEGAG |
| | | AWRCWECSDTIIPTTTYEFYVDA |
| 61 | 48 | TTVHQETRRHCPDGYSDIYGCGHYYSATGGHCCGEGAG |
| | | AWRCWECSDTIMPSTTYEFYVDA |
| 62 | 49 | TTVHQETRRNCPDGYSDIYGCGNRYAATGGHCCGEGAG |
| | | AWRCWECSDSIWPSSTYEFYVDA |
| 64 | 50 | STVHQDTRRHCPDGYSDIYACGHYYSATGGHCCGEGAG |
| | | AWRCWECSDTIMPSTTYEFYVDA |
| 65 | 51 | TTVHQESRRHCPDGYSDIYGCGHYYSSTGGHCCGEGAG |
| | | AWRCWECSDTISPSTTYDFHVDA |
| 66 | 52 | STVHQDTRRHCPDGYSDIYGCGHYYSATGGHCCGEGAG |
| | | AWRCWECSDTIMPSTSYEFYVDA |
| 68 | 53 | TTVHQETRRNCPDGYSNIYDCGHYYSSSGGHCCGEGAGA |
| | | WRCWECSDTISPSTTYEFYVDA |
| 71 | 54 | TTVHQETRRSCPDGYSDIYGCGHYYSSTGGHCCGEGAGA |
| | | WRCWECSDTISPRTRYEFAVDA |
| 74 | 55 | TTVHQETRRNCPDGYSDIKGCGNAYAATGGHCCGEGAG |
| | | AWRCWECSDTIAPSSTYEFYVDA |
| 78 | 56 | STVHQETRRSCPDGYSDIYGCGHYYSSTGGHCCGEGAGA |
| | | WRCWECSDTISPSTRYEFYVDA |
| 79 | 57 | TTVRQETRRNCPFGYSDIKGCGNRYAATGGHCCGEGAG |
| | | AWRCWECSDTIRPSSTYEFYVDA |
| 60 | 58 | VIVYQETIKSCREGYIDGGGCCLPGSCRGCACSYYDWLK |
| | | CPRDCRGTSEEYIYTYNFRVDA |
| 77 | 59 | GIVYQETIKSCPEGYIDGGGCCLPGSCRGCACTYYNVLK |
| | | CPRDCRGTSEEYIYRYKFHVDA |
| 10 | 60 | STVHQLTITTLGCPDGVSVVNTCGWLRCNCGDSIYCSRSA |
| | | DSGMWCGRCGDCTSTHTHQWHVDA |
| 11 | 61 | STVHQLTITTLGCPDGVSVVPTCGWLRCNCGEDLYCSRS |
| | | DEQGTWCGRCGDCTSTYTHQWHVDA |
| 18 | 62 | STVHQLTITTVGCPNGVTRVATCGWKRCHCGENIYCSRS |
| | | DDSGTWCGRCGDCTGTYTYQWHVDA |
| 19 | 63 | STVHQLTITTVGCPNGVPRVTTCGWKRCHCGENIYCSRS |
| | | DDSGTWCGRCGDCTGTYTYQWHVDA |
| 21 | 64 | GTVHQLTITTLGCPDGVSVVNTCGWNRCNCGDTTFCSRS |
| | | DDSGTWCGRCGDCSSTHTHQWHVDA |
| 22 | 65 | STVHQLTITTLGCPDGVSVVNTCGWKRCNCGDSIYCSRSA |
| | | DDDGWCGRCGDCTSTHTHQWHVDA |
| 23 | 66 | STVHQLTITTVGCPNGVTRVATCGWKRCHCSENIYCSRS |
| | | DDSGTWCGRCGDCTNTYTFQWHVDA |
| 47 | 67 | TTVHQKTIAKCPDGYTYSGDCGICDDCGGRTSRAYDCAG |
| | | DTSLYMCGRRSPTLLTYQFHVDV |
| 55 | 68 | TTVTPETIAKCPDGYTYSGDCGICDDCGGRTSRAYDCAG |
| | | DTSLYMCGRRSPTLLTYQFHVDV |
| 2 | 69 | ATVHQQTKKQTERSCPDGYTYINDCIGASGAVSRYDCWR |
| | | FRRMNGVCIDGTYSTTADTYTYEFHVDA |
| 3 | 70 | ATVHQQTKKQTERSCPDGYTYIVDCIGATGAVSRYDCWR |
| | | FRRMNGVCIDGTYSTTADTYTYEFHVDA |
| 31 | 71 | TTVHQKTRKSCPGGCRDTDGHDYDHWSCAGSDCCCFGT |
| | | DGGCGRWGIYCSHSYTYTYEYHVET |
| 33 | 72 | TTVHQKTRKSCPGGCRDTDGHDYDHWSCAGSDCCCFGT |
| | | DGGCGRWGVYCSHSYTYTYEYHVDT |
| 45 | 73 | TTVHQKTRKSCPGGCRDTDGHDYDHWSCAGSDCCCFGT |
| | | DGGCGRWGIYCSHSYTYTYEYHVDT |
| 8 | 74 | CTVQQKTHQVCPDGFNWGYGCAAGSSRFCTRHDWCCY |
| | | DERADSHTYGFCTGNRVTNTYEFHADA |
| 9 | 75 | TTVQQKTHQDCPDGFNWGYGCAAGSSLHCARHDWCCY |
| | | DDRVGRDTYGFCTGNRATTTYEFHVDA |
| 1 | 76 | TTVHQKTDQKRSSCPDGYSDCLVCGADRDGCSSGGCRGC |
| | | WTNAYYSSRTYYNTDEFHYKPNEFHVDM |
| 4 | 77 | TSVYQKTTKRFTCHDPSGGTWERADGATSCPGTHCCSYG |
| | | RDGIWHGYDRRRTYTEVFTYELDVEE |
| 5 | 78 | CTVYQKTETKKSCPDGYRFFQECRGTGTGCPGDDCVCY |
| | | DGRGGFRWRNGCTTYTYTYRHNLHVET |
| 6 | 79 | TTVYQETKIMRICPDDERRRWGCSDDSEGCSDSDCHIYD |
| | | GDGSVGCCDGYLNSREIYKYAFHIDA |
| 7 | 80 | VAVHQKTTERYSCPDGYSSCSSCRANDLDCRGVDCVNDR |
| | | VCRGDGGFFSSRGYIVTYNYDFRVDA |
| 12 | 81 | AAVHQETKTLRTCPPGLSDSNACPVGTWASRRTGCCSCC |
| | | DRFCGGYSTCTDYTDTVTYEWHVDT |
| 15 | 82 | TTVHQETKITSPACPDGYFYEYRCLVGGGCGWGCWNAA |
| | | GGRPNAAGSLDRSPIETVTYEFQVDA |
| 16 | 83 | TTVYQKTTKSTCPDGYIADGGCRKAGSWCSSVDCAGYG |
| | | EDGDYGGWRTSCCYFVASAYEFHVDT |
| 17 | 84 | GTVHQQTQEKCPDGYTFTANNCVTSSVRCSGRNCCGGD |
| | | SYGYYIGIGGICHYDYTYTYENYVEA |
| 24 | 85 | TIVHQETNKEKICRVDYVDSATCTWNCDCCRSRKSDCCA |
| | | YANSRSCWNTSGTYTYTYEFHVDA |
| 25 | 86 | TTVHQRTITRCPDDFGNTCRCSKGTCPCGEDACCGTNQ |
| | | YSFWGDCRDVGRTTFIETYEWNVDD |
| 26 | 87 | TTVYQNTRSKERSCPYGTGFDPTWCDSVLPCRRDGCWTT |
| | | VWGCCEGDVDGGETTPTYEFYVDA |
| 28 | 88 | TTVYQKTRSDCPAGYKQVYGCSAGNCGCRGNGCCNSGS |
| | | CGTWSEWGQYGCCNCHSSYEFHVDA |
| 30 | 89 | TTVHQTTRKTQSCPDGYTDIDGCSWRHGCCRYDCCSDRS |
| | | CSWCVDRDWSSYIVTATYELDIEA |
| 35 | 90 | TTVHQETKHTRSCPDGYTDRVGCPYLWTSCARGDCWRID |
| | | RGATANPAATTYTYTDTYDWHIET |
| 36 | 91 | ITAHQKTNKIPHCRDGYDYGGGCCVSSGVYGESCRSSGG |
| | | SDCDQWVGCESVTYTETYEWHVDA |
| 38 | 92 | TSVLQKTRHTCPDGYEYDTACGHGRCCCVGSSCRRNHTY |
| | | GDYRRWGLYNSYSPAYTYEFHVDT |
| 39 | 93 | TAVHQQTERSCPPDTTEHDCCGCGGRGCAWSGCYRKG |
| | | YGTGCRVCTSIQARDYIYTYKLHIDT |
| 40 | 94 | TTVHQNTIRSCPDGTDYAYGCRLGAWGCAGVGCCRGGA |
| | | VGAWGCYGGDTFNTDSYTYEFYVDA |
| 41 | 95 | TAVYQRTEARKSCPDGYNDVEARAHRSECSPNDCLRDGL |
| | | GVASGCAWYRAYILIETYEFYVEA |
| 42 | 96 | TTVYQKTRKLPSCREGTFYHAVCGGVVRCQVVDCDADG |
| | | GCCYNAIGQYFGVSYSYKYEWFVEA |
| 43 | 97 | ATVHQKTNKKQSCPDGYSDDDGRPDHWSCMDVDCWRP |
| | | ARGGWGSNCEHTNYIYTYTYEYHVDA |
| 48 | 98 | TIVHQKTKREERCPAGYSISACRDGIGCGATDCCADGATD |
| | | YAWGWECKSRIYGDSYEFHVDA |
| 49 | 99 | TTTVQRTHKTTSCPDGYHFIEPCHSGLCWREGACNGDGI |
| | | CANGLGRCRTVSETSTYEFYVDA |
| 50 | 100 | TTVYQRAQSKSCPDYCSCIFSYCSGADGCSSYGYCGHGGD |
| | | EGDGFNGGGSRVSYTYEFYVDS |
| 51 | 101 | TTVHQQTRTRCPDDYSYRSRGWIGSDCGGHGCWSDRDA |
| | | RRYDVYGNCNRVGEINTYEWYVDA |
| 53 | 102 | TTVHQRTKKKLVLSVMILMIVVTILILCRVEECCKNGVVNAY |
| | | GICEYAGGSATYTYEWYVDA |
| 56 | 103 | TTVHQKTITSCPDGYVYSYDCGICDDCGGRTSRAYDCAG |
| | | DTSLYMCGRRSPSSAYQFHVDR |
| 57 | 104 | TTVHQRTIKSGCPPGYKSGVDCSPGSECKWGCYAVDGRR |
| | | YGGYGADSGVGSTYTHEFYVDA |
| 58 | 105 | TTVHQRTKKTCPLGYDLNDRCDHFNTCRVEECCKNGVV |
| | | NAYGICEYAGGSATYTYEWYVDA |
| 63 | 106 | TTVHQKTQRPICPDDYTALNGWGCGEYRCCPKSGACCC |
| | | SGGGVHLLQSCSLETKYEFYVSA |
| 67 | 107 | LTVLQVTDRRASCPAGCQDECGSSENCYCFRYGIWCHGR |
| | | YSSGNSGTYSSNGYSSTWYADA |
| 69 | 108 | STVHHEAHKRCPEDYSDRDHCSCWAGCGDDDCWRVVA |
| | | GWRCSNYRYIGASYTHTYDFYADT |
| 70 | 109 | TTVHQKTKKSCPLGYAINDRCDDLKTCGPDECCLNGVVN |
| | | AYGICEYEGESATHTYEWYVDA |
| 72 | 110 | TLVYQKTKKSCPEGYEGAPDCGAFDYCRVDDCCCRSGY |
| | | GSCRRDSCRSGIRTSTYEFYVDT |
| 73 | 111 | TTVYQHTRNRCPDDYRDCGHCCCQYGCHAVGCWRRQG |
| | | GGFERCGEVDSQSPTYMYEFHVDA |
| 75 | 112 | ATVLQYTHKTCPDGYEFGKNCPDGHGCSGSDCWRCDSR |
| | | SAWWCTNYSWTDSIHAYELYVDA |
| 76 | 113 | TTVHQKTEKSCKGGTDCGAGCCADGDPCSSGRCRAWSS |
| | | TLRDYFYYPTSNYTYICDFHIDA |
| 80 | 114 | TIVFQKTTKSCPGVSAEGGVCCSGTACTVPECWWFHQG |
| | | HYSIPGGCTAATYTHTYESHVDA |
| 81 | 115 | TTVHQKTNQEKHCPDGYDYCRVTEDGYCCSAWTCMHW |
| | | RCAPGHKEYSVVSTTYTYEWYVDA |
| 82 | 116 | VTTHQKSRKICPDGCIYACSCREEWRCTVFDCVRPRDVP |
| | | NGRNACVSTCPSTSIYEFRVDA |
| 84 | 117 | STVYQETKRKCPDGYRVGTDCTPGKGCDYACHSRLGVR |
| | | WGGDGRDGGRGYIVSYELHIDA |
| 86 | 118 | TTVYQKTQKPTDGYSCGITCRKRCDCSFVGYCACSESVSG |
| | | DCTCYPRDSIPYRHEWYVDA |
| 88 | 119 | TTIFQKTRRNCPPSSTSDGDCRGGWTCRGGDCSRWRGYY |
| | | SSGNNYCCYDYTDTYEFYVDQ |
| 90 | 120 | TTVRQKTAKSCPWGYDNGHGCNCGNDVFACSECLRSGT |
| | | CSRYGRYEAYSYIVTYEFSVDA |
| 91 | 121 | STVHQKTRQSCPDDYPVKCERGCGRERCGNCGWACNGP |
| | | VGSPTCSYCRPYIYTYEFYVDA |
| 96 | 122 | TTVYQKTKETCPDGYIWAERCPGGWTSCRNACWLEGGD |
| | | SAGAYDEVTSTVHRYEFYVDT |
| 105 | 123 | TAVYQKTEEKNTCPDGHTWRHGYRCTGWSYGCFRGAG |
| | | NDCSDFGGDRITTYGYEWYVEN |
| 106 | 124 | TTVHQRTIRTCPDGYGYQDACGRWGGCVGRACCSSGGS |
| | | GCCDGSCGTMYIDNYDLYVDA |
| 111 | 125 | GIVVQRTYERRTCPDTFTYKDGCRRGGTLLNSRSGCYNV |
| | | YCNYHDAEVTYAHRWYVDA |
| 112 | 126 | VTVQQQTKLEYSCPNGYSSDAGCLAAWRCGDYDCCREN |
| | | AFRPCTGSIPTSNYEWHLEA |
| 113 | 127 | STVHQETIRTCPDGGTYARDCGRECAICGHCGCCQNAY |
| | | RRNWETCNTYTESINFHIDA |
| 114 | 128 | TAVVQRSLKKPSCPSGYTLWGDCEGDDGGEGGVCRCW |
| | | RPHSTVATPTYASTFQWHVDA |
| 116 | 129 | TTVYQKTNTERSCPEWVQTSRTCIYRSRCGQYVCWSLGE |
| | | DDCGVTCTDTTTYEWYVDA |
| 117 | 130 | ITVHQETIRTCPDAWRSSATCRGAYGEAYECCPSGSSMW |
| | | TSCVGCTTATFSYNLYVEA |
| 118 | 131 | TTVHQKTSAKRTCPDGWRPGSECGWEDRCCGEFCSRCD |
| | | WHGGWRAYMETQTYEFNVDS |
| 120 | 132 | STVHQQTNKRRQNCPDGYKYNGFCTPDGGCSRVSSWGW |
| | | DRSCISPTYTYTYEWYVEA |
| 121 | 133 | TTVYQSTRKTSRNCPDGGSPSVQCLDDTWACRIVDCYDD |
| | | GTYGTYRFTNTYDWYVDA |
| 122 | 134 | STVFQHTKTTCSCPDNWETSGDCAGSSGDCSDCTCWRLG |
| | | YGRTSSIATFNYEWYVEA |
| 123 | 135 | TTVHQKTESHRSCPGDRPVDCGDDYGTLGCCPFHVGCG |
| | | TWRCIEHIYTYTYQFHVDA |
| 124 | 136 | TTVYQSTRKTSRNCPDGGSPSVQCLDDTWACRIVDCYATV |
| | | LMVPIVLPTLYDWYVDA |
| 125 | 137 | ATVHQYTHRSCPVGYDGGGNCGRYVDTCWGSDCCRYR |
| | | RGIDYSCSSYSSSYEFYFEA |
| 127 | 138 | ASVHQETKRSCPDGYRRGLECSAEWRCRYYDCVECSYG |
| | | LCGHITRYIESYAWHVDT |
| 128 | 139 | TAVHQETKKQPPNCPDGSSLLSSCFDTGGCSLYSCGREGR |
| | | RRTYTYSYTYEWYVDA |
| 130 | 140 | GTVHQKTNDHTRCPDGYYQGWHMSLRRYVCARDGYNPE |
| | | RYYVEATHTYTYEFHIDA |
| 131 | 141 | TTVHQQTNTKNCPTWCGFAHSCILRYEACSDCDCSGGAG |
| | | DYAAPGLYHTYEFHVDA |
| 134 | 142 | IAVHQETKRSCPGGYIARCAGTYGCSAVPGCCDFSGDCL |
| | | WRADSLTLTYELHVDT |
| 135 | 143 | ATVHQKNNRKKKLVRMVVNLVSSVSTPVKFCRISECYEDHPT |
| | | TIYTYTYEFHVDA |
| 136 | 144 | TTVHQTTNRKKTCPDNYREVDGCDPYDCCLTTWCTNSY |
| | | CTRYIYEDSYEFYVTA |
| 137 | 145 | ATVHQKTTEKKTCPDGGEPSVICLDASEVCRISECYEDHP |
| | | TTIYTYTYEFHVDA |
| 138 | 146 | TTVHQKTKRSCPAYDSSGCGCVYYSPWNACICDKPGGPC |
| | | DGVNPITSYEFNVDA |
| 139 | 147 | TAVYQKTSESQRTCPSWCSLYMCGGYLACSACGCAENGR |
| | | YGNGITYTYEWHVDA |
| 144 | 148 | TTVHQKTTKTCPDGYVYNDPCDCWGRRNYDCCCEGGRE |
| | | FYTFVYSHEFNVHS |
| 145 | 149 | TSVLQSTKKQKSCPDGLSYRAWDDFCCPNVGRCLPPINTY |
| | | TYTHAFHIEA |
| 146 | 150 | TTLYQNTRKKGGCPEGTTYLGGSSETYRCGLEGRMRTYS |
| | | YTYSYEWYVDA |
| 150 | 151 | TSVLQSTKKQKTCPDGLSYRSWDGFCCPKVGRCLPTIDAY |
| | | INQFHIEA |
| 151 | 152 | TSVLQSTKEQKTCPDGLSYRSWDGFCCPKYGRCLAATSTY |
| | | TTEFYIEA |
| 153 | 153 | GAVYQKTNEQSSCPDGWRDTGTHCEDYGSWGYRDYTFTY |
| | | TYEFHVHN |
| 154 | 154 | TTVHQTTRPNTDSCPSGYSTTLHCCCGSWKCDWCDPTTY |
| | | KYELYVNA |

3. Ultralong CDR-H3 can Function Autonomously to Bind to Human Complement Component C5 Independently of Supporting Antibody Infrastructure (Scaffold).

To screen for C5 binding knob domains, from the 154 ultralong CDR-H3 sequences identified, ultralong CDR3 sequences were selected based on clonotype, copy number and cysteine pattern to make a representative set of 52 sequences are described in Table 5.

TABLE 5
52 sequences of ultralong
CDR3 sequences selected for screening

| SEQ | |
|---|---|
| ID | |
| NO: | Sequence |
| 1 | TSVLQSTKPQKSCPDGFSYRSWDDFCCPMVGRCLAPRNTYT |
| | TEFTIEA |
| 3 | VTVHQQTKRTCPRGYEYVSCWWGATCTYGGRCSGSRDDGSL |
| | TYEFHVDA |
| 6 | TTVHQETKKSCPEGYTYVWGCDDDSGGVGYGCAPNGASSCS |
| | FTYTYEFHIDA |
| 9 | TAVHQRTKRTCPEGLVYNSDQSRCCAADSGVCWEYWRGERV |
| | TRGFTYEWYVEA |
| 16 | SIVHQRTQTSKGCPEGWNDCGGNTYGYDCGRWGCGHYLNSG |
| | PRISAYQTTYNYEWYVDA |
| 14 | SIVHQKTQTSEGCPEGWSECGVGTYGYDCGRWGCGHYLNTG |
| | PLISGYVTTNKYEWHVEA |
| 15 | STVHQKAHTSVACPEGWSECGVAIYGYDCGRWGCGHFLNSG |
| | PNISPYVTTDAYEWYVDA |
| 13 | SIVHQKAHTSVTCPEGWSECGVAIYGYECGRWGCGHFLNSG |
| | PNISPYVSTHKYEWYVDA |
| 21 | STVHQKAHTSVACPEGWSECGVAIYGYDCGRWGCGHFLNSG |
| | PNISPYVSTHKYEWYVDA |
| 23 | TTVHQKAHTSVACPEGWSECGVAIYGYDCGRWGCGHFLNSG |
| | PNISPYVTTDAYEWYVDA |
| 29 | TTIQQLTERTCPEGSMLGSECNSHWSCEGCDCAKHCTWGGR |
| | CVDCSPYMSTHEWHIET |
| 31 | TSVYQKTDTIRHPCRDDSSYACVCRWTRGCSGTDCSGCTPD |
| | SDIDYGCDTIACNYTYQLYVDA |
| 34 | TTVHQHSNNKKTCPDGTSSHSACILGTGGCCLDQYYRRGIC |
| | GRVDACYEYSSSVNYEWYVDA |
| 36 | ATVHQRTERSCPDGSSDAESGVCSGCCRGWDCCSFEVDWVG |
| | CKGCTAYTYRTVYEHHVDA |
| 40 | TTVHQQTKTKKNPCRDVASPVCVCRWAEGCSGTDCSECTPD |
| | PDRDYGTCEIIACTHTYELHVDA |
| 43 | TTVIQKTATKQSCPDDYRDGGECCIYGRCSAEDCSVTGWEY |
| | YGSTLCRVPYITTHAYQWHVDA |
| 47 | TTVHQETRRNCPDGYSEINACGDRYKASGGLCCGEGAGAWR |
| | CWECSDTIIPTTTYEFYVDA |
| 48 | TTVHQETRRHCPDGYSDIYGCGHYYSATGGHCCGEGAGAWR |
| | CWECSDTIMPSTTYEFYVDA |
| 49 | TTVHQETRRNCPDGYSDIYGCGNRYAATGGHCCGEGAGAWR |
| | CWECSDSIWPSSTYEFYVDA |
| 58 | VIVYQETIKSCREGYIDGGGCCLPGSCRGCACSYYDWLKCP |
| | RDCRGTSEEYIYTYNFRVDA |
| 60 | STVHQLTITTLGCPDGVSVVNTCGWLRCNCGDSIYCSRSAD |
| | SGMWCGRCGDCTSTHTHQWHVDA |
| 61 | STVHQLTITTLGCPDGVSVVPTCGWLRCNCGEDLYCSRSDE |
| | QGTWCGRCGDCTSTYTHQWHVDA |
| 62 | STVHQLTITTVGCPNGVTRVATCGWKRCHCGENIYCSRSDD |
| | SGTWCGRCGDCTGTYTYQWHVDA |
| 67 | TTVHQKTIAKCPDGYTYSGDCGICDDCGGRTSRAYDCAGDT |
| | SLYMCGRRSPTLLTYQFHVDV |
| 69 | ATVHQQTKKQTERSCPDGYTYINDCIGASGAVSRYDCWRFR |
| | RMNGVCIDGTYSTTADTYTYEFHVDA |
| 71 | TTVHQKTRKSCPGGCRDTDGHDYDHWSCAGSDCCCFGTDGG |
| | CGRWGIYCSHSYTYTYEYHVET |
| 74 | CTVQQKTHQVCPDGFNWGYGCAAGSSRFCTRHDWCCYDERA |
| | DSHTYGFCTGNRVTNTYEFHADA |
| 98 | TIVHQKTKREERCPAGYSISACRDGIGCGATDCCADGATDY |
| | AWGWECKSRIYGDSYEFHVDA |
| 125 | GIVVQRTYERRTCPDTFTYKDGCRRGGTLLNSRSGCYNVYC |
| | NYHDAEVTYAHRWYVDA |
| 105 | TTVHQRTKKTCPLGYDLNDRCDHFNTCRVEECCKNGVVNAY |
| | GICEYAGGSATYTYEWYVDA |
| 85 | TIVHQETNKEKICRVDYVDSATCTWNCDCCRSRKSDCCAYA |
| | NSRSCWNTSGTYTYTYEFHVDA |
| 144 | TTVHQTTNRKKTCPDNYREVDGCDPYDCCLTTWCTNSYCTR |
| | YIYEDSYEFYVTA |
| 149 | TSVLQSTKKQKSCPDGLSYRAWDDFCCPNVGRCLPPINTYT |
| | YTHAFHIEA |
| 103 | TTVHQKTITSCPDGYVYSYDCGICDDCGGRTSRAYDCAGDT |
| | SLYMCGRRSPSSAYQFHVDR |
| 87 | TTVYQNTRSKERSCPYGTGFDPTWCDSVLPCRRDGCWTTVW |
| | GCCEGDVDGGETTPTYEFYVDA |
| 145 | ATVHQKTTEKKTCPDGGEPSVICLDASEVCRISECYEDHPT |
| | TIYTYTYEFHVDA |
| 104 | TTVHQRTIKSGCPPGYKSGVDCSPGSECKWGCYAVDGRRYG |
| | GYGADSGVGSTYTHEFYVDA |
| 88 | TTVYQKTRSDCPAGYKQVYGCSAGNCGCRGNGCCNSGSCGT |
| | WSEWGQYGCCNCHSSYEFHVDA |
| 77 | TSVYQKTTKRFTCHDPSGGTWERADGATSCPGTHCCSYGRD |
| | GIWHGYDRRRTYTEVFTYELDVEE |
| 109 | TTVHQKTKKSCPLGYAINDRCDDLKTCGPDECCLNGVVNAY |
| | GICEYEGESATHTYEWYVDA |
| 78 | CTVYQKTETKKSCPDGYRFFQECRGTGTGCPGDDCVCYDGR |
| | GGFRWRNGCTTYTYTYRHNLHVET |
| 117 | STVYQETKRKCPDGYRVGTDCTPGKGCDYACHSRLGVRWGG |
| | DGRDGGRGYIVSYELHIDA |
| 122 | TTVYQKTKETCPDGYIWAERCPGGWTSCRNACWLEGGDSAG |
| | AYDEVTSTVHRYEFYVDT |
| 86 | TTVHQRTITRCPDDFGNTCRCSKGTCPCGEDACCGTNQYSF |
| | WGDCRDVGRTTFIETYEWNVDD |
| 153 | GAVYQKTNEQSSCPDGWRDTGTHCEDYGSWGYRDYTFTYTY |
| | EFHVHN |
| 89 | TTVHQTTRKTQSCPDGYTDIDGCSWRHGCCRYDCCSDRSCS |
| | WCVDRDWSSYIVTATYELDIEA |
| 126 | VTVQQQTKLEYSCPNGYSSDAGCLAAWRCGDYDCCRENAFR |
| | PCTGSIPTSNYEWHLEA |
| 150 | TTLYQNTRKKGGCPEGTTYLGGSSETYRCGLEGRMRTYSYT |
| | YSYEWYVDA |
| 91 | ITAHQKTNKIPHCRDGYDYGGGCCVSSGVYGESCRSSGGSD |
| | CDQWVGCESVTYTETYEWHVDA |
| 93 | TAVHQQTERSCPPDTTEHDCCGCGGRGCAWSGCYRKGYGTG |
| | CRVCTSIQARDYIYTYKLHIDT |
| 133 | TTVYQSTRKTSRNCPDGGSPSVQCLDDTWACRIVDCYDDGT |
| | YGTYRFTNTYDWYVDA |
| 76 | TTVHQKTDQKRSSCPDGYSDCLVCGADRDGCSSGGCRGCWT |
| | NAYYSSRTYYNTDEFHYKPNEFHVDM |

Expression of Knob-TEV-HKH-ScFc Fusion Proteins:

We took entire ultralong CDR-H3, encompassing H93-H102 (Kabat), and expressed them recombinantly with a single-chain Fc (ScFc) tag at the C-terminus, and expressed as CDR-H3-ScFc fusions as 2 mL transient transfections of Expi293F cells. Standard methods were used to clone the sequences into a vector useful for mammalian expression.

To the CDR-H3 sequences described in Table 5, a C-terminal tag comprising Gly-Ser linkers (italics), a TEV protease cleavage site (underlined), a 10× poly-histidine sequence and a single chain Fc was appended.

The seven-amino-acid recognition site for TEV protease is Glu-Asn-Leu-Tyr-Phe-Gln-Gly with cleavage occurring between Gln and Gly.

The sequence of the scFc sequence is as follows (CH2-CH3-linker in italic-CH2-CH3):

(SEQ ID NO: 155)
PKSGDKTHTSPPCPAPELLGGPSVFLFPPKPKDTLMISRTPEVTCVV
VDVSHEDPEVKFNWYVDGVEVHNAKTKPREEQYNSTYRVVSVLTVLH
QDWLNGKEYKCKVSNKALPAPIEKTISKAKGQPREPQVYTLPPSRDE
LTKNQVSLTCLVKGFYPSDIAVEWESNGQPENNYKTTPPVLDSDGSF
FLYSKLTVDKSRWQQGNVFSCSVMHEALHNHYTQKSLSLSPGKGGSS
TASGSGSGGSGTAGSSGGAGSSGGSTTAGGSASGSGSTGSGTGGASS
GGASGASGEPKSSDKTHTSPPCPAPELLGGPSVFLFPPKPKDTLMIS
RTPEVTCVVVDVSHEDPEVKFNWYVDGVEVHNAKTKPREEQYNSTYR
VVSVLTVLHQDWLNGKEYKCKVSNKALPAPIEKTISKAKGQPREPQV
YTLPPSRDELTKNQVSLTCLVKGFYPSDIAVEWESNGQPENNYKTTP
PVLDSDGSFFLYSKLTVDKSRWQQGNVFSCSVMHEALHNHYTQKSLS
LSPGK

The sequence of the C-terminal Tag is shown below:

(SEQ ID NO: 156)
GSGSGSGSGSENLYFQGSGSHHHHHHHHHHGSGSPKSGDKTHTSPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSRDELTKNQVSLTCLVK
GFYPSDIAVEWESNGQPENNYKTTPPVLDSDGSFFLYSKLTVDKSRW
QQGNVFSCSVMHEALHNHYTQKSLSLSPGKGGSSTASGSGSGGSGTA
GSSGGAGSSGGSTTAGGSASGSGSTGSGTGGASSGGASGASGEPKSS
DKTHTSPPCPAPELLGGPSVFLFPPKPKDTLMISRTPEVTCVVVDVS
HEDPEVKFNWYVDGVEVHNAKTKPREEQYNSTYRVVSVLTVLHQDWL
NGKEYKCKVSNKALPAPIEKTISKAKGQPREPQVYTLPPSRDELTKN
QVSLTCLVKGFYPSDIAVEWESNGQPENNYKTTPPVLDSDGSFFLYS
KLTVDKSRWQQGNVFSCSVMHEALHNHYTQKSLSLSPGK

Plasmid DNA for each construct was amplified using miniprep kits (Qiagen). Individual 2.0 mL Expi293F cell cultures, at 3×10⁶ cells/mL, per construct, were set up in 48-well culture blocks using Expifectamine 293 Transfection kits (Invitrogen), as per the manufacturer's instructions. The cells were cultured for four days and centrifuged at 2500 rpm for 30 minutes.

C5 Binding ELISA:

Cell supernatants were screened in a C5 binding ELISA according to the following method: 96-well ELISA plates (Nunc Maxisorp) were coated with a 2 μg/mL solution of C5, in carbonate-bicarbonate buffer (Sigma). All washing steps comprised four wash cycles with PBS, 0.05% Tween 20. Blocking Buffer was PBS, 1% BSA (w/v). Cell Supernatants were plated as 1:10 and 1:100 dilutions in Assay Buffer (PBS, 0.05% Tween 20, 0.1% BSA [w/v]). To reveal, a 1/5,000 dilution of a goat anti-human Fc, HRP (Thermo Scientific) secondary antibody was used with ‘One-Step’ 3,3′,5,5′ Tetramethylbenzidine (Thermo Scientific). The reaction was stopped with the addition of a 2% (w/v) NaF solution and the OD measured at 630 and 390 nm wavelengths using a BMG labtech plate reader.

Results:

14 C5 binders were identified. Hit sequences are displayed in Table 6.

TABLE 6

| SEQ | |
|---|---|
| ID | |
| NO: | Ultralong CDR-H3 Sequence |
| 1 | TSVLQSTKPQKSCPDGFSYRSWDDFCCPMVGRCLAPRNTY |
| | TTEFTIEA |
| 21 | STVHQKAHTSVACPEGWSECGVAIYGYDCGRWGCGHFLNS |
| | GPNISPYVSTHKYEWYVDA |
| 23 | TTVHQKAHTSVACPEGWSECGVAIYGYDCGRWGCGHFLNS |
| | GPNISPYVTTDAYEWYVDA |
| 14 | SIVHQKTQTSEGCPEGWSECGVGTYGYDCGRWGCGHYLNT |
| | GPLISGYVTTNKYEWHVEA |
| 15 | STVHQKAHTSVACPEGWSECGVAIYGYDCGRWGCGHFLNS |
| | GPNISPYVTTDAYEWYVDA |
| 13 | SIVHQKAHTSVTCPEGWSECGVAIYGYECGRWGCGHFLNS |
| | GPNISPYVSTHKYEWYVDA |
| 58 | VIVYQETIKSCREGYIDGGGCCLPGSCRGCACSYYDWLKC |
| | PRDCRGTSEEYIYTYNFRVDA |
| 74 | CTVQQKTHQVCPDGFNWGYGCAAGSSRFCTRHDWCCYDER |
| | ADSHTYGFCTGNRVTNTYEFHADA |
| 86 | TTVHQRTITRCPDDFGNTCRCSKGTCPCGEDACCGTNQYS |
| | FWGDCRDVGRTTFIETYEWNVDD |
| 76 | CTTVHQKTDQKRSSCPDGYSDCLVCGADRDGCSSGGCRGC |
| | WTNAYYSSRTYYNTDEFHYKPNEFHVDM |
| 104 | TTVHQRTIKSGCPPGYKSGVDCSPGSECKWGCYAVDGRRY |
| | GGYGADSGVGSTYTHEFYVDA |
| 144 | TTVHQTTNRKKTCPDNYREVDGCDPYDCCLTTWCTNSYCT |
| | RYIYEDSYEFYVTA |
| 109 | TTVHQKTKKSCPLGYAINDRCDDLKTCGPDECCLNGVVNA |
| | YGICEYEGESATHTYEWYVDA |
| 149 | TSVLQSTKKQKSCPDGLSYRAWDDFCCPNVGRCLPPINTY |
| | TYTHAFHIEA |

For the hits, purified proteins were prepared according to the method described as follows. Using an Akta pure (GE Healthcare) Hi-Trap Nickel excel columns (GE Healthcare) were equilibrated with 10 column volumes (CV) of PBS. Cell supernatants were loaded and the column was washed with 7×CV of PBS, 0.5 M NaCl. The column was then washed with 7×CV of Buffer A (0.5 M NaCl, 0.02 M Imidazole, PBS pH 7.3). Protein samples were eluted by isocratic elution with 10×CV of Buffer B (0.5 M NaCl, 0.25 M Imidazole, PBS pH 7.3), as fractions. The column was washed with 0.1 M NaOH and re-equilibrated into PBS prior to subsequent loading. Post elution, the protein containing fractions were pooled and buffer exchanged into PBS, using PD-10 columns (GE Healthcare).

The purified proteins were titrated in an ELISA experiment for binding to C5 and a bovine serum albumin negative control. These data surprisingly show certain ultralong CDR-H3 are viable even in the absence of the supporting infrastructure of the parent antibody.

### Example 2: Isolated Antibody Fragments According to the Invention Confer Binding to Human Complement Component C5 when Inserted into CDR-H3 of a Fab

1. Production of a Fab Comprising a Knob Domain Inserted into its CDR-H3

To assess the importance of the β-stalk, knob domains were separated from the stalk and inserted into the CDR-H3 of a Fab, flanked by TEV protease sites to allow the knob domain peptide to be excised. We considered a knob domain peptide fragment, from the residue preceding the first cysteine residue of CDR-H3 to the residue following the last cysteine residue of CDR-H3, where the ultralong CDR-H3 has a minimum of two cysteines. Knob domain peptides for the 154 CDR-H3 of SEQ ID NO: 1 to 154 are shown in bold in Table 4 and listed in Table 7 below:

TABLE 7

| K | SEQ | |
|---|---|---|
| identifier | ID | |
| (knob) | NO: | Sequences of CDR-H3 knob domains |
| 149 | 157 | SCPDGFSYRSWDDFCCPMVGRCL |
| 152 | 158 | SCPDGFSYRSWDDFLLSYGWECL |
| 147 | 159 | TCPRGYEYVSCWWGATCTYGGRCS |
| 148 | 160 | TCPRGYEYVSCWWGATCTYGGRCS |
| 142 | 161 | SCPEGYTYVWGCDDDSGGVGYGCAPNGASSCS |
| 143 | 162 | SCPEGYTYVWGCDDDSGGVGYGCAPNGASSCS |
| 132 | 163 | NCPDGYGYQRHCTVGEDCTERCCDNYGLCT |
| 133 | 164 | NCPAGYGYQRHCTVGEDCTDSCCDRYGLCT |
| 140 | 165 | TCPEGLVYNSDQSRCCAADSGVCW |
| 141 | 166 | TCPEGLIYNSDQSRCCAADSGVCW |
| 126 | 167 | SCPANHSVRDMCSYGPDDCGRSCCTDGIYVRR |
| | | GSCS |
| 129 | 168 | SCPENHSVRDMCSYGPDDCGRSCCTDGIYVRR |
| | | GSCS |
| 92 | 169 | TCPEGWSECGVAIYGYECGRWGCG |
| 93 | 170 | GCPEGWSECGVGTYGYDCGRWGCG |
| 94 | 171 | ACPEGWSECGVAIYGYDCGRWGCG |
| 95 | 172 | GCPEGWNDCGGNTYGYDCGRWGCG |
| 97 | 173 | GCPEGWNECGEAIYGYDCGRWGCG |
| 98 | 174 | GCPEGWNDCGVNIYGYDCGRWGCG |
| 99 | 175 | GCPEGWNDCGRNTYGYDCGRWGCG |
| 100 | 176 | TCPEGWSECGVAIYGYECGRWGCG |
| 101 | 177 | ACPEGWSECGVAIYGYDCGRWGCG |
| 102 | 178 | ACPEGWSECGVAIYGYDCGRWGCG |
| 103 | 179 | ACPEGWSECGVAIYGYDCGRWGCG |
| 104 | 180 | ACPEGWSECGVAIYGYDCGRWGCG |
| 107 | 181 | TCPEGWSECGVAIYGYDCGRWGCG |
| 108 | 182 | GCPEGWSECGVGTYGYDCGRWGCG |
| 109 | 183 | ACPEGWSECGVAIYGYECGRWGCG |
| 110 | 184 | GCPEGWNDCGGNTYGYDCGRWGCG |
| 115 | 185 | TCPEGSMLGSECNSHWSCEGCDCAKHCTWGGR |
| | | CVDCS |
| 119 | 186 | TCPEGSMLGSECNSHWSCEACDCARHCTWGGR |
| | | CVDCS |
| 27 | 187 | PCRDDSSYACVCRWTRGCSGTDCSGCTPDSDI |
| | | DYGCDTIACN |
| 32 | 188 | PCRDDSSYACVCRWTRGCSGTDCSGCTPDSDI |
| | | DYGCDTIACN |
| 44 | 189 | PCRDDSSYACVCRWTRGCSGTDCSGCTPDSDI |
| | | DYGCDTIACN |
| 52 | 190 | TCPDGTSSHSACILGTGGCCLDQYYRRGICGR |
| | | VDACY |
| 54 | 191 | TCPDGSSSHSACKLGTGGCCLDGYYRRGICGRV |
| | | DACY |
| 83 | 192 | SCPDGSSDAESGVCSGCCRGWDCCSFEVDWVGC |
| | | KGCT |
| 85 | 193 | SCPDGSSDAESGVCSGCCRGWDCCSFEVDWVGC |
| | | KGCT |
| 87 | 194 | SCPDGSSDAESGVCSGCCRGWDCCSFEVDWVGC |
| | | KGCT |
| 89 | 195 | TCPDGSSDAESGVCSGCCGGWDCCSFKVDWVGC |
| | | KECT |
| 13 | 196 | PCRDVASPVCVCRWAEGCSGTDCSECTPDPDRD |
| | | YGTCEIIACT |
| 14 | 197 | PCRDVTSPVCVCRWAEGCSGTDCSDCTPDPDRD |
| | | YGTCEIIACT |
| 20 | 198 | PCRDVTSPVCVCRWAEGCSGTDCSDCTPDPDRD |
| | | YGTCEIIACT |
| 29 | 199 | SCPDDYRDGGECCIYGRCSAEDCSVTGWEYYGS |
| | | TLCR |
| 34 | 200 | SCPDDYRDGGECCIYGRCSAEDCSVTGWEYYGS |
| | | TLCR |
| 37 | 201 | SCPDDYRDGGECCIYERCSAEDCSVTGWEYYGS |
| | | TLCR |
| 46 | 202 | SCPDDYRDGGECCIYGRCSAEDCSVTGWEYYGS |
| | | TLCRVPYITTLCL |
| 59 | 203 | NCPDGYSEINACGDRYKASGGLCCGEGAGAWRC |
| | | WECS |
| 61 | 204 | HCPDGYSDIYGCGHYYSATGGHCCGEGAGAWRC |
| | | WECS |
| 62 | 205 | NCPDGYSDIYGCGNRYAATGGHCCGEGAGAWRC |
| | | WECS |
| 64 | 206 | HCPDGYSDIYACGHYYSATGGHCCGEGAGAWRC |
| | | WECS |
| 65 | 207 | HCPDGYSDIYGCGHYYSSTGGHCCGEGAGAWRC |
| | | WECS |
| 66 | 208 | HCPDGYSDIYGCGHYYSATGGHCCGEGAGAWRC |
| | | WECS |
| 68 | 209 | NCPDGYSNIYDCGHYYSSSGGHCCGEGAGAWRC |
| | | WECS |
| 71 | 210 | SCPDGYSDIYGCGHYYSSTGGHCCGEGAGAWRC |
| | | WECS |
| 74 | 211 | NCPDGYSDIKGCGNAYAATGGHCCGEGAGAWRC |
| | | WECS |
| 78 | 212 | SCPDGYSDIYGCGHYYSSTGGHCCGEGAGAWRC |
| | | WECS |
| 79 | 213 | NCPFGYSDIKGCGNRYAATGGHCCGEGAGAWRC |
| | | WECS |
| 60 | 214 | SCREGYIDGGGCCLPGSCRGCACSYYDWLKCPR |
| | | DCR |
| 77 | 215 | SCPEGYIDGGGCCLPGSCRGCACTYYNVLKCPR |
| | | DCR |
| 10 | 216 | GCPDGVSVVNTCGWLRCNCGDSIYCSRSADSGM |
| | | WCGRCGDCT |
| 11 | 217 | GCPDGVSVVPTCGWLRCNCGEDLYCSRSDEQGT |
| | | WCGRCGDCT |
| 18 | 218 | GCPNGVTRVATCGWKRCHCGENIYCSRSDDSGT |
| | | WCGRCGDCT |
| 19 | 219 | GCPNGVPRVTTCGWKRCHCGENIYCSRSDDSGT |
| | | WCGRCGDCT |
| 21 | 220 | GCPDGVSVVNTCGWNRCNCGDTTFCSRSDDSGT |
| | | WCGRCGDCS |
| 22 | 221 | GCPDGVSVVNTCGWKRCNCGDSIYCSRSADDDG |
| | | WCGRCGDCT |
| 23 | 222 | GCPNGVTRVATCGWKRCHCSENIYCSRSDDSGT |
| | | WCGRCGDCT |
| 47 | 223 | KCPDGYTYSGDCGICDDCGGRTSRAYDCAGDTS |
| | | LYMCG |
| 55 | 224 | KCPDGYTYSGDCGICDDCGGRTSRAYDCAGDTS |
| | | LYMCG |
| 2 | 225 | SCPDGYTYINDCIGASGAVSRYDCWRFRRMNGV |
| | | CI |
| 3 | 226 | SCPDGYTYIVDCIGATGAVSRYDCWRFRRMNGV |
| | | CI |
| 31 | 227 | SCPGGCRDTDGHDYDHWSCAGSDCCCFGTDGGC |
| | | GRWGIYCS |
| 33 | 228 | SCPGGCRDTDGHDYDHWSCAGSDCCCFGTDGGC |
| | | GRWGVYCS |
| 45 | 229 | SCPGGCRDTDGHDYDHWSCAGSDCCCFGTDGGC |
| | | GRWGIYCS |
| 8 | 230 | VCPDGFNWGYGCAAGSSRFCTRHDWCCYDERAD |
| | | SHTYGFCT |
| 9 | 231 | DCPDGFNWGYGCAAGSSLHCARHDWCCYDDRVG |
| | | RDTYGFCT |
| 1 | 232 | SCPDGYSDCLVCGADRDGCSSGGCRGCW |
| 4 | 233 | TCHDPSGGTWERADGATSCPGTHCCS |
| 5 | 234 | SCPDGYRFFQECRGTGTGCPGDDCVCYDGRGGF |
| | | RWRNGCT |
| 6 | 235 | ICPDDERRRWGCSDDSEGCSDSDCHIYDGDGSV |
| | | GCCD |
| 7 | 236 | SCPDGYSSCSSCRANDLDCRGVDCVNDRVCR |
| 12 | 237 | TCPPGLSDSNACPVGTWASRRTGCCSCCDRFCG |
| | | GYSTCT |
| 15 | 238 | ACPDGYFYEYRCLVGGGCGWGCW |
| 16 | 239 | TCPDGYIADGGCRKAGSWCSSVDCAGYGEDGDY |
| | | GGWRTSCCY |
| 17 | 240 | KCPDGYTFTANNCVTSSVRCSGRNCCGGDSYGY |
| | | YIGIGGICH |
| 24 | 241 | ICRVDYVDSATCTWNCDCCRSRKSDCCAYANSR |
| | | SCW |
| 25 | 242 | RCPDDFGNTCRCSKGTCPCGEDACCGTNQYSFW |
| | | GDCR |
| 26 | 243 | SCPYGTGFDPTWCDSVLPCRRDGCWTTVWGCCE |
| 28 | 244 | DCPAGYKQVYGCSAGNCGCRGNGCCNSGSCGTW |
| | | SEWGQYGCCNCH |
| 30 | 245 | SCPDGYTDIDGCSWRHGCCRYDCCSDRSCSWCV |
| 35 | 246 | SCPDGYTDRVGCPYLWTSCARGDCW |
| 36 | 247 | HCRDGYDYGGGCCVSSGVYGESCRSSGGSDCDQ |
| | | WVGCE |
| 38 | 248 | TCPDGYEYDTACGHGRCCCVGSSCR |
| 39 | 249 | SCPPDTTEHDCCGCGGRGCAWSGCYRKGYGTGC |
| | | RVCT |
| 40 | 250 | SCPDGTDYAYGCRLGAWGCAGVGCCRGGAVGAW |
| | | GCY |
| 41 | 251 | SCPDGYNDVEARAHRSECSPNDCLRDGLGVASG |
| | | CA |
| 42 | 252 | SCREGTFYHAVCGGVVRCQVVDCDADGGCCY |
| 43 | 253 | SCPDGYSDDDGRPDHWSCMDVDCWRPARGGWGS |
| | | NCE |
| 48 | 254 | RCPAGYSISACRDGIGCGATDCCADGATDYAWG |
| | | WECK |
| 49 | 255 | SCPDGYHFIEPCHSGLCWREGACNGDGICANGL |
| | | GRCR |
| 50 | 256 | SCPDYCSCIFSYCSGADGCSSYGYCG |
| 51 | 257 | RCPDDYSYRSRGWIGSDCGGHGCWSDRDARRYD |
| | | VYGNCN |
| 53 | 258 | LCRVEECCKNGVVNAYGICE |
| 56 | 259 | SCPDGYVYSYDCGICDDCGGRTSRAYDCAGDTS |
| | | LYMCG |
| 57 | 260 | GCPPGYKSGVDCSPGSECKWGCY |
| 58 | 261 | TCPLGYDLNDRCDHENTCRVEECCKNGVVNAYG |
| | | ICE |
| 63 | 262 | ICPDDYTALNGWGCGEYRCCPKSGACCCSGGGV |
| | | HLLQSCS |
| 67 | 263 | SCPAGCQDECGSSENCYCFRYGIWCH |
| 69 | 264 | RCPEDYSDRDHCSCWAGCGDDDCWRVVAGWRCS |
| 70 | 265 | SCPLGYAINDRCDDLKTCGPDECCLNGVVNAYG |
| | | ICE |
| 72 | 266 | SCPEGYEGAPDCGAFDYCRVDDCCCRSGYGSCR |
| | | RDSCR |
| 73 | 267 | RCPDDYRDCGHCCCQYGCHAVGCWRRQGGGFER |
| | | CG |
| 75 | 268 | TCPDGYEFGKNCPDGHGCSGSDCWRCDSRSAWW |
| | | CT |
| 76 | 269 | SCKGGTDCGAGCCADGDPCSSGRCRAWSSTLRD |
| | | YFYYPTSNYTYICD |
| 80 | 270 | SCPGVSAEGGVCCSGTACTVPECWWFHQGHYSI |
| | | PGGCT |
| 81 | 271 | HCPDGYDYCRVTEDGYCCSAWTCMHWRCA |
| 82 | 272 | ICPDGCIYACSCREEWRCTVFDCVRPRDVPNGR |
| | | NACVSTCP |
| 84 | 273 | KCPDGYRVGTDCTPGKGCDYACH |
| 86 | 274 | SCGITCRKRCDCSFVGYCACSESVSGDCTCY |
| 88 | 275 | NCPPSSTSDGDCRGGWTCRGGDCSRWRGYYSSG |
| | | NNYCCY |
| 90 | 276 | SCPWGYDNGHGCNCGNDVFACSECLRSGTCS |
| 91 | 277 | SCPDDYPVKCERGCGRERCGNCGWACNGPVGSP |
| | | TCSYCR |
| 96 | 278 | TCPDGYIWAERCPGGWTSCRNACW |
| 105 | 279 | TCPDGHTWRHGYRCTGWSYGCFRGAGNDCS |
| 106 | 280 | TCPDGYGYQDACGRWGGCVGRACCSSGGSGCCD |
| | | GSCG |
| 111 | 281 | TCPDTFTYKDGCRRGGTLLNSRSGCYNVYCN |
| 112 | 282 | SCPNGYSSDAGCLAAWRCGDYDCCRENAFRPCT |
| 113 | 283 | TCPDGGTYARDCGRECAICGHCGCCQNAYRRNW |
| | | ETCN |
| 114 | 284 | SCPSGYTLWGDCEGDDGGEGGVCRCW |
| 116 | 285 | SCPEWVQTSRTCIYRSRCGQYVCWSLGEDDCGV |
| | | TCT |
| 117 | 286 | TCPDAWRSSATCRGAYGEAYECCPSGSSMWTSC |
| | | VGCT |
| 118 | 287 | TCPDGWRPGSECGWEDRCCGEFCSRCD |
| 120 | 288 | NCPDGYKYNGFCTPDGGCSRVSSWGWDRSCI |
| 121 | 289 | NCPDGGSPSVQCLDDTWACRIVDCY |
| 122 | 290 | TCSCPDNWETSGDCAGSSGDCSDCTCW |
| 123 | 291 | SCPGDRPVDCGDDYGTLGCCPFHVGCGTWRCI |
| 124 | 292 | NCPDGGSPSVQCLDDTWACRIVDCYA |
| 125 | 293 | SCPVGYDGGGNCGRYVDTCWGSDCCRYRRGIDY |
| | | SCS |
| 127 | 294 | SCPDGYRRGLECSAEWRCRYYDCVECSYGLCG |
| 128 | 295 | NCPDGSSLLSSCFDTGGCSLYSCG |
| 130 | 296 | RCPDGYYQGWHMSLRRYVCA |
| 131 | 297 | NCPTWCGFAHSCILRYEACSDCDCS |
| 134 | 298 | SCPGGYIARCAGTYGCSAVPGCCDFSGDCL |
| 135 | 299 | FCRISECY |
| 136 | 300 | TCPDNYREVDGCDPYDCCLTTWCTNSYCT |
| 137 | 301 | TCPDGGEPSVICLDASEVCRISECY |
| 138 | 302 | SCPAYDSSGCGCVYYSPWNACICDKPGGPCD |
| 139 | 303 | TCPSWCSLYMCGGYLACSACGCA |
| 144 | 304 | TCPDGYVYNDPCDCWGRRNYDCCCE |
| 145 | 305 | SCPDGLSYRAWDDFCCPNVGRCL |
| 146 | 306 | GCPEGTTYLGGSSETYRCG |
| 150 | 307 | TCPDGLSYRSWDGFCCPKVGRCL |
| 151 | 308 | TCPDGLSYRSWDGFCCPKYGRCL |
| 153 | 309 | SCPDGWRDTGTHCE |
| 154 | 310 | SCPSGYSTTLHCCCGSWKCDWCD |

The regions flanking the knob domain fragment are shown in italics in Table 4.

For expression of knob domain peptides, the human Fab, PGT121, was selected as the vehicle. The CDR-H3 of PGT121, which binds complex-type N-glycans within the gp120 envelope of HIV, contains an extended anti-parallel β-stalk which was an ideal platform on which to present the knob domain peptide, while the natural antigen was unlikely to contribute inappropriately to C5 binding. The amino residues from Kabat H100c to H100h (Gly-Ile-Val-Ala-Phe-Asn) were deleted and replaced with a knob domain peptide sequence in between two TEV protease sites. From the set of 14 binders, six knob domains of diverse peptide sequences, with different numbers and arrangements of cysteines, were reformatted as cleavable PGT121-knob fusion proteins.

The PGT-121 heavy chain sequence, with a C-terminal His tag, a TEV protease cleavage sites in bold, and GS motif underlined, is as follows, the CDR-H3 residues deleted are shown in italics:

(SEQ ID NO: 311)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGIVAFNEWFTYFYMDVWGNGTQVTVSSASTKGPSVF
PLAPSSKSTSGGTAALGCLVKDYFPEPVTVSWNSGALTSGVHTFPAV
LQSSGLYSLSSVVTVPSSSLGTQTYICNVNHKPSNTKVDKRVEPKSC
DKTLEENLYFQGSGGSHHHHHHHHHH

The heavy chain sequences of the PGT-121-knob fusions were as follows, the inserted sequences are shown in italics, with the TEV protease cleavage sites in bold, and GS motif underlined:

PGT-121-K149
(SEQ ID NO: 312)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGS [symbol] GSCPDGFSYRSWDDFCCPMVGRCLAPRN
GS [symbol] GSEWFTYFYMDVWGNGTQVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKRVEPKSCDKTLEEN
LYFQGSGGSHHHHHHHHHH
K149 (portion)
(SEQ ID NO: 313)
SCPDGFSYRSWDDFCCPMVGRCLAPRN
PGT-121-K136
(SEQ ID NO: 314)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGS [symbol] TCPDNYREVDGCDPYDCCLTTWCTNSY
CTRYIENLYFQGSEWFTYFYMDVWGNGTQVTVSSASTKGPSVFPLAP
SSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTL
EENLYFQGSGGSHHHHHHHHHH
K136
(SEQ ID NO: 315)
TCPDNYREVDGCDPYDCCLTTWCTNSYCTRYI
PGT-121-K92
(SEQ ID NO: 316)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGS [symbol] GVTCPEGWSECGVAIYGYECGRWGCGHF
LNSGPNISPYVTTGSENLYFQGSEWFTYFYMDVWGNGTQVTVSSAST
KGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKR
VEPKSCDKTLEENLYFQGSGGSHHHHHHHHHH
K92
(SEQ ID NO: 317)
VTCPEGWSECGVAIYGYECGRWGCGHFLNSGPNISPYVTT

For K92, a Serine was mutated to Threonine T at the position underlined in the initial sequence of the ultralong CDR-H3, with no impact on the binding properties.

The same assays can be reproduced with the initial sequence:

(SEQ ID NO: 318)
VTCPEGWSECGVAIYGYECGRWGCGHFLNSGPNISPYVST
PGT-121-K57
(SEQ ID NO: 319)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGSENLYFQGSGCPPGYKSGVDCSPGSECKWGCYAVD
GRRYGGYGADSGVENLYFQGSEWFTYFYMDVWGNGTQVTVSSASTKG
PSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVE
PKSCDKTLEENLYFQGSGGSHHHHHHHHHH
K57
(SEQ ID NO: 320)
SGCPPGYKSGVDCSPGSECKWGCYAVDGRRYGGYGADSGVGS
For K57, the GS was removed from the C-terminal
and the TEV protease site appended directly to
the C-terminal, without a GS linker.
PGT-121-K8
(SEQ ID NO: 321)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGSENLYFQGVCPDGFNWGYGCAAGSSRFCTRHDWCC
YDERADSHTYGFCTGNRVENLYFQGSEWFTYFYMDVWGNGTQVTVSS
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKRVEPKSCDKTLEENLYFQGSGGSHHHHHHHHHH
K8
(SEQ ID NO: 322)
VCPDGFNWGYGCAAGSSRFCTRHDWCCYDERADSHTYGFCTGNRV
PGT-121-K60
(SEQ ID NO: 323)
QMQLQESGPGLVKPSETLSLTCSVSGASISDSYWSWIRRSPGKGLEW
IGYVHKSGDTNYSPSLKSRVNLSLDTSKNQVSLSLVAATAADSGKYY
CARTLHGRRIYGSENLYFQGKSCREGYIDGGGCCLPGSCRGCACSYY
DWLKCPRDCRGTSEEENLYFQGSEWFTYFYMDVWGNGTQVTVSSAST
KGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKR
VEPKSCDKTLEENLYFQGSGGSHHHHHHHHHH
K60
(SEQ ID NO: 324)
KSCREGYIDGGGCCLPGSCRGCACSYYDWLKCPRDCRGTSEE

Heavy chains were paired to the PGT-121 light chain, sequence as follows:

(SEQ ID NO: 325)
QSVLTQPPDISVAPGETARISCGEKSLGSRAVQWYQHRAGQAPSLII
YNNQDRPSGIPERFSGSPDSPFGTTATLTITSVEAGDEADYYCHIWD
SRVPTKWVFGGGTTLTVLGQPKAAPSVTLFPPSSEELQANKATLVCL
ISDFYPGAVTVAWKADSSPVKAGVETTTPSKQSNNKYAASSYLSLTP
EQWKSHRSYSCQVTHEGSTVEKTVAPTECS

2. Biacore Analysis of Binding to C5

Binding to C5 was measured using surface plasmon resonance (SPR) single-cycle kinetics as described below. C5 has been shown to be activated by extremes of pH and so single cycle kinetics, in which sequential injections of increasing concentrations of analyte are performed in the absence of harsh regeneration steps, was employed to maintain the integrity of the C5 protein on the sensor chip surface.

Biacore single-cycle kinetics. Using a Biacore 8K (GE Healthcare), C5 was immobilized on a CM5 chip by amine coupling. Flow cells were activated using EDC/NHS (flow rate, 10 μL/min; contact time, 30 s). C5, at 1 μg/mL in pH 4.5 Sodium-acetate buffer, was immobilized on flow cell two only (flow rate, 10 μL/min; contact time, 420 s). Finally, ethanolamine was applied to both flow cells (flow rate, 10 μL/min; contact time, 420 s). A final immobilization level of approximately 2,000 response units was obtained.

Single-cycle kinetics were measured using titrations in HBS-EP buffer. A high flow rate of 40 μL/min was used, with a contact time of 230 s and a dissociation time of 900 s. Binding to the reference surface was subtracted, and the data were fitted to a single-site binding model using Biacore evaluation software.

Results

As fusion proteins, the knob domains confer high affinity C5 binding to the PGT121 Fab.

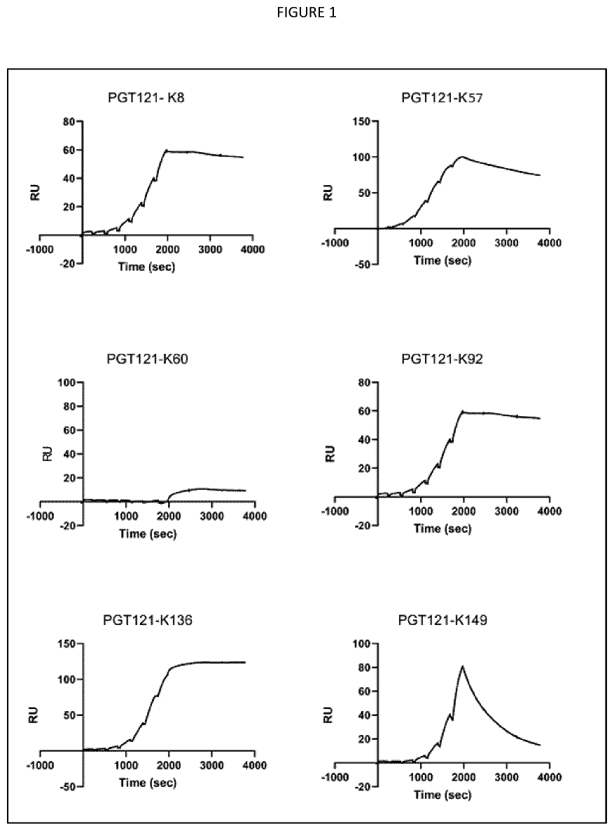

By SPR, five PGT121-knobs bound C5 with affinities in the picomolar to nanomolar range (FIG. 1 and Table 8), with only the K60 knob domain found to be non-functional after reformatting.

TABLE 8
Biacore single-cycle kinetics data on PGT121-knob domain fusion protein
Summary of kinetics from n = 3

| Construct of | mean | 95% CI | mean | 95% CI | mean | 95% CI |
|---|---|---|---|---|---|---|
| the invention | kₒₙ (1/Ms) | kₒₙ (1/Ms) | k_{off} (1/s) | k_{off} (1/s) | K_{D} (M) | K_{D} (M) |
| PGT121-K8 | 1.19E+04 | 1.07E+03 | 5.30E−05 | 4.08E−05 | 4.50E−09 | 3.61E−09 |
| PGT121-K57 | 3.23E+04 | 3.51E+03 | 1.32E−04 | 9.68E−06 | 4.10E−09 | 6.29E−10 |
| PGT121-K60 | — | — | — | — | ND | — |
| PGT121-K92 | 2.80E+04 | 2.38E+03 | <2.93E−05 | — | <1.00E−10 | — |
| PGT121-K136 | 9.52E+03 | 3.70E+03 | <1.00E−05 | — | <1.00E−10 | — |
| PGT121-K149 | 1.13E+04 | 2.72E+02 | 1.62E−03 | 1.22E−04 | 1.43E−07 | 8.40E−09 |

The PGT121-knob domain fusion proteins were counter screened for binding to human complement component C3. C3 is the closest human homologue to C5, sharing a conserved fold and a sequence homology of 26.5%. In these experiments, no cross reactivity was detected.

3. Purification of Knob Domain Peptides from the Fab Fusion Proteins

To obtain knob domain peptides, TEV protease was used to proteolytically excise the knob domains from the CDR-H3 of PGT121 Fab. Fab-knob peptide fusion proteins (10 mg/mL) were incubated with TEV protease, at a ratio of 1:100 (w/w), for a minimum of 2 hours at room temperature. Peptides can be purified and isolated using a Waters UV-directed FractionLynx system with a Waters)(Bridge Protein BEH C4 OBD Prep Column (300 Å, 5 μm, 19 mm×100 mm). An aqueous solvent of water, 0.1% trifluoroacetic acid (TFA) and an organic solvent of 100% MeCN was used. The column was run at 20 mL/min at 40° C. with a gradient of 5-50% organic solvent, over 10.8 minutes. The column was cleaned with three sharp ramps of 5-95% organic solvent. Fractions containing knob peptide were pooled and lyophilised using a Labconco Freezone freeze drier. For −80° C. storage and subsequent analysis, peptides were resuspended with 20 mM Tris pH 7.4.

TABLE 9
The sequences of the six isolated knob domain
peptides obtainable after cleavage are as follows:

| | SEQ | |
|---|---|---|
| K ref. | ID | Knob domain peptide |
| number | NO: | sequence from PGT-121 |
| K8 | 326 | GVCPDGFNWGYGCAAGSSRFCTRHDWCC |
| | | YDERADSHTYGFCTGNRVENLYFQ |
| K57 | 327 | GSGCPPGYKSGVDCSPGSECKWGCYAVD |
| | | GRRYGGYGADSGVENLYFQ |
| K60 | 328 | GKSCREGYIDGGGCCLPGSCRGCACSYY |
| | | DWLKCPRDCRGTSEEENLYFQ |
| K92 | 329 | GVTCPEGWSECGVAIYGYECGRWGCGHF |
| | | LNSGPNISPYVTTGSENLYFQ |
| K136 | 330 | GTCPDNYREVDGCDPYDCCLTTWCTNSY |
| | | CTRYIENLYFQ |
| K149 | 331 | GSCPDGFSYRSWDDFCCPMVGRCLAPRN |
| | | GSENLYFQ |

### Example 3: Isolated Antibody Fragments According to the Invention Confer Binding to Human Complement Component C5 when Inserted into the Framework 3 Region of the VH Domain of Fab which Binds to Albumin (645Fab

As described in WO2020/011868 (published on Jan. 16, 2020), Fab antibody fragments comprising an insert polypeptide within the framework 3 region of the V domain, notably the VH domain may provide a novel bispecific antibody format, in particular stable and capable of simultaneously binding two antigens. Advantageously, the CA645 Fab-knob fusion proteins as described herein may simultaneously bind C5 and albumin, which may confer an increased serum half-life to the knob domain peptide.

In addition to three CDR loops, antibody light and heavy chains, both conventional and single-chain camelid VHH, have a fourth loop which is formed by framework 3. The Kabat numbering system defines framework 3 as positions 66-94 in a heavy chain and positions 57-88 in a light chain.

The same methods as described in Example 2 were used to reformat the six ultralong CDR-H3 knob domains which bind to C5 as cleavable CA645 Fab-knob fusion proteins.

The light chain V-region or light chain of the CA645 Fab fusion proteins described in the following Examples comprised or had SEQ ID NO: 429 (CA645 VL domain (gL5)) or SEQ ID NO: 428 (CA645 light chain gL5) respectively. Alternative light chains or light V-regions may be used, for example light V-regions comprising the VL domain of SEQ ID NO: 441 or SEQ ID NO: 442.

Sequences of the 645 Fab Heavy chain knob domain fusions:

The 645 Fab heavy chain (gH5), with a K8 knob peptide (italic) inserted into its framework 3 region, with a C-terminal His Tag, TEV protease cleavage sites and GS motif (bold)

(SEQ ID NO: 332)
EVQLLESGGGLVQPGGSLRLSCAVSGIDLSNYAINWVRQAPGKGLEW
IGIIWASGTTFYATWAKGRFTISRDNSGGGGSENLYFQGSVCPDGFN
WGYGCAAGSSRFCTRHDWCCYDERADSHTYGFCTGNRVENLYFQGSG
GGSKNTVYLQMNSLRAEDTAVYYCARTVPGYSTAPYFDLWGQGTLVT
VSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSN
TKVDKKVEPKSCHHHHHHHHHH
K8 SEQ.
(SEQ ID NO: 322)
VCPDGFNWGYGCAAGSSRFCTRHDWCCYDERADSHTYGFCTGNRV

The 645 Fab heavy chain, with a K57 knob peptide (italic) inserted into its framework 3 region, with a C-terminal His Tag, TEV protease cleavage sites and GS motif (bold)

(SEQ ID NO: 333)
EVQLLESGGGLVQPGGSLRLSCAVSGIDLSNYAINWVRQAPGKGLEW
IGIIWASGTTFYATWAKGRFTISRDNSGGGGSENLYFQGSSGCPPGY
KSGVDCSPGSECKWGCYAVDGRRYGGYGADSGVENLYFQGSGGGSKN
TVYLQMNSLRAEDTAVYYCARTVPGYSTAPYFDLWGQGTLVTVSSAS
TKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDK
KVEPKSCHHHHHHHHHH
K57 SEQ.
(SEQ ID NO: 334)
GCPPGYKSGVDCSPGSECKWGCYAVDGRRYGGYGADSGV

The 645 Fab heavy chain, with a K60 knob peptide (italic) inserted into its framework 3 region, with a C-terminal His Tag, TEV protease cleavage sites and GS motif (bold)

(SEQ ID NO: 335)
EVQLLESGGGLVQPGGSLRLSCAVSGIDLSNYAINWVRQAPGKGLEW
IGIIWASGTTFYATWAKGRFTISRDNSGGGGSENLYFQGSKSCREGY
IDGGGCCLPGSCRGCACSYYDWLKCPRDCRGTSEEENLYFQGSGGGS
KNTVYLQMNSLRAEDTAVYYCARTVPGYSTAPYFDLWGQGTLVTVSS
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCHHHHHHHHHH
K60
(SEQ ID NO: 336)
KSCREGYIDGGGCCLPGSCRGCACSYYDWLKCPRDCRGTSEE

The 645 Fab heavy chain, with a K92 knob peptide (italic) inserted into its framework 3 region, with a C-terminal His Tag, TEV protease cleavage sites and GS motif (bold)

(SEQ ID NO: 337)
EVQLLESGGGLVQPGGSLRLSCAVSGIDLSNYAINWVRQAPGKGLEWIG
IIWASGTTFYATWAKGRFTISRDNSGGGGSENLYFQGSGSVTCPEGWSE
CGVAIYGYECGRWGCGHFLNSGPNISPYVTTGSENLYFQGSGGGSKNTV
YLQMNSLRAEDTAVYYCARTVPGYSTAPYFDLWGQGTLVTVSSASTKGP
SVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCH
HHHHHHHHH
K92 SEQ.
(SEQ ID NO: 317)
VTCPEGWSECGVAIYGYECGRWGCGHFLNSGPNISPYVTT

The 645 Fab heavy chain, with a K136 knob peptide (italic) inserted into its framework 3 region, with a C-terminal His Tag, TEV protease cleavage sites and GS motif (bold)

(SEQ ID NO: 338)
EVQLLESGGGLVQPGGSLRLSCAVSGIDLSNYAINWVRQAPGKGLEWIG
IIWASGTTFYATWAKGRFTISRDNSGGGGSENLYFQGSTCPDNYREVDG
CDPYDCCLTTWCTNSYCTRYIENLYFQGSGGGSKNTVYLQMNSLRAEDT
AVYYCARTVPGYSTAPYFDLWGQGTLVTVSSASTKGPSVFPLAPSSKST
SGGTAALGCLVKDYFPEPVTVSWNSGALTSGVHTFPAVLQSSGLYSLSS
VVTVPSSSLGTQTYICNVNHKPSNTKVDKKVEPKSCHHHHHHHHHH
K136 SEQ.
(SEQ ID NO: 339)
STCPDNYREVDGCDPYDCCLTTWCTNSYCTRYI

The 645 Fab heavy chain, with a K149 knob peptide (italic) inserted into its framework 3 region, with a C-terminal His Tag, TEV protease cleavage sites and GS motif (bold)

(SEQ ID NO: 340)
EVQLLESGGGLVQPGGSLRLSCAVSGIDLSNYAINWVRQAPGKGLEWIG
IIWASGTTFYATWAKGRFTISRDNSGGGGSENLYFQGSGSCPDGFSYRS
WDDFCCPMVGRCLAPRNGSENLYFQGSGGGSKNTVYLQMNSLRAEDTAV
YYCARTVPGYSTAPYFDLWGQGTLVTVSSASTKGPSVFPLAPSSKSTSG
GTAALGCLVKDYFPEPVTVSWNSGALTSGVHTFPAVLQSSGLYSLSSVV
TVPSSSLGTQTYICNVNHKPSNTKVDKKVEPKSCHHHHHHHHHH
K149 SEQ.
SEQ ID NO: 313
SCPDGFSYRSWDDFCCPMVGRCLAPRN

The knob domain peptides were purified and isolated as described above, the sequences of the isolated peptides are provided below in table 10:

TABLE 10

| | SEQ ID | |
|---|---|---|
| ID | NO: | Knob domain peptide sequence from 645 Fab |
| K8 | 341 | GSVCPDGFNWGYGCAAGSSRFCTRHDWCCYDERADSHTYGFCT |
| | | GNRVENLYFQ |
| K57 | 342 | GSSGCPPGYKSGVDCSPGSECKWGCYAVDGRRYGGYGADSGVE |
| | | NLYFQ |
| K60 | 343 | GSKSCREGYIDGGGCCLPGSCRGCACSYYDWLKCPRDCRGTSEE |
| | | ENLYFQ |
| K92 | 344 | GSGSVTCPEGWSECGVAIYGYECGRWGCGHFLNSGPNISPYVTT |
| | | GSENLYFQ |
| K136 | 345 | GSTCPDNYREVDGCDPYDCCLTTWCTNSYCTRYIENLYFQ |
| K149 | 346 | GSGSCPDGFSYRSWDDFCCPMVGRCLAPRNGSENLYFQ |

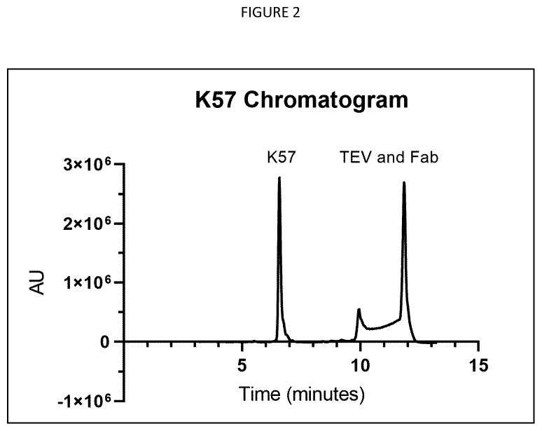

An example chromatography trace from preparative scale purification of the K57 peptide is shown in FIG. 2.

Binding to Human C5

We measured individual rate constants (kₒₙ and k_{off}) and equilibrium dissociation constants (K_{D}) for knob domain peptides binding to C5 by the Biacore single-cycle kinetics method described above.

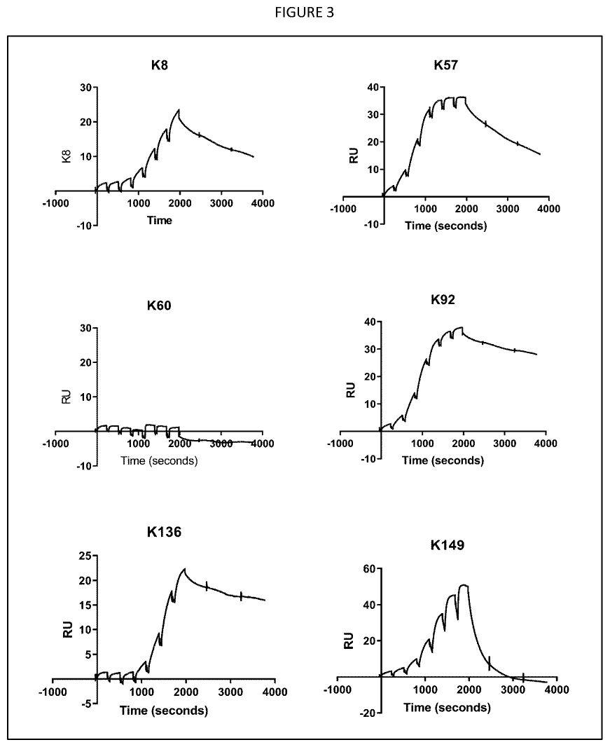

The peptides display high affinity binding, KD<40 nM, when measured by SPR single-cycle kinetics (FIG. 3 and Table 11). The only knob domain peptide found not to bind was K60.

TABLE 11
Biacore single-cycle kinetics data on isolated knob domain peptides
Summary of kinetics from n = 3

| | mean | 95% CI | mean | 95% CI | mean | 95% CI |
|---|---|---|---|---|---|---|
| Construct | kₐ (1/Ms) | kₐ (1/Ms) | k_{d} (1/s) | k_{d} (1/s) | K_{D} (M) | K_{D} (M) |
| K8 | 2.61E+04 | 1.94E+04 | 3.57E−04 | 1.28E−04 | 1.73E−08 | 1.95E−08 |
| K57 | 2.98E+05 | 5.29E+04 | 4.08E−04 | 9.12E−05 | 1.40E−09 | 1.59E−09 |
| K60 | — | — | — | — | ND | — |
| K92 | 1.24E+05 | 5.10E+04 | 1.13E−04 | — | <8.5E−10 | — |
| K136 | 1.94E+04 | 4.10E+03 | 1.23E−04 | 5.04E−05 | 6.40E−09 | 7.24E−09 |
| K149 | 9.24E+04 | 1.22E+04 | 3.35E−03 | 1.01E−03 | 3.65E−08 | 4.13E−08 |

The knob domain peptides were counter screened for cross reactivity to human complement component C3, and to ovalbumin, with no evidence of binding to either protein observed.

Binding to Mouse and Rabbit C5 Protein

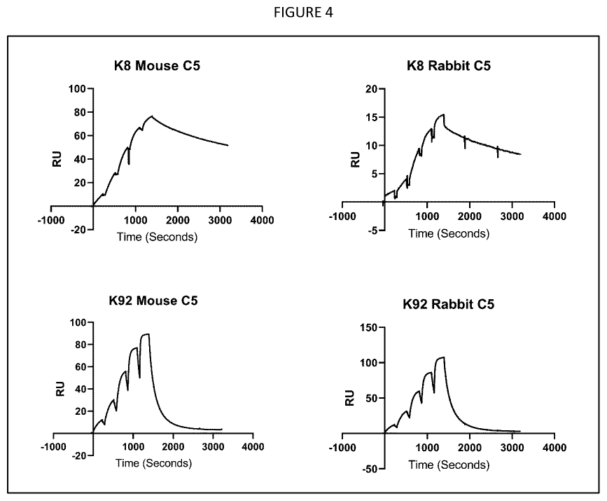

The isolated K8, K57, K92 and K149 knob domain peptides were tested for cross-reactivity against mouse and rabbit C5 protein. C5 was purified from human serum or animal serum (TCS biosciences), using methods described for example in Macpherson et al, J Biol Chem. 2018 Sep. 7; 293(36): 14112-14121. Cross reactivity to mouse C5 was observed for the K8 and K92 knob domain peptides (FIG. 4). The K57 and K149 peptides were specific for human C5 and did not cross react with the mouse or rabbit proteins. We report individual rate constants (kₒₙ and k_{off}) and equilibrium dissociation constants (K_{D}) for the K8, and K92 binding to mouse and rabbit C5 in table 12.

TABLE 12
Biacore single-cycle kinetics data knob domain peptides.
Data from n = 1 experiment.

| | Mouse C5 | | | Rabbit C5 | | |
|---|---|---|---|---|---|---|
| | kₐ (1/Ms) | k_{d} (1/s) | K_{D} (M) | kₐ (1/Ms) | k_{d} (1/s) | K_{D} (M) |
| K8 | 4.04e+4 | 1.90e−4 | 4.69e−9 | 3.24e+4 | 2.96e−4 | 9.12e−9 |
| K92 | 7.72e+4 | 4.87e−3 | 6.31e−8 | 6.08e+4 | 4.66e−3 | 7.66e−8 |

Complement Activation Assays

To elucidate the functional consequences of the knob domain peptides binding to human C5, we performed complement activation assays using the SVAR complement activation ELISA which measured CP (Classical Pathway) and AP (Alternative Pathway) activation in human serum, through formation of the C5b neo-epitope. We also developed orthogonal ELISA assays which measured total complement and AP specific mediated C3b and MAC (Membrane Attack Complex) deposition. For all activation conditions, we also tracked C5a release via ELISA.

Methods

For the C3 and C9 ELISAs, microtiter plates (e.g. MaxiSorp; Nunc) were incubated with 50 ml of a solution containing either 2.5 μg/ml aggregated human IgG (Sigma-Aldrich) for the CP, or 20 mg/ml zymosan (Sigma-Aldrich) in 75 mM sodium carbonate (pH 9.6) for the AP, overnight at 4° C. As a negative control, wells were coated with 1% (w/v) BSA/PBS. Microtiter wells were washed four times with 250 ml of wash buffer (50 mM Tris-HCl, 150 mM NaCl and 0.1% Tween 20 (pH 8) between each step of the procedure. Wells were blocked using 250 uL of 1% (w/v) BSA/PBS for 2 h at room temperature. Normal human serum (NETS) was diluted in either Gelatin veronal buffer with calcium and magnesium (0.1% gelatin, 5 mM Veronal, 145 mM NaCl, 0.025 NaN3, 0.15 mM calcium chloride, 1 mM magnesium chloride, pH 7.3; for CP) or Mg-EGTA (2.5 mM veronal buffer [pH 7.3] containing 70 mM of NaCl, 140 mM of glucose, 0.1% gelatin, 7 mM of MgCl2, and 10 mM of EGTA; for AP). NHS was used at a concentration of 1% in CP or 5% in AP. NHS was mixed with serially diluted concentrations of peptides (16 μM-15.6 nM) in appropriate buffer and preincubated on ice for 30 min. Peptide-NHS solutions were then incubated in the wells of microtiter plates for 35 min for CP/LP (both C3b and C9 detection) or 35 min for AP (C3b) or 60 min for AP (C9). Complement activation was assessed through detection of deposited complement activation factors using specific Abs against C3b (rat anti-human C3d; e.g. from Hycult) and MAC (goat anti-human C9; e.g. CompTech). Bound primary Abs were detected with HRP-conjugated goat anti-rat (Abcam)) or rabbit anti-goat (Dako) secondary Abs. Bound HRP-conjugated antibodies were detected using TMB One solution (Eco-TEK) with absorbance measured at OD450.

For the C5b ELISA, assays were run using the CP and AP Complement functional ELISA kits (SVAR), as per the manufacturer's protocol. For sample preparation: serum was diluted as per the respective protocol for the CP and AP assays. Titrations of peptides were prepared and allowed to incubate for 15 minutes at room temperature, prior to plating.

For the C5a ELISA, assays were run using the Complement C5a Human ELISA Kit (Invitrogen), as per the manufacturer's protocol. For sample preparation: at the end of the 37° C. incubation of the serum/peptide samples on the C5b ELISA assay plate, 50 μL of the diluted, activated serum was transferred to a C5a ELISA assay plate containing 50 μL/well of Assay Buffer. All subsequent experimental steps were performed as described in the protocol.

Results

These assays revealed that the K57 knob domain peptide is a potent and fully efficacious inhibitor of C5 activation, preventing release of C5a, formation of the C5b neo-epitope and the MAC. There was no effect on C3b deposition, suggesting the peptide is inhibiting complement activation downstream of C3. By contrast, the K149 peptide is a high-affinity silent binder to C5 with no detectible effect on C5a release, formation of C5b neo-epitope or MAC deposition, even at peptide concentrations in excess of 100×K_{D}.

We also identified knob domain peptides which exerted more nuanced allosteric effects on C5. The K92 peptide was a non-competitive C5 inhibitor preventing C5 activation by the AP. We observed a decrease in C5a release, decreased formation of C5b neo-epitope and decreased MAC deposition in assays where the AP component was isolated. Intriguingly, no effect was observed in the assays for which the AP component was not isolated, or in assays for which the CP component was isolated, suggesting that the K92 peptide does not inhibit C5 activation by the CP C5 convertase but it does partially inhibit activation by the AP C5 convertase.

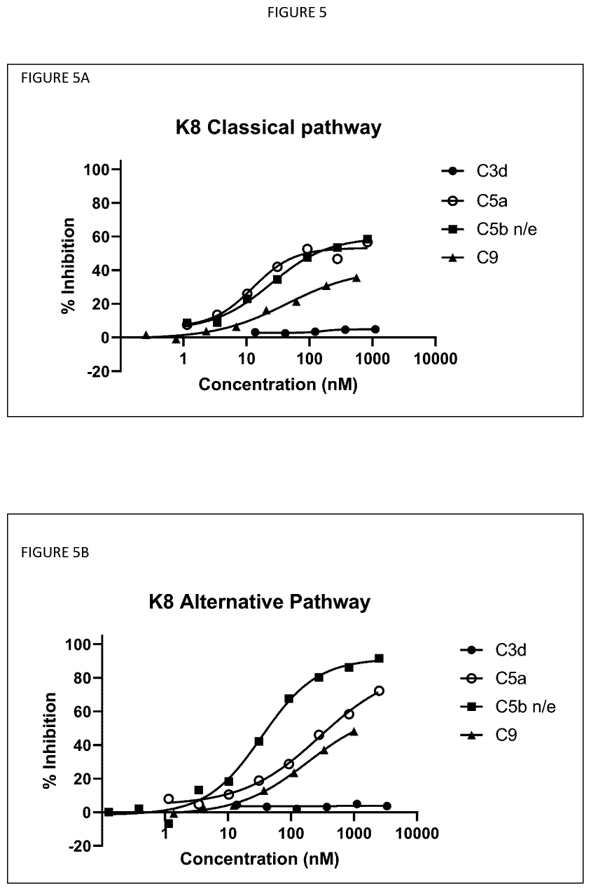

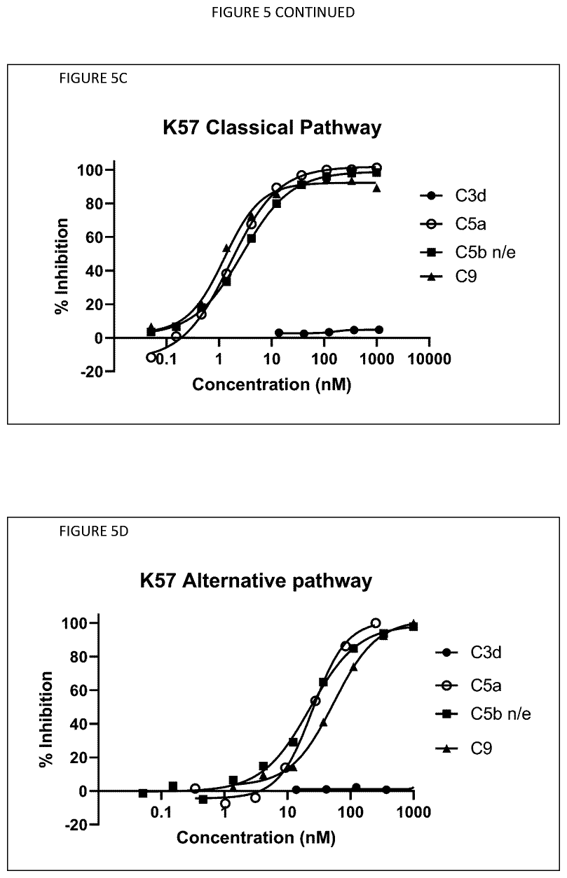

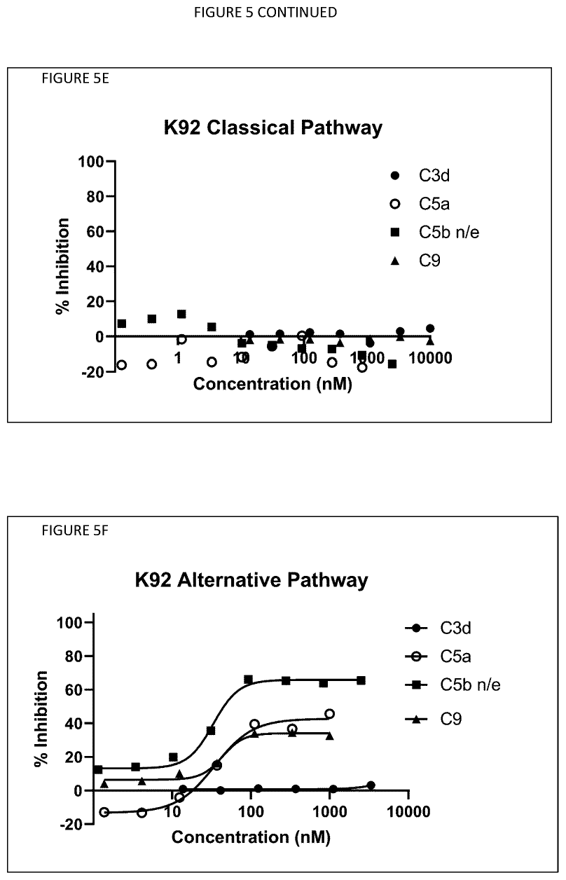

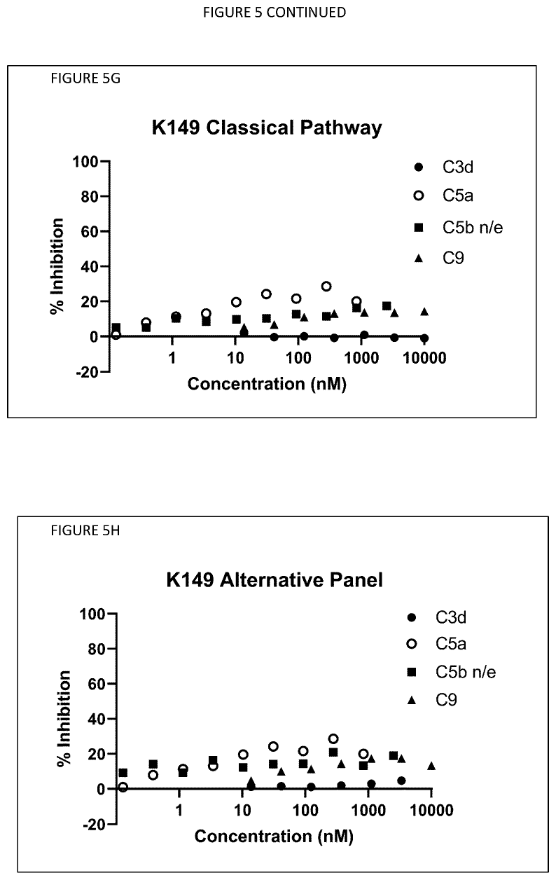

Similarly, the K8 peptide was a non-competitive inhibitor of the AP but also demonstrated non-competitive inhibition of the CP; decreasing C5a release, formation of C5b neo-epitope and MAC deposition in both CP and AP driven assays. For both the K8 and K92 peptide no effect on C3b deposition was detected. Our complement ELISA data is displayed in FIG. 5 and in Tables 13-18.

TABLE 13
Classical pathway C5b deposition ELISA.
Data from n = 3 experiments, unless specified.

| | Geomean | | Average | |
|---|---|---|---|---|
| Construct | IC50 (nM) | Range (nM) | Emax (%) | Range (%) |
| K8^{a} | 9.3 | 3.0-24.5 | 68.2 | 59.5-79.56 |
| K57 | 3.6 | 2.4-7.3 | 100.6 | 99.1-101.7 |
| K92 | ND* | ND* | ND* | ND* |
| K149 | ND* | ND* | ND* | ND* |

*ND = Not detected.
^{a}Data are an average from n = 6 experiments

TABLE 14
Alternative pathway C5b deposition ELISA
Data from n = 3 independent titrations, unless specified.

| | Geomean | | Average | |
|---|---|---|---|---|
| Construct | IC50 (nM) | Range (nM) | Emax (%) | Range (%) |
| K8 ^{a} | 29.2 | 25.4-34.2 | 91.8 | 91.4-92.3 |
| K57 | 30.4 | 22.7-52.1 | 110.3 | 97.9-134.0 |
| K92^{b} | 32.4 | 31.5-33.1 | 62.14 | 58.0-67.7 |
| K149 | ND* | ND* | ND* | ND* |

*ND = Not detected.
^{a} Data are an average from n = 5 independent titrations
^{b}Data are an average from n = 4 independent titrations

TABLE 15
Inhibition of classical pathway mediated C5a release.
Data from n = 3 independent titrations, unless otherwise stated

| | Geomean | | Average | |
|---|---|---|---|---|
| Construct | IC50 (nM) | Range (nM) | Emax (%) | Range (%) |
| K8 ^{a} | 9.9 | 3.9-18.7 | 57.7 | 51.5-73.2 |
| K57 | 4.0 | 2.8-5.6 | 98.7 | 95.9-100.6 |
| K92 | ND* | ND* | ND | ND* |
| K149 | ND* | ND* | ND* | ND* |

*ND = Not detected.
^{a} Data are an average from n = 5 independent titrations

TABLE 16
Inhibition of alternative pathway mediated C5a release
Data from n = 3 independent titrations, unless otherwise stated

| | Geomean | | Average | |
|---|---|---|---|---|
| Construct | IC50 (nM) | Range (nM) | Emax (%) | Range (%) |
| K8^{a} | 150.2 | 49.3-85.86 | 71.8 | 49.3-85.9 |
| K57 | 26.6 | 25.0-27.7 | 97.8 | 95.1-101.3 |
| K92 | 43.3 | 39.9-45.3 | 43.3 | 39.9-45.2 |
| K149 | ND* | ND* | ND* | ND* |

*ND = Not detected.
^{a}Data are an average from n = 4 independent titrations

TABLE 17
Inhibition of classical pathway mediated C9 deposition.
Data from n = 3 independent titrations, unless otherwise stated.

| | Geomean | | Average | |
|---|---|---|---|---|
| Construct | IC50 (nM) | Range (nM) | Emax (%) | Range (%) |
| K8^{a} | 190.5 | — | 63.4 | — |
| K57 | 1.9 | 1.3-2.5 | 97.9 | 92.4-97.9 |
| K92 | ND* | ND* | ND* | ND* |
| K149 | ND* | ND* | ND* | ND* |

*ND = Not detected.
^{a}Data from n = 1 titration.

TABLE 18
Inhibition of alternative pathway mediated C9 deposition
Data from n = 3 independent titrations

| | Geomean | | Average | |
|---|---|---|---|---|
| Construct | IC50 (nM) | Range (nM) | Emax (%) | Range (%) |
| K8 | 232.4 | 131.3-521.5 | 73.3 | 61.2-90.74 |
| K57 | 24.5 | 20.6-27.6 | 100.7 | 100.1-100.9 |
| K92^{a} | 43.5 | — | 34.1 | — |
| K149 | ND* | ND* | ND* | ND* |

*ND = Not detected.
^{a}Data from n = 1 titration.

Knob Domain Peptides Inhibit of Complement Mediated Bacterial Lysis ‘Killing Assay’

We explored the effects of our peptides in bacterial killing assays, using the Escherichia coli strain DC10B which is susceptible to complement mediated lysis when incubated even in dilute human serum (as described for example in (as described for example in Monk I R, et al.; Transforming the untransformable: application of direct transformation to manipulate genetically Staphylococcus aureus and Staphylococcus epidermidis. mBio. Vol. 3 (2); 2012, and commercially available).

Methods

Normal human serum (NHS) was prepared using freshly drawn blood from 8 healthy donors using serum vacutainer collection tubes (BD). Blood was kept at room temperature and allowed to clot for 30 min followed by a 1 h incubation on ice. After two rounds of centrifugation (700×g; 4° C., 8 min), serum fractions were collected, pooled and stored in aliquots at −80° C. Heat-inactivated sera (DNHS) was prepared through incubation at 56° C. for 30 min.

Escherichia coli strain DC10B was cultured to exponential phase (OD600 0.4-0.5) in LB broth with shaking (180 rpm) at 37° C. Bacteria was harvested by centrifugation, washed once in PBS and resuspended to an OD600=0.1 (correlating to approximately 107 CFU/ml) in Gelatin Veronal Buffer with calcium and magnesium (“GVB++”; 5 mM veronal buffer [pH 7.3], 0.1% [w/v] gelatin, 140 mM NaCl, 1 mM MgCl2 and 0.15 mM CaCl2). Peptides at specific concentrations or PBS (control) were incubated with varying percentages of pooled NHS in GVB++ buffer for 30 min on ice. Next, 50 μL of bacteria was added to 50 μL of peptide/PBS NHS solutions and incubated for 20 min at 37° C. Following incubation, aliquots were removed, serially diluted and spread onto LB agar plates. Plates were incubated for 18 h at 37° C., following which the remaining CFU were enumerated and survival calculated compared to CFU at time 0. Controls consisted of DNHS and serum treated with the complement C5 inhibitor OmCI (10 mg/ml; 0.625 mM).

Results

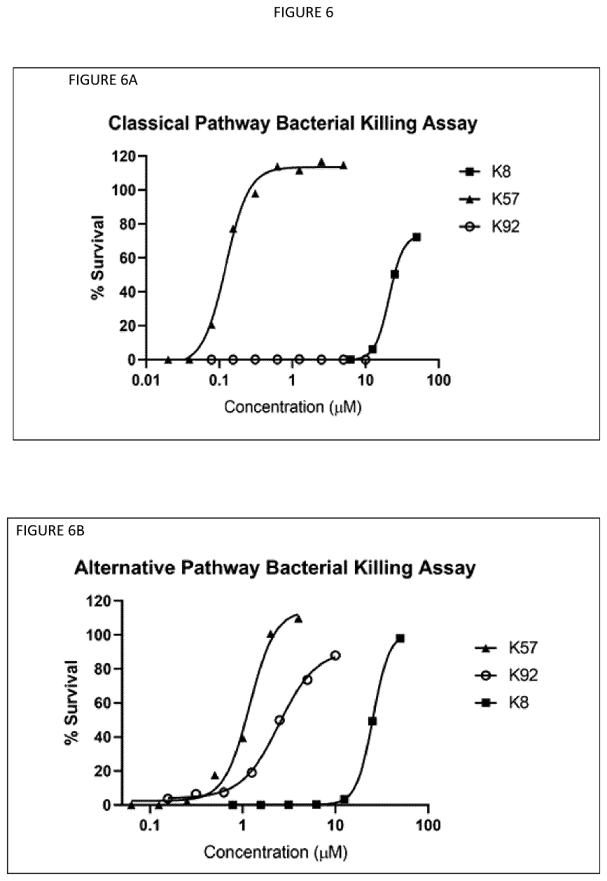

The K57 knob domain peptide was fully efficacious in preventing complement mediated lysis. We report K57 IC₅₀ values of 115 nM and 1.22 μM for CP and AP mediated killing assay, respectively, data are shown in Tables 19-20 and FIG. 6.

The K8 knob domain was able to inhibit in both the CP and AP driven killing assays we report K8 IC₅₀ values of 16.9 μM and 25.8 μM for CP and AP mediated killing assay, respectively. Consistent with our pathway specific ELISA data, the K92 peptide was only able to inhibit the AP driven killing assay achieving an IC₅₀ of 2.465 μM and an Emax of 91.0%.

TABLE 19
Inhibition of classical pathway mediated bacterial lysis.
Data from n = 3 independent titrations

| | Geomean | | Average | |
|---|---|---|---|---|
| Construct | IC50 (nM) | Range (nM) | Emax (%) | Range (%) |
| K8 | 16,900 | 13,100-21,000 | 99.5 | 73.6-121.0 |
| K57 | 115 | 82.6-150.6 | 111.2% | 104.3-113.6 |
| K92 | ND* | ND* | ND* | ND* |

*ND = Not detected.

TABLE 20
Inhibition of alternative pathway mediated bacterial lysis
Data from n = 3 independent titrations

| | Geomean | | Average | |
|---|---|---|---|---|
| Construct | IC50 (nM) | Range (nM) | Emax (%) | Range (%) |
| K8 | 25,800 | 25,300-26,700 | 98.1 | 93.6-101.5 |
| K57 | 1022 | 971.9-1571 | 122.4 | 114.6-129.1 |
| K92^{a} | 2,465 | — | 91.0 | — |

^{a}data from n = 1 experiment.

### Example 4: Production of Isolated Antibody Fragments of the Invention by Chemical, Notably Solid Phase, Peptide Synthesis

As shown hereinafter, functional isolated knob domains of ultralong CDR-H3 can be chemically synthesised by solid phase peptide synthesis. In order to form a disulphide bond between two Cysteine residues within the knob domain peptides, knob domain peptides were synthesised by two methods: 1) a site-directed method, whereby cysteines were specifically protected and deprotected to form disulphides bonds in a preordained manner; and 2) by a free energy method, whereby formation of disulphide bonds was allowed under free energy, in the presence of glutathione redox buffer.

Peptides Synthesis

All the peptides were synthesised using solid phase synthesis, employing the Fmoc technique (for example described in Atherton, E., and R. C. Sheppard. 1989. Fluorenylmethoxycarbonylpolyamide solid phase peptide synthesis: general principles and development. In Solid Phase Peptide Synthesis: A Practical Approach. IRL Press, Eynsham, Oxford, pp. 25-37). Synthesis was performed in a sequential manner in the C to N direction on robotic synthesisers (Symphony, Protein Technologies). The syntheses were started upon appropriate polystyrene supports (nova biochem) with the first amino acid attached via a Wang linkage to the carboxyl, substitution of 0.3 mM/g. Chain elongation was facilitated by using a twenty minute double coupling strategy with a 3 fold molar excess of reagents to the loading of the resins with N-alpha protected amino acids dissolved in DMF (side chains orthogonally protected with suitable protecting groups for Fmoc chemistry) and the coupling reagent TBTU, in the presence of DIPEA. The temporary amino protection was removed by two, 5-minute treatments with 20% piperidine in DMF. After the peptide sequences were complete the peptidyl resins were treated with a mixture of TFA, ethane dithiol and tri isopropyl silane, 95:3:2 for 3 hours to cleave the peptides and all protecting groups. The peptides were isolated by filtration and trituration with diethyl ether. The peptides were dissolved in acetonitrile water and freeze dried before purification.

1) Formation of Disulphide Bonds by a Site-Directed Method

Using special protecting groups, it is possible to cyclise between two specific cysteines in a peptide, thus it is possible to have more than one disulphide in a peptide. Only the K149 method was synthesised by the site directed method to create two different disulphide bonded forms, K149A and K149B. Site-directed disulphide bond formation was as follows:

K149A
(SEQ ID NO: 347)
SC¹PDGFSYRSWDDFC¹C²PMVGRC²LAPRN
K149B
(SEQ ID NO: 348)
SC¹PDGFSYRSWDDFC²C¹PMVGRC²LAPRN

The peptide K149 was synthesised using an orthoganol protection strategy for the cysteine residues. For K149A, residues C2 and C15 were protected using ACM (acetamidomethyl) on the side chain whilst C16 and C22 were protected with a trityl group on the side chain. For K149B, residues C2 and C16 were protected using ACM (acetamidomethyl) on the side chain whilst C15 and C22 were protected with a trityl group on the side chain

The peptide was synthesised using standard Fmoc solid phase chemistry, as described above. After TFA (trifluoroacetic) cleavage, which removed all side chain protecting groups with the exception of the ACM, the linear peptide was purified using rHPLC on a C18 column.

The first cyclisation reaction was performed under mild oxidising conditions using Potassium Ferracyanide and the progress of the reaction between C16 and C22 (K149A) or C15 and C22 (K149B) was monitored by LC-MS. When this was seen to have gone to completion the peptide was purified to remove any residual linear peptide and then treated with an excess of Iodine. Iodine promoted the concomitant removal of the ACM protecting groups and the final oxidation of cysteine residues C2 and C15 (K149A) or C2 and C16 (K149B). The reaction was monitored by LC-MS and the di-cyclic peptide repurified by HPLC.

2) Synthesis and Formation of Disulphide Bonds by the Free Energy Method. The Peptides are Displayed Below in Table 21.

TABLE 21

| | SEQ ID | |
|---|---|---|
| ID | NO: | Knob domain peptide sequence |
| K8 | 322 | VCPDGFNWGYGCAAGSSRFCTRHDWCCYDERADS |
| | | HTYGFCTGNRV |
| K57 | 334 | GCPPGYKSGVDCSPGSECKWGCYAVDGRRYGGYG |
| | | ADSGV |
| K92 | 349 | TCPEGWSECGVAIYGYECGRWGCGHFLNSGPNIS |
| | | PYVTTGS |
| K149 | 350 | SCPDGFSYRSWDDFCCPMVGRCLAPRNGS |

It was found that the most expedient and high yielding way to obtain these cyclic peptides with greater than two disulphide bonds was to RP-HPLC purify the linear sequence and immediately initiate cyclisation without a freeze drying step, otherwise a lot of insoluble polymeric material resulted.

Cyclisation was achieved by using thermodynamic controlled air oxidation to obtain the minimum energy form of the disulphides in the sequence, employing a mixture of reduced and oxidised glutathione.

Crude peptides were dissolved in DMSO/water and treated with TCEP to ensure the cysteine residues were fully reduced. The peptides were RP-HPLC purified, upon a varian prostar system equipped with two 210 pumps and a 355 uv spectrophotometer. Running buffers were for pump A, solvent A, 0.1% (v/v ammonium acetate in water, pH 7.5-7.8), and for pump B, solvent B, 100% acetonitrile. The peptide was introduced to a prep RP-HPLC column (C18 Axia, 22 mm×250 mm, 5 micron partical, size 110 angstrom pore size, Phenomenex), and the linear sequence eluted from the column by running a gradient between solvents A and B, 5% B to 65% B over 60 minutes. Linear peptide was identified by ESMS.

The solution of the linear peptide (approximately 50 mL) was added to 500 mL of a cyclisation buffer, (0.2 M phosphate buffer, pH 7.5, containing 1 mM EDTA, 5 mM reduced glutathione, and 0.5 mM oxidised glutathione). The solution was stirred at room temperature for 48 hours. After which, a small sample was analysed by analytical HPLC to assess the level of cyclisation.

When the cyclisation was deemed sufficiently complete, the whole buffer containing the peptide was pumped onto a preparative RP HPLC column (C-18 Axia as above). The cyclic peptide was eluted using a gradient between solvent A (0.1% TFA in water) and solvent B (0.1% TFA in acetonitrile) of 5% B to 65% B in 60 minutes. Fractions identified as the correct compound were freeze dried before analysis.

Binding to C5

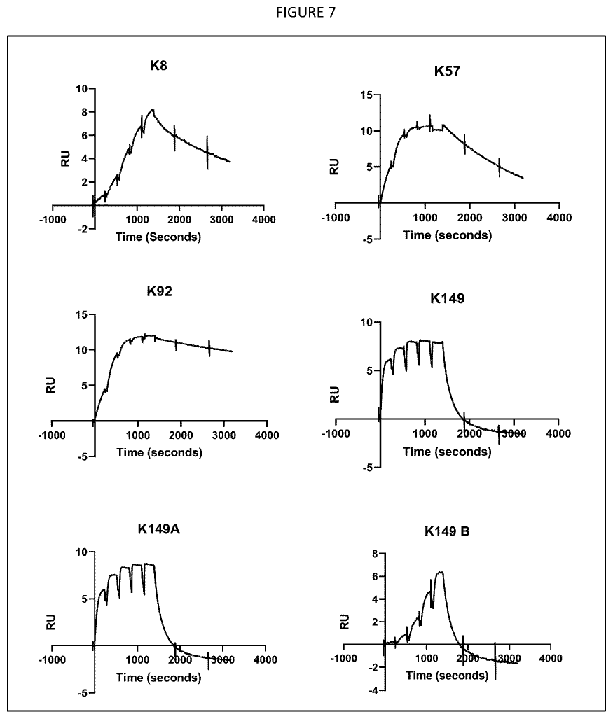

We used SPR single cycle kinetics to measure binding to human C5 protein. The results are presented in FIG. 7, and Table 22 below.

For peptides produced by glutathione cyclisation, all examples bound to C5 with affinities broadly comparable to when expressed recombinantly. These data suggest that functional knob domain peptides can be derived by chemical synthesis, whereby the minimum energy disulphide bond form is obtained. For the site directed methods, both the K149A and K149B peptides bound C5 but the K149A peptide was 41-fold more potent than K149B and equipotent to K149 produced by glutathione cycling. The K149A disulphide arrangement is both the lower energy form and the form for which binding free energy for C5 is lowest.

TABLE 22
Binding of chemically derived knob domain
peptides to C5 by SPR single cycle kinetics
Data from n = 1 experiments.

| Construct | ka (1/Ms) | kd (1/s) | KD (M) |
|---|---|---|---|
| K8 | 3.29E+04 | 3.97E−04 | 1.21E−08 |
| K57 | 2.45E+05 | 5.89E−04 | 2.41E−09 |
| K92 | 1.60E+05 | 1.03E−04 | 6.43E−10 |
| K149 | 1.54E+06 | 6.85E−03 | 4.46E−09 |
| K149A | 1.17E+06 | 7.23E−03 | 6.17E−09 |
| K149B | 2.97E+04 | 7.32E−03 | 2.46E−07 |

Functional Activity Assays

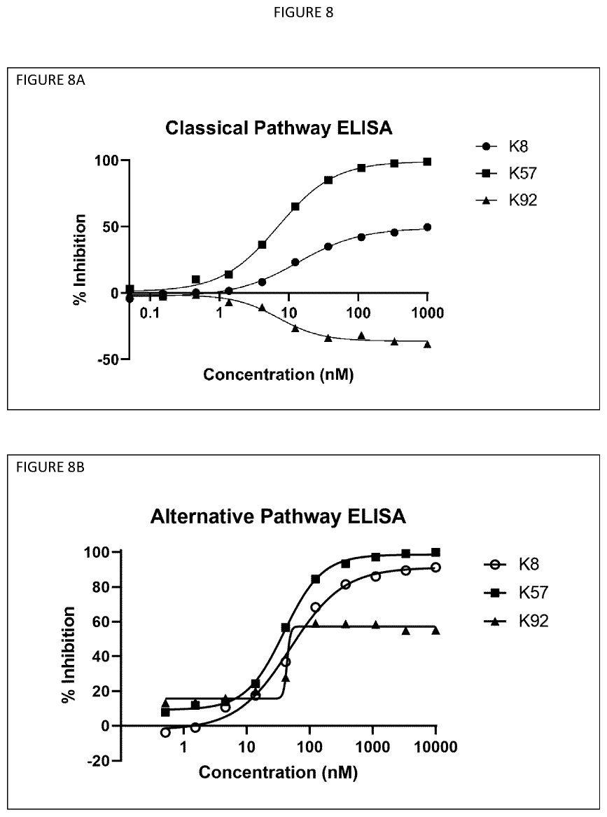

Functional activity of chemically derived knob domain peptides was confirmed in a complement activation ELISAs which measured inhibition of C5b neoepitope deposition. These data are displayed in FIG. 8 and Tables 23-24.

TABLE 23
Classical pathway C5b deposition ELISA.
Data from n = 3 independent titrations, unless specified.

| | Geomean | | Average | |
|---|---|---|---|---|
| | IC50 (nM) | Range (nM) | Emax (%) | Range (%) |
| K8 | 9.8 | 7.6-13.5 | 47.3 | 45.9-48.9 |
| K57 | 6.6 | 6.3-7.0 | 98.7 | 98.2-99.2 |
| K92 | 6.6 | 6.1-7.1 | −33.9 | −31.6-36.3 |

TABLE 24
Alternative pathway C5b neoepitope ELISA
Data from n = 3 independent titrations, unless specified.

| | Geomean | | Average | |
|---|---|---|---|---|
| | IC50 (nM) | Range (nM) | Emax (%) | Range (%) |
| K8 | 46.1 | 44.6-48.5 | 91.5 | 91.2-91.9 |
| K57 | 36.5 | 35.0-38.8 | 98.9 | 98.6-99.3 |
| K92^{a} | 46.2 | 43.5-49.1 | 56.8 | 56.5-57.2 |

^{a}Data are from n = 2 independent titrations

### Example 5: Crystal Structure

To elucidate the structural mechanisms for the allosteric modulation of C5 by a knob domain peptide, we solved the crystal structure of the C5-K8 complex. As an example, a resolution of 2.3 Å can be used.

C5 at 6.1 mg/ml (20 mM Tris, 75 mM NaCl, pH 7.35) was mixed at a 1:1 molar ratio with K8 knob domain peptide. Drops were set up by the vapor-diffusion method at 18° C. with a 1:1 mixture of mother liquor (v/v). crystals were grown in a mother liquor of: 0.1 M ADA, 14% Ethanol (v/v), pH 6.0. Prior to flash freezing in liquid nitrogen, C5-K8 crystals were cryoprotected in mother liquor with 30% MPD (v/v).

Data were collected at the Diamond Light Source (Harwell, UK). The C5-K8 structure was solved using the automated molecular replacement pipeline Balbes (F. Long, et al. “BALBES: a Molecular Replacement Pipeline” Acta Cryst. D64 125-132(2008)) using the apo C5 structure (PDB 3CU7), minus the C345c domain. A backbone model of the K8 peptide was produced using ARP-wARP (software suite commonly used for automated model building in X-ray crystallography) which informed manual model building in Coot, within the CCP4 suite (Collaborative Computational Project, Number 4) (M. D. Winn et al. Acta. Cryst. D67, 235-242 (2011)). For example, Langer G, Cohen S X, Lamzin V S, Perrakis A. (2008) Automated macromolecular model building for x-ray crystallography using ARP/wARP version 7. Nat. Protoc. 3, 1171-1179. The model was subjected to multiple rounds of refinement in Phenix.

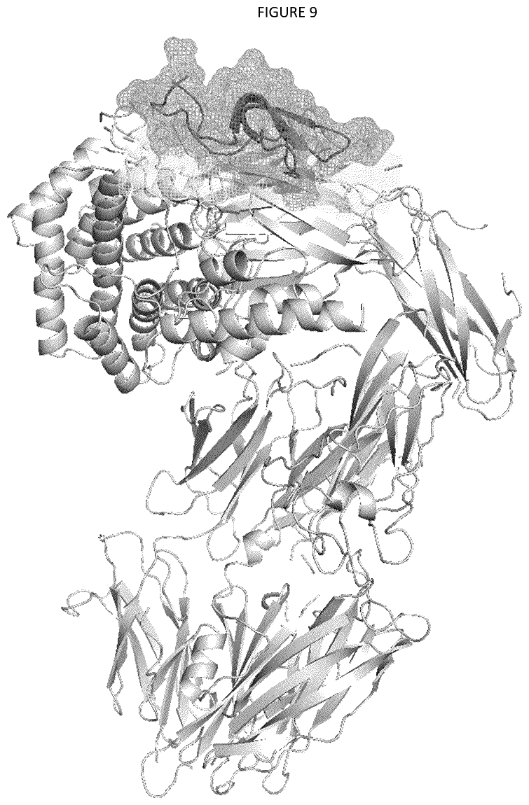

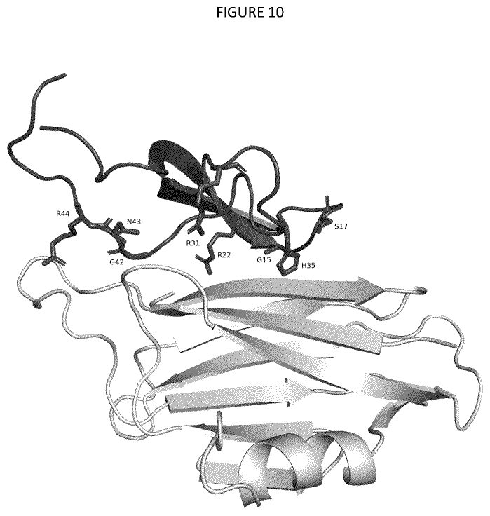

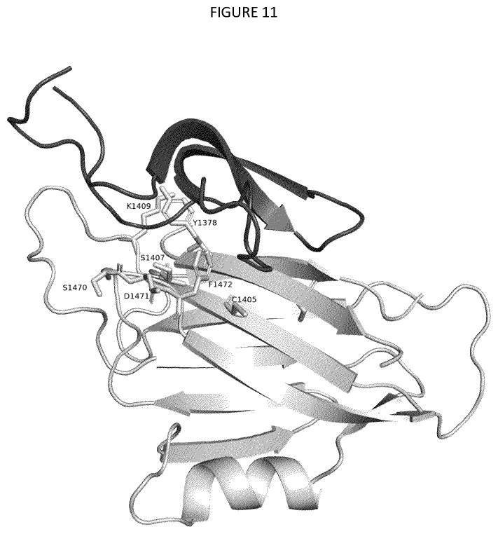

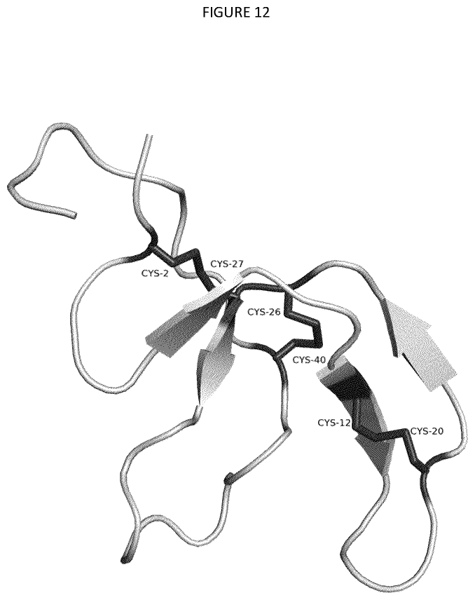

The structural data are shown in FIGS. 9, 10 and 11. The structure shows that the K8 peptide binds to a previously unrecognised site for regulating C5 on the MG8 domain of the α-chain of C5. The K8 peptide mediated a crystal contact which ensured low local B factors, clearly enunciating the disulphide bond arrangement and the intra and inter chain interactions of peptide backbone and side chains. The disulphide bonding arrangement of the K8 peptide was elucidated and is shown in FIG. 12. PDBePISA (Interactive service at the European Bioinformatics Institute) was used to examine the molecular interface between K8 peptide and C5. The hydrogen bond and salt bridge interactions are listed in Tables 25-28.

TABLE 25
K8 residues participating in C5-K8 interacting surface

| | Accessible | Buried |
|---|---|---|
| | Surface Area | surface Area |
| K8 residue | (Å²) | (Å²) |
| PHE 6 | 29.06 | 9.92 |
| CYS 12 | 10.07 | 0.72 |
| ALA 13 | 43.87 | 41.61 |
| ALA 14 | 45.81 | 43.80 |
| GLY 15 | 39.59 | 36.32 |
| SER 16 | 63.11 | 35.54 |
| SER 17 | 105.95 | 23.19 |
| PHE 19 | 152.02 | 5.47 |
| THR 21 | 114.31 | 18.39 |
| ARG 22 | 164.89 | 104.41 |
| ASP 24 | 73.55 | 32.64 |
| TRP 25 | 152.22 | 0.61 |
| CYS 26 | 12.73 | 6.70 |
| ASP 29 | 0.17 | 0.17 |
| ARG 31 | 111.41 | 27.35 |
| HIS 35 | 167.52 | 87.76 |
| THR 36 | 52.94 | 48.03 |
| PHE 39 | 47.59 | 33.43 |
| CYS 40 | 33.06 | 33.06 |
| THR 41 | 45.53 | 43.44 |
| GLY 42 | 73.55 | 69.16 |
| ASN 43 | 51.40 | 31.58 |
| ARG 44 | 194.88 | 121.93 |

TABLE 26
C5 residues participating in C5-K8 interacting surface

| | Accessible | Buried |
|---|---|---|
| | Surface Area | surface Area |
| C5 residue | (Å²) | (Å²) |
| CYS 1405 | 10.92 | 10.59 |
| VAL 1403 | 11.27 | 10.72 |
| GLU 1373 | 48.57 | 11.24 |
| CYS 1505 | 12.55 | 11.90 |
| GLN 1384 | 51.09 | 11.94 |
| GLU 1414 | 15.10 | 12.65 |
| SER 1371 | 93.64 | 13.69 |
| LYS 1409 | 77.51 | 14.87 |
| SER 1416 | 70.93 | 16.84 |
| ILE 1381 | 21.88 | 18.63 |
| SER 1469 | 24.11 | 23.21 |
| CYS 1375 | 34.69 | 26.68 |
| PRO 1410 | 48.78 | 29.31 |
| ARG 1412 | 234.23 | 29.38 |
| LEU 1379 | 30.77 | 30.77 |
| ASP 1382 | 55.82 | 35.51 |
| SER 1470 | 102.26 | 38.13 |
| MET 1507 | 47.36 | 42.05 |
| ASP 1471 | 90.78 | 55.49 |
| PHE 1472 | 94.51 | 72.66 |
| PHE 1377 | 0.98 | 0.49 |
| VAL 1374 | 125.23 | 0.67 |
| THR 1506 | 11.56 | 1.84 |
| TYR 1408 | 4.67 | 2.52 |
| SER 1411 | 37.40 | 5.31 |
| SER 1415 | 10.05 | 5.69 |
| ASN 1513 | 119.77 | 6.38 |
| PHE 1508 | 55.84 | 6.53 |
| SER 1407 | 7.41 | 7.41 |
| THR 1370 | 27.08 | 8.84 |
| ARG 1476 | 70.96 | 9.17 |
| CYS 1474 | 22.20 | 9.51 |
| TYR 1378 | 120.32 | 112.49 |
| LYS 1380 | 115.48 | 112.74 |

TABLE 27
Sidechain and backbone hydrogen bond interactions with C5

| K8 peptide | H-bonding |
|---|---|
| residue | partner on C5 |
| GLY 15 | LEU 1379 |
| GLY 15 | MET 1507 |
| SER 17 | ASP 1382 |
| ARG 22 | GLU 1373 |
| ARG 22 | GLU 1373 |
| ARG 22 | SER 1371 |
| ARG 22 | THR 1370 |
| ARG 22 | CYS 1375 |
| ARG 31 | ASP 1471 |
| ARG 31 | ASP 1471 |
| HIS 35 | CYS 1405 |
| GLY 42 | SER 1407 |
| ASN 43 | SER 1469 |
| ASN 43 | SER 1470 |
| ARG 44 | SER 1411 |
| ARG 44 | GLU 1414 |
| ALA 13 | LEU 1379 |
| GLY 15 | ILE 1381 |
| SER 17 | LYS 1380 |
| ASP 24 | TYR 1378 |
| ASP 24 | LYS 1409 |
| HIS 35 | LYS 138 |

TABLE 28
K8 residues participating in salt bridge interactions with C5

| Peptide residue | C5 residue |
|---|---|
| ARG 31 | ASP 1471 |
| ARG 31 | ASP 1471 |
| ASP 24 | LYS 1409 |

### Example 6: Isolated Antibody Fragments According to the Invention Confer Binding to Binding to Human Complement Component C5 when Inserted into the N- or C-Terminus or Framework Turns of a VHH to Create a Single Chain Bi-Specific Antibody

We produced VHH knob domain fusion proteins by inserting the K57 knob domain peptide into the framework turns (loop 1, loop 2, and loop 3) of a VHH antibody, at the opposing end to the CDRs, to make a single chain bi-specific antibody. The K57 knob domain was recombinantly fused to the hC3nb1 VHH, which binds C3 and C3b, and for which a crystal structure has been published (Protein Data Bank (PDB) code: 6EHG).

The sequences are shown below, constructs were expressed with a C-terminal single-chain Fc tag (not shown):

hC3nb1
(SEQ ID NO: 351)
QVQLVETGGGLVQAGGSLRLSCAASGSIFSLNAMGWFRQAPGKEREFVA
TINRSGGRTYYADSVKGRFTISRDNGKNMVYLQMHSLKPEDTAIYYCAA
GTGWSPQTDNEYNYWGQGTQVTVSS
K57 peptide (linker in italics)
(SEQ ID NO: 352)
SGGGSGCPPGYKSGVDCSPGSECKWGCYAVDGRRYGGYGADSGVSGGGS
hC3nb1-K57 loop 1 (peptide and linker in italics)
(SEQ ID NO: 353)
QVQLVETGGGLVQASGGGSGCPPGYKSGVDCSPGSECKWGCYAVDGRRY
GGYGADSGVSGGGSGSLRLSCAASGSIFSLNAMGWFRQAPGKEREFVAT
INRSGGRTYYADSVKGRFTISRDNGKNMVYLQMHSLKPEDTAIYYCAAG
TGWSPQTDNEYNYWGQGTQVTVSS
hC3nb1-K57 loop 2 (peptide and linker in italics)
(SEQ ID NO: 354)
QVQLVETGGGLVQAGGSLRLSCAASGSIFSLNAMGWFRQAPSGGGSGCP
PGYKSGVDCSPGSECKWGCYAVDGRRYGGYGADSGVSGGGSKEREFVAT
INRSGGRTYYADSVKGRFTISRDNGKNMVYLQMHSLKPEDTAIYYCAAG
TGWSPQTDNEYNYWGQGTQVTVSS
hC3nb1-K57 loop 2 delta proline (peptide and
linker in italics)
(SEQ ID NO: 355)
QVQLVETGGGLVQAGGSLRLSCAASGSIFSLNAMGWFRQASGGGSGCPP
GYKSGVDCSPGSECKWGCYAVDGRRYGGYGADSGVSGGGSKEREFVATI
NRSGGRTYYADSVKGRFTISRDNGKNMVYLQMHSLKPEDTAIYYCAAGT
GWSPQTDNEYNYWGQGTQVTVSS
hC3nb1-K57 loop 3 (peptide and linker in italics)
(SEQ ID NO: 356)
QVQLVETGGGLVQAGGSLRLSCAASGSIFSLNAMGWFRQAPGKEREFVA
TINRSGGRTYYADSVKGSGGGSGCPPGYKSGVDCSPGSECKWGCYAVDG
RRYGGYGADSGVSGGGSRFTISRDNGKNMVYLQMHSLKPEDTAIYYCAA
GTGWSPQTDNEYNYWGQGTQVTVSS
hC3nb1-K57 C-term (peptide and linker in italics)
(SEQ ID NO: 357)
QVQLVETGGGLVQAGGSLRLSCAASGSIFSLNAMGWFRQAPGKEREFVA
TINRSGGRTYYADSVKGRFTISRDNGKNMVYLQMHSLKPEDTAIYYCAA
GTGWSPQTDNEYNYWGQGTQVTVSSGGGSGCPPGYKSGVDCSPGSECKW
GCYAVDGRRYGGYGADSGV

Additionally, constructs were made where the entire K57 ultralong CDR-H3 was fused as the N- and C-terminus as follows:

K57 ultralong CDR-H3
(SEQ ID NO: 358)
TTVHQRTIKSGCPPGYKSGVDCSPGSECKWGCYAVDGRRYGGYGADSGV
GSTYTHEFYVDAWGQG
K57 ultralong CDR-H3-hC3nb1 (CDR-H3 and linker
in italics)
(SEQ ID NO: 359)
TTVHQRTIKSGCPPGYKSGVDCSPGSECKWGCYAVDGRRYGGYGADSGV
GSTYTHEFYVDAWGQGSSSSGSSSSGQVQLVETGGGLVQAGGSLRLSCA
ASGSIFSLNAMGWFRQAPGKEREFVATINRSGGRTYYADSVKGRFTISR
DNGKNMVYLQMHSLKPEDTAIYYCAAGTGWSPQTDNEYNYWGQGTQVTV
SS
hC3nb1-K57 ultralong CDR-H3 (CDR-H3 and linker
in italics)
(SEQ ID NO: 360)
QVQLVETGGGLVQAGGSLRLSCAASGSIFSLNAMGWFRQAPGKEREFVA
TINRSGGRTYYADSVKGRFTISRDNGKNMVYLQMHSLKPEDTAIYYCAA
GTGWSPQTDNEYNYWGQGTQVTVSSGSSSSTTVHQRTIKSGCPPGYKSG
VDCSPGSECKWGCYAVDGRRYGGYGADSGVGSTYTHEFYVDAWGQG

We measured binding to C3 and C5 by SPR multi-cycle kinetics and we report full binding kinetics and equilibrium dissociation constants (KD) for binding to both proteins.

TABLE 29
Summary of C3 binding kinetics from Biacore single-cycle kinetics.
Data from n = 3 independent experiments

| | Mean | Kₒₙ 95% | Mean | K_{off} 95% | Mean | K_{D} 95% |
|---|---|---|---|---|---|---|
| Construct | Kₒₙ | CI | K_{off} | CI | K_{D} | CI |
| hC3Nb1 WT | 8.41E+05 | 2.52E+05 | 1.37E−04 | 8.08E−05 | 1.63E−10 | 9.70E−11 |
| hC3Nb1 Loop 1 | 4.61E+05 | 6.08E+05 | 3.24E−04 | 1.93E−04 | 8.15E−09 | 1.50E−08 |
| hC3Nb1 Loop 2 | 5.09E+05 | 7.45E+05 | 1.31E−04 | 7.81E−05 | 1.80E−09 | 3.08E−09 |
| hC3Nb1Loop2ΔP | 7.32E+05 | 1.01E+06 | 2.01E−04 | 1.52E−04 | 1.51E−09 | 2.57E−09 |
| hC3Nb1 Loop 3 | 6.73E+05 | 9.89E+05 | 9.36E−04 | 1.54E−03 | 1.08E−07 | 2.12E−07 |
| hC3Nb1 C-Term | 5.07E+05 | 2.00E+05 | 1.42E−04 | 4.64E−05 | 3.02E−10 | 1.65E−10 |
| hC3Nb1 N-Term | 5.47E+05 | 4.26E+05 | 1.52E−04 | 5.62E−05 | 3.99E−10 | 3.93E−10 |
| K57 | ND | — | ND | — | ND | — |

TABLE 30
Summary of C5 binding kinetics from Biacore single-cycle kinetics.
Data from n = 3 independent experiments.

| | Mean | Kₒₙ 95% | Mean | K_{off} 95% | Mean | K_{D} 95% |
|---|---|---|---|---|---|---|
| Construct | Kₒₙ | CI | K_{off} | CI | K_{D} | CI |
| hC3Nb1 | ND | — | ND | — | ND | — |
| hC3Nb1 Loop 1 | 8.74E+04 | 9.95E+04 | 4.89E−04 | 6.26E−04 | 1.31E−08 | 1.79E−08 |
| hC3Nb1 Loop 2 | 2.85E+05 | 1.25E+04 | 9.09E−05 | 2.67E−05 | 3.21E−10 | 1.09E−10 |
| hC3Nb1Loop2ΔP | 1.76E+05 | 4.57E+04 | 1.48E−04 | 1.28E−05 | 8.64E−10 | 1.79E−10 |
| hC3Nb1 Loop 3 | 2.37E+05 | 3.23E+04 | 1.37E−04 | 3.69E−05 | 5.96E−10 | 2.44E−10 |
| hC3Nb1 C-Term | 4.52E+05 | 9.42E+04 | 1.19E−04 | 1.65E−05 | 2.71E−10 | 8.39E−11 |
| hC3Nb1 N-Term | 6.18E+05 | 4.84E+04 | 1.18E−04 | 1.52E−05 | 1.92E−10 | 3.84E−11 |
| K57 | 3.53E+05 | 8.09E+04 | 3.10E−04 | 1.93E−04 | 8.42E−10 | 3.26E−10 |

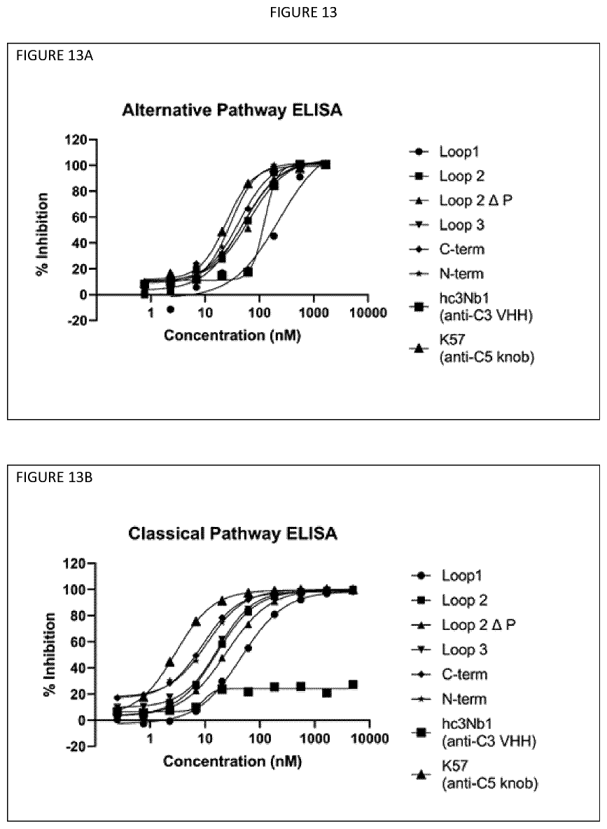

Finally, we tested our constructs in AP and CP complement activation ELISAs, which measured C5b neoepitope deposition. These data are displayed in FIG. 13.

### Example 7: Isolated Antibody Fragments According to the Invention Fused to Effector Molecules for Improving Half-Life In Vivo

As disclosed, the isolated antibody fragments according to the invention may be fused to an effector molecule which may increase the half-life of the isolated antibody fragment in vivo. Examples of suitable effector molecules of this type include Fc fragments and albumin.

In the present example, knob domain peptides were inserted into albumin, and Fc fragments. Advantageously, the resulting fusion proteins may confer an improved half-life to the knob domain peptides, useful in therapy.

7.1. Human and Rat Albumin Knob Domain Fusion Proteins

By virtue of the proximity of the N- and C-terminals, knob domain peptides can be engineered within loop or turn motifs in the middle of polypeptide chains, without perturbing the global protein fold.

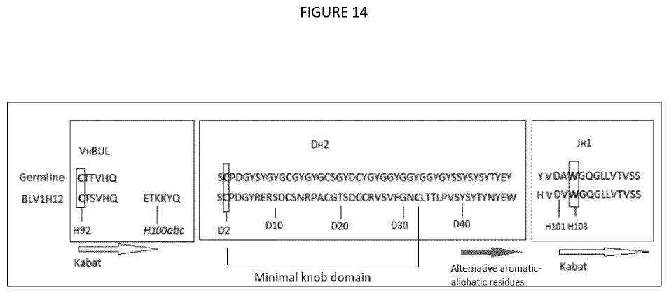

Fusion proteins were expressed which incorporated the K57 and K92 knob domain peptides, flanked by a linker sequence, in various sites throughout the polypeptide chain of the Homo sapiens' and Rattus norvegicus' serum albumin proteins, as shown in FIG. 14. The sites were selected on the basis that they were unlikely to impede binding to the neonatal Fc receptor. On this basis, these fusion proteins may exhibit binding to human C5 protein and an extended serum half-life in vivo.

K57 knob domain (shown in italics):
(SEQ ID NO: 334)
GCPPGYKSGVDCSPGSECKWGCYAVDGRRYGGYGADSGV
K92 knob domain (shown in italics):
(SEQ ID NO: 450)
TCPEGWSECGVAIYGYECGRWGCGHFLNSGPNISPYVST
Linker (shown in bold): SGGGS
Rat serum albumin-K57 site 1
(SEQ ID NO: 451)
RGVFRREAHKSEIAHRFKDLGEQHFKGLVLIAFSQYLQKCPYEEHIKLVQEVTDFAK
TCVADENASGGGSGCPPGYKSGVDCSPGSECKWGCYAVDGRRYGGYGADSGVSGGGS
ENCDKSIHTLFGDKLCAIPKLRDNYGELADCCAKQEPERNECFLQHKDDNPNLPPFQ
RPEAEAMCTSFQENPTSFLGHYLHEVARRHPYFYAPELLYYAEKYNEVLTQCCTESD
KAACLTPKLDAVKEKALVAAVRQRMKCSSMQRFGERAFKAWAVARMSQRFPNAE
FAEITKLATDVTKINKECCHGDLLECADDRAELAKYMCENQATISSKLQACCDKPVL
QKSQCLAEIEHDNIPADLPSIAADFVEDKEVCKNYAEAKDVFLGTFLYEYSRRHPDY
SVSLLLRLAKKYEATLEKCCAEGDPPACYGTVLAEFQPLVEEPKNLVKTNCELYEKL
GEYGFQNAVLVRYTQKAPQVSTPTLVEAARNLGRVGTKCCTLPEAQRLPCVEDYLS
AILNRLCVLHEKTPVSEKVTKCCSGSLVERRPCFSALTVDETYVPKEFKAETFTFHSDI
CTLPDKEKQIKKQTALAELVKHKPKATEDQLKTVMGDFAQFVDKCCKAADKDNCF
ATEGPNLVARSKEALA
Human serum albumin-K57 site 1
(SEQ ID NO: 452)
RGVFRRDAHKSEVAHRFKDLGEENFKALVLIAFAQYLQQCPFEDHVKLVNEVTEFA
KTCVADESASGGGSGCPPGYKSGVDCSPGSECKWGCYAVDGRRYGGYGADSGVSGGG
SENCDKSLHTLFGDKLCTVATLRETYGEMADCCAKQEPERNECFLQHKDDNPNLPR
LVRPEVDVMCTAFHDNEETFLKKYLYEIARRHPYFYAPELLFFAKRYKAAFTECCQA
ADKAACLLPKLDELRDEGKASSAKQRLKCASLQKFGERAFKAWAVARLSQRFPKAE
FAEVSKLVTDLTKVHTECCHGDLLECADDRADLAKYICENQDSISSKLKECCEKPLL
EKSHCIAEVENDEMPADLPSLAADFVESKDVCKNYAEAKDVFLGMFLYEYARRHPD
YSVVLLLRLAKTYETTLEKCCAAADPHECYAKVFDEFKPLVEEPQNLIKQNCELFEQ
LGEYKFQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNAETFTFH
ADICTLSEKERQIKKQTALVELVKHKPKATKEQLKAVMDDFAAFVEKCCKADDKET
CFAEEGKKLVAASQAALGL
Rat serum albumin-K92 site 1
(SEQ ID NO: 453)
RGVFRREAHKSEIAHRFKDLGEQHFKGLVLIAFSQYLQKCPYEEHIKLVQEVTDFAK
TCVADENASGGGSTCPEGWSECGVAIYGYECGRWGCGHFLNSGPNISPYVSTSGGGSE
NCDKSIHTLFGDKLCAIPKLRDNYGELADCCAKQEPERNECFLQHKDDNPNLPPFQR
PEAEAMCTSFQENPTSFLGHYLHEVARRHPYFYAPELLYYAEKYNEVLTQCCTESDK
AACLTPKLDAVKEKALVAAVRQRMKCSSMQRFGERAFKAWAVARMSQRFPNAEF
AEITKLATDVTKINKECCHGDLLECADDRAELAKYMCENQATISSKLQACCDKPVL
QKSQCLAEIEHDNIPADLPSIAADFVEDKEVCKNYAEAKDVFLGTFLYEYSRRHPDY
SVSLLLRLAKKYEATLEKCCAEGDPPACYGTVLAEFQPLVEEPKNLVKTNCELYEKL
GEYGFQNAVLVRYTQKAPQVSTPTLVEAARNLGRVGTKCCTLPEAQRLPCVEDYLS
AILNRLCVLHEKTPVSEKVTKCCSGSLVERRPCFSALTVDETYVPKEFKAETFTFHSDI
CTLPDKEKQIKKQTALAELVKHKPKATEDQLKTVMGDFAQFVDKCCKAADKDNCF
ATEGPNLVARSKEALA
Human serum albumin-K92 site 1
(SEQ ID NO: 454)
RGVFRRDAHKSEVAHRFKDLGEENFKALVLIAFAQYLQQCPFEDHVKLVNEVTEFA
KTCVADESASGGGSTCPEGWSECGVAIYGYECGRWGCGHFLNSGPNISPYVSTSGGGS
ENCDKSLHTLFGDKLCTVATLRETYGEMADCCAKQEPERNECFLQHKDDNPNLPRL
VRPEVDVMCTAFHDNEETFLKKYLYEIARRHPYFYAPELLFFAKRYKAAFTECCQA
ADKAACLLPKLDELRDEGKASSAKQRLKCASLQKFGERAFKAWAVARLSQRFPKAE
FAEVSKLVTDLTKVHTECCHGDLLECADDRADLAKYICENQDSISSKLKECCEKPLL
EKSHCIAEVENDEMPADLPSLAADFVESKDVCKNYAEAKDVFLGMFLYEYARRHPD
YSVVLLLRLAKTYETTLEKCCAAADPHECYAKVFDEFKPLVEEPQNLIKQNCELFEQ
LGEYKFQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNAETFTFH
ADICTLSEKERQIKKQTALVELVKHKPKATKEQLKAVMDDFAAFVEKCCKADDKET
CFAEEGKKLVAASQAALGL
Rat serum albumin-K57 site 2
(SEQ ID NO: 455)
RGVFRREAHKSEIAHRFKDLGEQHFKGLVLIAFSQYLQKCPYEEHIKLVQEVTDFAK
TCVADENAENCDKSIHTLFGDKLCAIPKLRDNYGELADCCAKQEPERNECFLQHKD
DNPNLPPFQRPEAEAMCTSFQENPTSFLGHYLHEVARRHPYFYAPELLYYAEKYNEV
LTQCCTESGGGSGCPPGYKSGVDCSPGSECKWGCYAVDGRRYGGYGADSGVSGGGSS
DKAACLTPKLDAVKEKALVAAVRQRMKCSSMQRFGERAFKAWAVARMSQRFPNA
EFAEITKLATDVTKINKECCHGDLLECADDRAELAKYMCENQATISSKLQACCDKPV
LQKSQCLAEIEHDNIPADLPSIAADFVEDKEVCKNYAEAKDVFLGTFLYEYSRRHPD
YSVSLLLRLAKKYEATLEKCCAEGDPPACYGTVLAEFQPLVEEPKNLVKTNCELYEK
LGEYGFQNAVLVRYTQKAPQVSTPTLVEAARNLGRVGTKCCTLPEAQRLPCVEDYL
SAILNRLCVLHEKTPVSEKVTKCCSGSLVERRPCFSALTVDETYVPKEFKAETFTFHS
DICTLPDKEKQIKKQTALAELVKHKPKATEDQLKTVMGDFAQFVDKCCKAADKDN
CFATEGPNLVARSKEALA
Human serum albumin-K57 site 2
(SEQ ID NO: 456)
RGVFRRDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKA
AFTECCQASGGGSGCPPGYKSGVDCSPGSECKWGCYAVDGRRYGGYGADSGVSGGGS
ADKAACLLPKLDELRDEGKASSAKQRLKCASLQKFGERAFKAWAVARLSQRFPKAE
FAEVSKLVTDLTKVHTECCHGDLLECADDRADLAKYICENQDSISSKLKECCEKPLL
EKSHCIAEVENDEMPADLPSLAADFVESKDVCKNYAEAKDVFLGMFLYEYARRHPD
YSVVLLLRLAKTYETTLEKCCAAADPHECYAKVFDEFKPLVEEPQNLIKQNCELFEQ
LGEYKFQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNAETFTFH
ADICTLSEKERQIKKQTALVELVKHKPKATKEQLKAVMDDFAAFVEKCCKADDKET
CFAEEGKKLVAASQAALGL
Rat serum albumin-K92 site 2
(SEQ ID NO: 457)
RGVFRREAHKSEIAHRFKDLGEQHFKGLVLIAFSQYLQKCPYEEHIKLVQEVTDFAK
TCVADENAENCDKSIHTLFGDKLCAIPKLRDNYGELADCCAKQEPERNECFLQHKD
DNPNLPPFQRPEAEAMCTSFQENPTSFLGHYLHEVARRHPYFYAPELLYYAEKYNEV
LTQCCTESGGGSTCPEGWSECGVAIYGYECGRWGCGHFLNSGPNISPYVSTSGGGSSDK
AACLTPKLDAVKEKALVAAVRQRMKCSSMQRFGERAFKAWAVARMSQRFPNAEF
AEITKLATDVTKINKECCHGDLLECADDRAELAKYMCENQATISSKLQACCDKPVL
QKSQCLAEIEHDNIPADLPSIAADFVEDKEVCKNYAEAKDVFLGTFLYEYSRRHPDY
SVSLLLRLAKKYEATLEKCCAEGDPPACYGTVLAEFQPLVEEPKNLVKTNCELYEKL
GEYGFQNAVLVRYTQKAPQVSTPTLVEAARNLGRVGTKCCTLPEAQRLPCVEDYLS
AILNRLCVLHEKTPVSEKVTKCCSGSLVERRPCFSALTVDETYVPKEFKAETFTFHSDI
CTLPDKEKQIKKQTALAELVKHKPKATEDQLKTVMGDFAQFVDKCCKAADKDNCF
ATEGPNLVARSKEALA
Human serum albumin-K92 site 2
(SEQ ID NO: 458)
RGVFRRDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKA
AFTECCQASGGGSTCPEGWSECGVAIYGYECGRWGCGHFLNSGPNISPYVSTSGGGSA
DKAACLLPKLDELRDEGKASSAKQRLKCASLQKFGERAFKAWAVARLSQRFPKAEF
AEVSKLVTDLTKVHTECCHGDLLECADDRADLAKYICENQDSISSKLKECCEKPLLE
KSHCIAEVENDEMPADLPSLAADFVESKDVCKNYAEAKDVFLGMFLYEYARRHPDY
SVVLLLRLAKTYETTLEKCCAAADPHECYAKVFDEFKPLVEEPQNLIKQNCELFEQL
GEYKFQNALLVRYTKKVPQVSTPTLVEVSRNLGKVGSKCCKHPEAKRMPCAEDYLS
VVLNQLCVLHEKTPVSDRVTKCCTESLVNRRPCFSALEVDETYVPKEFNAETFTFHA
DICTLSEKERQIKKQTALVELVKHKPKATKEQLKAVMDDFAAFVEKCCKADDKETC
FAEEGKKLVAASQAALGL
Rat serum albumin-K57 site 3
(SEQ ID NO: 459)
RGVFRREAHKSEIAHRFKDLGEQHFKGLVLIAFSQYLQKCPYEEHIKLVQEVTDFAK
TCVADENAENCDKSIHTLFGDKLCAIPKLRDNYGELADCCAKQEPERNECFLQHKD
DNPNLPPFQRPEAEAMCTSFQENPTSFLGHYLHEVARRHPYFYAPELLYYAEKYNEV
LTQCCTESDKAACLTPKLDAVKEKALVAAVRQRMKCSSMQRFGERAFKAWAVAR
MSQRFPNAEFAEITKLATDVTKINKECCHGDLLECADDRAELAKYMCENQATISSKL
QACCDKPVLQKSQCLAEIEHDNIPADLPSIAADFVEDKEVCKNYAEAKDVFLGTFLY
EYSRRHPDYSVSLLLRLAKKYEATLEKCCAESGGGSGCPPGYKSGVDCSPGSECKWG
CYAVDGRRYGGYGADSGVSGGGSGDPPACYGTVLAEFQPLVEEPKNLVKTNCELYEK
LGEYGFQNAVLVRYTQKAPQVSTPTLVEAARNLGRVGTKCCTLPEAQRLPCVEDYL
SAILNRLCVLHEKTPVSEKVTKCCSGSLVERRPCFSALTVDETYVPKEFKAETFTFHS
DICTLPDKEKQIKKQTALAELVKHKPKATEDQLKTVMGDFAQFVDKCCKAADKDN
CFATEGPNLVARSKEALA
Human serum albumin-K57 site 3
(SEQ ID NO: 460)
RGVFRRDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKA
AFTECCQAADKAACLLPKLDELRDEGKASSAKQRLKCASLQKFGERAFKAWAVAR
LSQRFPKAEFAEVSKLVTDLTKVHTECCHGDLLECADDRADLAKYICENQDSISSKL
KECCEKPLLEKSHCIAEVENDEMPADLPSLAADFVESKDVCKNYAEAKDVFLGMFL
YEYARRHPDYSVVLLLRLAKTYETTLEKCCAASGGGSGCPPGYKSGVDCSPGSECK
WGCYAVDGRRYGGYGADSGVSGGGSADPHECYAKVFDEFKPLVEEPQNLIKQNCELF
EQLGEYKFQNALLVRYTKKVPQVSTPTLVEVSRNLGKVGSKCCKHPEAKRMPCAED
YLSVVLNQLCVLHEKTPVSDRVTKCCTESLVNRRPCFSALEVDETYVPKEFNAETFT
FHADICTLSEKERQIKKQTALVELVKHKPKATKEQLKAVMDDFAAFVEKCCKADDK
ETCFAEEGKKLVAASQAALGL
Rat serum albumin-K92 site 3
(SEQ ID NO: 461)
RGVFRREAHKSEIAHRFKDLGEQHFKGLVLIAFSQYLQKCPYEEHIKLVQEVTDFAK
TCVADENAENCDKSIHTLFGDKLCAIPKLRDNYGELADCCAKQEPERNECFLQHKD
DNPNLPPFQRPEAEAMCTSFQENPTSFLGHYLHEVARRHPYFYAPELLYYAEKYNEV
LTQCCTESDKAACLTPKLDAVKEKALVAAVRQRMKCSSMQRFGERAFKAWAVAR
MSQRFPNAEFAEITKLATDVTKINKECCHGDLLECADDRAELAKYMCENQATISSKL
QACCDKPVLQKSQCLAEIEHDNIPADLPSIAADFVEDKEVCKNYAEAKDVFLGTFLY
EYSRRHPDYSVSLLLRLAKKYEATLEKCCAESGGGSTCPEGWSECGVAIYGYECGRW
GCGHFLNSGPNISPYVSTSGGGSGDPPACYGTVLAEFQPLVEEPKNLVKTNCELYEKL
GEYGFQNAVLVRYTQKAPQVSTPTLVEAARNLGRVGTKCCTLPEAQRLPCVEDYLS
AILNRLCVLHEKTPVSEKVTKCCSGSLVERRPCFSALTVDETYVPKEFKAETFTFHSDI
CTLPDKEKQIKKQTALAELVKHKPKATEDQLKTVMGDFAQFVDKCCKAADKDNCF
ATEGPNLVARSKEALA
Human serum albumin-K92 site 3
(SEQ ID NO: 462)
RGVFRRDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKA
AFTECCQAADKAACLLPKLDELRDEGKASSAKQRLKCASLQKFGERAFKAWAVAR
LSQRFPKAEFAEVSKLVTDLTKVHTECCHGDLLECADDRADLAKYICENQDSISSKL
KECCEKPLLEKSHCIAEVENDEMPADLPSLAADFVESKDVCKNYAEAKDVFLGMFL
YEYARRHPDYSVVLLLRLAKTYETTLEKCCAASGGGSTCPEGWSECGVAIYGYECGR
WGCGHFLNSGPNISPYVSTSGGGSADPHECYAKVFDEFKPLVEEPQNLIKQNCELFEQ
LGEYKFQNALLVRYTKKVPQVSTPTLVEVSRNLGKVGSKCCKHPEAKRMPCAEDYL
SVVLNQLCVLHEKTPVSDRVTKCCTESLVNRRPCFSALEVDETYVPKEFNAETFTFH
ADICTLSEKERQIKKQTALVELVKHKPKATKEQLKAVMDDFAAFVEKCCKADDKET
CFAEEGKKLVAASQAALGL
Rat serum albumin-K57 site 4
(SEQ ID NO: 463)
RGVFRREAHKSEIAHRFKDLGEQHFKGLVLIAFSQYLQKCPYEEHIKLVQEVTDFAK
TCVADENAENCDKSIHTLFGDKLCAIPKLRDNYGELADCCAKQEPERNECFLQHKD
DNPNLPPFQRPEAEAMCTSFQENPTSFLGHYLHEVARRHPYFYAPELLYYAEKYNEV
LTQCCTESDKAACLTPKLDAVKEKALVAAVRQRMKCSSMQRFGERAFKAWAVAR
MSQRFPNAEFAEITKLATDVTKINKECCHGDLLECADDRAELAKYMCENQATISSKL
QACCDKPVLQKSQCLAEIEHDNIPADLPSIAADFVEDKEVCKNYAEAKDVFLGTFLY
EYSRRHPDYSVSLLLRLAKKYEATLEKCCAEGDPPACYGTVLAEFQPLVEEPKNLVK
TNCELYEKLGEYGFQNAVLVRYTQKAPQVSTPTVEAARNLGRVGTKCCTLPEAQR
LPCVEDYLSAILNRLCVLHEKTPVSEKVTKCCSGSLVERRPCFSALTVDETYVPKEFK
AETFTFHSDICTLPDKEKQIKKQTALAELVKHKPKATEDQLKTVMGDFAQFVDKCC
KAASGGGSGCPPGYKSGVDCSPGSECKWGCYAVDGRRYGGYGADSGVSGGGSDKDN
CFATEGPNLVARSKEALA
Human serum albumin-K57 site 4
(SEQ ID NO: 464)
RGVFRRDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKA
AFTECCQAADKAACLLPKLDELRDEGKASSAKQRLKCASLQKFGERAFKAWAVAR
LSQRFPKAEFAEVSKLVTDLTKVHTECCHGDLLECADDRADLAKYICENQDSISSKL
KECCEKPLLEKSHCIAEVENDEMPADLPSLAADFVESKDVCKNYAEAKDVFLGMFL
YEYARRHPDYSVVLLLRLAKTYETTLEKCCAAADPHECYAKVFDEFKPLVEEPQNLI
KQNCELFEQLGEYKFQNALLVRYTKKVPQVSTPTLVEVSRNLGKVGSKCCKHPEAK
RMPCAEDYLSVVLNQLCVLHEKTPVSDRVTKCCTESLVNRRPCFSALEVDETYVPKE
FNAETFTFHADICTLSEKERQIKKQTALVELVKHKPKATKEQLKAVMDDFAAFVEKC
CKADSGGGSGCPPGYKSGVDCSPGSECKWGCYAVDGRRYGGYGADSGVSGGGSDKET
CFAEEGKKLVAASQAALGL
Rat serum albumin-K92 site 4
(SEQ ID NO: 465)
RGVFRREAHKSEIAHRFKDLGEQHFKGLVLIAFSQYLQKCPYEEHIKLVQEVTDFAK
TCVADENAENCDKSIHTLFGDKLCAIPKLRDNYGELADCCAKQEPERNECFLQHKD
DNPNLPPFQRPEAEAMCTSFQENPTSFLGHYLHEVARRHPYFYAPELLYYAEKYNEV
LTQCCTESDKAACLTPKLDAVKEKALVAAVRQRMKCSSMQRFGERAFKAWAVAR
MSQRFPNAEFAEITKLATDVTKINKECCHGDLLECADDRAELAKYMCENQATISSKL
QACCDKPVLQKSQCLAEIEHDNIPADLPSIAADFVEDKEVCKNYAEAKDVFLGTFLY
EYSRRHPDYSVSLLLRLAKKYEATLEKCCAEGDPPACYGTVLAEFQPLVEEPKNLVK
TNCELYEKLGEYGFQNAVLVRYTQKAPQVSTPTLVEAARNLGRVGTKCCTLPEAQR
LPCVEDYLSAILNRLCVLHEKTPVSEKVTKCCSGSLVERRPCFSALTVDETYVPKEFK
AETFTFHSDICTLPDKEKQIKKQTALAELVKHKPKATEDQLKTVMGDFAQFVDKCC
KAASGGGSTCPEGWSECGVAIYGYECGRWGCGHFLNSGPNISPYVSTSGGGSDKDNCF
ATEGPNLVARSKEALA
Human serum albumin-K92 site 4
(SEQ ID NO: 466)
RGVFRRDAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNECFLQHK
DDNPNLPRLVRPEVDVMCTAFHDNEETFLKKYLYEIARRHPYFYAPELLFFAKRYKA
AFTECCQAADKAACLLPKLDELRDEGKASSAKQRLKCASLQKFGERAFKAWAVAR
LSQRFPKAEFAEVSKLVTDLTKVHTECCHGDLLECADDRADLAKYICENQDSISSKL
KECCEKPLLEKSHCIAEVENDEMPADLPSLAADFVESKDVCKNYAEAKDVFLGMFL
YEYARRHPDYSVVLLLRLAKTYETTLEKCCAAADPHECYAKVFDEFKPLVEEPQNLI
KQNCELFEQLGEYKFQNALLVRYTKKVPQVSTPTLVEVSRNLGKVGSKCCKHPEAK
RMPCAEDYLSVVLNQLCVLHEKTPVSDRVTKCCTESLVNRRPCFSALEVDETYVPKE
FNAETFTFHADICTLSEKERQIKKQTALVELVKHKPKATKEQLKAVMDDFAAFVEKC
CKADSGGGSTCPEGWSECGVAIYGYECGRWGCGHFLNSGPNISPYVSTSGGGSDKETC
FAEEGKKLVAASQAALGL

7.2. Human and Rat IgG1 Fc-Knob Domain Fusion Proteins

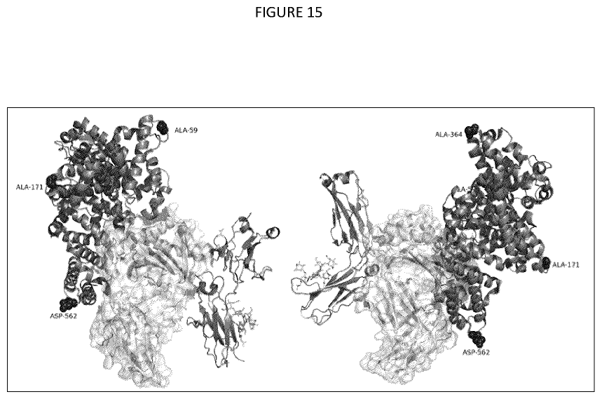

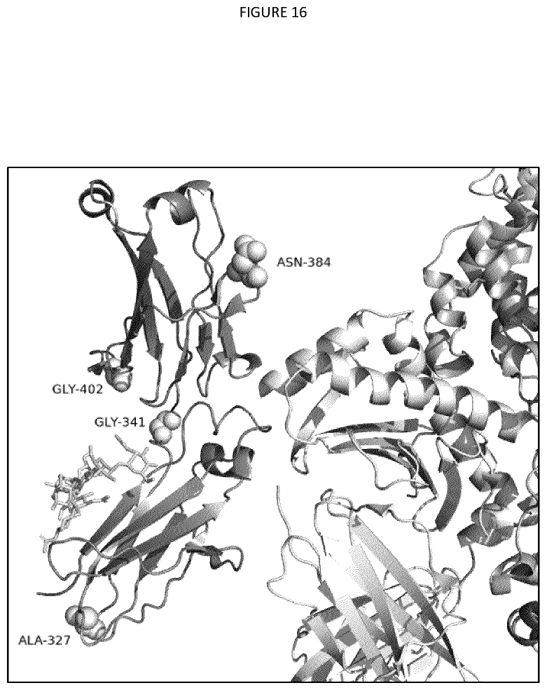

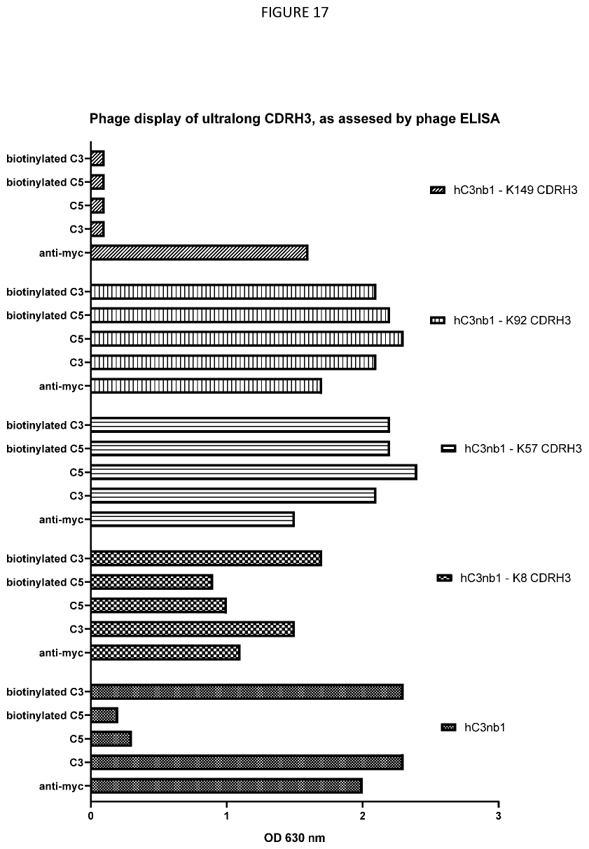

Fusion proteins were designed which incorporated the K149 knob domain peptide, flanked by a linker sequence, into various sites in the middle of the polypeptide chain of the Homo sapiens and Rattus norvegicus immunoglobulin gamma-1 heavy chain constant region, as shown in FIG. 15. The sites were selected on the basis that they were unlikely to impede binding to the neonatal Fc receptor. On this basis, these K149-IgG1 fusion proteins may exhibit binding to human C5 protein and an extended serum half-life in vivo.

K149 sequence (shown in italics):
(SEQ ID NO: 313)
SCPDGFSYRSWDDFCCPMVGRCLAPRN
Linker sequences (shown in bold): SGGGGS
Rat IgG1 heavy chain constant region Site 1-K149
(SEQ ID NO: 467)
AETTAPSVYPLAPGTALKSNSMVTLGCLVKGYFPEPVTVTWNSGALSSGVHTFPAVL
QSGLYTLTSSVTVPSSTWPSQTVTCNVAHPASSTKVDKKIVPRNCGGDCKPCICTGSE
VSSVFIFPPKPKDVLTITLTPKVTCVVVDISQDDPEVHFSWFVDDVEVHTAQTRPPEE
QFNSTFRSVSELPILHQDWLNGRTFRCKVTSASGGGGSSCPDGFSYRSWDDFCCPMV
GRCLAPRNSGGGGSAFPSPIEKTISKPEGRTQVPHVYTMSPTKEEMTQNEVSITCMVK
GFYPPDIYVEWQMNGQPQENYKNTPPTMDTDGSYFLYSKLNVKKEKWQQGNTFTC
SVLHEGLHNHHTEKSLSHSPGK
Rat IgG1 heavy chain constant region Site 2-K149
(SEQ ID NO: 468)
AETTAPSVYPLAPGTALKSNSMVTLGCLVKGYFPEPVTVTWNSGALSSGVHTFPAVL
QSGLYTLTSSVTVPSSTWPSQTVTCNVAHPASSTKVDKKIVPRNCGGDCKPCICTGSE
VSSVFIFPPKPKDVLTITLTPKVTCVVVDISQDDPEVHFSWFVDDVEVHTAQTRPPEE
QFNSTFRSVSELPILHQDWLNGRTFRCKVTSAAFPSPIEKTISKPESGGGGSSCPDGFS
YRSWDDFCCPMVGRCLAPRNSGGGGSGRTQVPHVYTMSPTKEEMTQNEVSITCMVK
GFYPPDIYVEWQMNGQPQENYKNTPPTMDTDGSYFLYSKLNVKKEKWQQGNTFTC
SVLHEGLHNHHTEKSLSHSPGK
Rat IgG1 heavy chain constant region Site 3-K149
(SEQ ID NO: 469)
AETTAPSVYPLAPGTALKSNSMVTLGCLVKGYFPEPVTVTWNSGALSSGVHTFPAVL
QSGLYTLTSSVTVPSSTWPSQTVTCNVAHPASSTKVDKKIVPRNCGGDCKPCICTGSE
VSSVFIFPPKPKDVLTITLTPKVTCVVVDISQDDPEVHFSWFVDDVEVHTAQTRPPEE
QFNSTFRSVSELPILHQDWLNGRTFRCKVTSAAFPSPIEKTISKPEGRTQVPHVYTMSP
TKEEMTQNEVSITCMVKGFYPPDIYVEWQMNSGGGGSSCPDGFSYRSWDDFCCPMV
GRCLAPRNSGGGGSGQPQENYKNTPPTMDTDGSYFLYSKLNVKKEKWQQGNTFTC
SVLHEGLHNHHTEKSLSHSPGK
Rat IgG1 heavy chain constant region Site 4-K149
(SEQ ID NO: 470)
AETTAPSVYPLAPGTALKSNSMVTLGCLVKGYFPEPVTVTWNSGALSSGVHTFPAVL
QSGLYTLTSSVTVPSSTWPSQTVTCNVAHPASSTKVDKKIVPRNCGGDCKPCICTGSE
VSSVFIFPPKPKDVLTITLTPKVTCVVVDISQDDPEVHFSWFVDDVEVHTAQTRPPEE
QFNSTFRSVSELPILHQDWLNGRTFRCKVTSAAFPSPIEKTISKPEGRTQVPHVYTMSP
TKEEMTQNEVSITCMVKGFYPPDIYVEWQMNGQPQENYKNTPPTMDTDGSGGGGS
SCPDGFSYRSWDDFCCPMVGRCLAPRNSGGGGSSYFLYSKLNVKKEKWQQGNTFTC
SVLHEGLHNHHTEKSLSHSPGK
Human IgG1 heavy chain constant region Site 1-K149
(SEQ ID NO: 471)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKASGGGGSSCPDGFSYRS
WDDFCCPMVGRCLAPRNSGGGGSLPAPIEKTISKAKGQPREPQVYTLPPSRDELTKN
QVSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 heavy chain constant region Site 2-K149
(SEQ ID NO: 472)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGSGG
GGSSCPDGFSYRSWDDFCCPMVGRCLAPRNSGGGGSQPREPQVYTLPPSRDELTKNQ
VSLTCLVKGFYPSDIAVEWESNGQPENNYKTTPPVLDSDGSFFLYSKLTVDKSRW
QQGNVFSCSVMHEALHNHYTQKSLSLSPGK
Human IgG1 heavy chain constant region Site 3-K149
(SEQ ID NO: 473)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNSGGGGSSCPDGFSYRSW
DDFCCPMVGRCLAPRNSGGGGSGQPENNYKTTPPVLDSDGSFFLYSKLTVDKSRW
QQGNVFSCSVMHEALHNHYTQKSLSLSPGK
Human IgG1 heavy chain constant region Site 4-K149
(SEQ ID NO: 474)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSD
GSGGGGSSCPDGFSYRSWDDFCCPMVGRCLAPRNSGGGGSSFFLYSKLTVDKSRWQ
QGNVFSCSVMHEALHNHYTQKSLSLSPGK

7.3. Synthesis of the Fusion Proteins, Expression in Expi293F Cells, and Binding to C5 Synthesis and Expression in Expi293F Cells:

Custom synthesis and cloning into a pMH expression vector, containing a human cytomegalovirus (CMV) promoter and an in-frame C-terminal 10× Histidine tag, may be performed by ATUM. For all constructs, a Mus musculus immunoglobulin heavy-chain leader sequence may be used: MEWSWVFLFFLSVTTGVHS (SEQ ID NO: 475).

Plasmid DNA is amplified using QIAGEN Plasmid Plus Giga Kits, and quantified by A260. Individual Expi293F cell cultures, at 3×10⁶ cells/mL, per construct, are set up using Expifectamine 293 Transfection kits (Invitrogen), as per the manufacturer's instructions. The cells are cultured for four days, centrifuged at 4000 rpm for one hour and filtered through a 0.22 μm filter. Using an Akta pure (GE Healthcare), Hi-Trap Nickel excel columns (e.g. GE Healthcare) are equilibrated with 10 column volumes (CV) of PBS. Cell supernatants are loaded at 1.0 mL/min and the column are washed with 7×CV of PBS, 0.5 M NaCl. The column is then washed with 7×CV of Buffer A (0.5 M NaCl, 0.02 M Imidazole, PBS pH 7.3). Protein samples are eluted by isocratic elution with 10×CV of Buffer B (0.5 M NaCl, 0.25 M Imidazole, PBS pH 7.3). Post elution, the protein containing fractions are pooled and buffer exchanged into PBS, using PD-10 columns (GE Healthcare) or Slide-a-lyzer dialysis cassettes (Thermo Fisher). Proteins are visualised by SDS-PAGE, quantified by A₂₈₀ and aliquoted for storage at −80° C.

Biacore Single-Cycle Kinetics:

The ability of the fusion proteins to specifically bind C5 can be assessed by Biacore. Using a Biacore 8K (GE Healthcare), C5 protein is immobilized on a CM5 Series S sensor chip by amine coupling. Flow cells are activated using a minimal immobilisation protocol: EDC/NHS was mixed at 1:2 ratio (flow rate, 10 μL/min; contact time, 30 s). C5, at 1 μg/mL in pH 4.5 Sodium-acetate buffer, is immobilized on flow cell two only (flow rate, 10 μL/min; contact time, 420 s). Finally, ethanolamine is applied to both flow cells (flow rate, 10 μL/min; contact time, 420 s). A final immobilization level of approximately 100-200 response units is obtained.

Single-cycle kinetics are measured using a 7-point, 3-fold titration, from a highest concentration of 1 μM (spanning a range of 1 μM to 1.4 nM) in HBS-EP buffer (GE healthcare). A flow rate of 40 μL/min is used, with a contact time of 230 s and a dissociation time of 1800 s. Binding to the reference surface is subtracted, and the data are fitted to a single-site binding model using Biacore evaluation software.

### Example 8: Phage Display Libraries of Isolated Antibody Fragments According to the Invention

Phage display libraries of knob domain peptides as disclosed herein could be generated by any suitable method, such as linking the knob domains directly or via a spacer to pIII of M13, for example within the entire bovine CDR-H3 or alternatively within the framework of another protein, such as an antibody fragment e.g. scFv, Fab or VHH.

To enable phage display of ultralong CDR-H3 sequences on the pIII of M13 filamentous bacteriophage, hC3nb1, a camelid VHH which bound complement component C3, was engineered to accommodate bovine ultralong CDR-H3 sequences. The CDR-H3 sequences were inserted between residues H74 and H75 (Kabat numbering system), into a non-binding VH framework 3 loop. When displayed on phage, the modified hC3nb1 retained binding to C3 via it's canonical CDRs, in a manner which is well explained by the co-crystal structure (pdb accession code: 6EHG). In most cases binding to C5 was also observed. The sequences, methods and results are presented below.

Sequences:
hC3nb1 sequence, with the insertion site shown in bold:
(SEQ ID NO: 351)
QVQLVETGGGLVQAGGSLRLSCAASGSIFSLNAMGWFRQAPGKEREFVATINRSGG
RTYYADSVKGRFTISRDNGKNMVYLQMHSLKPEDTAIYYCAAGTGWSPQTDNEYN
YWGQGTQVTVSS
hC3nb1-K8
(SEQ ID NO: 476)
QVQLVETGGGLVQAGGSLRLSCAASGSIFSLNAMGWFRQAPGKEREFVATINRSGG
RTYYADSVKGRFTISRDNG [symbol] [symbol]
KNMVYLQMHSLKPEDTAIYYCAAGTGWS
PQTDNEYNYWGQGTQVTVSS
K8 CDR-H3:
(SEQ ID NO: 477)

[symbol]

[symbol]

[symbol]

hC3nb1-K57
(SEQ ID NO: 478)
QVQLVETGGGLVQAGGSLRLSCAASGSIFSLNAMGWFRQAPGKEREFVATINRSGG
RTYYADSVKGRFTISRDNG [symbol] [symbol] [symbol]
KNMVYLQMHSLKPEDTAIYYCAAGTGWSPQTD
NEYNYWGQGTQVTVSS
K57 CDR-H3:
(SEQ ID NO: 104)

[symbol]

[symbol]

[symbol]

hC3nb1-K92
(SEQ ID NO: 479)
QVQLVETGGGLVQAGGSLRLSCAASGSIFSLNAMGWFRQAPGKEREFVATINRSGG
RTYYADSVKGRFTISRDNG [symbol] [symbol] [symbol]
KNMVYLQMHSLKPEDTAIYYCAAGTGWSPQTDNE
YNYWGQGTQVTVSS
K92 CDR-H3:
(SEQ ID NO: 13)

[symbol]

[symbol]

[symbol]

hC3nb1-K149
(SEQ ID NO: 480)
QVQLVETGGGLVQAGGSLRLSCAASGSIFSLNAMGWFRQAPGKEREFVATINRSGG
RTYYADSVKGRFTISRDNG [symbol] [symbol] [symbol]
KNMVYLQMHSLKPEDTAIYYCAAGTGWSPQTDNEYNYWGQGTQV
TVSS
K149 CDR-H3:
(SEQ ID NO: 1)

[symbol]

[symbol]

Methods

Bovine CDRH3, hC3nb1 and hC3nb1-ultralong CDR-H3 were cloned into a phagemid vector by TWIST Biosciences. The phagemid vector contains a pelB leader sequence, ahead of the CDRH3, hC3nb1 or hC3nb1-ultralong CDR-H3 sequence for display. This is followed by a poly-histidine and c-myc-Tag, which is fused directly to the pIII. The entire pIII fusion protein is under the control of glucose repressible lac promoter. Upon superinfection with helper phage, an M13 replication origin will result in the synthesis of single stranded phagemid DNA, encoding the pIII fusion display construct. Each construct was prepared with and without co-expression of FKBP-type peptidyl-prolyl cis-trans isomerase (FkpA), accession number: P45523 (FKBA_ECOLI).

The constructs were transformed into TG-1 cells using a heat-shock method. Briefly, 1 μL of plasmid DNA was added to 50 μl of competent cells, mixed briefly, and incubated on ice for 30 minutes. The cells were heat shocked at 42° C. for 30 seconds and incubated on ice for 2 minutes. 200 μL of media was added and 50 μL was plated on 2TY media+100 μg/ml Carbenicillin+2% Glucose and incubated overnight at 37° C.

Individual colonies were picked from the plates and placed into individual 5.0 mL cultures of 2TY media+100 μg/ml Carbenicillin+2% Glucose. The culture was incubated at 37° C. with shaking until an OD of ˜0.5 was reached, whereupon they were superinfected with M13K07 helper phage at a MOI of 10 (New England Biolabs). The cultures were centrifuged and resuspended in the absence of glucose (2TY media+100 μg/ml Carbenicillin+50 ug/ml kanamycin) allowing leakiness of the lac promoter and therefore expression of the recombinant VHH-pIII fusion protein from the phagemid. Finally, the cells were grown overnight at 30° C.

Complement C5 and C3 were non-site specifically labelled with biotin to permit immobilisation onto an ELISA plate. Stocks of amine-reactive EZ-link biotin (e.g. Thermo Scientific) were prepared in DMSO and frozen at −20° C. Biotin was added at a ten-fold molar excess to C5 and C3 in PBS. The solution was incubated for 60 minutes at room temperature. Unreacted biotin was removed using two sequential 0.5 mL Zeba desalting columns (Thermo Fisher), as per the manufacturer's instructions.

96-well ELISA plates were coated with 100 μL/well of either: anti c-Myc antibody (e.g. Novus clone #9E10), C3 protein or C5 protein in PBS (10 μg/mL). Additional plates were coated with streptavidin in PBS (5 μg/mL) and incubated overnight at 2-8° C. After coating, the overnight phage rescue cultures were centrifuged at 4,500 rpm for 10 minutes. To block the phage, 500 supernatant was removed and put into a fresh block, containing 500 μL per well 6% Marvel milk (w/v) powder in PBS. The coating solution was removed from the ELISA plate by washing with four cycles of Wash Buffer (PBS, 0.1% Tween 20), at 300 μL per well, using a plate washer (BMG labtech). Blocking Buffer (PBS, 3% Marvel milk powder (w/v)), was added (100 μL/well) and the plate was incubated for 1 hour at room temperature. The blocked streptavidin plates were washed again and 100 μL/well of C5-biotin (5 μg/mL), C3-biotin (5 μg/mL), or Assay Buffer (lx PBS) were added and incubated for 30 minutes. All plates were then washed and the blocked phage supernatant was added (100 μL/well) and incubated for 1 hour with shaking (400 rpm). The plates were washed again and an anti-M13 HRP Ab, at 1:11000 dilution in blocking Buffer (PBS, 3% Marvel milk powder (w/v), was added (100 μL/well); and incubated for 1 hour at room temperature with shaking (400 rpm). Finally, the plates were washed and 100 μL per well of ‘One Step’ TMB (Thermo Scientific) was added and incubated for approximately 5 minutes. The plate was read on BMG Labtech a plate reader at 630 nm.

Results

The results are presented in FIG. 16. The hC3nb1 VHH was successfully displayed on phage, as indicated by the anti-myc tag and binding to human C3, both when immobilised directly to the plate and when biotinylated C3 was captured on a streptavidin plate. No cross-reactivity to C5 protein was observed.

The hC3nb1-K8, hC3nb1-K57 and hC3nb1-K92 proteins all displayed binding to human C5, both when immobilised directly to the plate and when biotinylated C5 was captured on a streptavidin plate. This suggests that the ultralong CDR-H3 adopt a native fold on the VHH, achieving successful surface display. Only the hC3nb1-K149 protein did not bind either form of C3 or C5.

Co-expression of FkpA is not prerequisite for ultralong CDR-H3 display with the hC3nb1 fusion proteins but it may offer a modest increase in display levels, potentially accounting for a small increase of signal in the phage ELISA (data not shown).

### Example 9: Förster Resonance Energy Transfer (FRET) Assays

To provide further evidence for high affinity binding of knob domain peptides, steady state Förster resonance energy transfer (FRET) assays were developed. This was achieved by labelling C5 with a terbium chelate donor fluorophore and each of the active PGT121 knob fusion proteins as described in Example 2, with an AlexaFluor 647 acceptor fluorophore. Titrations of the PGT121 knob fusion proteins, in the presence and absence of a saturating concentration of unlabelled C5 protein, were used to derive apparent K_{D} (K_{D} app) for the interaction with C5, after an incubation of 24 hours.

Methods:

Förster resonance energy transfer (FRET) C5 purified from serum was labelled with an amine reactive terbium chelate (molecular probes, life technologies), as per the manufacturer's instructions. Briefly, C5 at 1.15 mg/mL was buffer exchanged into a 50 mM Bicine, 100 mM NaCl, pH 8.2 buffer using a zeba column (Thermo Scientific). Terbium was reconstituted in DMSO at 5 mM and added to 1% final (v/v) to create an approximate 10-fold molar excess to C5. After a one-hour incubation at room temperature, unbound dye was removed by two sequential buffer exchanges into 20 mM Tris, 100 mM NaCl, pH 7.4, again using zeba columns (Thermo Scientific). The labelling ratio was quantified by UV spectroscopy, the final molar ratio of dye to protein was 4:1. Next, PGT121 knob domain fusion proteins as described in Example 2 were labelled with an amine reactive AlexaFluor 647 (AF647) dye (molecular probes, life technologies), using the same protocol as above but with a 30-minute incubation with dye. After removal of unbound dye, UV spectroscopy quantified the final molar ratio of dye to protein as 2:1

For determination of PGT121 knob domain fusion KD app, C5 Tb was plated into a black, low volume 384-well assay plate (Corning) to give a final assay concentration (FAC) of 1 nM, either HBS-EP buffer (GE healthcare) or unlabelled C5 (1 μM FAC) were added. Eight point, three-fold titrations of PGT121 knob domain fusion proteins were prepared in HBS-EP buffer to give a range of 100 nM-0.046 nM or 500 nM-0.22 nM (FAC). The plates were wrapped in foil and incubated for 48 hours, with shaking. The plates were read on an Envision plate reader (Perkin Elmer) at 2, 24 and 48 hour intervals (HTRF laser, Excitation 330 nm and Emission 665/615 nm). For fitting, background was subtracted, and curves fitted using prism software to a 4-parameter logistic model.

Results:

Despite modification of both proteins by labelling, the PGT121 fusion proteins bound C5 with high affinity (Tables 40 and 41), in a manner consistent with our SPR experiments.

TABLE 40
FRET assay K_{D} app for PGT121 fusion proteins
binding to C5-Tb after a 2-hour incubation

| | | | | Geomean |
|---|---|---|---|---|
| | n1 | n2 | n3 | K_{D} app (nM) |
| PGT121 K8 | 6.3 | 17.9 | 4.7 | 8.1 |
| PGT121 K57 | 4.9 | 3.0 | 3.4 | 3.7 |
| PGT121 K92 | 14.0 | 10.3 | 15.5 | 13.1 |
| PGT121 K136 | NR | 46.3 | 115.0 | 73.0 |
| PGT121 K149 | 34.9 | 57.6 | 30.1 | 39.3 |

TABLE 41
FRET assay K_{D} app for PGT121 fusion proteins
binding to C5-Tb after a 24-hour incubation

| | | | | Geomean |
|---|---|---|---|---|
| | n1 | n2 | n3 | K_{D} app (nM) |
| PGT121 K8 | 6.6 | 5.0 | 4.7 | 5.4 |
| PGT121 K57 | 3.3 | 2.3 | 3.4 | 3.0 |
| PGT121 K92 | 19.3 | 7.7 | 11.3 | 11.9 |
| PGT121 K136 | 46.3 | 23.4 | 58.1 | 39.8 |
| PGT121 K149 | 126.6 | 90.6 | 24.9 | 65.9 |

Although the PGT121-K92 and PGT121-K136 fusion proteins, which displayed particularly tight-binding in the biacore mediated by a slow k_{off}, were lower affinity by the steady state method.

Competition Assay

Next, titrations of knob domains were tested in a competition assay format, using displacement of the parent PGT121 knob domain fusion protein as a readout for binding.

C5-Tb was plated to 1 nM (FAC) and titrations of knob domain peptide prepared in HBS-EP buffer, to give a range of 1000 nM-0.46 nM (FAC). PGT121 knob domain fusion-AF647 proteins were prepared to give the following concentrations, which equate to the K_{D} app measured in the previous experiment: PGT121-K8 AF647 5 nM (FAC), PGT121-K57 AF647 3 nM (FAC), PGT121-K92 AF647 12 nM (FAC), PGT121-136 AF647 40 nM (FAC) and PGT121-149 AF647 66 nM (FAC). The plates were wrapped in foil, incubated for 24 hours, with shaking, and read on an Envision plate reader (HTRF laser, Excitation 330 nm and Emission 665/615 nm). Curves were fitted using prism software to a 4-parameter logistic model. IC50 values were converted to inhibitory constants (Ki)

IC₅₀ values were measured and inhibition constants (Ki) were derived for each of the knob domain peptides using the Cheng-Prusoff equation:

$$Ki = IC 50 \div ( 1 + \frac{[ R ]}{{K}_{D}} )$$

The results are presented in Table 42 below.

TABLE 42
Competition FRET assays 24-hour incubation

| | | | | Geomean | Ki |
|---|---|---|---|---|---|
| | n1 | n2 | n3 | IC50 (nM) | (nM) |
| K8 | 20.7 | 31.2 | 26.1 | 25.6 | 12.8 |
| K57 | 4.0 | 3.2 | 3.5 | 3.6 | 1.8 |
| K92 | 7.6 | 7.4 | 7.3 | 7.4 | 3.7 |
| K136 | 44.9 | 31.2 | 38.8 | 37.9 | 18.9 |
| K149 | 54.3 | 45.5 | 61.7 | 53.4 | 26.7 |

These provide further evidence for high affinity, sub-50 nM, interactions and consolidate the observations by SPR in Example 2. In all the FRET experiments the Hill slopes were within the expected range (0.5-2), this is indicative of reversible binding as defined by the law of mass action, while nM displacement of the PGT121 knob domain fusion proteins suggests the interactions are indeed site-specific.

### Example 10

Additional experiments were conducted to characterise knob domain produced by chemical peptide synthesis.

1-Knob Domain Peptides

K149A and K149B and their production method are described in Example 4 (sometimes suffixed _{chem}SD for Site Directed). K8, K57, K92, K149 were produced using the free energy method and are displayed below in Table 43 (sometimes suffixed _{chem}FE for Free Energy). For peptides produced by both methods, liquid chromatography/mass spectrometry (LC/MS) confirmed that masses consistent with predicted amino acid sequences with complete formation of bonds were unanimously present.

TABLE 43
sequences of the knob domain tested in the present Example

| | SEQ ID | |
|---|---|---|
| ID | NO: | Knob domain peptide sequence |
| K8 | 322 | VCPDGFNWGYGCAAGSSRFCTRHDWCCYDERADSHTYGFCTG |
| | | NRV |
| K57 | 481 | GCPPGYKSGVDCSPGSECKWGCYAVDGRRYGGYGADSGVGS |
| K92 | 450 | TCPEGWSECGVAIYGYECGRWGCGHFLNSGPNISPYVST |
| K149 | 313 | SCPDGFSYRSWDDFCCPMVGRCLAPRN |

Chemical Variants

K8 _{chem}FE cyclic: To create a small, cyclic antibody fragment, head-to-tail cyclisation of the K8_{chem}FE knob domain was performed, resulting in a cyclic peptide referred as “K8 _{chem}FE cyclic”). The N- and C-termini of knob domains are in close proximity and this may mean that, amongst antibody fragments, they are uniquely amenable to cyclisation.

K92 _{chem}FE W21A/W6A/Y14A/Y16A/F26A: A series of π-π stacking interactions span one side of K92, encompassing residues: Y14, Y16 F26, H25, W21, W6 and P3. To evaluate the importance of stacking interactions to maintain tertiary structure, a series of alanine mutants, Y14A, Y16A, F26A, H25A, W21A and W6A, was synthesised.

K92 _{chem}FE W21H: Based on the structure, it was thought that a W21H mutation might introduce an additional electrostatic interaction between the polar nitrogen of the histidine imidazole ring and the N77_{C5} (numbering based on the mature C5 sequence), possibly leading to an improved binding affinity.

K57 _{chem}FE-Palmitoyl: To explore the effect of fatty acid conjugation, a palmitoylated form of K57_{chem} was synthesised.

2-Binding to C5

Binding of knob domain peptides to human C5 was measured by SPR, using a multi-cycle kinetics method as follows:

Kinetics were measured using a Biacore 8K (GE Healthcare) with a CM5 chip, which was prepared as follows: EDC/NHS was mixed at 1:1 ratio (flow rate, 10 μL/min; contact time, 30 seconds), human C5 protein at 1 μg/mL in pH 4.5 sodium-acetate buffer, were injected over flow cell one only (flow rate, 10 μL/min; contact time, 60 seconds). Final immobilization levels in the range of 2000-3000 response units (RU) were obtained, to yield theoretical Rmax values of ˜50-60 RU. Serial dilutions of peptide were prepared in HBS-EP buffer and injected (flow rate, 30 μL/min; contact time, 240 seconds; dissociation time, 6000 seconds). After each injection, the surface was regenerated with two sequential injections of 2M MgCl₂ (flow rate, μL/min; contact time, 30 seconds). Binding to the reference surface was subtracted, and the data were fitted to a single site binding model, using Biacore evaluation software.

The results are presented in Table 44 below.

TABLE 44
Summary of Biacore multi-cycle kinetics data from n = 3 experiments

| | mean | | mean | | mean | mean |
|---|---|---|---|---|---|---|
| | Kₒₙ | Kₒₙ | K_{off} | K_{off} | K_{D} | stoichiometric |
| knob domain | (Ms⁻¹) | SE | (s⁻¹) | SD | (M) | ratio* |
| K149_{chem}SD-A | 7.13E+05 | 1.08E+05 | 4.84E−03 | 1.08E−03 | 7.31E−09 | 0.82 |
| K149_{chem}SD-B | 1.56E+04 | 9.53E+01 | 4.17E−03 | 8.02E−04 | 2.69E−07 | 0.81 |
| K149_{chem}FE | 8.61E+05 | 1.60E+05 | 4.48E−03 | 1.57E−03 | 5.57E−09 | 0.85 |
| K57_{chem}FE | 2.60E+05 | 2.71E+04 | 5.79E−04 | 1.14E−04 | 2.28E−09 | 0.65 |
| K8_{chem}FE | 7.05E+04 | 8.23E+02 | 3.85E−04 | 3.25E−05 | 5.46E−09 | 0.25 |
| K92_{chem}FE | 2.83E+05 | 3.79E+04 | 1.01E−04 | 6.32E−05 | 4.11E−10 | 0.40 |
| K8_{chem}FE | 9.40E+04 | 1.19E+04 | 2.59E−03 | 8.01E−04 | 2.85E−08 | 0.28 |
| cyclic | | | | | | |
| K92_{chem}FE | 1.74E+04 | 7.10E+02 | 8.48E−03 | 1.13E−03 | 4.97E−07 | 0.62 |
| W21A | | | | | | |
| K92_{chem}FE | 7.98E+03 | 4.18E+02 | 1.57E−03 | 2.02E−03 | 2.19E−07 | 0.60 |
| W6A | | | | | | |
| K92_{chem}FE | 2.72E+04 | 1.10E+03 | 1.00E−04 | 6.24E−05 | 3.62E−09 | 0.41 |
| Y14A | | | | | | |
| K92_{chem}FE | 1.56E+07 | 9.01E+06 | 7.86E−01 | 1.36E+00 | 2.21E−07 | 0.66 |
| Y16A | | | | | | |
| K92_{chem}FE | 1.84E+05 | 2.58E+04 | 3.28E−03 | 7.12E−04 | 1.88E−08 | 0.65 |
| F26A | | | | | | |
| K92_{chem}FE | 4.25E+05 | 3.64E+04 | 6.12E−04 | 5.05E−05 | 1.48E−09 | 0.69 |
| W21H | | | | | | |
| K57_{chem}FE - | 5.11E+04 | 4.63E+03 | 4.77E−04 | 1.42E−04 | 9.52E−09 | 1.12 |
| Palmitoyl | | | | | | |

*mean stoichiometric ratio is calculated by dividing measured Rₘₐₓ value with theoretical Rₘₐₓ

The chemical knob domains bound C5 with high affinity (Table 44), equivalent to values previously reported for the biological peptides. For K149, which has two disulphide bonds, the adjacent cysteines C15 and C16 are unable to pair, giving rise to only two potential disulphide bonding arrangements: K149_{chem}SD-A (C2-C15, C16-C22) and K149_{chem}SD-B (C2-C16, C15-C22). While both forms bound C5, K149B displayed approximately 35-fold lower affinity. When produced by the free energy method (henceforth suffixed _{chem}FE), K149 (K149_{chem}FE) bound C5 with equal affinity to the higher affinity K149_{chem}SD-A form.

As mispairing of K149 disulphide bonds was tolerated to a certain extent, next the effect of removing disulphide bonds entirely was tested, through reduction and capping of cysteines with iodoacetamide (IAM). Following LC/MS analysis, to confirm uniform capping had occurred, binding to C5 was again measured by SPR. For K149_{chem}, loss of disulphide bonds entirely abrogated binding, while for K92 there was a substantial drop in affinity from 411 pM to 2 μM, mainly mediated by prolongation of the on rate. Loss of disulphide bonds in K57 also affected affinity but a K_{D} of 76 nM was retained. In both cases, decreases in affinity were predominantly mediated by a decrease in on rate, potentially due to a loss of tertiary structure.

Chemical Mutants:

K8 _{chem}FE cyclic: This fragment bound human C5.

K92 _{chem}FE W21A/W6A/Y14A/Y16A/F26A: When tested for binding to C5 by SPR, removal of any aromatic residue was highly detrimental to binding, while the H25A mutation entirely prevented folding of the peptide. All mutations lower affinity relative to wildtype K92_{chem}FE, including residues distal to the paratope, such as W6, and residues which do not sustain molecular interactions with C5, such as Y16; highlighting the importance of non-covalent tertiary structure in maintaining binding integrity.

K92 _{chem}FE W21H: While this mutation did not actually improve the affinity of K92_{chem}FE, replacing W21 with a histidine was tolerated such that the mutant could fold and yielded a much-lower loss of affinity, relative to removal of the aromatics in the other sites; suggesting the histidine was partially able to maintain the stacking interactions required for the folding and maintenance of tertiary structure. Notably, W21H was able to sustain affinity due to much improved value of km, relative to the other mutants.

K57 _{chem}FE-Palmitoyl: binding to C5 was unaffected by conjugation at the N-terminus.

3-Functional Activity

Having demonstrated binding to C5, biological function was evaluated in a range of complement ELISAs and erythrocyte haemolysis assays that were specific for either alternative (AP) or classical pathway (CP) activation.

Complement ELISA: assays were run using the CP and AP Complement functional ELISA kits (SVAR, COMPL 300 RUO), as per the manufacturer's protocol. For sample preparation, serum was diluted as per the respective protocol for the CP and AP assays; serial dilutions of peptides were prepared and allowed to incubate with serum for 15 minutes at room temperature, prior to plating.

Haemolysis assays: For AP, 50 μL of 24% normal human sera (Complement Technology), 50 of 20 mM MgEGTA (Complement Technology), and 48 μL of GVB0 buffer (0.1% gelatin, mM Veronal, 145 mM NaCl, 0.025% NaN3, pH 7.3, Complement Technology) were aliquoted into a single well of a 96-well tissue culture plate (USA Scientific) then mixed with 2 μL of inhibitors serially diluted in DMSO. Following equilibration for 15 minutes at room temperature, 50 μL of rabbit erythrocytes (Complement Technology), at 2.5×10⁷ per well, were added to the plates, which were then incubated at 37° C. for 30 minutes. Plates were centrifuged at 1,000×g for 3 min and 100 μL of supernatant was collected, transferred to another 96-well tissue culture treated plate, and absorbance was measured at 412 nm. For CP, μL of 4% normal human sera, 48 μL of GVB++ buffer (0.1% gelatin, 5 mM Veronal, 145 mM NaCl, 0.025% NaN₃, pH 7.3 with 0.15 mM CaCl₂ and 0.5 mM MgCl₂, Complement Technology) and 2 μL of inhibitors serially diluted in DMSO were aliquoted into a single well and equilibrated as described. Next, 100 μL of antibody-sensitized sheep red blood cells (Complement Technology) at 5×10⁷ per well were added to the plate, which was then incubated at 37° C. for 1 hour. Samples were subsequently processed as described.

Results

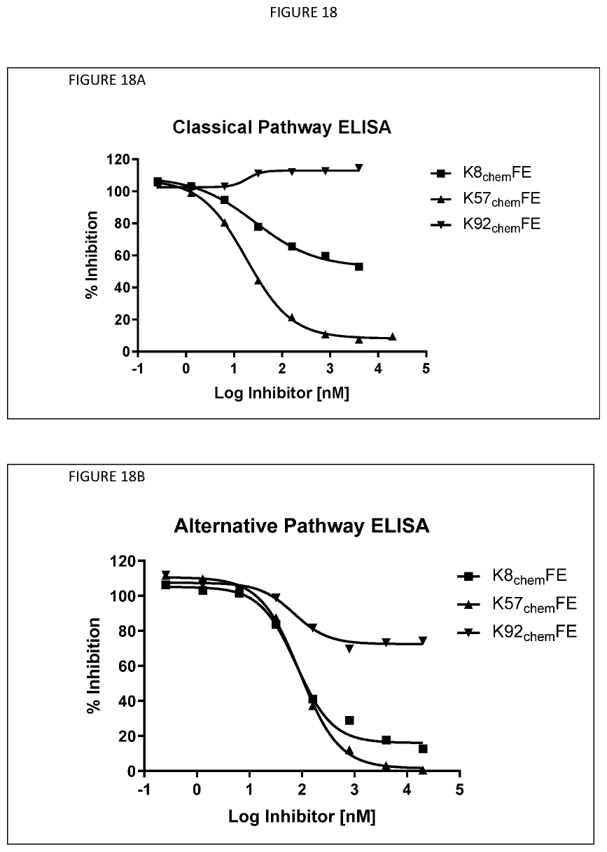

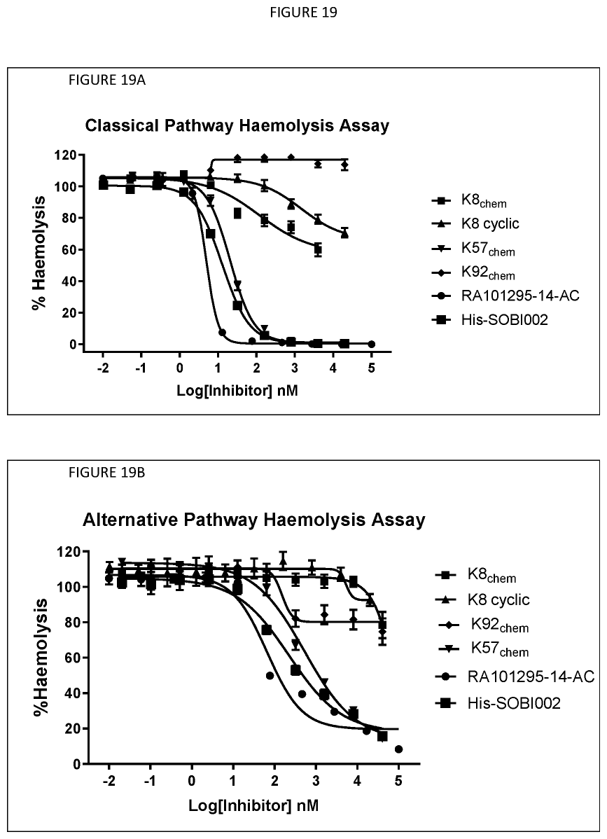

The results of the CP ELISA assays are presented in FIG. 18A. The results of the AP ELISA assays are presented in FIG. 18B. The results of the CP and AP haemolysis assays are presented in FIG. 19A and FIG. 19B respectively.

In complement activation ELISAs which tracked C5b deposition, K57_{chem}FE was a potent and fully efficacious inhibitor of both the CP and AP; chemical K8 (K8_{chem}FE) was a partial inhibitor of the CP and AP; while chemical K92 (K92_{chem}FE) partially inhibited the AP and showed modest, dose dependent enhancement of the CP. Consistent with the results obtained with biologically derived K149, K149_{chem}FE was a non-functional, silent binder of C5.

Behaviour in erythrocyte haemolysis assays was consistent with the ELISAs. K57_{chem}FE was a potent and fully efficacious inhibitor of complement mediated cell lysis, K92_{chem}FE was active solely in the AP-driven haemolysis assay and K8 was a partial inhibitor for the CP and weakly active in the AP assay. Importantly, these observations in the ELISA and haemolysis assays closely mirror those previously reported with the biological forms of the peptides. In haemolysis assays, K57_{chem}FE was broadly equivalent to two clinical C5 inhibitors: RA101295-14, a close analogue of the UCB-Ra Pharma macrocyclic peptide Zilucoplan, which is currently in phase III trials, and SOBI002, an affibody from Swedish Orphan Biovitrum, which was discontinued after showing transient adverse effects in a phase 1 trial.

K8 _{chem}FE cyclic: was a functionally active complement inhibitor, with only a modest loss of potency relative to K8_{chem}FE (FIG. 19).

4-Cross-Reactivity

As target binding may influence pharmacokinetics, we tested the knob domains for cross reactivity with C5 protein from Rattus norvegicus (rat).

Method

Kinetics were measured using a Biacore 8K (GE Healthcare) with a CM5 chip, which was prepared as follows: EDC/NHS was mixed at 1:1 ratio (flow rate, 10 μL/min; contact time, 30 seconds), rat C5 protein at 1 ug/mL in pH 4.5 sodium-acetate buffer, were injected over flow cell one only (flow rate, 10 μL/min; contact time, 60 seconds). Final immobilization levels in the range of 2000-3000 response units (RU) were obtained, to yield theoretical Rmax values of ˜50-60 RU. Serial dilutions of peptide were prepared in HBS-EP buffer and injected (flow rate, 30 μL/min; contact time, 240 seconds; dissociation time, 6000 seconds). After each injection, the surface was regenerated with two sequential injections of 2M MgCl₂ (flow rate, μL/min; contact time, 30 seconds). Binding to the reference surface was subtracted, and the data were fitted to a single site binding model, using Biacore evaluation software.

Results

By SPR, K8_{chem} was cross reactive with rat C5 protein, as well as C5 from other species (data not shown), while K57_{chem} was specific for human C5.

To test if knob domains are resistant to proteolysis by virtue of their abundant disulphide bonds, we used mass spectrometry to track the stability of K8_{chem}FE, K57_{chem}FE and K57_{chem}FE-palmitoyl in human, rat and Mus musculus (mouse) plasma over a period of 24 hours.

Method

The stability in rat/mouse/human Lithium Heparin plasma was assessed at a concentration level of 1.25 μg/mL for K57_{chem}FE-Palmitoyl, 6.25 μg/mL for K57 and 8 ng/mL for K8, over a 24 hour period at room temperature. A calibration line and suitable quality control samples were prepared and frozen at −80° C., alongside further separate spikes for assessment. These separate spikes were placed into the freezer after the original calibration line at time intervals of 0.116, 0.25, 0.5, 1, 2, 4, 6 and 24 hours. Upon a minimum of 1 hour freezing for the final 24 hour spike. These were extracted via protein precipitation alongside the original calibration line.

Results

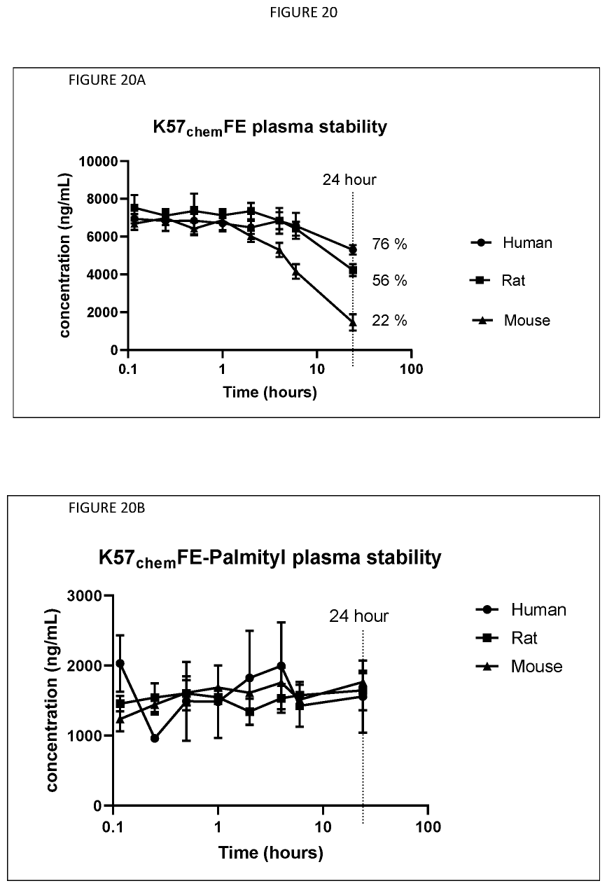

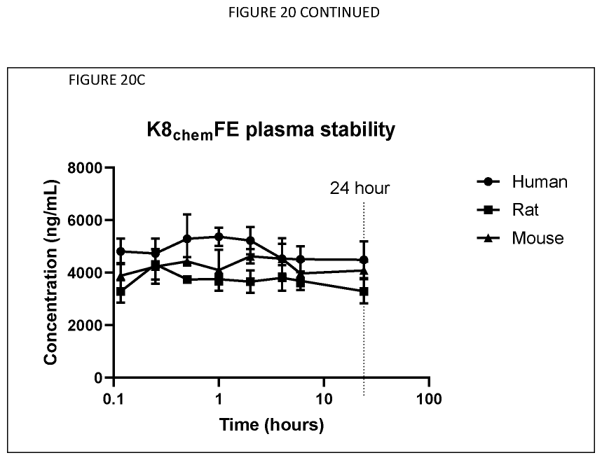

The unmodified knob domains were exceptionally stable in human plasma, with >75% peptide remaining intact after 10 hours (FIG. 20).

6-Pharmacokinetics

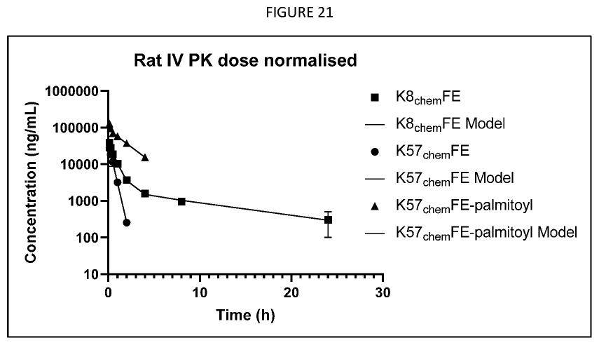

The pharmacokinetics of K8_{chem}FE, K57_{chem}FE and K57_{chem}FE-palmitoyl were measured following dosing via an intravenous bolus to Sprague Dawley rats (FIG. 21).

Method

Plasma pharmacokinetics was studied for each peptide in male Sprague-Dawley rats of a body weight between 324 and 425 g. Drug was administered intravenously via the tail vein. Doses administered were 10 mg/kg for peptides K8_{chem}FE, K57_{chem}FE and 1 mg/kg for K57_{chem}FE-Palmitoyl. Blood samples were taken at 7 minutes, 15 minutes, 30 minutes, followed by 1, 2, 4, 8 and 24 hours. Blood was collected into Li Heparin tubes and spun to prepare plasma samples for bioanalysis. Bioanalytical data was analysed on an individual animal basis using Pharsight Phoenix 64 Build 8.1. Non-compartmental analysis was conducted and mean pharmacokinetic parameters for each drug calculated.

Results

Following administration at 10 mg/kg, K57_{chem}FE was subject to renal clearance and eliminated in a rapid manner typical of peptides and low molecular weight proteins (t_{1/2}=17 mins/plasma clearance [C1p]=10.6 ml/min/kg). In contrast, K8_{chem}FE tightly bound rat C5 protein and adopted target-like kinetics, resulting in markedly improved exposure (t_{1/2}=˜9 hours/C1p=3.3 ml/min/kg). Due to reduced solubility, K57_{chem}FE-palmitoyl was tested at a dose of 1 mg/kg; the conjugation of a palmitic fatty acid extended exposure, relative to unmodified K57_{chem} (t_{1/2}=1.6 hours/C1p=0.8 ml/min/kg). This suggests that conjugation, potentially in combination with cyclisation or other chemical modifications, is a viable route to extend the biological exposure of chemical knob domains.

### Example 11: Crystal Structure of the C5-K92 Complex

A crystal structure of K92 knob domain isolated from bovine (K92_{bio}) or produced by chemical synthesis (K92_{chem}FE) in complex with C5 was also solved at a resolution of 2.75 Å and 2.57 Å respectively as described in Example 5.

A mFo-DFc simulated annealing omit map of the C5-K92_{chem}FE complex showed clear electron density for the peptide, while the final structure shows the fold and disulphide bond arrangement of C5-K92_{chem}FE to be contiguous to K92_{bio}. Analysis with the macromolecular structure analysis tool PDBPiSA, confirmed that the molecular interactions which sustain binding to C5 are consistent between K92_{chem}FE and K92_{bio}.

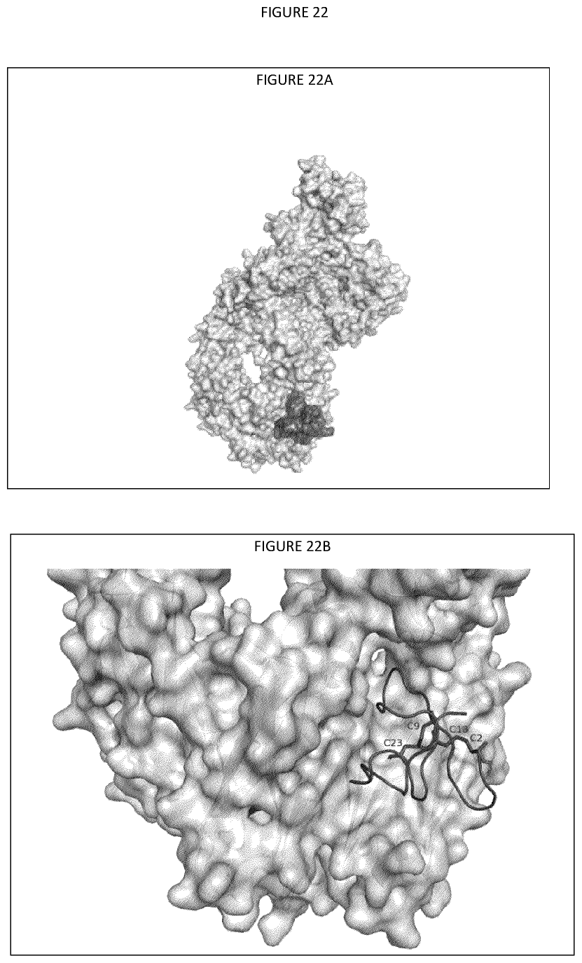

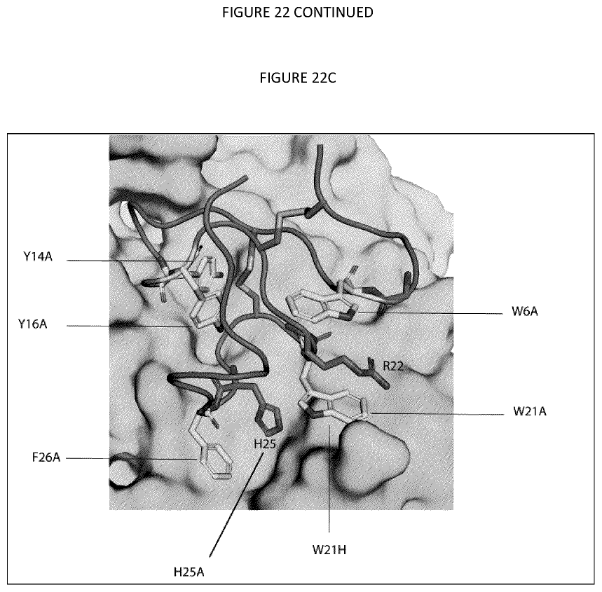

FIG. 22A shows K92_{chem}FE in complex with C5. FIG. 22B shows the disulphide arrangement on the K92. FIG. 22C shows the position of the mutations of K92 mentioned in the previous example.

### Example 12: Construction of Ultralong CDR-H3 Phage Libraries for the Discovery of Knob Domain Peptides

The following method enables the generation of antigen-enriched ultralong CDR-H3 libraries which may be performed for any antigen of interest. Phage display libraries of ultralong CDR-H3 could be generated by any suitable method, such as linking the ultralong CDR-H3 directly or via a spacer to pIII of M13, for example fused to hC3nb1 VHH.

As an example, to enable the creation of antigen-enriched CDR-H3 libraries, bovine immunisations with human C5 and with human and mouse serum albumin proteins were performed. Ultralong CDR-H3 sequences were specifically amplified and fused directly to bacteriophage minor coat protein gene g3p (or pIII) to permit enrichment against the antigen using phage display. The phagemid vector contains a pelB leader sequence, ahead of the ultralong CDR-H3 sequence for display. This is followed by a poly-histidine and c-myc-tag, which is fused directly to the g3p. The entire open reading frame encoding the fusion protein is under the control of glucose repressible lac promoter. Upon superinfection with helper phage, an M13 replication origin will result in the synthesis and packaging of single stranded phagemid DNA, encoding the phage display construct within the phage virion. By this method, ultralong CDRH3 sequences could be displayed upon the surface of phage virions to retain genotype to phenotype link, and via phage bio-panning, those antibody fragments which bound the immunised antigens were isolated.

Methods

Immunisation:

Holstein Friesian cattle were immunised with purified human C5 or serum albumin protein antigens, one cow per immunogen, with one prime and two boosts at four-week intervals. For C5, two adult Friesian cows were immunised with Complement C5 (CompTech). For early immunisations, 1.25 mg of C5 was mixed 1:1 (v/v) with Adjuvant Fama (GERBU Biotechnik). Three subcutaneous injections into the shoulder were performed at 1-month intervals. Ten days post immunisation, 10 mL of blood was taken to allow testing of the serum antibody titre. For subsequent boosts, 1.25 mg C5 was emulsified 1:1 with Freund's complete adjuvant (Sigma)—and, for the final shot, Montanide (Seppic)—immediately prior to subcutaneous injection into the shoulder. For human and mouse serum albumin (HSA/MSA respectively), three immunisations were performed at four-week intervals, where 1 mg of total protein, 0.5 mg each HSA and MSA, was homogeneously mixed 1:1 (v/v) with Montanide adjuvant (Seppic) prior to administration into the left shoulder.

Harvesting of Immune Material and ELISA Assay:

Seven days following the second and third immunisations, serum samples were taken to assess target-specific responses using ELISA upon protein adsorbed directly to Nunc Maxisorb plates at 2 μg/ml in PBS. Serum was diluted into PBS supplemented with 1% BSA (w/v), and the response determined using a secondary antibody anti-Bovine H+L-HRP conjugate (Stratech). After determination of a selective serum titre response, ten days after final boost additional sampling was taken from these cows: 500 mL blood, a sample of spleen ˜2 cm³ and a single draining lymph node taken proximal to the site of immunisation.

Identification of Ultralong CDR-H3 Sequences Binding to the Antigen of Interest:

An RNeasy plus midi kit (Qiagen) was used to purify total RNA from 5×10⁷ cells purified from the lymph node, as per the manufacturer's protocol. RNA was immediately used in a RT-PCR reaction using Super script IV vilo Master Mix (Invitrogen).

Primary PCR

The cDNA encoding CDR-H3 regions was selectively amplified via PCR. The primers anneal to the framework 3 and framework 4 of the heavy chain, thereby amplifying the CDR-H3 region regardless of changes in length (standard or ultralong CDR-H3). The primers used (read from 5′ to 3′) were: forward primer: GGACTCGGCCACMTAYTACTG (SEQ ID NO: 446), and reverse primer: GCTCGAGACGGTGAYCAG (SEQ ID NO: 447). PCR was performed using Phusion Green Hotstart II Master mix (Thermo scientific). Primary PCR DNA was column purified before being used in a secondary PCR.

Secondary PCR

Primers sets derived from ultralong CDR-H3 ascending and descending stalk sequences were used to specifically amplify ultralong sequences from the primary PCR DNA. 7 ascending stalk primers were used individually in separate PCR reactions whilst 6 descending stalk primers were pooled. Ascending primers were used at a concentration of 10 μM and each descending primer was at a concentration of 10 μM in the pooled solution. Primer sets also contained SfiI and NotI restriction enzyme sites which allowed for ligation into vector.

The primers used (read from 5′ to 3′) were as follows:

Ascending primer set:
(SEQ ID NO: 482)
CTCGCGGCCCAGCCGGCCATGGCCACTACTGTGCACCAAAAAACA
(SEQ ID NO: 483)
CTCGCGGCCCAGCCGGCCATGGCCACTACTGTGCACCAAAGAACC
(SEQ ID NO: 484)
CTCGCGGCCCAGCCGGCCATGGCCACTACTGTGCACCAAAAAACG
(SEQ ID NO: 485)
CTCGCGGCCCAGCCGGCCATGGCCACTACTGTGCACCAACAAACT
(SEQ ID NO: 486)
CTCGCGGCCCAGCCGGCCATGGCCACTACTGTGCACCAACAGACC
(SEQ ID NO: 487)
CTCGCGGCCCAGCCGGCCATGGCCACTACTGTGGTCCAGAAAACA
(SEQ ID NO: 488)
CTCGCGGCCCAGCCGGCCATGGCCACTACTGTAGTCCAACGAACA
Descending primer set:
(SEQ ID NO: 489)
TGATGGGCGGCCGCGGCATCGACGTACCATTCGTA
(SEQ ID NO: 490)
TGATGGGCGGCCGCGGTATCGACGTACCATTCGTA
(SEQ ID NO: 491)
TGATGGGCGGCCGCGGCTTCGACGTACAATTCGTA
(SEQ ID NO: 492)
TGATGGGCGGCCGCGGCATTGACGTAGAATTCGTA
(SEQ ID NO: 493)
TGATGGGCGGCCGCGGCCTCGATGTCAAATTCGTA
(SEQ ID NO: 494)
TGATGGGCGGCCGCGGTTTCGACGTGGTATTCGTA

PCR was performed using Phusion Green Hotstart II Master mix (Thermo scientific). The secondary PCR product was column purified before being used for cloning into the phagemid vector.

Library Construction

A phagemid vector (derivative of pUC119) was used throughout research. Both the phagemid vector and the secondary PCR product were digested using NotI and SfiI. Once digested, the vector and CDR-H3 inserts were ligated using a 1:3 molar ratio of vector to insert. The precipitated ligation was used to transform E. coli TG1 cells (Lucigen) using electroporation. Recovered samples were plated out onto selective medium, 2TY Agar supplemented with 1% Glucose, 100 μg/mL Carbenicillin, and incubated at 30° C. overnight.

Phage Rescue

The culture was harvested by addition of selective liquid media, 2TY supplemented with 1% Glucose and 100 μg/mL Carbenicillin, and scraping of the biomass into liquid culture. The collected culture was used to seed fresh selective medium at an OD₆₀₀ of 0.1 AU. The culture was incubated at 37° C. until it reached an approximate OD₆₀₀ of 0.5 AU. M13K07 helper phage was added at a multiplicity of infection (MOI) of 20 and the culture was left standing at 37° C. for 1 hour. The culture was then centrifuged, and the pellet resuspended in 2TY media supplemented with 50 μg/mL Kanamycin and 100 μg/mL Carbenicillin and incubated at 30° C. overnight.

Following overnight incubation, the culture supernatant was recovered by centrifugation, the phage pellet resuspended in 20 mL of PBS. A further round of precipitation was performed, and the phage pellet resuspended in PBS supplemented with 20% glycerol at a final concentration of approximately 10¹² PFU/mL. Purified phage aliquots were stored at −80° C. until required.

Phage Bio-Panning

For the C5 libraries, a single round of enrichment was performed with either human or rat C5 protein. For the albumin library two round of enrichment were performed with human and mouse serum albumin. Phage were blocked for 30 minutes. Biotinylated antigen was added to the blocked phage at a concentration of 100 nM and incubated at room temperature with mixing.

Streptavidin Dynabeads (Thermofisher) were resuspended in the blocked phage solution, incubated for 10 minutes and washed four times with 1 mL of 0.1% Tween 20 in PBS. After washing, beads were pelleted, and supernatant removed. 50011.1 of 0.1 M hydrochloric acid was added to elute phage from beads before incubation with mid-log E. coli TG1 cells to allow for bacterial infection.

The cells were grown on solid selective medium, 2TY Agar supplemented with 1% glucose and 100 μg/mL Carbenicillin for recovery of enriched sub-libraries and for single colonies for screening.

Monoclonal Phage Screening ELISA

Colonies were picked into a 96-well culture plate containing selective medium 2TY supplemented with 1% Glucose and 100 μg/ml Carbenicillin and was incubated at 37° C. until wells reached mid-log phase of growth prior to addition of M13K07 helper phage. The block was incubated without shaking for 1 hour to achieve infection and phage production using continued culture in selective media supplemented with 50 μg/ml Kanamycin and 100 μg/ml Carbenicillin.

Binding to antigen was assessed by monoclonal phage ELISA, 96-well flat bottom Nunc MaxiSorp plates (Thermofisher) were coated with a 2.5 μg/ml solution of human, mouse, or rat serum albumin, human C5 or mouse C5 diluted with PBS, and left overnight at 4° C. Negative control plate and Anti-myc tag plate were also prepared.

Monoclonal phage rescue supernatants were blocked with an equal volume of a 2% milk (w/v) in PBS, for serum albumin proteins, or 2% BSA and 2% milk (w/v) in PBS solution. The coating solution was removed from coated Nunc plates and each plate was blocked with 1% BSA in PBS (for C5 libraries) or 1% milk powder (for albumin library). Both phage and Nunc plates were blocked for 1 hour at room temperature. Nunc plates were washed using a 96-well microplate washer (BioTek) with a PBS solution contain 0.1% Tween20 (Sigma Aldrich). 100 of blocked phage solution was added to each well of Nunc MaxiSorp plate and left shaking at room temperature for 1 hour. Plates were washed and dried as previously described. 100 of an Anti-M13-Horse radish peroxidase conjugated antibody (GE Healthcare) diluted (1:5,000) in a 1% BSA or milk solution was added to each well and left shaking at room temperature for 1 hour. Plates were washed again and 50 μL of TMB solution (Merck Millipore) was added to each well. Plates were left shaking for 10 minutes. Absorbance was measured at 630 nm and 490 nm. Binding signals were obtained where Absorbance at 630 nm was >3-times the signal upon irrelevant antigen.

For sequencing, 0.5 μl of cell containing media from enriched monoclonal rescue blocks was added to the corresponding wells in the PCR plate containing: 20.75 μL of DPEC-treated water; 2.5 μL of 10× Standard Taq buffer (New England Biolabs); 0.5 μL of forward and reverse 10 primer stock and 0.25 μL of Taq DNA polymerase (5000 u/mL—New England Biolabs).

The primers used to amplify the insert within the phagemid vector and annealing to the phagemid vector (read from 5′ to 3′) were as follows:

Forward:
(SEQ ID NO: 495)
GTTGGCCGATTCATTAATGCAG
Reverse:
(SEQ ID NO: 496)
ACAGACAGCCCTCATAGTTAGC

The plate was heated to 95° C. for five minutes in a thermocycler and then heated for thirty-five cycles of: (95° C. for 40 seconds; 55° C. for 40 seconds; 68° C. for 100 seconds and 72° C. for 2 minutes). Finally, 1 μL Illustra ExoProStar was added to each well to remove unused dNTP's and primers before sequencing. The plate was placed in a thermocycler at 37° C. for 40 minutes and 80° C. for 15 minutes, prior to Sanger sequencing, performed at Macrogen.

Results

By monoclonal phage ELISA the following ultralong CDR-H3 sequences were identified as binders (OD>0.2 AU):

TABLE 45
Anti-serum albumin ultralong CDR-H3

| SEQ ID | Sequence | |
|---|---|---|
| NO: | of the anti-serum albumin ultralong CDR-H3 | Specificity |
| 497 | TTVHQKTTRQTSCPDGYIAGDSSCYRWRCRGNNCC | Mouse serum |
| | KYGENRLLNYYDYTCVPYRDTYEWYVD | albumin only |
| 498 | TTVHQQTHQDQTCPDGYTRTNYYCRRDGCGSWCN | Human serum |
| | GAERQQPCIRGPCCCDLTYRTAYEYHVET | albumin only |
| 499 | TTVHQQTQKHCPDDDTDRDGCSRPDSRGGSGCGSY | Mouse and rat |
| | GRYGDQGGACCPLTYEFDV | serum albumin |
| 500 | TTVHQQTQERCPDDYTDRGGCSIPYNCGGSRCCAY | Mouse and rat |
| | GRNGGYGGISCSRTYEFYVN | serum albumin |
| 501 | TTVHQQTQERCPDDYTDRGGCSIPYNCGGSRCCAY | Mouse and rat |
| | GRNGGYGGNTCSRTYELYVE | serum albumin |
| 502 | TTVHQQTQERCPDDYTDRGGCSIPYTCGGSRCCAY | Mouse and rat |
| | GRNGGYGGVSCSRTYEFYVN | serum albumin |
| 503 | TTVHQQTQERCPDDYTDRGGCSIPYNCGGSRCCAY | Mouse and rat |
| | GRNGGYGGISCSRTYEWYVD | serum albumin |
| 504 | TTVHQQTQERCPDDYTDRGGCSIPYSCGDSRCCAY | Mouse and rat |
| | GRNGGYGGVSCSRTYEFYVN | serum albumin |
| 505 | TTVHQQTQERCPDDYTDRGGCSIPYNCGGSRCCAY | Mouse and rat |
| | GRYGDYGGISCSRTYEFYVN | serum albumin |
| 506 | TTVHQQTQERCPDDYTDRGGCSIPYNCGGSRCCAY | Mouse and rat |
| | GRNGGYGGVSCSRTYEFYVN | serum albumin |
| 507 | TTVHQQTQERCPDNYTDRGGCSIPYSCGDSRCCAY | Mouse and rat |
| | GRNGGYGGVSCSRTYEFYVN | serum albumin |
| 508 | TTVHQQTQERCPDNYTDRGGCSIPYTCGGSRCCAY | Mouse and rat |
| | GRNGGYGGVSCSRTYEFYVN | serum albumin |

TABLE 46
Sequences of the ultralong CDR-H3 knob domains specific to albumin

| SEQ ID | Sequence of the anti-serum | |
|---|---|---|
| NO: | albumin ultralong CDR-H3knob domains | Specificity |
| 509 | SCPDGYIAGDSSCYRWRCRGNNCCKYGENRLLN | Mouse serum albumin |
| | YYDYTCVPYRDT | only |
| 510 | TCPDGYTRTNYYCRRDGCGSWCNGAERQQPCIR | Human serum albumin |
| | GPCCCDLTYRTA | only |
| 511 | HCPDDDTDRDGCSRPDSRGGSGCGSYGRYGDQG | Mouse and rat serum |
| | GACCPLT | albumin |
| 512 | RCPDDYTDRGGCSIPYNCGGSRCCAYGRNGGYG | Mouse and rat serum |
| | GISCSRT | albumin |
| 513 | RCPDDYTDRGGCSIPYNCGGSRCCAYGRNGGYG | Mouse and rat serum |
| | GNTCSRT | albumin |
| 514 | RCPDDYTDRGGCSIPYTCGGSRCCAYGRNGGYG | Mouse and rat serum |
| | GVSCSRT | albumin |
| 515 | RCPDDYTDRGGCSIPYNCGGSRCCAYGRNGGYG | Mouse and rat serum |
| | GISCSRT | albumin |
| 516 | RCPDDYTDRGGCSIPYSCGDSRCCAYGRNGGYG | Mouse and rat serum |
| | GVSCSRT | albumin |
| 517 | RCPDDYTDRGGCSIPYNCGGSRCCAYGRYGDYG | Mouse and rat serum |
| | GISCSRT | albumin |
| 518 | RCPDDYTDRGGCSIPYNCGGSRCCAYGRNGGYG | Mouse and rat serum |
| | GVSCSRT | albumin |
| 519 | RCPDNYTDRGGCSIPYSCGDSRCCAYGRNGGYG | Mouse and rat serum |
| | GVSCSRT | albumin |
| 520 | RCPDNYTDRGGCSIPYTCGGSRCCAYGRNGGYG | Mouse and rat serum |
| | GVSCSRT | albumin |

TABLE 47
Anti-C5 ultralong CDR-H3

| SEQ ID | Sequence | |
|---|---|---|
| NO: | of the anti-C5 ultralong CDR-H3 | Specificity |
| 521 | TTVHQKTKKTCPLGYNLNDRCDHFNTCRVEKCC | Human and rat |
| | QNGVVNAYGICEYAGGNATYTYQWYVH | C5 |
| 522 | TTVHQRTKKTCPLGYAINDRCDDLKTCGPDECCL | Human and rat |
| | NGVVNAYGICEYEGESATHTYEWYVD | C5 |
| 523 | TTVHQKTEPSCPYGYIYTGGCHTTYGCGNYVCYP | Human and rat |
| | GSGPPRVGDVSVSYTYEWYVD | C5 |
| 524 | TTVHQRTKKTCPLGYDLNDRCDHENTCRVEECCK | Human and rat |
| | NGVVNAYGICEYAGGSATYTYELYVE | C5 |
| 525 | TTVVQKTQKSEALCPDGYTRSGRPGCYYGCPDST | Human and rat |
| | CCSRTRTLHVSEHCIAPAYTYNYEWYVD | C5 |
| 526 | TTVHQRTLKNRNCPDGYGYQRHCTVGEDCTEGC | Human and rat |
| | CDNYGRCTTYTDTYTYELYVE | C5 |
| 527 | TTVHQKTNRQESCPGSSGDRTICERSWSCGGYHCS | Human and rat |
| | AYDTWGAGGSSDCGTCTYTYTYEWYVD | C5 |
| 528 | TTVHQRTKKTCPLGYDLNDRCDHFNTCRVEECCK | Human and rat |
| | NGVVNAYGICEYAGGSATYTYKWYVD | C5 |
| 529 | TTVHQRTVKSGRPPGTVAGVHCSPGSDCSWGCYD | Human |
| | RDDRRVDGAGADSGLGSTSTYEFYVN | |
| 530 | TTVHQQTNLRQRSCPDGYKDNRFCSPDGGCSAVS | Human |
| | HWGWDSSCVSYTYTDTYEWYVD | |
| 531 | TTVHQRTVKSGCPTGTVAGVHCSPGSDCSWGCYD | Human |
| | KDDRRVDGAGVASGLGSTYTYEFYVN | |
| 532 | TTVHQRTIKSGCPPGYKSGVDCSPGSECKWGCYA | Human |
| | VDGRRYGGYGADSGVGSTYTHEFYVN | |
| 533 | TTVVQRTHKTTSCPDGYHFIEPCHSGLCWREGAC | Human |
| | NGDGICANGLGRCRTVSETSTYELYVE | |
| 534 | TTVHQRTVKSGCPTGTVAGVHCSPGSDCSWGCYD | Human |
| | KDDRRVDGAGAASGLGSTYTYEYHVE | |
| 535 | TTVHQKTRSRCPDGCFLSSYCPVGYACSGFACCDC | Human |
| | GGYDYAGGVRGGRCSVRSTTYEWYVD | |
| 536 | TTVHQQTNKRRQNCPDGYKYNGFCTPDGGCSRVS | Human |
| | SWGWDRSCISPTYTYTYEWYVD | |
| 537 | TTVHQRTVKSGCPPGTVAGVHCSPGSDCSWGCYD | Human |
| | RDDRRVDGAGADSGLGSTSTYEFYVN | |
| 538 | TTVVQRTHKKTSCPDGYHFIEPCHSGLCWREGAC | Human |
| | NGDGICANGLGRCRTVSETSTYEFYVN | |
| 539 | TTVVQRTHKTTSCPDGYHFIEPCHSGLCWREGAC | Human |
| | NGDGICANGLGRCRTVSETSTYEWYVD | |
| 540 | TTVHQRTVKSGCPTGTVAGVHCSPGSDCSWGCYD | Human |
| | ADDRRVDGYGADSGVGSPYTYEYHVE | |
| 541 | TTVHQRTIKSGCPPGYKSGVDCSPGSECKWGCYA | Human |
| | VDGRRYGGYGADSGVGSTYTHEYHVE | |
| 542 | TTVVQRTHKTTSCPDGYHFIEPCHSGLCWREGAC | Human |
| | NGDGICANGLGRCRTVSETSTYEFYVN | |
| 543 | TTVHQRTLKNRNCPDGYGYQRHCTVGEDCTDHC | Human |
| | CDAYGLCTSYTYTYTYEWYVD | |
| 544 | TTVHQRTLKNRNCPDGYGYQRHCTVGEDCTDSCC | Human |
| | DRYGLCTTSTETYTYELYVE | |
| 545 | TTVHQRTLKNRNRPEGYGYQRHCTVGEECTDSCC | Human |
| | DRYGLCTTSTETYTYEWYVD | |
| 546 | TTVHQRTKKTCPLGYDLNDRCDHFNTCRVEECCK | Human |
| | NGVVNAYGICEYAGGSATYTYEWYVE | |
| 547 | TTVHQRTLKNRNCPDGYGYQRHCTVGEDCTDSCC | Human |
| | DRYGLCTTSTETYTYEFYVN | |
| 548 | TTVHQRTLKNRNCPDGYGYQRHCTVGEDCTDSCC | Human |
| | DRYGLCTTSTETYTYEWYVD | |
| 549 | TTVHQRTLKNRNCPDGYGYQRHCTVGEDCTDSCC | Human |
| | DRYGLCTTSTETYTYEWYVD | |
| 550 | TTVHQRTLHNRNCPDGYGYQRHCTVGEDCTERC | Human |
| | CDNYGLCTSYTDTYTYEFYVN | |
| 551 | TTVHQRTKKTCPLGYDLNYRCDHENTCRVEECCK | Human and Rat |
| | NGVVNAYGICEYAGGSATYTYEWYVD | |
| 552 | TTVHQRTKEERTCPSGCSWFSGCWDTYRCGPSVC | Human and Rat |
| | CRDGRYGCAAIICRDTYEWYVD | |
| 553 | TTVHQKTKKTCPRGYHYNDRCEFFNTCRVEECCL | Human and Rat |
| | NGVVNTYGICEYEGGSATYTYEWYVD | |
| 554 | TTVHQKTKKTCPRGYHYNDRCDFFNTCRVEECCL | Human and Rat |
| | NGVVNTYGICEYEGGSATYTYEWYVD | |
| 555 | TTVHQKTKKTCPRGYHYNDRCEFFNTCRVEECCL | Human and Rat |
| | NGVVNTYGICEYEGGSATYTYEWYVD | |
| 556 | TTVHQKTTRVNSCPDGYGYGDGYCYDSGCSASDC | Human and Rat |
| | YGVDALYSYGHCGCSIYTERPRYEWYVD | |
| 557 | TTVHLKTKKSCPLGYAINDRCDDLKTCGPDECCL | Human and Rat |
| | NGVVNAYGICEYEGESATHTYEWYVD | |
| 558 | TTVHQKTKKSCPLGYAINDRCDDLKTCGPDECCL | Human and Rat |
| | NGVVNAYGICEYEGESATHTYELYVE | |
| 559 | TTVHQRTAKRCPSNNEDATACRYSSVCGDYVCEG | Human |
| | LSESYAQGWGACRRYACRDSYEWYVD | |
| 560 | TTVHQRTAKRCPSNNEDATACRYSSVCGDYVCEG | Human |
| | LSESYAQGWGACRRYACRDSYEWYVD | |
| 561 | TTVHQQTNKRRQNCPDGYEYNGFCTPDGGCSRVS | Human |
| | NWGWDRSCISPTYTYTYEWYVE | |
| 562 | TTVHQQTTKKSSCPDGYCDCNGCGYGNGCSRGGC | Human |
| | FDFRLYSGYSADIVVSTTYTHDFYID | |
| 563 | TTVHQQTKKQKSCPDGWGHSDDCNCACSANAYA | Human |
| | CCKRDWLLPGPSCECSTYCVSHTYQWYV | |
| 564 | TTVHQHTRKSGSCPDGWSDCHGSCDGVGCTGSDC | Human |
| | VRYNARGYGRHACSGYAYTYSYEFYVN | |
| 565 | TTVHQRTIKSGCPPGYKSGVDCSPGSECKWGCYA | Human |
| | VDGRRYGGYGADSGVGSTYTHELYVE | |
| 566 | TTVVQRTKRTCPEGLVYNSDQSRCCAADSGVCWE | Human |
| | YWRGERVTRGFTYEWYVD | |
| 567 | TTVHQRTTKRCPSNNEDATACRYSSVCGDYVCEG | Human |
| | LSESYAQGWGACRRYACRDSYEWYVD | |
| 568 | TTVVQRTRKIVTCPDGYSYSEGCGKGDDCGGVHC | Human |
| | CANGGVTCWYRHCCSTGTDTYSYEWYVD | |
| 569 | TTVHQRTAKRCPSNNEEATACRYSSVCGDYVCEG | Human |
| | LSESYAQGWGACRRYACRDSYEWYVD | |
| 570 | TTVHQRTIKSGCPPGYKSGVDCSPGSEYKWGCYA | Human |
| | VDGRRYGGYGADSGVGSTYTHEFYVN | |
| 571 | TTVHQRTIKSGCPPGYKSGVDCSPGSECKWGCYA | Human |
| | VDGRRYGGYGADSGVGSTYTHEWYVD | |

TABLE 48
Anti-C5 ultralong CDR-H3 knob domains
The minimal sequence of the knob domains as defined in the present
application are highlighted in bold in the Table below.

| SEQ ID | Sequence of | Specificity |
|---|---|---|
| NO: | the anti-C5 ultralong CDR-H3 knob domains | |
| 572 | TCPLGYNLNDRCDHFNTCRVEKCCQNGVVNAYGI | Human and rat |
| | CEYAGGNAT | C5 |
| 573 | TCPLGYAINDRCDDLKTCGPDECCLNGVVNAYGIC | Human and rat |
| | EYEGESAT | C5 |
| 574 | SCPYGYIYTGGCHTTYGCGNYVCYPGSGPPRVGDV | Human and rat |
| | SVS | C5 |
| 575 | TCPLGYDLNDRCDHFNTCRVEECCKNGVVNAYGI | Human and rat |
| | CEYAGGSAT | C5 |
| 576 | LCPDGYTRSGRPGCYYGCPDSTCCSRTRTLHVSEH | Human and rat |
| | CIAPA | C5 |
| 577 | NCPDGYGYQRHCTVGEDCTEGCCDNYGRCTTYTD | Human and rat |
| | | C5 |
| 578 | SCPGSSGDRTICERSWSCGGYHCSAYDTWGAGGSS | Human and rat |
| | DCGTCT | C5 |
| 579 | HCSPGSDCSWGCYDRDDRRVDGAGADSGLGSTST | Human |
| 580 | SCPDGYKDNRFCSPDGGCSAVSHWGWDSSCVS | Human |
| 581 | HCSPGSDCSWGCYDKDDRRVDGAGVASGLGST | Human |
| 582 | GCPPGYKSGVDCSPGSECKWGCYAVDGRRYGGYG | Human |
| | ADSGVGST | |
| 583 | SCPDGYHFIEPCHSGLCWREGACNGDGICANGLG | Human |
| | RCRTVSETST | |
| 584 | GCPTGTVAGVHCSPGSDCSWGCYDKDDRRVDGAG | Human |
| | AASGLGST | |
| 585 | RCPDGCFLSSYCPVGYACSGFACCDCGGYDYAGG | Human |
| | VRGGRCSVRSTT | |
| 586 | NCPDGYKYNGFCTPDGGCSRVSSWGWDRSCISPT | Human |
| 587 | GCPPGTVAGVHCSPGSDCSWGCYDRDDRRVDGAG | Human |
| | ADSGLGSTST | |
| 588 | SCPDGYHFIEPCHSGLCWREGACNGDGICANGLG | Human |
| | RCRTVSETST | |
| 589 | GCPTGTVAGVHCSPGSDCSWGCYDADDRRVDGYG | Human |
| | ADSGVGSP | |
| 590 | NCPDGYGYQRHCTVGEDCTDHCCDAYGLCTS | Human |
| 591 | NCPDGYGYQRHCTVGEDCTDSCCDRYGLCTTSTET | Human |
| 592 | HCTVGEECTDSCCDRYGLCTTSTET | Human |
| 593 | NCPDGYGYQRHCTVGEDCTERCCDNYGLCTSYTD | Human |
| | T | |
| 594 | TCPLGYDLNYRCDHFNTCRVEECCKNGVVNAYGI | Human and Rat |
| | CEYAGGSAT | |
| 595 | TCPSGCSWFSGCWDTYRCGPSVCCRDGRYGCAAII | Human and Rat |
| | CRDT | |
| 596 | TCPRGYHYNDRCEFFNTCRVEECCLNGVVNTYGIC | Human and Rat |
| | EYEGGSAT | |
| 597 | TCPRGYHYNDRCDFFNTCRVEECCLNGVVNTYGI | Human and Rat |
| | CEYEGGSAT | |
| 598 | SCPDGYGYGDGYCYDSGCSASDCYGVDALYSYGH | Human and Rat |
| | CGCSIYTERPR | |
| 599 | SCPLGYAINDRCDDLKTCGPDECCLNGVVNAYGIC | Human and Rat |
| | EYEGESAT | |
| 600 | RCPSNNEDATACRYSSVCGDYVCEGLSESYAQGW | Human |
| | GACRRYACRDS | |
| 601 | NCPDGYEYNGFCTPDGGCSRVSNWGWDRSCISPT | Human |
| 602 | SCPDGYCDCNGCGYGNGCSRGGCFDFRLYSGYSAD | Human |
| | IVVSTT | |
| 603 | SCPDGWGHSDDCNCACSANAYACCKRDWLLPGPS | Human |
| | CECSTYCVS | |
| 604 | SCPDGWSDCHGSCDGVGCTGSDCVRYNARGYGR | Human |
| | HACSG | |
| 605 | GCPPGYKSGVDCSPGSECKWGCYAVDGRRYGGYG | Human |
| | ADSGVGST | |
| 606 | TCPEGLVYNSDQSRCCAADSGVCWEYWRGERVTR | Human |
| | GFT | |
| 607 | TCPDGYSYSEGCGKGDDCGGVHCCANGGVTCWY | Human |
| | RHCCSTGTDT | |
| 608 | RCPSNNEEATACRYSSVCGDYVCEGLSESYAQGW | Human |
| | GACRRYACRDS | |
| 609 | SGCPPGYKSGVDCSPGSEYKWGCYAVDGRRYGGY | Human |
| | GADSGVGST | |

1. The isolated antibody fragment of claim 57, wherein the fragment is the knob domain of a bovine ultralong CDR-H3 or a portion thereof.

2. An isolated antibody fragment according to claim 1, which comprises at least two, or at least four, or at least six, or at least eight, or at least ten cysteine residues and/or comprises at least one, or at least two, or at least three, or at least four, or at least five disulphide bonds.

3. (canceled)

4. An isolated antibody fragment according to claim 1, which comprises a (Z₁) X₁ C X₂ motif at its N-terminal extremity, wherein:
a. Z₁ is present or absent, and when Z₁ is present, Z₁ represents 1 amino acid or 2, 3, 4, or 5 independently selected amino acids; and,
b. X₁ is any amino acid residue; and,
c. C is cysteine; and,
d. X₂ is an amino acid selected from the list consisting of Proline, Arginine, Histidine, Lysine, Glycine and Serine; and/or
wherein said isolated antibody fragment comprises a (AB)n and/or (BA)n motif, wherein A is any amino acid residue, B is an aromatic amino acid selected from the group consisting of: tyrosine (Y), phenylalanine (F), tryptophan (W), and histidine (H), and wherein n is 1, 2, 3 or 4.

5. (canceled)

6. An isolated antibody fragment according to claim 57, which is 5 amino acids in length or more, 10 amino acids in length or more, 15 amino acids in length or more, 20 amino acids in length or more, 25 amino acids in length or more, 30 amino acids in length or more, 35 amino acids in length or more, 40 amino acids in length or more, 45 amino acids in length or more, and which is up to 55 amino acids in length.

7. (canceled)

8. An isolated antibody fragment according to claim 57, which further comprises a bridging moiety between two amino acids.

9. (canceled)

10. (canceled)

11. An isolated antibody fragment according to claim 57, which is fully bovine, chimeric, or synthetic.

12. (canceled)

13. (canceled)

14. An isolated antibody fragment according to claim 57, wherein the antigen of interest is the component C5 of the Complement.

15. An isolated antibody fragment according to claim 14, which has a sequence selected from the list consisting of SEQ ID NO: 157 to SEQ ID NO: 310, SEQ ID NO: 313, SEQ ID NO: 315, SEQ ID NO: 317, SEQ ID NO: 318, SEQ ID NO: 320, SEQ ID NO: 322, SEQ ID NO: 324, SEQ ID NO: 326 to SEQ ID NO: 331, SEQ ID NO: 334, SEQ ID NO: 336, SEQ ID NO: 339, SEQ ID NO: 341 to SEQ ID NO: 350, SEQ ID NO: 352, and SEQ ID NO: 572 to SEQ ID NO: 609 or any one of the same with at least 95%, 96%, 97%, 98% or 99% similarity or identity.

16. An isolated antibody fragment according to claim 57, wherein the antigen of interest is human serum albumin.

17. An isolated antibody fragment according to claim 16, which has the sequence SEQ ID NO: 510.

18. A polypeptide comprising at least one isolated antibody fragment as defined in claim 57.

19. (canceled)

20. (canceled)

21. (canceled)

22. (canceled)

23. An isolated antibody fragment according to claim 57, or a polypeptide comprising the isolated antibody fragment, wherein said fragment or polypeptide is fused to one or more effector molecules, optionally via a linker.

24. (canceled)

25. (canceled)

26. (canceled)

27. (canceled)

28. An isolated antibody fragment or a polypeptide according to claim 23, wherein the effector molecule is albumin or a protein comprising an albumin binding domain.

29. An isolated antibody fragment or a polypeptide according to claim 28, wherein the albumin binding domain comprises SEQ ID NO: 435 for CDR-H1, SEQ ID NO: 436 for CDR-H2, SEQ ID NO: 437 for CDR-H3, SEQ ID NO: 430 for CDR-L1, SEQ ID NO: 431 for CDR-L2 and SEQ ID NO: 432 for CDR-L3; or a heavy chain variable domain selected from SEQ ID NO: 434 and SEQ ID NO: 444 and a light chain variable domain selected from SEQ ID NO: 429 and SEQ ID NO: 443.

30. A pharmaceutical composition comprising an isolated antibody fragment as defined in claim 1, or a polypeptide comprising the isolated antibody fragment, in combination with one or more of a pharmaceutically acceptable excipient, diluent or carrier.

31. (canceled)

32. A polynucleotide encoding an isolated antibody fragment as defined in claim 57, or a polypeptide comprising the isolated antibody fragment.

33. A vector comprising a polynucleotide according to claim 32.

34. A host cell comprising the polynucleotide of claim 32 or a vector comprising the polynucleotide.

35. A process for producing an isolated antibody fragment as defined in claim 57, or a polypeptide comprising the isolated antibody fragment, said process comprising expressing the isolated antibody fragment, or the polypeptide from a host cell and/or said process comprising a step of chemical synthesis.

36. (canceled)

37. (canceled)

38. (canceled)

39. (canceled)

40. (canceled)

41. (canceled)

42. (canceled)

43. (canceled)

44. A library comprising at least one isolated antibody fragment as defined in claim 57, wherein the library is a synthetic library, a phage library, a naïve library, an immune library, a naïve library prepared from cattle, or an immune library prepared from cattle.

45. (canceled)

46. (canceled)

47. (canceled)

48. (canceled)

49. (canceled)

50. A phage display library, comprising a plurality of recombinant phages;
each of the plurality of recombinant phages comprising an M13-derived expression vector, wherein the M13-derived expression vector comprises a polynucleotide sequence encoding an isolated antibody fragment as defined in claim 57, optionally displayed within the full sequence of ultralong CDR-H3.

51. (canceled)

52. A method for generating a phage display library of ultralong CDR-H3 sequences, said method comprising:
a) immunising a bovine with an immunogenic composition, and;
b) isolating total RNA from PBMC or secondary lymphoid organ, and;
c) amplifying the cDNA sequences of the ultralong CDR-H3, and;
d) fusing the sequences obtained in c) to the sequence coding for the pIII protein of a M13 phage within a phagemid vector, and;
e) transforming host bacteria with the phagemid vector obtained at step d) in combination with a helper phage co-infection, and;
f) culturing the bacteria obtained at step e), and;
g) recovering the phages from the culture medium of the bacteria,
wherein the immunogenic composition comprises an antigen of interest or immunogenic portion thereof, or DNA encoding the same.

53. (canceled)

54. (canceled)

55. (canceled)

56. A method for producing an isolated antibody fragment which binds to an antigen of interest as defined in claim 57, said method comprising:
a) generating a phage display library of isolated antibody fragments; and,
b) enriching the phage display library against the antigen of interest to produce an enriched population of phage which bind the antigen of interest; and,
c) sequencing an isolated antibody fragment from the enriched population of phage obtained in step b); and,
expressing or synthesizing an isolated antibody fragment obtained in step c).

57. An isolated antibody fragment which binds to an antigen of interest and which comprises a sequence of the knob domain of a bovine ultralong CDR-H3 or a portion or a variant thereof and which does not comprise the stalk domain of the bovine ultralong CDR-H3.

58. A polypeptide comprising at least two isolated antibody fragments as defined in claim 57, wherein the antibody fragments are optionally linked together by a linker.


SEQUENCE LISTING (609 sequences)

SEQ ID NO: 1 - protein, 48 aa, Artificial Sequence
  Ultralong CDR-H3 K149
TSVLQSTKPQKSCPDGFSYRSWDDFCCPMVGRCLAPRNTYTTEFTIEA

SEQ ID NO: 2 - protein, 48 aa, Artificial Sequence
  Ultralong CDR-H3 K152
TSVLQSTKPQKSCPDGFSYRSWDDFLLSYGWECLAPRNTYTTEFTIEA

SEQ ID NO: 3 - protein, 49 aa, Artificial Sequence
  Ultralong CDR-H3 K147
VTVHQQTKRTCPRGYEYVSCWWGATCTYGGRCSGSRDDGSLTYEFHVDA

SEQ ID NO: 4 - protein, 49 aa, Artificial Sequence
  Ultralong CDR-H3 K148
VTVHQQTKRTCPRGYEYVSCWWGATCTYGGRCSAVGDDGSLTYEFHVDA

SEQ ID NO: 5 - protein, 52 aa, Artificial Sequence
  Ultralong CDR-H3 K142
TTVHQEPKKSCPEGYTYVWGCDDDSGGVGYGCAPNGASSCSFTYTYEFHIDA

SEQ ID NO: 6 - protein, 52 aa, Artificial Sequence
  Ultralong CDR-H3 K143
TTVHQETKKSCPEGYTYVWGCDDDSGGVGYGCAPNGASSCSFTYTYEFHIDA

SEQ ID NO: 7 - protein, 55 aa, Artificial Sequence
  Ultralong CDR-H3 K132
TTVHQRTLHNRNCPDGYGYQRHCTVGEDCTERCCDNYGLCTSYTDTYTYEFNVNA

SEQ ID NO: 8 - protein, 55 aa, Artificial Sequence
  Ultralong CDR-H3 K133
TTVHQRTLKNRNCPAGYGYQRHCTVGEDCTDSCCDRYGLCTTSTETYTYEFNVDA

SEQ ID NO: 9 - protein, 53 aa, Artificial Sequence
  Ultralong CDR-H3 K140
TAVHQRTKRTCPEGLVYNSDQSRCCAADSGVCWEYWRGERVTRGFTYEWYVEA

SEQ ID NO: 10 - protein, 53 aa, Artificial Sequence
  Ultralong CDR-H3 K141
TAVHQRTKRTCPEGLIYNSDQSRCCAADSGVCWEYWRGERVTRGFTYEWYVEA

SEQ ID NO: 11 - protein, 56 aa, Artificial Sequence
  Ultralong CDR-H3 K126
TTVHQQTHKKRSCPANHSVRDMCSYGPDDCGRSCCTDGIYVRRGSCSSAYEFHVDA

SEQ ID NO: 12 - protein, 56 aa, Artificial Sequence
  Ultralong CDR-H3 K129
TTVHQQTHKKRSCPENHSVRDMCSYGPDDCGRSCCTDGIYVRRGSCSSAYEFHVDA

SEQ ID NO: 13 - protein, 59 aa, Artificial Sequence
  Ultralong CDR-H3 K92
SIVHQKAHTSVTCPEGWSECGVAIYGYECGRWGCGHFLNSGPNISPYVSTHKYEWYVDA

SEQ ID NO: 14 - protein, 59 aa, Artificial Sequence
  Ultralong CDR-H3 K93
SIVHQKTQTSEGCPEGWSECGVGTYGYDCGRWGCGHYLNTGPLISGYVTTNKYEWHVEA

SEQ ID NO: 15 - protein, 59 aa, Artificial Sequence
  Ultralong CDR-H3 K94
STVHQKAHTSVACPEGWSECGVAIYGYDCGRWGCGHFLNSGPNISPYVTTDAYEWYVDA

SEQ ID NO: 16 - protein, 59 aa, Artificial Sequence
  Ultralong CDR-H3 K95
SIVHQRTQTSKGCPEGWNDCGGNTYGYDCGRWGCGHYLNSGPRISAYQTTYNYEWYVDA

SEQ ID NO: 17 - protein, 59 aa, Artificial Sequence
  Ultralong CDR-H3 K97
TIVHQKTQTREGCPEGWNECGEAIYGYDCGRWGCGHFLNTGPRISGYVTTYSYEWFVDT

SEQ ID NO: 18 - protein, 59 aa, Artificial Sequence
  Ultralong CDR-H3 K98
SIVHQKTQTSKGCPEGWNDCGVNIYGYDCGRWGCGHFLNSGPRISAYQTTYNYEWYVDA

SEQ ID NO: 19 - protein, 59 aa, Artificial Sequence
  Ultralong CDR-H3 K99
SIVHQRTQTRTGCPEGWNDCGRNTYGYDCGRWGCGHFLNSGPRISDYLTTYNYEWYVDA

SEQ ID NO: 20 - protein, 59 aa, Artificial Sequence
  Ultralong CDR-H3 K100
SIVHQKAHTSVTCPEGWSECGVAIYGYECGRWGCGHFLNSGPNISPYVTTDAYEWYVDA

SEQ ID NO: 21 - protein, 59 aa, Artificial Sequence
  Ultralong CDR-H3 K101
STVHQKAHTSVACPEGWSECGVAIYGYDCGRWGCGHFLNSGPNISPYVSTHKYEWYVDA

SEQ ID NO: 22 - protein, 59 aa, Artificial Sequence
  Ultralong CDR-H3 K102
STVHQKAHTSVACPEGWSECGVAIYGYDCGRWGCGHFLNSGSKYQSYVTTDAYEWYVDA

SEQ ID NO: 23 - protein, 59 aa, Artificial Sequence
  Ultralong CDR-H3 K103
TTVHQKAHTSVACPEGWSECGVAIYGYDCGRWGCGHFLNSGPNISPYVTTDAYEWYVDA

SEQ ID NO: 24 - protein, 59 aa, Artificial Sequence
  Ultralong CDR-H3 K104
SIVHQKAHTSVACPEGWSECGVAIYGYDCGRWGCGHFLNSGPNISPYVTTDAYEWYVDA

SEQ ID NO: 25 - protein, 59 aa, Artificial Sequence
  Ultralong CDR-H3 K107
SIVHQKAHTSVTCPEGWSECGVAIYGYDCGRWGCGHFLNSGPNISPYVTTDAYEWYVDA

SEQ ID NO: 26 - protein, 59 aa, Artificial Sequence
  Ultralong CDR-H3 K108
SIVHQKTQTSEGCPEGWSECGVGTYGYDCGRWGCGHYLNTGPLISGYVTTNKYEWHVDA

SEQ ID NO: 27 - protein, 59 aa, Artificial Sequence
  Ultralong CDR-H3 K109
STVHQKAHTSVACPEGWSECGVAIYGYECGRWGCGHFLNSGPNISPYVSTHKYEWYVDA

SEQ ID NO: 28 - protein, 59 aa, Artificial Sequence
  Ultralong CDR-H3 K110
SIVHQRTQTSKGCPEGWNDCGGNTYGYDCGRWGCGHFLNSGPNISPYVTTDAYEWYVDA

SEQ ID NO: 29 - protein, 58 aa, Artificial Sequence
  Ultralong CDR-H3 K115
TTIQQLTERTCPEGSMLGSECNSHWSCEGCDCAKHCTWGGRCVDCSPYMSTHEWHIET

SEQ ID NO: 30 - protein, 58 aa, Artificial Sequence
  Ultralong CDR-H3 K119
TTIQQSTERTCPEGSMLGSECNSHWSCEACDCARHCTWGGRCVDCSPYMSTYEWHIET

SEQ ID NO: 31 - protein, 63 aa, Artificial Sequence
  Ultralong CDR-H3 K27
TSVYQKTDTIRHPCRDDSSYACVCRWTRGCSGTDCSGCTPDSDIDYGCDTIACNYTYQLYVDA

SEQ ID NO: 32 - protein, 63 aa, Artificial Sequence
  Ultralong CDR-H3 K32
TTVYQKTDTKKHPCRDDSSYACVCRWTRGCSGTDCSGCTPDSDIDYGCDTIACNYTYQLYVDT

SEQ ID NO: 33 - protein, 63 aa, Artificial Sequence
  Ultralong CDR-H3 K44
TTVVPENRHKKHPCRDDSSYACVCRWTRGCSGTDCSGCTPDSDIDYGCDTIACNYTYQLYVDT

SEQ ID NO: 34 - protein, 62 aa, Artificial Sequence
  Ultralong CDR-H3 K52
TTVHQHSNNKKTCPDGTSSHSACILGTGGCCLDQYYRRGICGRVDACYEYSSSVNYEWYVDA

SEQ ID NO: 35 - protein, 62 aa, Artificial Sequence
  Ultralong CDR-H3 K54
TTVHQHTNNKKTCPDGSSSHSACKLGTGGCCLDGYYRRGICGRVDACYEYSSSVNYEWYVDA

SEQ ID NO: 36 - protein, 60 aa, Artificial Sequence
  Ultralong CDR-H3 K83
ATVHQRTERSCPDGSSDAESGVCSGCCRGWDCCSFEVDWVGCKGCTAYTYRTVYEHHVDA

SEQ ID NO: 37 - protein, 60 aa, Artificial Sequence
  Ultralong CDR-H3 K85
ATVHQRTERSCPDGSSDAESGVCSGCCRGWDCCSFEVDWVGCKGCTAYTYRTIHEHHVDA

SEQ ID NO: 38 - protein, 60 aa, Artificial Sequence
  Ultralong CDR-H3 K87
ATVHQRTERSCPDGSSDAESGVCSGCCRGWDCCSFEVDWVGCKGCTAYTYRSIYEHHVDA

SEQ ID NO: 39 - protein, 60 aa, Artificial Sequence
  Ultralong CDR-H3 K89
VTVHQRAERTCPDGSSDAESGVCSGCCGGWDCCSFKVDWVGCKECTAYPYNTRYEHHVDA

SEQ ID NO: 40 - protein, 64 aa, Artificial Sequence
  Ultralong CDR-H3 K13
TTVHQQTKTKKNPCRDVASPVCVCRWAEGCSGTDCSECTPDPDRDYGTCEIIACTHTYELHVDA

SEQ ID NO: 41 - protein, 64 aa, Artificial Sequence
  Ultralong CDR-H3 K14
TTVHQKTKTKKNPCRDVTSPVCVCRWAEGCSGTDCSDCTPDPDRDYGTCEIIACTHAYELHVDA

SEQ ID NO: 42 - protein, 64 aa, Artificial Sequence
  Ultralong CDR-H3 K20
TTVHQKTKTKKNPCRDVTSPVCVCRWAEGCSGTDCSDCTPDPDRDYGTCEIIACTHTYELHVDA

SEQ ID NO: 43 - protein, 63 aa, Artificial Sequence
  Ultralong CDR-H3 K29
TTVIQKTATKQSCPDDYRDGGECCIYGRCSAEDCSVTGWEYYGSTLCRVPYITTHAYQWHVDA

SEQ ID NO: 44 - protein, 63 aa, Artificial Sequence
  Ultralong CDR-H3 K34
TTVIQKTATKQSCPDDYRDGGECCIYGRCSAEDCSVTGWEYYGSTLCRVPYITTHSYQWHVDA

SEQ ID NO: 45 - protein, 63 aa, Artificial Sequence
  Ultralong CDR-H3 K37
TTVIQKTATKQSCPDDYRDGGECCIYERCSAEDCSVTGWEYYGSTLCRVPYITTLAYQWHVDA

SEQ ID NO: 46 - protein, 63 aa, Artificial Sequence
  Ultralong CDR-H3 K46
TTVIQKTATKQSCPDDYRDGGECCIYGRCSAEDCSVTGWEYYGSTLCRVPYITTLCLPVARRA

SEQ ID NO: 47 - protein, 61 aa, Artificial Sequence
  Ultralong CDR-H3 K59
TTVHQETRRNCPDGYSEINACGDRYKASGGLCCGEGAGAWRCWECSDTIIPTTTYEFYVDA

SEQ ID NO: 48 - protein, 61 aa, Artificial Sequence
  Ultralong CDR-H3 K61
TTVHQETRRHCPDGYSDIYGCGHYYSATGGHCCGEGAGAWRCWECSDTIMPSTTYEFYVDA

SEQ ID NO: 49 - protein, 61 aa, Artificial Sequence
  Ultralong CDR-H3 K62
TTVHQETRRNCPDGYSDIYGCGNRYAATGGHCCGEGAGAWRCWECSDSIWPSSTYEFYVDA

SEQ ID NO: 50 - protein, 61 aa, Artificial Sequence
  Ultralong CDR-H3 K64
STVHQDTRRHCPDGYSDIYACGHYYSATGGHCCGEGAGAWRCWECSDTIMPSTTYEFYVDA

SEQ ID NO: 51 - protein, 61 aa, Artificial Sequence
  Ultralong CDR-H3 K65
TTVHQESRRHCPDGYSDIYGCGHYYSSTGGHCCGEGAGAWRCWECSDTISPSTTYDFHVDA

SEQ ID NO: 52 - protein, 61 aa, Artificial Sequence
  Ultralong CDR-H3 K66
STVHQDTRRHCPDGYSDIYGCGHYYSATGGHCCGEGAGAWRCWECSDTIMPSTSYEFYVDA

SEQ ID NO: 53 - protein, 61 aa, Artificial Sequence
  Ultralong CDR-H3 K68
TTVHQETRRNCPDGYSNIYDCGHYYSSSGGHCCGEGAGAWRCWECSDTISPSTTYEFYVDA

SEQ ID NO: 54 - protein, 61 aa, Artificial Sequence
  Ultralong CDR-H3 K71
TTVHQETRRSCPDGYSDIYGCGHYYSSTGGHCCGEGAGAWRCWECSDTISPRTRYEFAVDA

SEQ ID NO: 55 - protein, 61 aa, Artificial Sequence
  Ultralong CDR-H3 K74
TTVHQETRRNCPDGYSDIKGCGNAYAATGGHCCGEGAGAWRCWECSDTIAPSSTYEFYVDA

SEQ ID NO: 56 - protein, 61 aa, Artificial Sequence
  Ultralong CDR-H3 K78
STVHQETRRSCPDGYSDIYGCGHYYSSTGGHCCGEGAGAWRCWECSDTISPSTRYEFYVDA

SEQ ID NO: 57 - protein, 61 aa, Artificial Sequence
  Ultralong CDR-H3 K79
TTVRQETRRNCPFGYSDIKGCGNRYAATGGHCCGEGAGAWRCWECSDTIRPSSTYEFYVDA

SEQ ID NO: 58 - protein, 61 aa, Artificial Sequence
  Ultralong CDR-H3 K60
VIVYQETIKSCREGYIDGGGCCLPGSCRGCACSYYDWLKCPRDCRGTSEEYIYTYNFRVDA

SEQ ID NO: 59 - protein, 61 aa, Artificial Sequence
  Ultralong CDR-H3 K77
GIVYQETIKSCPEGYIDGGGCCLPGSCRGCACTYYNVLKCPRDCRGTSEEYIYRYKFHVDA

SEQ ID NO: 60 - protein, 64 aa, Artificial Sequence
  Ultralong CDR-H3 K10
STVHQLTITTLGCPDGVSVVNTCGWLRCNCGDSIYCSRSADSGMWCGRCGDCTSTHTHQWHVDA

SEQ ID NO: 61 - protein, 64 aa, Artificial Sequence
  Ultralong CDR-H3 K11
STVHQLTITTLGCPDGVSVVPTCGWLRCNCGEDLYCSRSDEQGTWCGRCGDCTSTYTHQWHVDA

SEQ ID NO: 62 - protein, 64 aa, Artificial Sequence
  Ultralong CDR-H3 K18
STVHQLTITTVGCPNGVTRVATCGWKRCHCGENIYCSRSDDSGTWCGRCGDCTGTYTYQWHVDA

SEQ ID NO: 63 - protein, 64 aa, Artificial Sequence
  Ultralong CDR-H3 K19
STVHQLTITTVGCPNGVPRVTTCGWKRCHCGENIYCSRSDDSGTWCGRCGDCTGTYTYQWHVDA

SEQ ID NO: 64 - protein, 64 aa, Artificial Sequence
  Ultralong CDR-H3 K21
GTVHQLTITTLGCPDGVSVVNTCGWNRCNCGDTTFCSRSDDSGTWCGRCGDCSSTHTHQWHVDA

SEQ ID NO: 65 - protein, 64 aa, Artificial Sequence
  Ultralong CDR-H3 K22
STVHQLTITTLGCPDGVSVVNTCGWKRCNCGDSIYCSRSADDDGWCGRCGDCTSTHTHQWHVDA

SEQ ID NO: 66 - protein, 64 aa, Artificial Sequence
  Ultralong CDR-H3 K23
STVHQLTITTVGCPNGVTRVATCGWKRCHCSENIYCSRSDDSGTWCGRCGDCTNTYTFQWHVDA

SEQ ID NO: 67 - protein, 62 aa, Artificial Sequence
  Ultralong CDR-H3 K47
TTVHQKTIAKCPDGYTYSGDCGICDDCGGRTSRAYDCAGDTSLYMCGRRSPTLLTYQFHVDV

SEQ ID NO: 68 - protein, 62 aa, Artificial Sequence
  Ultralong CDR-H3 K55
TTVTPETIAKCPDGYTYSGDCGICDDCGGRTSRAYDCAGDTSLYMCGRRSPTLLTYQFHVDV

SEQ ID NO: 69 - protein, 67 aa, Artificial Sequence
  Ultralong CDR-H3 K2
ATVHQQTKKQTERSCPDGYTYINDCIGASGAVSRYDCWRFRRMNGVCIDGTYSTTADTYTYEFHVDA

SEQ ID NO: 70 - protein, 67 aa, Artificial Sequence
  Ultralong CDR-H3 K3
ATVHQQTKKQTERSCPDGYTYIVDCIGATGAVSRYDCWRFRRMNGVCIDGTYSTTADTYTYEFHVDA

SEQ ID NO: 71 - protein, 63 aa, Artificial Sequence
  Ultralong CDR-H3 K31
TTVHQKTRKSCPGGCRDTDGHDYDHWSCAGSDCCCFGTDGGCGRWGIYCSHSYTYTYEYHVET

SEQ ID NO: 72 - protein, 63 aa, Artificial Sequence
  Ultralong CDR-H3 K33
TTVHQKTRKSCPGGCRDTDGHDYDHWSCAGSDCCCFGTDGGCGRWGVYCSHSYTYTYEYHVDT

SEQ ID NO: 73 - protein, 63 aa, Artificial Sequence
  Ultralong CDR-H3 K45
TTVHQKTRKSCPGGCRDTDGHDYDHWSCAGSDCCCFGTDGGCGRWGIYCSHSYTYTYEYHVDT

SEQ ID NO: 74 - protein, 64 aa, Artificial Sequence
  Ultralong CDR-H3 K8
CTVQQKTHQVCPDGFNWGYGCAAGSSRFCTRHDWCCYDERADSHTYGFCTGNRVTNTYEFHADA

SEQ ID NO: 75 - protein, 64 aa, Artificial Sequence
  Ultralong CDR-H3 K9
TTVQQKTHQDCPDGFNWGYGCAAGSSLHCARHDWCCYDDRVGRDTYGFCTGNRATTTYEFHVDA

SEQ ID NO: 76 - protein, 67 aa, Artificial Sequence
  Ultralong CDR-H3 K1
TTVHQKTDQKRSSCPDGYSDCLVCGADRDGCSSGGCRGCWTNAYYSSRTYYNTDEFHYKPNEFHVDM

SEQ ID NO: 77 - protein, 65 aa, Artificial Sequence
  Ultralong CDR-H3 K4
TSVYQKTTKRFTCHDPSGGTWERADGATSCPGTHCCSYGRDGIWHGYDRRRTYTEVFTYELDVEE

SEQ ID NO: 78 - protein, 65 aa, Artificial Sequence
  Ultralong CDR-H3 K5
CTVYQKTETKKSCPDGYRFFQECRGTGTGCPGDDCVCYDGRGGFRWRNGCTTYTYTYRHNLHVET

SEQ ID NO: 79 - protein, 65 aa, Artificial Sequence
  Ultralong CDR-H3 K6
TTVYQETKIMRICPDDERRRWGCSDDSEGCSDSDCHIYDGDGSVGCCDGYLNSREIYKYAFHIDA

SEQ ID NO: 80 - protein, 65 aa, Artificial Sequence
  Ultralong CDR-H3 K7
VAVHQKTTERYSCPDGYSSCSSCRANDLDCRGVDCVNDRVCRGDGGFFSSRGYIVTYNYDFRVDA

SEQ ID NO: 81 - protein, 64 aa, Artificial Sequence
  Ultralong CDR-H3 K12
AAVHQETKTLRTCPPGLSDSNACPVGTWASRRTGCCSCCDRFCGGYSTCTDYTDTYTYEWHVDT

SEQ ID NO: 82 - protein, 64 aa, Artificial Sequence
  Ultralong CDR-H3 K15
TTVHQETKITSPACPDGYFYEYRCLVGGGCGWGCWNAAGGRPNAAGSLDRSPIETVTYEFQVDA

SEQ ID NO: 83 - protein, 64 aa, Artificial Sequence
  Ultralong CDR-H3 K16
TTVYQKTTKSTCPDGYIADGGCRKAGSWCSSVDCAGYGEDGDYGGWRTSCCYFVASAYEFHVDT

SEQ ID NO: 84 - protein, 64 aa, Artificial Sequence
  Ultralong CDR-H3 K17
GTVHQQTQEKCPDGYTFTANNCVTSSVRCSGRNCCGGDSYGYYIGIGGICHYDYTYTYENYVEA

SEQ ID NO: 85 - protein, 63 aa, Artificial Sequence
  Ultralong CDR-H3 K24
TIVHQETNKEKICRVDYVDSATCTWNCDCCRSRKSDCCAYANSRSCWNTSGTYTYTYEFHVDA

SEQ ID NO: 86 - protein, 63 aa, Artificial Sequence
  Ultralong CDR-H3 K25
TTVHQRTITRCPDDFGNTCRCSKGTCPCGEDACCGTNQYSFWGDCRDVGRTTFIETYEWNVDD

SEQ ID NO: 87 - protein, 63 aa, Artificial Sequence
  Ultralong CDR-H3 K26
TTVYQNTRSKERSCPYGTGFDPTWCDSVLPCRRDGCWTTVWGCCEGDVDGGETTPTYEFYVDA

SEQ ID NO: 88 - protein, 63 aa, Artificial Sequence
  Ultralong CDR-H3 K28
TTVYQKTRSDCPAGYKQVYGCSAGNCGCRGNGCCNSGSCGTWSEWGQYGCCNCHSSYEFHVDA

SEQ ID NO: 89 - protein, 63 aa, Artificial Sequence
  Ultralong CDR-H3 K30
TTVHQTTRKTQSCPDGYTDIDGCSWRHGCCRYDCCSDRSCSWCVDRDWSSYIVTATYELDIEA

SEQ ID NO: 90 - protein, 63 aa, Artificial Sequence
  Ultralong CDR-H3 K35
TTVHQETKHTRSCPDGYTDRVGCPYLWTSCARGDCWRIDRGATANPAATTYTYTDTYDWHIET

SEQ ID NO: 91 - protein, 63 aa, Artificial Sequence
  Ultralong CDR-H3 K36
ITAHQKTNKIPHCRDGYDYGGGCCVSSGVYGESCRSSGGSDCDQWVGCESVTYTETYEWHVDA

SEQ ID NO: 92 - protein, 63 aa, Artificial Sequence
  Ultralong CDR-H3 K38
TSVLQKTRHTCPDGYEYDTACGHGRCCCVGSSCRRNHTYGDYRRWGLYNSYSPAYTYEFHVDT

SEQ ID NO: 93 - protein, 63 aa, Artificial Sequence
  Ultralong CDR-H3 K39
TAVHQQTERSCPPDTTEHDCCGCGGRGCAWSGCYRKGYGTGCRVCTSIQARDYIYTYKLHIDT

SEQ ID NO: 94 - protein, 63 aa, Artificial Sequence
  Ultralong CDR-H3 K40
TTVHQNTIRSCPDGTDYAYGCRLGAWGCAGVGCCRGGAVGAWGCYGGDTFNTDSYTYEFYVDA

SEQ ID NO: 95 - protein, 63 aa, Artificial Sequence
  Ultralong CDR-H3 K41
TAVYQRTEARKSCPDGYNDVEARAHRSECSPNDCLRDGLGVASGCAWYRAYILIETYEFYVEA

SEQ ID NO: 96 - protein, 63 aa, Artificial Sequence
  Ultralong CDR-H3 K42
TTVYQKTRKLPSCREGTFYHAVCGGVVRCQVVDCDADGGCCYNAIGQYFGVSYSYKYEWFVEA

SEQ ID NO: 97 - protein, 63 aa, Artificial Sequence
  Ultralong CDR-H3 K43
ATVHQKTNKKQSCPDGYSDDDGRPDHWSCMDVDCWRPARGGWGSNCEHTNYIYTYTYEYHVDA

SEQ ID NO: 98 - protein, 62 aa, Artificial Sequence
  Ultralong CDR-H3 K48
TIVHQKTKREERCPAGYSISACRDGIGCGATDCCADGATDYAWGWECKSRIYGDSYEFHVDA

SEQ ID NO: 99 - protein, 62 aa, Artificial Sequence
  Ultralong CDR-H3 K49
TTTVQRTHKTTSCPDGYHFIEPCHSGLCWREGACNGDGICANGLGRCRTVSETSTYEFYVDA

SEQ ID NO: 100 - protein, 62 aa, Artificial Sequence
  Ultralong CDR-H3 K50
TTVYQRAQSKSCPDYCSCIFSYCSGADGCSSYGYCGHGGDEGDGFNGGGSRVSYTYEFYVDS

SEQ ID NO: 101 - protein, 62 aa, Artificial Sequence
  Ultralong CDR-H3 K51
TTVHQQTRTRCPDDYSYRSRGWIGSDCGGHGCWSDRDARRYDVYGNCNRVGEINTYEWYVDA

SEQ ID NO: 102 - protein, 62 aa, Artificial Sequence
  Ultralong CDR-H3 K53
TTVHQRTKKKLVLSVMILMIVVTILILCRVEECCKNGVVNAYGICEYAGGSATYTYEWYVDA

SEQ ID NO: 103 - protein, 61 aa, Artificial Sequence
  Ultralong CDR-H3 K56
TTVHQKTITSCPDGYVYSYDCGICDDCGGRTSRAYDCAGDTSLYMCGRRSPSSAYQFHVDR

SEQ ID NO: 104 - protein, 61 aa, Artificial Sequence
  Ultralong CDR-H3 K57
TTVHQRTIKSGCPPGYKSGVDCSPGSECKWGCYAVDGRRYGGYGADSGVGSTYTHEFYVDA

SEQ ID NO: 105 - protein, 61 aa, Artificial Sequence
  Ultralong CDR-H3 K58
TTVHQRTKKTCPLGYDLNDRCDHFNTCRVEECCKNGVVNAYGICEYAGGSATYTYEWYVDA

SEQ ID NO: 106 - protein, 61 aa, Artificial Sequence
  Ultralong CDR-H3 K63
TTVHQKTQRPICPDDYTALNGWGCGEYRCCPKSGACCCSGGGVHLLQSCSLETKYEFYVSA

SEQ ID NO: 107 - protein, 61 aa, Artificial Sequence
  Ultralong CDR-H3 K67
LTVLQVTDRRASCPAGCQDECGSSENCYCFRYGIWCHGRYSSGNSGTYSSNGYSSTWYADA

SEQ ID NO: 108 - protein, 61 aa, Artificial Sequence
  Ultralong CDR-H3 K69
STVHHEAHKRCPEDYSDRDHCSCWAGCGDDDCWRVVAGWRCSNYRYIGASYTHTYDFYADT

SEQ ID NO: 109 - protein, 61 aa, Artificial Sequence
  Ultralong CDR-H3 K70
TTVHQKTKKSCPLGYAINDRCDDLKTCGPDECCLNGVVNAYGICEYEGESATHTYEWYVDA

SEQ ID NO: 110 - protein, 61 aa, Artificial Sequence
  Ultralong CDR-H3 K72
TLVYQKTKKSCPEGYEGAPDCGAFDYCRVDDCCCRSGYGSCRRDSCRSGIRTSTYEFYVDT

SEQ ID NO: 111 - protein, 61 aa, Artificial Sequence
  Ultralong CDR-H3 K73
TTVYQHTRNRCPDDYRDCGHCCCQYGCHAVGCWRRQGGGFERCGEVDSQSPTYMYEFHVDA

SEQ ID NO: 112 - protein, 61 aa, Artificial Sequence
  Ultralong CDR-H3 K75
ATVLQYTHKTCPDGYEFGKNCPDGHGCSGSDCWRCDSRSAWWCTNYSWTDSIHAYELYVDA

SEQ ID NO: 113 - protein, 61 aa, Artificial Sequence
  Ultralong CDR-H3 K76
TTVHQKTEKSCKGGTDCGAGCCADGDPCSSGRCRAWSSTLRDYFYYPTSNYTYICDFHIDA

SEQ ID NO: 114 - protein, 61 aa, Artificial Sequence
  Ultralong CDR-H3 K80
TIVFQKTTKSCPGVSAEGGVCCSGTACTVPECWWFHQGHYSIPGGCTAATYTHTYESHVDA

SEQ ID NO: 115 - protein, 61 aa, Artificial Sequence
  Ultralong CDR-H3 K81
TTVHQKTNQEKHCPDGYDYCRVTEDGYCCSAWTCMHWRCAPGHKEYSVVSTTYTYEWYVDA

SEQ ID NO: 116 - protein, 61 aa, Artificial Sequence
  Ultralong CDR-H3 K82
VTTHQKSRKICPDGCIYACSCREEWRCTVFDCVRPRDVPNGRNACVSTCPSTSIYEFRVDA

SEQ ID NO: 117 - protein, 60 aa, Artificial Sequence
  Ultralong CDR-H3 K84
STVYQETKRKCPDGYRVGTDCTPGKGCDYACHSRLGVRWGGDGRDGGRGYIVSYELHIDA

SEQ ID NO: 118 - protein, 60 aa, Artificial Sequence
  Ultralong CDR-H3 K86
TTVYQKTQKPTDGYSCGITCRKRCDCSFVGYCACSESVSGDCTCYPRDSIPYRHEWYVDA

SEQ ID NO: 119 - protein, 60 aa, Artificial Sequence
  Ultralong CDR-H3 K88
TTIFQKTRRNCPPSSTSDGDCRGGWTCRGGDCSRWRGYYSSGNNYCCYDYTDTYEFYVDQ

SEQ ID NO: 120 - protein, 60 aa, Artificial Sequence
  Ultralong CDR-H3 K90
TTVRQKTAKSCPWGYDNGHGCNCGNDVFACSECLRSGTCSRYGRYEAYSYIVTYEFSVDA

SEQ ID NO: 121 - protein, 60 aa, Artificial Sequence
  Ultralong CDR-H3 K91
STVHQKTRQSCPDDYPVKCERGCGRERCGNCGWACNGPVGSPTCSYCRPYIYTYEFYVDA

SEQ ID NO: 122 - protein, 59 aa, Artificial Sequence
  Ultralong CDR-H3 K96
TTVYQKTKETCPDGYIWAERCPGGWTSCRNACWLEGGDSAGAYDEVTSTVHRYEFYVDT

SEQ ID NO: 123 - protein, 59 aa, Artificial Sequence
  Ultralong CDR-H3 K105
TAVYQKTEEKNTCPDGHTWRHGYRCTGWSYGCFRGAGNDCSDFGGDRITTYGYEWYVEN

SEQ ID NO: 124 - protein, 59 aa, Artificial Sequence
  Ultralong CDR-H3 K106
TTVHQRTIRTCPDGYGYQDACGRWGGCVGRACCSSGGSGCCDGSCGTMYIDNYDLYVDA

SEQ ID NO: 125 - protein, 58 aa, Artificial Sequence
  Ultralong CDR-H3 K111
GIVVQRTYERRTCPDTFTYKDGCRRGGTLLNSRSGCYNVYCNYHDAEVTYAHRWYVDA

SEQ ID NO: 126 - protein, 58 aa, Artificial Sequence
  Ultralong CDR-H3 K112
VTVQQQTKLEYSCPNGYSSDAGCLAAWRCGDYDCCRENAFRPCTGSIPTSNYEWHLEA

SEQ ID NO: 127 - protein, 58 aa, Artificial Sequence
  Ultralong CDR-H3 K113
STVHQETIRTCPDGGTYARDCGRECAICGHCGCCQNAYRRNWETCNTYTESINFHIDA

SEQ ID NO: 128 - protein, 58 aa, Artificial Sequence
  Ultralong CDR-H3 K114
TAVVQRSLKKPSCPSGYTLWGDCEGDDGGEGGVCRCWRPHSTVATPTYASTFQWHVDA

SEQ ID NO: 129 - protein, 58 aa, Artificial Sequence
  Ultralong CDR-H3 K116
TTVYQKTNTERSCPEWVQTSRTCIYRSRCGQYVCWSLGEDDCGVTCTDTTTYEWYVDA

SEQ ID NO: 130 - protein, 58 aa, Artificial Sequence
  Ultralong CDR-H3 K117
ITVHQETIRTCPDAWRSSATCRGAYGEAYECCPSGSSMWTSCVGCTTATFSYNLYVEA

SEQ ID NO: 131 - protein, 58 aa, Artificial Sequence
  Ultralong CDR-H3 K118
TTVHQKTSAKRTCPDGWRPGSECGWEDRCCGEFCSRCDWHGGWRAYMETQTYEFNVDS

SEQ ID NO: 132 - protein, 57 aa, Artificial Sequence
  Ultralong CDR-H3 K120
STVHQQTNKRRQNCPDGYKYNGFCTPDGGCSRVSSWGWDRSCISPTYTYTYEWYVEA

SEQ ID NO: 133 - protein, 57 aa, Artificial Sequence
  Ultralong CDR-H3 K121
TTVYQSTRKTSRNCPDGGSPSVQCLDDTWACRIVDCYDDGTYGTYRFTNTYDWYVDA

SEQ ID NO: 134 - protein, 57 aa, Artificial Sequence
  Ultralong CDR-H3 K122
STVFQHTKTTCSCPDNWETSGDCAGSSGDCSDCTCWRLGYGRTSSIATFNYEWYVEA

SEQ ID NO: 135 - protein, 57 aa, Artificial Sequence
  Ultralong CDR-H3 K123
TTVHQKTESHRSCPGDRPVDCGDDYGTLGCCPFHVGCGTWRCIEHIYTYTYQFHVDA

SEQ ID NO: 136 - protein, 57 aa, Artificial Sequence
  Ultralong CDR-H3 K124
TTVYQSTRKTSRNCPDGGSPSVQCLDDTWACRIVDCYATVLMVPIVLPTLYDWYVDA

SEQ ID NO: 137 - protein, 57 aa, Artificial Sequence
  Ultralong CDR-H3 K125
ATVHQYTHRSCPVGYDGGGNCGRYVDTCWGSDCCRYRRGIDYSCSSYSSSYEFYFEA

SEQ ID NO: 138 - protein, 56 aa, Artificial Sequence
  Ultralong CDR-H3 K127
ASVHQETKRSCPDGYRRGLECSAEWRCRYYDCVECSYGLCGHITRYIESYAWHVDT

SEQ ID NO: 139 - protein, 56 aa, Artificial Sequence
  Ultralong CDR-H3 K128
TAVHQETKKQPPNCPDGSSLLSSCFDTGGCSLYSCGREGRRRTYTYSYTYEWYVDA

SEQ ID NO: 140 - protein, 56 aa, Artificial Sequence
  Ultralong CDR-H3 K130
GTVHQKTNDHTRCPDGYYQGWHMSLRRYVCARDGYNPERYYVEATHTYTYEFHIDA

SEQ ID NO: 141 - protein, 56 aa, Artificial Sequence
  Ultralong CDR-H3 K131
TTVHQQTNTKNCPTWCGFAHSCILRYEACSDCDCSGGAGDYAAPGLYHTYEFHVDA

SEQ ID NO: 142 - protein, 55 aa, Artificial Sequence
  Ultralong CDR-H3 K134
IAVHQETKRSCPGGYIARCAGTYGCSAVPGCCDFSGDCLWRADSLTLTYELHVDT

SEQ ID NO: 143 - protein, 55 aa, Artificial Sequence
  Ultralong CDR-H3 K135
ATVHQKNNRKKKLVRMVVNLVSSVSTPVKFCRISECYEDHPTTIYTYTYEFHVDA

SEQ ID NO: 144 - protein, 54 aa, Artificial Sequence
  Ultralong CDR-H3 K136
TTVHQTTNRKKTCPDNYREVDGCDPYDCCLTTWCTNSYCTRYIYEDSYEFYVTA

SEQ ID NO: 145 - protein, 54 aa, Artificial Sequence
  Ultralong CDR-H3 K137
ATVHQKTTEKKTCPDGGEPSVICLDASEVCRISECYEDHPTTIYTYTYEFHVDA

SEQ ID NO: 146 - protein, 54 aa, Artificial Sequence
  Ultralong CDR-H3 K138
TTVHQKTKRSCPAYDSSGCGCVYYSPWNACICDKPGGPCDGVNPITSYEFNVDA

SEQ ID NO: 147 - protein, 54 aa, Artificial Sequence
  Ultralong CDR-H3 K139
TAVYQKTSESQRTCPSWCSLYMCGGYLACSACGCAENGRYGNGITYTYEWHVDA

SEQ ID NO: 148 - protein, 52 aa, Artificial Sequence
  Ultralong CDR-H3 K144
TTVHQKTTKTCPDGYVYNDPCDCWGRRNYDCCCEGGREFYTFVYSHEFNVHS

SEQ ID NO: 149 - protein, 50 aa, Artificial Sequence
  Ultralong CDR-H3 K145
TSVLQSTKKQKSCPDGLSYRAWDDFCCPNVGRCLPPINTYTYTHAFHIEA

SEQ ID NO: 150 - protein, 50 aa, Artificial Sequence
  Ultralong CDR-H3 K146
TTLYQNTRKKGGCPEGTTYLGGSSETYRCGLEGRMRTYSYTYSYEWYVDA

SEQ ID NO: 151 - protein, 48 aa, Artificial Sequence
  Ultralong CDR-H3 K150
TSVLQSTKKQKTCPDGLSYRSWDGFCCPKVGRCLPTIDAYINQFHIEA

SEQ ID NO: 152 - protein, 48 aa, Artificial Sequence
  Ultralong CDR-H3 K151
TSVLQSTKEQKTCPDGLSYRSWDGFCCPKYGRCLAATSTYTTEFYIEA

SEQ ID NO: 153 - protein, 47 aa, Artificial Sequence
  Ultralong CDR-H3 K153
GAVYQKTNEQSSCPDGWRDTGTHCEDYGSWGYRDYTFTYTYEFHVHN

SEQ ID NO: 154 - protein, 47 aa, Artificial Sequence
  Ultralong CDR-H3 K154
TTVHQTTRPNTDSCPSGYSTTLHCCCGSWKCDWCDPTTYKYELYVNA

SEQ ID NO: 155 - protein, 522 aa, Artificial Sequence
  scFc
PKSGDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSTASGSGSGGSGTAGSSGGAGSSGGSTTAGGSASGSGSTGSGTGGASSGGASGASGEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 556 aa, Artificial Sequence
  C terminal Tag
GSGSGSGSGSENLYFQGSGSHHHHHHHHHHGSGSPKSGDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSSTASGSGSGGSGTAGSSGGAGSSGGSTTAGGSASGSGSTGSGTGGASSGGASGASGEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 23 aa, Artificial Sequence
  CDR-H3 knob domain K149
SCPDGFSYRSWDDFCCPMVGRCL

SEQ ID NO: 158 - protein, 23 aa, Artificial Sequence
  CDR-H3 knob domain K152
SCPDGFSYRSWDDFLLSYGWECL

SEQ ID NO: 159 - protein, 24 aa, Artificial Sequence
  CDR-H3 knob domain K147
TCPRGYEYVSCWWGATCTYGGRCS

SEQ ID NO: 160 - protein, 24 aa, Artificial Sequence
  CDR-H3 knob domain K148
TCPRGYEYVSCWWGATCTYGGRCS

SEQ ID NO: 161 - protein, 32 aa, Artificial Sequence
  CDR-H3 knob domain K142
SCPEGYTYVWGCDDDSGGVGYGCAPNGASSCS

SEQ ID NO: 162 - protein, 32 aa, Artificial Sequence; identical to SEQ ID NO: 161
  CDR-H3 knob domain K143

SEQ ID NO: 163 - protein, 30 aa, Artificial Sequence
  CDR-H3 knob domain K132
NCPDGYGYQRHCTVGEDCTERCCDNYGLCT

SEQ ID NO: 164 - protein, 30 aa, Artificial Sequence
  CDR-H3 knob domain K133
NCPAGYGYQRHCTVGEDCTDSCCDRYGLCT

SEQ ID NO: 165 - protein, 24 aa, Artificial Sequence
  CDR-H3 knob domain K140
TCPEGLVYNSDQSRCCAADSGVCW

SEQ ID NO: 166 - protein, 24 aa, Artificial Sequence
  CDR-H3 knob domain K141
TCPEGLIYNSDQSRCCAADSGVCW

SEQ ID NO: 167 - protein, 36 aa, Artificial Sequence
  CDR-H3 knob domain K126
SCPANHSVRDMCSYGPDDCGRSCCTDGIYVRRGSCS

SEQ ID NO: 168 - protein, 36 aa, Artificial Sequence
  CDR-H3 knob domain K129
SCPENHSVRDMCSYGPDDCGRSCCTDGIYVRRGSCS

SEQ ID NO: 169 - protein, 24 aa, Artificial Sequence
  CDR-H3 knob domain K92
TCPEGWSECGVAIYGYECGRWGCG

SEQ ID NO: 170 - protein, 24 aa, Artificial Sequence
  CDR-H3 knob domain K93
GCPEGWSECGVGTYGYDCGRWGCG

SEQ ID NO: 171 - protein, 24 aa, Artificial Sequence
  CDR-H3 knob domain K94
ACPEGWSECGVAIYGYDCGRWGCG

SEQ ID NO: 172 - protein, 24 aa, Artificial Sequence
  CDR-H3 knob domain K95
GCPEGWNDCGGNTYGYDCGRWGCG

SEQ ID NO: 173 - protein, 24 aa, Artificial Sequence
  CDR-H3 knob domain K97
GCPEGWNECGEAIYGYDCGRWGCG

SEQ ID NO: 174 - protein, 24 aa, Artificial Sequence
  CDR-H3 knob domain K98
GCPEGWNDCGVNIYGYDCGRWGCG

SEQ ID NO: 175 - protein, 24 aa, Artificial Sequence
  CDR-H3 knob domain K99
GCPEGWNDCGRNTYGYDCGRWGCG

SEQ ID NO: 176 - protein, 24 aa, Artificial Sequence
  CDR-H3 knob domain K100
TCPEGWSECGVAIYGYECGRWGCG

SEQ ID NO: 177 - protein, 24 aa, Artificial Sequence
  CDR-H3 knob domain K101
ACPEGWSECGVAIYGYDCGRWGCG

SEQ ID NO: 178 - protein, 24 aa, Artificial Sequence
  CDR-H3 knob domain K102
ACPEGWSECGVAIYGYDCGRWGCG

SEQ ID NO: 179 - protein, 24 aa, Artificial Sequence
  CDR-H3 knob domain K103
ACPEGWSECGVAIYGYDCGRWGCG

SEQ ID NO: 180 - protein, 24 aa, Artificial Sequence
  CDR-H3 knob domain K104
ACPEGWSECGVAIYGYDCGRWGCG

SEQ ID NO: 181 - protein, 24 aa, Artificial Sequence
  CDR-H3 knob domain K107
TCPEGWSECGVAIYGYDCGRWGCG

SEQ ID NO: 182 - protein, 24 aa, Artificial Sequence
  CDR-H3 knob domain K108
GCPEGWSECGVGTYGYDCGRWGCG

SEQ ID NO: 183 - protein, 24 aa, Artificial Sequence
  CDR-H3 knob domain K109
ACPEGWSECGVAIYGYECGRWGCG

SEQ ID NO: 184 - protein, 24 aa, Artificial Sequence
  CDR-H3 knob domain K110
GCPEGWNDCGGNTYGYDCGRWGCG

SEQ ID NO: 185 - protein, 37 aa, Artificial Sequence
  CDR-H3 knob domain K115
TCPEGSMLGSECNSHWSCEGCDCAKHCTWGGRCVDCS

SEQ ID NO: 186 - protein, 37 aa, Artificial Sequence
  CDR-H3 knob domain K119
TCPEGSMLGSECNSHWSCEACDCARHCTWGGRCVDCS

SEQ ID NO: 187 - protein, 42 aa, Artificial Sequence
  CDR-H3 knob domain K27
PCRDDSSYACVCRWTRGCSGTDCSGCTPDSDIDYGCDTIACN

SEQ ID NO: 188 - protein, 42 aa, Artificial Sequence; identical to SEQ ID NO: 187
  CDR-H3 knob domain K32

SEQ ID NO: 189 - protein, 42 aa, Artificial Sequence; identical to SEQ ID NO: 187
  CDR-H3 knob domain K44

SEQ ID NO: 190 - protein, 37 aa, Artificial Sequence
  CDR-H3 knob domain K52
TCPDGTSSHSACILGTGGCCLDQYYRRGICGRVDACY

SEQ ID NO: 191 - protein, 37 aa, Artificial Sequence
  CDR-H3 knob domain K54
TCPDGSSSHSACKLGTGGCCLDGYYRRGICGRVDACY

SEQ ID NO: 192 - protein, 37 aa, Artificial Sequence
  CDR-H3 knob domain K83
SCPDGSSDAESGVCSGCCRGWDCCSFEVDWVGCKGCT

SEQ ID NO: 193 - protein, 37 aa, Artificial Sequence; identical to SEQ ID NO: 192
  CDR-H3 knob domain K85

SEQ ID NO: 194 - protein, 37 aa, Artificial Sequence; identical to SEQ ID NO: 192
  CDR-H3 knob domain K87

SEQ ID NO: 195 - protein, 37 aa, Artificial Sequence
  CDR-H3 knob domain K89
TCPDGSSDAESGVCSGCCGGWDCCSFKVDWVGCKECT

SEQ ID NO: 196 - protein, 43 aa, Artificial Sequence
  CDR-H3 knob domain K13
PCRDVASPVCVCRWAEGCSGTDCSECTPDPDRDYGTCEIIACT

SEQ ID NO: 197 - protein, 43 aa, Artificial Sequence
  CDR-H3 knob domain K14
PCRDVTSPVCVCRWAEGCSGTDCSDCTPDPDRDYGTCEIIACT

SEQ ID NO: 198 - protein, 43 aa, Artificial Sequence; identical to SEQ ID NO: 197
  CDR-H3 knob domain K20

SEQ ID NO: 199 - protein, 37 aa, Artificial Sequence
  CDR-H3 knob domain K29
SCPDDYRDGGECCIYGRCSAEDCSVTGWEYYGSTLCR

SEQ ID NO: 200 - protein, 37 aa, Artificial Sequence; identical to SEQ ID NO: 199
  CDR-H3 knob domain K34

SEQ ID NO: 201 - protein, 37 aa, Artificial Sequence
  CDR-H3 knob domain K37
SCPDDYRDGGECCIYERCSAEDCSVTGWEYYGSTLCR

SEQ ID NO: 202 - protein, 46 aa, Artificial Sequence
  CDR-H3 knob domain K46
SCPDDYRDGGECCIYGRCSAEDCSVTGWEYYGSTLCRVPYITTLCL

SEQ ID NO: 203 - protein, 37 aa, Artificial Sequence
  CDR-H3 knob domain K59
NCPDGYSEINACGDRYKASGGLCCGEGAGAWRCWECS

SEQ ID NO: 204 - protein, 37 aa, Artificial Sequence
  CDR-H3 knob domain K61
HCPDGYSDIYGCGHYYSATGGHCCGEGAGAWRCWECS

SEQ ID NO: 205 - protein, 37 aa, Artificial Sequence
  CDR-H3 knob domain K62
NCPDGYSDIYGCGNRYAATGGHCCGEGAGAWRCWECS

SEQ ID NO: 206 - protein, 37 aa, Artificial Sequence
  CDR-H3 knob domain K64
HCPDGYSDIYACGHYYSATGGHCCGEGAGAWRCWECS

SEQ ID NO: 207 - protein, 37 aa, Artificial Sequence
  CDR-H3 knob domain K65
HCPDGYSDIYGCGHYYSSTGGHCCGEGAGAWRCWECS

SEQ ID NO: 208 - protein, 37 aa, Artificial Sequence; identical to SEQ ID NO: 204
  CDR-H3 knob domain K66

SEQ ID NO: 209 - protein, 37 aa, Artificial Sequence
  CDR-H3 knob domain K68
NCPDGYSNIYDCGHYYSSSGGHCCGEGAGAWRCWECS

SEQ ID NO: 210 - protein, 37 aa, Artificial Sequence
  CDR-H3 knob domain K71
SCPDGYSDIYGCGHYYSSTGGHCCGEGAGAWRCWECS

SEQ ID NO: 211 - protein, 37 aa, Artificial Sequence
  CDR-H3 knob domain K74
NCPDGYSDIKGCGNAYAATGGHCCGEGAGAWRCWECS

SEQ ID NO: 212 - protein, 37 aa, Artificial Sequence; identical to SEQ ID NO: 210
  CDR-H3 knob domain K78

SEQ ID NO: 213 - protein, 37 aa, Artificial Sequence
  CDR-H3 knob domain K79
NCPFGYSDIKGCGNRYAATGGHCCGEGAGAWRCWECS

SEQ ID NO: 214 - protein, 36 aa, Artificial Sequence
  CDR-H3 knob domain K60
SCREGYIDGGGCCLPGSCRGCACSYYDWLKCPRDCR

SEQ ID NO: 215 - protein, 36 aa, Artificial Sequence
  CDR-H3 knob domain K77
SCPEGYIDGGGCCLPGSCRGCACTYYNVLKCPRDCR

SEQ ID NO: 216 - protein, 42 aa, Artificial Sequence
  CDR-H3 knob domain K10
GCPDGVSVVNTCGWLRCNCGDSIYCSRSADSGMWCGRCGDCT

SEQ ID NO: 217 - protein, 42 aa, Artificial Sequence
  CDR-H3 knob domain K11
GCPDGVSVVPTCGWLRCNCGEDLYCSRSDEQGTWCGRCGDCT

SEQ ID NO: 218 - protein, 42 aa, Artificial Sequence
  CDR-H3 knob domain K18
GCPNGVTRVATCGWKRCHCGENIYCSRSDDSGTWCGRCGDCT

SEQ ID NO: 219 - protein, 42 aa, Artificial Sequence
  CDR-H3 knob domain K19
GCPNGVPRVTTCGWKRCHCGENIYCSRSDDSGTWCGRCGDCT

SEQ ID NO: 220 - protein, 42 aa, Artificial Sequence
  CDR-H3 knob domain K21
GCPDGVSVVNTCGWNRCNCGDTTFCSRSDDSGTWCGRCGDCS

SEQ ID NO: 221 - protein, 42 aa, Artificial Sequence
  CDR-H3 knob domain K22
GCPDGVSVVNTCGWKRCNCGDSIYCSRSADDDGWCGRCGDCT

SEQ ID NO: 222 - protein, 42 aa, Artificial Sequence
  CDR-H3 knob domain K23
GCPNGVTRVATCGWKRCHCSENIYCSRSDDSGTWCGRCGDCT

SEQ ID NO: 223 - protein, 38 aa, Artificial Sequence
  CDR-H3 knob domain K47
KCPDGYTYSGDCGICDDCGGRTSRAYDCAGDTSLYMCG

SEQ ID NO: 224 - protein, 38 aa, Artificial Sequence; identical to SEQ ID NO: 223
  CDR-H3 knob domain K55

SEQ ID NO: 225 - protein, 35 aa, Artificial Sequence
  CDR-H3 knob domain K2
SCPDGYTYINDCIGASGAVSRYDCWRFRRMNGVCI

SEQ ID NO: 226 - protein, 35 aa, Artificial Sequence
  CDR-H3 knob domain K3
SCPDGYTYIVDCIGATGAVSRYDCWRFRRMNGVCI

SEQ ID NO: 227 - protein, 41 aa, Artificial Sequence
  CDR-H3 knob domain K31
SCPGGCRDTDGHDYDHWSCAGSDCCCFGTDGGCGRWGIYCS

SEQ ID NO: 228 - protein, 41 aa, Artificial Sequence
  CDR-H3 knob domain K33
SCPGGCRDTDGHDYDHWSCAGSDCCCFGTDGGCGRWGVYCS

SEQ ID NO: 229 - protein, 41 aa, Artificial Sequence; identical to SEQ ID NO: 227
  CDR-H3 knob domain K45

SEQ ID NO: 230 - protein, 41 aa, Artificial Sequence
  CDR-H3 knob domain K8
VCPDGFNWGYGCAAGSSRFCTRHDWCCYDERADSHTYGFCT

SEQ ID NO: 231 - protein, 41 aa, Artificial Sequence
  CDR-H3 knob domain K9
DCPDGFNWGYGCAAGSSLHCARHDWCCYDDRVGRDTYGFCT

SEQ ID NO: 232 - protein, 28 aa, Artificial Sequence
  CDR-H3 knob domain K1
SCPDGYSDCLVCGADRDGCSSGGCRGCW

SEQ ID NO: 233 - protein, 26 aa, Artificial Sequence
  CDR-H3 knob domain K4
TCHDPSGGTWERADGATSCPGTHCCS

SEQ ID NO: 234 - protein, 40 aa, Artificial Sequence
  CDR-H3 knob domain K5
SCPDGYRFFQECRGTGTGCPGDDCVCYDGRGGFRWRNGCT

SEQ ID NO: 235 - protein, 37 aa, Artificial Sequence
  CDR-H3 knob domain K6
ICPDDERRRWGCSDDSEGCSDSDCHIYDGDGSVGCCD

SEQ ID NO: 236 - protein, 31 aa, Artificial Sequence
  CDR-H3 knob domain K7
SCPDGYSSCSSCRANDLDCRGVDCVNDRVCR

SEQ ID NO: 237 - protein, 39 aa, Artificial Sequence
  CDR-H3 knob domain K12
TCPPGLSDSNACPVGTWASRRTGCCSCCDRFCGGYSTCT

SEQ ID NO: 238 - protein, 23 aa, Artificial Sequence
  CDR-H3 knob domain K15
ACPDGYFYEYRCLVGGGCGWGCW

SEQ ID NO: 239 - protein, 42 aa, Artificial Sequence
  CDR-H3 knob domain K16
TCPDGYIADGGCRKAGSWCSSVDCAGYGEDGDYGGWRTSCCY

SEQ ID NO: 240 - protein, 42 aa, Artificial Sequence
  CDR-H3 knob domain K17
KCPDGYTFTANNCVTSSVRCSGRNCCGGDSYGYYIGIGGICH

SEQ ID NO: 241 - protein, 36 aa, Artificial Sequence
  CDR-H3 knob domain K24
ICRVDYVDSATCTWNCDCCRSRKSDCCAYANSRSCW

SEQ ID NO: 242 - protein, 37 aa, Artificial Sequence
  CDR-H3 knob domain K25
RCPDDFGNTCRCSKGTCPCGEDACCGTNQYSFWGDCR

SEQ ID NO: 243 - protein, 33 aa, Artificial Sequence
  CDR-H3 knob domain K26
SCPYGTGFDPTWCDSVLPCRRDGCWTTVWGCCE

SEQ ID NO: 244 - protein, 45 aa, Artificial Sequence
  CDR-H3 knob domain K28
DCPAGYKQVYGCSAGNCGCRGNGCCNSGSCGTWSEWGQYGCCNCH

SEQ ID NO: 245 - protein, 33 aa, Artificial Sequence
  CDR-H3 knob domain K30
SCPDGYTDIDGCSWRHGCCRYDCCSDRSCSWCV

SEQ ID NO: 246 - protein, 25 aa, Artificial Sequence
  CDR-H3 knob domain K35
SCPDGYTDRVGCPYLWTSCARGDCW

SEQ ID NO: 247 - protein, 38 aa, Artificial Sequence
  CDR-H3 knob domain K36
HCRDGYDYGGGCCVSSGVYGESCRSSGGSDCDQWVGCE

SEQ ID NO: 248 - protein, 25 aa, Artificial Sequence
  CDR-H3 knob domain K38
TCPDGYEYDTACGHGRCCCVGSSCR

SEQ ID NO: 249 - protein, 37 aa, Artificial Sequence
  CDR-H3 knob domain K39
SCPPDTTEHDCCGCGGRGCAWSGCYRKGYGTGCRVCT

SEQ ID NO: 250 - protein, 36 aa, Artificial Sequence
  CDR-H3 knob domain K40
SCPDGTDYAYGCRLGAWGCAGVGCCRGGAVGAWGCY

SEQ ID NO: 251 - protein, 35 aa, Artificial Sequence
  CDR-H3 knob domain K41
SCPDGYNDVEARAHRSECSPNDCLRDGLGVASGCA

SEQ ID NO: 252 - protein, 31 aa, Artificial Sequence
  CDR-H3 knob domain K42
SCREGTFYHAVCGGVVRCQVVDCDADGGCCY

SEQ ID NO: 253 - protein, 36 aa, Artificial Sequence
  CDR-H3 knob domain K43
SCPDGYSDDDGRPDHWSCMDVDCWRPARGGWGSNCE

SEQ ID NO: 254 - protein, 37 aa, Artificial Sequence
  CDR-H3 knob domain K48
RCPAGYSISACRDGIGCGATDCCADGATDYAWGWECK

SEQ ID NO: 255 - protein, 37 aa, Artificial Sequence
  CDR-H3 knob domain K49
SCPDGYHFIEPCHSGLCWREGACNGDGICANGLGRCR

SEQ ID NO: 256 - protein, 26 aa, Artificial Sequence
  CDR-H3 knob domain K50
SCPDYCSCIFSYCSGADGCSSYGYCG

SEQ ID NO: 257 - protein, 39 aa, Artificial Sequence
  CDR-H3 knob domain K51
RCPDDYSYRSRGWIGSDCGGHGCWSDRDARRYDVYGNCN

SEQ ID NO: 258 - protein, 20 aa, Artificial Sequence
  CDR-H3 knob domain K53
LCRVEECCKNGVVNAYGICE

SEQ ID NO: 259 - protein, 38 aa, Artificial Sequence
  CDR-H3 knob domain K56
SCPDGYVYSYDCGICDDCGGRTSRAYDCAGDTSLYMCG

SEQ ID NO: 260 - protein, 23 aa, Artificial Sequence
  CDR-H3 knob domain K57
GCPPGYKSGVDCSPGSECKWGCY

SEQ ID NO: 261 - protein, 36 aa, Artificial Sequence
  CDR-H3 knob domain K58
TCPLGYDLNDRCDHFNTCRVEECCKNGVVNAYGICE

SEQ ID NO: 262 - protein, 40 aa, Artificial Sequence
  CDR-H3 knob domain K63
ICPDDYTALNGWGCGEYRCCPKSGACCCSGGGVHLLQSCS

SEQ ID NO: 263 - protein, 26 aa, Artificial Sequence
  CDR-H3 knob domain K67
SCPAGCQDECGSSENCYCFRYGIWCH

SEQ ID NO: 264 - protein, 33 aa, Artificial Sequence
  CDR-H3 knob domain K69
RCPEDYSDRDHCSCWAGCGDDDCWRVVAGWRCS

SEQ ID NO: 265 - protein, 36 aa, Artificial Sequence
  CDR-H3 knob domain K70
SCPLGYAINDRCDDLKTCGPDECCLNGVVNAYGICE

SEQ ID NO: 266 - protein, 38 aa, Artificial Sequence
  CDR-H3 knob domain K72
SCPEGYEGAPDCGAFDYCRVDDCCCRSGYGSCRRDSCR

SEQ ID NO: 267 - protein, 35 aa, Artificial Sequence
  CDR-H3 knob domain K73
RCPDDYRDCGHCCCQYGCHAVGCWRRQGGGFERCG

SEQ ID NO: 268 - protein, 35 aa, Artificial Sequence
  CDR-H3 knob domain K75
TCPDGYEFGKNCPDGHGCSGSDCWRCDSRSAWWCT

SEQ ID NO: 269 - protein, 47 aa, Artificial Sequence
  CDR-H3 knob domain K76
SCKGGTDCGAGCCADGDPCSSGRCRAWSSTLRDYFYYPTSNYTYICD

SEQ ID NO: 270 - protein, 38 aa, Artificial Sequence
  CDR-H3 knob domain K80
SCPGVSAEGGVCCSGTACTVPECWWFHQGHYSIPGGCT

SEQ ID NO: 271 - protein, 29 aa, Artificial Sequence
  CDR-H3 knob domain K81
HCPDGYDYCRVTEDGYCCSAWTCMHWRCA

SEQ ID NO: 272 - protein, 41 aa, Artificial Sequence
  CDR-H3 knob domain K82
ICPDGCIYACSCREEWRCTVFDCVRPRDVPNGRNACVSTCP

SEQ ID NO: 273 - protein, 23 aa, Artificial Sequence
  CDR-H3 knob domain K84
KCPDGYRVGTDCTPGKGCDYACH

SEQ ID NO: 274 - protein, 31 aa, Artificial Sequence
  CDR-H3 knob domain K86
SCGITCRKRCDCSFVGYCACSESVSGDCTCY

SEQ ID NO: 275 - protein, 39 aa, Artificial Sequence
  CDR-H3 knob domain K88
NCPPSSTSDGDCRGGWTCRGGDCSRWRGYYSSGNNYCCY

SEQ ID NO: 276 - protein, 31 aa, Artificial Sequence
  CDR-H3 knob domain K90
SCPWGYDNGHGCNCGNDVFACSECLRSGTCS

SEQ ID NO: 277 - protein, 39 aa, Artificial Sequence
  CDR-H3 knob domain K91
SCPDDYPVKCERGCGRERCGNCGWACNGPVGSPTCSYCR

SEQ ID NO: 278 - protein, 24 aa, Artificial Sequence
  CDR-H3 knob domain K96
TCPDGYIWAERCPGGWTSCRNACW

SEQ ID NO: 279 - protein, 30 aa, Artificial Sequence
  CDR-H3 knob domain K105
TCPDGHTWRHGYRCTGWSYGCFRGAGNDCS

SEQ ID NO: 280 - protein, 37 aa, Artificial Sequence
  CDR-H3 knob domain K106
TCPDGYGYQDACGRWGGCVGRACCSSGGSGCCDGSCG

SEQ ID NO: 281 - protein, 31 aa, Artificial Sequence
  CDR-H3 knob domain K111
TCPDTFTYKDGCRRGGTLLNSRSGCYNVYCN

SEQ ID NO: 282 - protein, 33 aa, Artificial Sequence
  CDR-H3 knob domain K112
SCPNGYSSDAGCLAAWRCGDYDCCRENAFRPCT

SEQ ID NO: 283 - protein, 37 aa, Artificial Sequence
  CDR-H3 knob domain K113
TCPDGGTYARDCGRECAICGHCGCCQNAYRRNWETCN

SEQ ID NO: 284 - protein, 26 aa, Artificial Sequence
  CDR-H3 knob domain K114
SCPSGYTLWGDCEGDDGGEGGVCRCW

SEQ ID NO: 285 - protein, 36 aa, Artificial Sequence
  CDR-H3 knob domain K116
SCPEWVQTSRTCIYRSRCGQYVCWSLGEDDCGVTCT

SEQ ID NO: 286 - protein, 37 aa, Artificial Sequence
  CDR-H3 knob domain K117
TCPDAWRSSATCRGAYGEAYECCPSGSSMWTSCVGCT

SEQ ID NO: 287 - protein, 27 aa, Artificial Sequence
  CDR-H3 knob domain K118
TCPDGWRPGSECGWEDRCCGEFCSRCD

SEQ ID NO: 288 - protein, 31 aa, Artificial Sequence
  CDR-H3 knob domain K120
NCPDGYKYNGFCTPDGGCSRVSSWGWDRSCI

SEQ ID NO: 289 - protein, 25 aa, Artificial Sequence
  CDR-H3 knob domain K121
NCPDGGSPSVQCLDDTWACRIVDCY

SEQ ID NO: 290 - protein, 27 aa, Artificial Sequence
  CDR-H3 knob domain K122
TCSCPDNWETSGDCAGSSGDCSDCTCW

SEQ ID NO: 291 - protein, 32 aa, Artificial Sequence
  CDR-H3 knob domain K123
SCPGDRPVDCGDDYGTLGCCPFHVGCGTWRCI

SEQ ID NO: 292 - protein, 26 aa, Artificial Sequence
  CDR-H3 knob domain K124
NCPDGGSPSVQCLDDTWACRIVDCYA

SEQ ID NO: 293 - protein, 36 aa, Artificial Sequence
  CDR-H3 knob domain K125
SCPVGYDGGGNCGRYVDTCWGSDCCRYRRGIDYSCS

SEQ ID NO: 294 - protein, 32 aa, Artificial Sequence
  CDR-H3 knob domain K127
SCPDGYRRGLECSAEWRCRYYDCVECSYGLCG

SEQ ID NO: 295 - protein, 24 aa, Artificial Sequence
  CDR-H3 knob domain K128
NCPDGSSLLSSCFDTGGCSLYSCG

SEQ ID NO: 296 - protein, 20 aa, Artificial Sequence
  CDR-H3 knob domain K130
RCPDGYYQGWHMSLRRYVCA

SEQ ID NO: 297 - protein, 25 aa, Artificial Sequence
  CDR-H3 knob domain K131
NCPTWCGFAHSCILRYEACSDCDCS

SEQ ID NO: 298 - protein, 30 aa, Artificial Sequence
  CDR-H3 knob domain K134
SCPGGYIARCAGTYGCSAVPGCCDFSGDCL

SEQ ID NO: 299 - protein, 8 aa, Artificial Sequence
  CDR-H3 knob domain K135
FCRISECY

SEQ ID NO: 300 - protein, 29 aa, Artificial Sequence
  CDR-H3 knob domain K136
TCPDNYREVDGCDPYDCCLTTWCTNSYCT

SEQ ID NO: 301 - protein, 25 aa, Artificial Sequence
  CDR-H3 knob domain K137
TCPDGGEPSVICLDASEVCRISECY

SEQ ID NO: 302 - protein, 31 aa, Artificial Sequence
  CDR-H3 knob domain K138
SCPAYDSSGCGCVYYSPWNACICDKPGGPCD

SEQ ID NO: 303 - protein, 23 aa, Artificial Sequence
  CDR-H3 knob domain K139
TCPSWCSLYMCGGYLACSACGCA

SEQ ID NO: 304 - protein, 25 aa, Artificial Sequence
  CDR-H3 knob domain K144
TCPDGYVYNDPCDCWGRRNYDCCCE

SEQ ID NO: 305 - protein, 23 aa, Artificial Sequence
  CDR-H3 knob domain K145
SCPDGLSYRAWDDFCCPNVGRCL

SEQ ID NO: 306 - protein, 19 aa, Artificial Sequence
  CDR-H3 knob domain K146
GCPEGTTYLGGSSETYRCG

SEQ ID NO: 307 - protein, 23 aa, Artificial Sequence
  CDR-H3 knob domain K150
TCPDGLSYRSWDGFCCPKVGRCL

SEQ ID NO: 308 - protein, 23 aa, Artificial Sequence
  CDR-H3 knob domain K151
TCPDGLSYRSWDGFCCPKYGRCL

SEQ ID NO: 309 - protein, 14 aa, Artificial Sequence
  CDR-H3 knob domain K153
SCPDGWRDTGTHCE

SEQ ID NO: 310 - protein, 23 aa, Artificial Sequence
  CDR-H3 knob domain K154
SCPSGYSTTLHCCCGSWKCDWCD

SEQ ID NO: 311 - protein, 261 aa, Artificial Sequence
  PGT-121 HC
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 312 - protein, 301 aa, Artificial Sequence
  PGT-121-K149
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGSENLYFQGSCPDGFSYRSWDDFCCPMVGRCLAPRNGSENLYFQGSEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 313 - protein, 27 aa, Artificial Sequence
  K149
SCPDGFSYRSWDDFCCPMVGRCLAPRN

SEQ ID NO: 314 - protein, 304 aa, Artificial Sequence
  PGT-121-K136
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGSENLYFQGTCPDNYREVDGCDPYDCCLTTWCTNSYCTRYIENLYFQGSEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 315 - protein, 32 aa, Artificial Sequence
  K136
TCPDNYREVDGCDPYDCCLTTWCTNSYCTRYI

SEQ ID NO: 316 - protein, 314 aa, Artificial Sequence
  PGT-121-K92
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGSENLYFQGVTCPEGWSECGVAIYGYECGRWGCGHFLNSGPNISPYVTTGSENLYFQGSEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 317 - protein, 40 aa, Artificial Sequence
  K92
VTCPEGWSECGVAIYGYECGRWGCGHFLNSGPNISPYVTT

SEQ ID NO: 318 - protein, 40 aa, Artificial Sequence
  K92
VTCPEGWSECGVAIYGYECGRWGCGHFLNSGPNISPYVST

SEQ ID NO: 319 - protein, 312 aa, Artificial Sequence
  PGT-121-K57
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGSENLYFQGSGCPPGYKSGVDCSPGSECKWGCYAVDGRRYGGYGADSGVENLYFQGSEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 320 - protein, 42 aa, Artificial Sequence
  K57
SGCPPGYKSGVDCSPGSECKWGCYAVDGRRYGGYGADSGVGS

SEQ ID NO: 321 - protein, 317 aa, Artificial Sequence
  PGT-121-K8
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGSENLYFQGVCPDGFNWGYGCAAGSSRFCTRHDWCCYDERADSHTYGFCTGNRVENLYFQGSEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 322 - protein, 45 aa, Artificial Sequence
  K8
VCPDGFNWGYGCAAGSSRFCTRHDWCCYDERADSHTYGFCTGNRV

SEQ ID NO: 323 - protein, 314 aa, Artificial Sequence
  PGT-121-K60
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGSENLYFQGKSCREGYIDGGGCCLPGSCRGCACSYYDWLKCPRDCRGTSEEENLYFQGSEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTLEENLYFQGSGGSHHHHHHHHHH

SEQ ID NO: 324 - protein, 42 aa, Artificial Sequence
  K60
KSCREGYIDGGGCCLPGSCRGCACSYYDWLKCPRDCRGTSEE

SEQ ID NO: 325 - protein, 218 aa, Artificial Sequence
  PGT-121 light chain
QSVLTQPPDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 326 - protein, 52 aa, Artificial Sequence
  K8 isolated from PGT-121
GVCPDGFNWGYGCAAGSSRFCTRHDWCCYDERADSHTYGFCTGNRVENLYFQ

SEQ ID NO: 327 - protein, 47 aa, Artificial Sequence
  K57 isolated from PGT-121
GSGCPPGYKSGVDCSPGSECKWGCYAVDGRRYGGYGADSGVENLYFQ

SEQ ID NO: 328 - protein, 49 aa, Artificial Sequence
  K60 isolated from PGT-121
GKSCREGYIDGGGCCLPGSCRGCACSYYDWLKCPRDCRGTSEEENLYFQ

SEQ ID NO: 329 - protein, 49 aa, Artificial Sequence
  K92 isolated from PGT-121
GVTCPEGWSECGVAIYGYECGRWGCGHFLNSGPNISPYVTTGSENLYFQ

SEQ ID NO: 330 - protein, 39 aa, Artificial Sequence
  K136 isolated from PGT-121
GTCPDNYREVDGCDPYDCCLTTWCTNSYCTRYIENLYFQ

SEQ ID NO: 331 - protein, 36 aa, Artificial Sequence
  K149 isolated from PGT-121
GSCPDGFSYRSWDDFCCPMVGRCLAPRNGSENLYFQ

SEQ ID NO: 332 - protein, 304 aa, Artificial Sequence
  645 Fab HC-K8
EVQLLESGGGLVQPGGSLRLSCAVSGIDLSNYAINWVRQAPGKGLEWIGIIWASGTTFYATWAKGRFTISRDNSGGGGSENLYFQGSVCPDGFNWGYGCAAGSSRFCTRHDWCCYDERADSHTYGFCTGNRVENLYFQGSGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHHHH

SEQ ID NO: 333 - protein, 299 aa, Artificial Sequence
  645 Fab HC-K57
EVQLLESGGGLVQPGGSLRLSCAVSGIDLSNYAINWVRQAPGKGLEWIGIIWASGTTFYATWAKGRFTISRDNSGGGGSENLYFQGSSGCPPGYKSGVDCSPGSECKWGCYAVDGRRYGGYGADSGVENLYFQGSGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHHHH

SEQ ID NO: 334 - protein, 39 aa, Artificial Sequence
  K57
GCPPGYKSGVDCSPGSECKWGCYAVDGRRYGGYGADSGV

SEQ ID NO: 335 - protein, 301 aa, Artificial Sequence
  645 Fab HC-K60
EVQLLESGGGLVQPGGSLRLSCAVSGIDLSNYAINWVRQAPGKGLEWIGIIWASGTTFYATWAKGRFTISRDNSGGGGSENLYFQGSKSCREGYIDGGGCCLPGSCRGCACSYYDWLKCPRDCRGTSEEENLYFQGSGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHHHH

SEQ ID NO: 336 - protein, 42 aa, Artificial Sequence; identical to SEQ ID NO: 324
  K60

SEQ ID NO: 337 - protein, 303 aa, Artificial Sequence
  645 Fab HC-K92
EVQLLESGGGLVQPGGSLRLSCAVSGIDLSNYAINWVRQAPGKGLEWIGIIWASGTTFYATWAKGRFTISRDNSGGGGSENLYFQGSGSVTCPEGWSECGVAIYGYECGRWGCGHFLNSGPNISPYVTTGSENLYFQGSGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHHHH

SEQ ID NO: 338 - protein, 291 aa, Artificial Sequence
  645 Fab HC-K136
EVQLLESGGGLVQPGGSLRLSCAVSGIDLSNYAINWVRQAPGKGLEWIGIIWASGTTFYATWAKGRFTISRDNSGGGGSENLYFQGSTCPDNYREVDGCDPYDCCLTTWCTNSYCTRYIENLYFQGSGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHHHH

SEQ ID NO: 339 - protein, 33 aa, Artificial Sequence
  K136
STCPDNYREVDGCDPYDCCLTTWCTNSYCTRYI

SEQ ID NO: 340 - protein, 289 aa, Artificial Sequence
  645 Fab HC-K149
EVQLLESGGGLVQPGGSLRLSCAVSGIDLSNYAINWVRQAPGKGLEWIGIIWASGTTFYATWAKGRFTISRDNSGGGGSENLYFQGSGSCPDGFSYRSWDDFCCPMVGRCLAPRNGSENLYFQGSGGGSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHHHHHH

SEQ ID NO: 341 - protein, 53 aa, Artificial Sequence
  K8 isolated from 645 Fab
GSVCPDGFNWGYGCAAGSSRFCTRHDWCCYDERADSHTYGFCTGNRVENLYFQ

SEQ ID NO: 342 - protein, 48 aa, Artificial Sequence
  K57 isolated from 645 Fab
GSSGCPPGYKSGVDCSPGSECKWGCYAVDGRRYGGYGADSGVENLYFQ

SEQ ID NO: 343 - protein, 50 aa, Artificial Sequence
  K60 isolated from 645 Fab
GSKSCREGYIDGGGCCLPGSCRGCACSYYDWLKCPRDCRGTSEEENLYFQ

SEQ ID NO: 344 - protein, 52 aa, Artificial Sequence
  K92 isolated from 645 Fab
GSGSVTCPEGWSECGVAIYGYECGRWGCGHFLNSGPNISPYVTTGSENLYFQ

SEQ ID NO: 345 - protein, 40 aa, Artificial Sequence
  K136 isolated from 645 Fab
GSTCPDNYREVDGCDPYDCCLTTWCTNSYCTRYIENLYFQ

SEQ ID NO: 346 - protein, 38 aa, Artificial Sequence
  K149 isolated from 645 Fab
GSGSCPDGFSYRSWDDFCCPMVGRCLAPRNGSENLYFQ

SEQ ID NO: 347 - protein, 27 aa, Artificial Sequence
  K149A
  DISULFID (2)..(15)
  DISULFID (16)..(22)
SCPDGFSYRSWDDFCCPMVGRCLAPRN

SEQ ID NO: 348 - protein, 27 aa, Artificial Sequence
  K149B
  DISULFID (2)..(16)
  DISULFID (15)..(22)
SCPDGFSYRSWDDFCCPMVGRCLAPRN

SEQ ID NO: 349 - protein, 41 aa, Artificial Sequence
  K92
TCPEGWSECGVAIYGYECGRWGCGHFLNSGPNISPYVTTGS

SEQ ID NO: 350 - protein, 29 aa, Artificial Sequence
  K149
SCPDGFSYRSWDDFCCPMVGRCLAPRNGS

SEQ ID NO: 351 - protein, 123 aa, Artificial Sequence
  hC3nb1
QVQLVETGGGLVQAGGSLRLSCAASGSIFSLNAMGWFRQAPGKEREFVATINRSGGRTYYADSVKGRFTISRDNGKNMVYLQMHSLKPEDTAIYYCAAGTGWSPQTDNEYNYWGQGTQVTVSS

SEQ ID NO: 352 - protein, 49 aa, Artificial Sequence
  K57
SGGGSGCPPGYKSGVDCSPGSECKWGCYAVDGRRYGGYGADSGVSGGGS

SEQ ID NO: 353 - protein, 171 aa, Artificial Sequence
  hC3nb1 - K57 loop 1
QVQLVETGGGLVQASGGGSGCPPGYKSGVDCSPGSECKWGCYAVDGRRYGGYGADSGVSGGGSGSLRLSCAASGSIFSLNAMGWFRQAPGKEREFVATINRSGGRTYYADSVKGRFTISRDNGKNMVYLQMHSLKPEDTAIYYCAAGTGWSPQTDNEYNYWGQGTQVTVSS

SEQ ID NO: 354 - protein, 171 aa, Artificial Sequence
  hC3nb1 - K57 loop 2
QVQLVETGGGLVQAGGSLRLSCAASGSIFSLNAMGWFRQAPSGGGSGCPPGYKSGVDCSPGSECKWGCYAVDGRRYGGYGADSGVSGGGSKEREFVATINRSGGRTYYADSVKGRFTISRDNGKNMVYLQMHSLKPEDTAIYYCAAGTGWSPQTDNEYNYWGQGTQVTVSS

SEQ ID NO: 355 - protein, 170 aa, Artificial Sequence
  hC3nb1 - K57 loop 2 delta proline
QVQLVETGGGLVQAGGSLRLSCAASGSIFSLNAMGWFRQASGGGSGCPPGYKSGVDCSPGSECKWGCYAVDGRRYGGYGADSGVSGGGSKEREFVATINRSGGRTYYADSVKGRFTISRDNGKNMVYLQMHSLKPEDTAIYYCAAGTGWSPQTDNEYNYWGQGTQVTVSS

SEQ ID NO: 356 - protein, 172 aa, Artificial Sequence
  hC3nb1 - K57 loop 3
QVQLVETGGGLVQAGGSLRLSCAASGSIFSLNAMGWFRQAPGKEREFVATINRSGGRTYYADSVKGSGGGSGCPPGYKSGVDCSPGSECKWGCYAVDGRRYGGYGADSGVSGGGSRFTISRDNGKNMVYLQMHSLKPEDTAIYYCAAGTGWSPQTDNEYNYWGQGTQVTVSS

SEQ ID NO: 357 - protein, 166 aa, Artificial Sequence
  hC3nb1- K57 C-term
QVQLVETGGGLVQAGGSLRLSCAASGSIFSLNAMGWFRQAPGKEREFVATINRSGGRTYYADSVKGRFTISRDNGKNMVYLQMHSLKPEDTAIYYCAAGTGWSPQTDNEYNYWGQGTQVTVSSGGGSGCPPGYKSGVDCSPGSECKWGCYAVDGRRYGGYGADSGV

SEQ ID NO: 358 - protein, 65 aa, Artificial Sequence
  K57 ultralong CDR-H3
TTVHQRTIKSGCPPGYKSGVDCSPGSECKWGCYAVDGRRYGGYGADSGVGSTYTHEFYVDAWGQG

SEQ ID NO: 359 - protein, 198 aa, Artificial Sequence
  K57 ultralong CDR-H3-hC3nb1
TTVHQRTIKSGCPPGYKSGVDCSPGSECKWGCYAVDGRRYGGYGADSGVGSTYTHEFYVDAWGQGSSSSGSSSSGQVQLVETGGGLVQAGGSLRLSCAASGSIFSLNAMGWFRQAPGKEREFVATINRSGGRTYYADSVKGRFTISRDNGKNMVYLQMHSLKPEDTAIYYCAAGTGWSPQTDNEYNYWGQGTQVTVSS

SEQ ID NO: 360 - protein, 193 aa, Artificial Sequence
  hC3nb1- K57 ultralong CDR-H3
QVQLVETGGGLVQAGGSLRLSCAASGSIFSLNAMGWFRQAPGKEREFVATINRSGGRTYYADSVKGRFTISRDNGKNMVYLQMHSLKPEDTAIYYCAAGTGWSPQTDNEYNYWGQGTQVTVSSGSSSSTTVHQRTIKSGCPPGYKSGVDCSPGSECKWGCYAVDGRRYGGYGADSGVGSTYTHEFYVDAWGQG

SEQ ID NO: 361 - protein, 11 aa, Artificial Sequence
  Flexible linker
SGGGGTGGGGS

SEQ ID NO: 362 - protein, 16 aa, Artificial Sequence
  Flexible linker
SGGGGSGGGGTGGGGS

SEQ ID NO: 363 - protein, 7 aa, Artificial Sequence
  Flexible linker
SGGGGSE

SEQ ID NO: 364 - protein, 6 aa, Artificial Sequence
  Flexible linker
DKTHTS

SEQ ID NO: 365 - protein, 6 aa, Artificial Sequence
  Flexible linker
SGGGGS

SEQ ID NO: 366 - protein, 11 aa, Artificial Sequence
  Flexible linker
SGGGGSGGGGS

SEQ ID NO: 367 - protein, 16 aa, Artificial Sequence
  Flexible linker
SGGGGSGGGGSGGGGS

SEQ ID NO: 368 - protein, 21 aa, Artificial Sequence
  Flexible linker
SGGGGSGGGGSGGGGSGGGGS

SEQ ID NO: 369 - protein, 26 aa, Artificial Sequence
  Flexible linker
SGGGGSGGGGSGGGGSGGGGSGGGGS

SEQ ID NO: 370 - protein, 11 aa, Artificial Sequence
  Flexible linker
AAAGSGGASAS

SEQ ID NO: 371 - protein, 16 aa, Artificial Sequence
  Flexible linker
  misc_feature (7)..(7): Xaa can be any naturally occurring amino acid
AAAGSGXGGGSGASAS

SEQ ID NO: 372 - protein, 21 aa, Artificial Sequence
  Flexible linker
  misc_feature (7)..(7): Xaa can be any naturally occurring amino acid
  misc_feature (12)..(12): Xaa can be any naturally occurring amino acid
AAAGSGXGGGSXGGGSGASAS

SEQ ID NO: 373 - protein, 26 aa, Artificial Sequence
  Flexible linker
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
AAAGSGXGGGSXGGGSXGGGSGASAS

SEQ ID NO: 374 - protein, 31 aa, Artificial Sequence
  Flexible linker
  misc_feature (7)..(7): Xaa can be any naturally occurring amino acid
  misc_feature (12)..(12): Xaa can be any naturally occurring amino acid
  misc_feature (17)..(17): Xaa can be any naturally occurring amino acid
  misc_feature (22)..(22): Xaa can be any naturally occurring amino acid
AAAGSGXGGGSXGGGSXGGGSXGGGSGASAS

SEQ ID NO: 375 - protein, 13 aa, Artificial Sequence
  Flexible linker
  misc_feature (7)..(7): Xaa can be any naturally occurring amino acid
AAAGSGXSGASAS

SEQ ID NO: 376 - protein, 28 aa, Artificial Sequence
  Flexible linker
PGGNRGTTTTRRPATTTGSSPGPTQSHY

SEQ ID NO: 377 - protein, 11 aa, Artificial Sequence
  Flexible linker
ATTTGSSPGPT

SEQ ID NO: 378 - protein, 6 aa, Artificial Sequence
  Flexible linker
ATTTGS

SEQ ID NO: 379 - protein, 21 aa, Artificial Sequence
  Flexible linker
EPSGPISTINSPPSKESHKSP

SEQ ID NO: 380 - protein, 15 aa, Artificial Sequence
  Flexible linker
GTVAAPSVFIFPPSD

SEQ ID NO: 381 - protein, 15 aa, Artificial Sequence
  Flexible linker
GGGGIAPSMVGGGGS

SEQ ID NO: 382 - protein, 15 aa, Artificial Sequence
  Flexible linker
GGGGKVEGAGGGGGS

SEQ ID NO: 383 - protein, 15 aa, Artificial Sequence
  Flexible linker
GGGGSMKSHDGGGGS

SEQ ID NO: 384 - protein, 15 aa, Artificial Sequence
  Flexible linker
GGGGNLITIVGGGGS

SEQ ID NO: 385 - protein, 15 aa, Artificial Sequence
  Flexible linker
GGGGVVPSLPGGGGS

SEQ ID NO: 386 - protein, 12 aa, Artificial Sequence
  Flexible linker
GGEKSIPGGGGS

SEQ ID NO: 387 - protein, 18 aa, Artificial Sequence
  Flexible linker
RPLSYRPPFPFGFPSVRP

SEQ ID NO: 388 - protein, 18 aa, Artificial Sequence
  Flexible linker
YPRSIYIRRRHPSPSLTT

SEQ ID NO: 389 - protein, 18 aa, Artificial Sequence
  Flexible linker
TPSHLSHILPSFGLPTFN

SEQ ID NO: 390 - protein, 18 aa, Artificial Sequence
  Flexible linker
RPVSPFTFPRLSNSWLPA

SEQ ID NO: 391 - protein, 18 aa, Artificial Sequence
  Flexible linker
SPAAHFPRSIPRPGPIRT

SEQ ID NO: 392 - protein, 18 aa, Artificial Sequence
  Flexible linker
APGPSAPSHRSLPSRAFG

SEQ ID NO: 393 - protein, 18 aa, Artificial Sequence
  Flexible linker
PRNSIHFLHPLLVAPLGA

SEQ ID NO: 394 - protein, 18 aa, Artificial Sequence
  Flexible linker
MPSLSGVLQVRYLSPPDL

SEQ ID NO: 395 - protein, 18 aa, Artificial Sequence
  Flexible linker
SPQYPSPLTLTLPPHPSL

SEQ ID NO: 396 - protein, 18 aa, Artificial Sequence
  Flexible linker
NPSLNPPSYLHRAPSRIS

SEQ ID NO: 397 - protein, 17 aa, Artificial Sequence
  Flexible linker
LPWRTSLLPSLPLRRRP

SEQ ID NO: 398 - protein, 18 aa, Artificial Sequence
  Flexible linker
PPLFAKGPVGLLSRSFPP

SEQ ID NO: 399 - protein, 18 aa, Artificial Sequence
  Flexible linker
VPPAPVVSLRSAHARPPY

SEQ ID NO: 400 - protein, 17 aa, Artificial Sequence
  Flexible linker
LRPTPPRVRSYTCCPTP

SEQ ID NO: 401 - protein, 18 aa, Artificial Sequence
  Flexible linker
PNVAHVLPLLTVPWDNLR

SEQ ID NO: 402 - protein, 18 aa, Artificial Sequence
  Flexible linker
CNPLLPLCARSPAVRTFP

SEQ ID NO: 403 - protein, 8 aa, Artificial Sequence
  Hinge linker
DKTHTCAA

SEQ ID NO: 404 - protein, 11 aa, Artificial Sequence
  Hinge linker
DKTHTCPPCPA

SEQ ID NO: 405 - protein, 18 aa, Artificial Sequence
  Hinge linker
DKTHTCPPCPATCPPCPA

SEQ ID NO: 406 - protein, 25 aa, Artificial Sequence
  Hinge linker
DKTHTCPPCPATCPPCPATCPPCPA

SEQ ID NO: 407 - protein, 30 aa, Artificial Sequence
  Hinge linker
DKTHTCPPCPAGKPTLYNSLVMSDTAGTCY

SEQ ID NO: 408 - protein, 31 aa, Artificial Sequence
  Hinge linker
DKTHTCPPCPAGKPTHVNVSVVMAEVDGTCY

SEQ ID NO: 409 - protein, 15 aa, Artificial Sequence
  Hinge linker
DKTHTCCVECPPCPA

SEQ ID NO: 410 - protein, 26 aa, Artificial Sequence
  Hinge linker
DKTHTCPRCPEPKSCDTPPPCPRCPA

SEQ ID NO: 411 - protein, 11 aa, Artificial Sequence
  Hinge linker
DKTHTCPSCPA

SEQ ID NO: 412 - protein, 11 aa, Artificial Sequence
  Rigid linker
GAPAPAAPAPA

SEQ ID NO: 413 - protein, 4 aa, Artificial Sequence
  Rigid linker
PPPP

SEQ ID NO: 414 - protein, 11 aa, Artificial Sequence
  Albumin binding peptide
DLCLRDWGCLW

SEQ ID NO: 415 - protein, 11 aa, Artificial Sequence
  Albumin binding peptide
DICLPRWGCLW

SEQ ID NO: 416 - protein, 15 aa, Artificial Sequence
  Albumin binding peptid
MEDICLPRWGCLWGD

SEQ ID NO: 417 - protein, 20 aa, Artificial Sequence
  Albumin binding peptide
QRLMEDICLPRWGCLWEDDE

SEQ ID NO: 418 - protein, 20 aa, Artificial Sequence
  Albumin binding peptide
QGLIGDICLPRWGCLWGRSV

SEQ ID NO: 419 - protein, 21 aa, Artificial Sequence
  Albumin binding peptide
QGLIGDICLPRWGCLWGRSVK

SEQ ID NO: 420 - protein, 15 aa, Artificial Sequence
  Albumin binding peptide
EDICLPRWGCLWEDD

SEQ ID NO: 421 - protein, 18 aa, Artificial Sequence
  Albumin binding peptide
RLMEDICLPRWGCLWEDD

SEQ ID NO: 422 - protein, 16 aa, Artificial Sequence
  Albumin binding peptide
MEDICLPRWGCLWEDD

SEQ ID NO: 423 - protein, 15 aa, Artificial Sequence
  Albumin binding peptide
MEDICLPRWGCLWED

SEQ ID NO: 424 - protein, 18 aa, Artificial Sequence
  Albumin binding peptide
RLMEDICLARWGCLWEDD

SEQ ID NO: 425 - protein, 20 aa, Artificial Sequence
  Albumin binding peptide
EVRSFCTRWPAEKSCKPLRG

SEQ ID NO: 426 - protein, 20 aa, Artificial Sequence
  Albumin binding peptide
RAPESFVCYWETICFERSEQ

SEQ ID NO: 427 - protein, 11 aa, Artificial Sequence
  Albumin binding peptide
EMCYFPGICWM

SEQ ID NO: 428 - protein, 217 aa, Artificial Sequence
  CA645 Fab Light Chain (gL5)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 429 - protein, 110 aa, Artificial Sequence
  CA645 VL domain (gL5)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIK

SEQ ID NO: 430 - protein, 12 aa, Artificial Sequence
  CDR-L1
QSSPSVWSNFLS

SEQ ID NO: 431 - protein, 7 aa, Artificial Sequence
  CDR-L2
EASKLTS

SEQ ID NO: 432 - protein, 11 aa, Artificial Sequence
  CDR-L3
GGGYSSISDTT

SEQ ID NO: 433 - protein, 224 aa, Artificial Sequence
  CA645 Fab Heavy Chain (gH5)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 434 - protein, 121 aa, Artificial Sequence
  CA645 VH domain (gH5)
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 435 - protein, 10 aa, Artificial Sequence
  CDR-H1
GIDLSNYAIN

SEQ ID NO: 436 - protein, 16 aa, Artificial Sequence
  CDR-H2
IIWASGTTFYATWAKG

SEQ ID NO: 437 - protein, 13 aa, Artificial Sequence
  CDR-H3
TVPGYSTAPYFDL

SEQ ID NO: 438 - protein, 119 aa, Artificial Sequence
  CA645 VH domain (gH1)
EVQLLESGGGLVQPGGSLRLSCAVSGIDLSNYAINWVRQAPGKGLEWIGIIWASGTTFYATWAKGRFTISRDSTTVYLQMNSLRAEDTAVYYCARTVPGYSTAPYFDLWGQGTLVTVSS

SEQ ID NO: 439 - protein, 121 aa, Artificial Sequence
  CA645 VH domain (gH37)
EVQLLESGGGLVQPGGSLRLSCAVSGIDLSNYAINWVRQAPGKGLEWIGIIWASGTTAYATWAKGRFTISRDNSKNTVYLQMNSLRAEDTAVYYCARTVPGYSTAPYFDLWGQGTLVTVSS

SEQ ID NO: 440 - protein, 120 aa, Artificial Sequence
  CA645 VH domain (gH47)
EVQLLESGGGLVQPGGSLRLSCAVSGIDLSNYAINWVRQAPGKGLEWIGIIWASGTTFYATWAKGRFTISRDNSKNTVYLQMNSLRAEDAVYYCARTVPGYSAAPYFDLWGQGTLVTVSS

SEQ ID NO: 441 - protein, 110 aa, Artificial Sequence
  CA645 VL domain (gL1)
DIVMTQSPSSVSASVGDRVTITCQSSPSVWSNFLSWYQQKPGKAPKLLIYEASKLTSGVPSRFKGSGSGTDFTLTISSLQPEDFATYYCGGGYSSISDTTFGGGTKVEIK

SEQ ID NO: 442 - protein, 110 aa, Artificial Sequence; identical to SEQ ID NO: 429
  CA645 VL domain (gL4)

SEQ ID NO: 443 - protein, 110 aa, Artificial Sequence
  CA645-Cys VL domain (gL5)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIK

SEQ ID NO: 444 - protein, 121 aa, Artificial Sequence
  CA645-Cys VH domain (gH5)
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 445 - protein, 112 aa, Artificial Sequence
  CA645-Cys VL (gL4)
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 446 - DNA, 21 bp, Artificial Sequence
  Primer
ggactcggccacmtaytactg

SEQ ID NO: 447 - DNA, 18 bp, Artificial Sequence
  Primer
gctcgagacggtgaycag

SEQ ID NO: 448 - DNA, 61 bp, Artificial Sequence
  Primer
ccatctcatccctgcgtgtctccgactcagtaaggagaacggactcggccacmtaytactg

SEQ ID NO: 449 - DNA, 41 bp, Artificial Sequence
  Primer
cctctctatgggcagtcggtgatgctcgagacggtgaycag

SEQ ID NO: 450 - protein, 39 aa, Artificial Sequence
  K92 knob domain
TCPEGWSECGVAIYGYECGRWGCGHFLNSGPNISPYVST

SEQ ID NO: 451 - protein, 639 aa, Artificial Sequence
  Rat serum albumin - K57 site 1
RGVFRREAHKSEIAHRFKDLGEQHFKGLVLIAFSQYLQKCPYEEHIKLVQEVTDFAKTCVADENASGGGSGCPPGYKSGVDCSPGSECKWGCYAVDGRRYGGYGADSGVSGGGSENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 452 - protein, 640 aa, Artificial Sequence
  Human serum albumin- K57 site 1
RGVFRRDAHKSEVAHRFKDLGEENFKALVLIAFAQYLQQCPFEDHVKLVNEVTEFAKTCVADESASGGGSGCPPGYKSGVDCSPGSECKWGCYAVDGRRYGGYGADSGVSGGGSENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 453 - protein, 639 aa, Artificial Sequence
  Rat serum albumin - K92 site 1
RGVFRREAHKSEIAHRFKDLGEQHFKGLVLIAFSQYLQKCPYEEHIKLVQEVTDFAKTCVADENASGGGSTCPEGWSECGVAIYGYECGRWGCGHFLNSGPNISPYVSTSGGGSENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 454 - protein, 640 aa, Artificial Sequence
  Human serum albumin- K92 site 1
RGVFRRDAHKSEVAHRFKDLGEENFKALVLIAFAQYLQQCPFEDHVKLVNEVTEFAKTCVADESASGGGSTCPEGWSECGVAIYGYECGRWGCGHFLNSGPNISPYVSTSGGGSENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 455 - protein, 639 aa, Artificial Sequence
  Rat serum albumin - K57 site 2
RGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESGGGSGCPPGYKSGVDCSPGSECKWGCYAVDGRRYGGYGADSGVSGGGSSDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 456 - protein, 640 aa, Artificial Sequence
  Human serum albumin - K57 site 2
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQASGGGSGCPPGYKSGVDCSPGSECKWGCYAVDGRRYGGYGADSGVSGGGSADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 457 - protein, 639 aa, Artificial Sequence
  Rat serum albumin - K92 site 2
RGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESGGGSTCPEGWSECGVAIYGYECGRWGCGHFLNSGPNISPYVSTSGGGSSDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 458 - protein, 640 aa, Artificial Sequence
  Human serum albumin - K92 site 2
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQASGGGSTCPEGWSECGVAIYGYECGRWGCGHFLNSGPNISPYVSTSGGGSADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 459 - protein, 639 aa, Artificial Sequence
  Rat serum albumin - K57 site 3
RGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAESGGGSGCPPGYKSGVDCSPGSECKWGCYAVDGRRYGGYGADSGVSGGGSGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 460 - protein, 640 aa, Artificial Sequence
  Human serum albumin - K57 site 3
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAASGGGSGCPPGYKSGVDCSPGSECKWGCYAVDGRRYGGYGADSGVSGGGSADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 461 - protein, 639 aa, Artificial Sequence
  Rat serum albumin - K92 site 3
RGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAESGGGSTCPEGWSECGVAIYGYECGRWGCGHFLNSGPNISPYVSTSGGGSGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 462 - protein, 640 aa, Artificial Sequence
  Human serum albumin - K92 site 3
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAASGGGSTCPEGWSECGVAIYGYECGRWGCGHFLNSGPNISPYVSTSGGGSADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 463 - protein, 639 aa, Artificial Sequence
  Rat serum albumin - K57 site 4
RGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAASGGGSGCPPGYKSGVDCSPGSECKWGCYAVDGRRYGGYGADSGVSGGGSDKDNCFATEGPNLVARSKEALA

SEQ ID NO: 464 - protein, 640 aa, Artificial Sequence
  Human serum albumin - K57 site 4
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADSGGGSGCPPGYKSGVDCSPGSECKWGCYAVDGRRYGGYGADSGVSGGGSDKETCFAEEGKKLVAASQAALGL

SEQ ID NO: 465 - protein, 639 aa, Artificial Sequence
  Rat serum albumin - K92 site 4
RGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDVTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAVLVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAASGGGSTCPEGWSECGVAIYGYECGRWGCGHFLNSGPNISPYVSTSGGGSDKDNCFATEGPNLVARSKEALA

SEQ ID NO: 466 - protein, 640 aa, Artificial Sequence
  Human serum albumin - K92 site 4
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADSGGGSTCPEGWSECGVAIYGYECGRWGCGHFLNSGPNISPYVSTSGGGSDKETCFAEEGKKLVAASQAALGL

SEQ ID NO: 467 - protein, 365 aa, Artificial Sequence
  Rat IgG1 heavy chain constant region Site 1 - K149
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSASGGGGSSCPDGFSYRSWDDFCCPMVGRCLAPRNSGGGGSAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 468 - protein, 365 aa, Artificial Sequence
  Rat IgG1 heavy chain constant region Site 2 - K149
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPESGGGGSSCPDGFSYRSWDDFCCPMVGRCLAPRNSGGGGSGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 469 - protein, 365 aa, Artificial Sequence
  Rat IgG1 heavy chain constant region Site 3 - K149
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNSGGGGSSCPDGFSYRSWDDFCCPMVGRCLAPRNSGGGGSGQPQENYKNTPPTMDTDGSYFLYSKLNVKKEKWQQGNTFTCSVLHEGLHNHHTEKSLSHSPGK

SEQ ID NO: 470 - protein, 365 aa, Artificial Sequence
  Rat IgG1 heavy chain constant region Site 4 - K149
AETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSGGGGSSCPDGFSYRSWDDFCCPMVGRCLAPRNSGGGGSSYFLYSKLNVKKEKWQQGNTFTCSVLHEGLHNHHTEKSLSHSPGK

SEQ ID NO: 471 - protein, 369 aa, Artificial Sequence
  Human IgG1 heavy chain constant region Site 1 - K149
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKASGGGGSSCPDGFSYRSWDDFCCPMVGRCLAPRNSGGGGSLPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 472 - protein, 369 aa, Artificial Sequence
  Human IgG1 heavy chain constant region Site 2 - K149
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGSGGGGSSCPDGFSYRSWDDFCCPMVGRCLAPRNSGGGGSQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 473 - protein, 369 aa, Artificial Sequence
  Human IgG1 heavy chain constant region Site 3 - K149
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNSGGGGSSCPDGFSYRSWDDFCCPMVGRCLAPRNSGGGGSGQPENNYKTTPPVLDSDGSFFLYSKLTVDKSRWQQGNVFSCSVMHEALHNHYTQKSLSLSPGK

SEQ ID NO: 474 - protein, 369 aa, Artificial Sequence
  Human IgG1 heavy chain constant region Site 4 - K149
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSGGGGSSCPDGFSYRSWDDFCCPMVGRCLAPRNSGGGGSSFFLYSKLTVDKSRWQQGNVFSCSVMHEALHNHYTQKSLSLSPGK

SEQ ID NO: 475 - protein, 19 aa, Artificial Sequence
  Mus musculus immunoglobulin heavy-chain leader sequence
MEWSWVFLFFLSVTTGVHS

SEQ ID NO: 476 - protein, 186 aa, Artificial Sequence
  hC3nb1-K8
QVQLVETGGGLVQAGGSLRLSCAASGSIFSLNAMGWFRQAPGKEREFVATINRSGGRTYYADSVKGRFTISRDNGTVQQKTHQVCPDGFNWGYGCAAGSSRFCTRHDWCCYDERADSHTYGFCTGNRVTNTYEFHADAKNMVYLQMHSLKPEDTAIYYCAAGTGWSPQTDNEYNYWGQGTQVTVSS

SEQ ID NO: 477 - protein, 63 aa, Artificial Sequence
  K8 CDR-H3
TVQQKTHQVCPDGFNWGYGCAAGSSRFCTRHDWCCYDERADSHTYGFCTGNRVTNTYEFHADA

SEQ ID NO: 478 - protein, 184 aa, Artificial Sequence
  hC3nb1-K57
QVQLVETGGGLVQAGGSLRLSCAASGSIFSLNAMGWFRQAPGKEREFVATINRSGGRTYYADSVKGRFTISRDNGTTVHQRTIKSGCPPGYKSGVDCSPGSECKWGCYAVDGRRYGGYGADSGVGSTYTHEFYVDAKNMVYLQMHSLKPEDTAIYYCAAGTGWSPQTDNEYNYWGQGTQVTVSS

SEQ ID NO: 479 - protein, 182 aa, Artificial Sequence
  hC3nb1-K92
QVQLVETGGGLVQAGGSLRLSCAASGSIFSLNAMGWFRQAPGKEREFVATINRSGGRTYYADSVKGRFTISRDNGSIVHQKAHTSVTCPEGWSECGVAIYGYECGRWGCGHFLNSGPNISPYVSTHKYEWYVDAKNMVYLQMHSLKPEDTAIYYCAAGTGWSPQTDNEYNYWGQGTQVTVSS

SEQ ID NO: 480 - protein, 171 aa, Artificial Sequence
  hC3nb1-K149
QVQLVETGGGLVQAGGSLRLSCAASGSIFSLNAMGWFRQAPGKEREFVATINRSGGRTYYADSVKGRFTISRDNGTSVLQSTKPQKSCPDGFSYRSWDDFCCPMVGRCLAPRNTYTTEFTIEAKNMVYLQMHSLKPEDTAIYYCAAGTGWSPQTDNEYNYWGQGTQVTVSS

SEQ ID NO: 481 - protein, 41 aa, Artificial Sequence
  K57 knob domain peptide
GCPPGYKSGVDCSPGSECKWGCYAVDGRRYGGYGADSGVGS

SEQ ID NO: 482 - DNA, 45 bp, Artificial Sequence
  Primer
ctcgcggcccagccggccatggccactactgtgcaccaaaaaaca

SEQ ID NO: 483 - DNA, 45 bp, Artificial Sequence
  Primer
ctcgcggcccagccggccatggccactactgtgcaccaaagaacc

SEQ ID NO: 484 - DNA, 45 bp, Artificial Sequence
  Primer
ctcgcggcccagccggccatggccactactgtgcaccaaaaaacg

SEQ ID NO: 485 - DNA, 45 bp, Artificial Sequence
  Primer
ctcgcggcccagccggccatggccactactgtgcaccaacaaact

SEQ ID NO: 486 - DNA, 45 bp, Artificial Sequence
  Primer
ctcgcggcccagccggccatggccactactgtgcaccaacagacc

SEQ ID NO: 487 - DNA, 45 bp, Artificial Sequence
  Primer
ctcgcggcccagccggccatggccactactgtggtccagaaaaca

SEQ ID NO: 488 - DNA, 45 bp, Artificial Sequence
  Primer
ctcgcggcccagccggccatggccactactgtagtccaacgaaca

SEQ ID NO: 489 - DNA, 35 bp, Artificial Sequence
  Primer
tgatgggcggccgcggcatcgacgtaccattcgta

SEQ ID NO: 490 - DNA, 35 bp, Artificial Sequence
  Primer
tgatgggcggccgcggtatcgacgtaccattcgta

SEQ ID NO: 491 - DNA, 35 bp, Artificial Sequence
  Primer
tgatgggcggccgcggcttcgacgtacaattcgta

SEQ ID NO: 492 - DNA, 35 bp, Artificial Sequence
  Primer
tgatgggcggccgcggcattgacgtagaattcgta

SEQ ID NO: 493 - DNA, 35 bp, Artificial Sequence
  Primer
tgatgggcggccgcggcctcgatgtcaaattcgta

SEQ ID NO: 494 - DNA, 35 bp, Artificial Sequence
  Primer
tgatgggcggccgcggtttcgacgtggtattcgta

SEQ ID NO: 495 - DNA, 22 bp, Artificial Sequence
  Primer
gttggccgattcattaatgcag

SEQ ID NO: 496 - DNA, 22 bp, Artificial Sequence
  Primer
acagacagccctcatagttagc

SEQ ID NO: 497 - protein, 62 aa, Artificial Sequence
  Anti-serum albumin ultralong CDR-H3
TTVHQKTTRQTSCPDGYIAGDSSCYRWRCRGNNCCKYGENRLLNYYDYTCVPYRDTYEWYVD

SEQ ID NO: 498 - protein, 63 aa, Artificial Sequence
  Anti-serum albumin ultralong CDR-H3
TTVHQQTHQDQTCPDGYTRTNYYCRRDGCGSWCNGAERQQPCIRGPCCCDLTYRTAYEYHVET

SEQ ID NO: 499 - protein, 54 aa, Artificial Sequence
  Anti-serum albumin ultralong CDR-H3
TTVHQQTQKHCPDDDTDRDGCSRPDSRGGSGCGSYGRYGDQGGACCPLTYEFDV

SEQ ID NO: 500 - protein, 55 aa, Artificial Sequence
  Anti-serum albumin ultralong CDR-H3
TTVHQQTQERCPDDYTDRGGCSIPYNCGGSRCCAYGRNGGYGGISCSRTYEFYVN

SEQ ID NO: 501 - protein, 55 aa, Artificial Sequence
  Anti-serum albumin ultralong CDR-H3
TTVHQQTQERCPDDYTDRGGCSIPYNCGGSRCCAYGRNGGYGGNTCSRTYELYVE

SEQ ID NO: 502 - protein, 55 aa, Artificial Sequence
  Anti-serum albumin ultralong CDR-H3
TTVHQQTQERCPDDYTDRGGCSIPYTCGGSRCCAYGRNGGYGGVSCSRTYEFYVN

SEQ ID NO: 503 - protein, 55 aa, Artificial Sequence
  Anti-serum albumin ultralong CDR-H3
TTVHQQTQERCPDDYTDRGGCSIPYNCGGSRCCAYGRNGGYGGISCSRTYEWYVD

SEQ ID NO: 504 - protein, 55 aa, Artificial Sequence
  Anti-serum albumin ultralong CDR-H3
TTVHQQTQERCPDDYTDRGGCSIPYSCGDSRCCAYGRNGGYGGVSCSRTYEFYVN

SEQ ID NO: 505 - protein, 55 aa, Artificial Sequence
  Anti-serum albumin ultralong CDR-H3
TTVHQQTQERCPDDYTDRGGCSIPYNCGGSRCCAYGRYGDYGGISCSRTYEFYVN

SEQ ID NO: 506 - protein, 55 aa, Artificial Sequence
  Anti-serum albumin ultralong CDR-H3
TTVHQQTQERCPDDYTDRGGCSIPYNCGGSRCCAYGRNGGYGGVSCSRTYEFYVN

SEQ ID NO: 507 - protein, 55 aa, Artificial Sequence
  Anti-serum albumin ultralong CDR-H3
TTVHQQTQERCPDNYTDRGGCSIPYSCGDSRCCAYGRNGGYGGVSCSRTYEFYVN

SEQ ID NO: 508 - protein, 55 aa, Artificial Sequence
  Anti-serum albumin ultralong CDR-H3
TTVHQQTQERCPDNYTDRGGCSIPYTCGGSRCCAYGRNGGYGGVSCSRTYEFYVN

SEQ ID NO: 509 - protein, 45 aa, Artificial Sequence
  Anti-serum albumin ultralong CDR-H3 knob domain
SCPDGYIAGDSSCYRWRCRGNNCCKYGENRLLNYYDYTCVPYRDT

SEQ ID NO: 510 - protein, 45 aa, Artificial Sequence
  Anti-serum albumin ultralong CDR-H3 knob domain
TCPDGYTRTNYYCRRDGCGSWCNGAERQQPCIRGPCCCDLTYRTA

SEQ ID NO: 511 - protein, 40 aa, Artificial Sequence
  Anti-serum albumin ultralong CDR-H3 knob domain
HCPDDDTDRDGCSRPDSRGGSGCGSYGRYGDQGGACCPLT

SEQ ID NO: 512 - protein, 40 aa, Artificial Sequence
  Anti-serum albumin ultralong CDR-H3 knob domain
RCPDDYTDRGGCSIPYNCGGSRCCAYGRNGGYGGISCSRT

SEQ ID NO: 513 - protein, 40 aa, Artificial Sequence
  Anti-serum albumin ultralong CDR-H3 knob domain
RCPDDYTDRGGCSIPYNCGGSRCCAYGRNGGYGGNTCSRT

SEQ ID NO: 514 - protein, 40 aa, Artificial Sequence
  Anti-serum albumin ultralong CDR-H3 knob domain
RCPDDYTDRGGCSIPYTCGGSRCCAYGRNGGYGGVSCSRT

SEQ ID NO: 515 - protein, 40 aa, Artificial Sequence; identical to SEQ ID NO: 512
  Anti-serum albumin ultralong CDR-H3 knob domain

SEQ ID NO: 516 - protein, 40 aa, Artificial Sequence
  Anti-serum albumin ultralong CDR-H3 knob domain
RCPDDYTDRGGCSIPYSCGDSRCCAYGRNGGYGGVSCSRT

SEQ ID NO: 517 - protein, 40 aa, Artificial Sequence
  Anti-serum albumin ultralong CDR-H3 knob domain
RCPDDYTDRGGCSIPYNCGGSRCCAYGRYGDYGGISCSRT

SEQ ID NO: 518 - protein, 40 aa, Artificial Sequence
  Anti-serum albumin ultralong CDR-H3 knob domain
RCPDDYTDRGGCSIPYNCGGSRCCAYGRNGGYGGVSCSRT

SEQ ID NO: 519 - protein, 40 aa, Artificial Sequence
  Anti-serum albumin ultralong CDR-H3 knob domain
RCPDNYTDRGGCSIPYSCGDSRCCAYGRNGGYGGVSCSRT

SEQ ID NO: 520 - protein, 40 aa, Artificial Sequence
  Anti-serum albumin ultralong CDR-H3 knob domain
RCPDNYTDRGGCSIPYTCGGSRCCAYGRNGGYGGVSCSRT

SEQ ID NO: 521 - protein, 60 aa, Artificial Sequence
  Anti-C5 ultralong CDR-H3
TTVHQKTKKTCPLGYNLNDRCDHFNTCRVEKCCQNGVVNAYGICEYAGGNATYTYQWYVH

SEQ ID NO: 522 - protein, 60 aa, Artificial Sequence
  Anti-C5 ultralong CDR-H3
TTVHQRTKKTCPLGYAINDRCDDLKTCGPDECCLNGVVNAYGICEYEGESATHTYEWYVD

SEQ ID NO: 523 - protein, 55 aa, Artificial Sequence
  Anti-C5 ultralong CDR-H3
TTVHQKTEPSCPYGYIYTGGCHTTYGCGNYVCYPGSGPPRVGDVSVSYTYEWYVD

SEQ ID NO: 524 - protein, 60 aa, Artificial Sequence
  Anti-C5 ultralong CDR-H3
TTVHQRTKKTCPLGYDLNDRCDHFNTCRVEECCKNGVVNAYGICEYAGGSATYTYELYVE

SEQ ID NO: 525 - protein, 62 aa, Artificial Sequence
  Anti-C5 ultralong CDR-H3
TTVVQKTQKSEALCPDGYTRSGRPGCYYGCPDSTCCSRTRTLHVSEHCIAPAYTYNYEWYVD

SEQ ID NO: 526 - protein, 54 aa, Artificial Sequence
  Anti-C5 ultralong CDR-H3
TTVHQRTLKNRNCPDGYGYQRHCTVGEDCTEGCCDNYGRCTTYTDTYTYELYVE

SEQ ID NO: 527 - protein, 62 aa, Artificial Sequence
  Anti-C5 ultralong CDR-H3
TTVHQKTNRQESCPGSSGDRTICERSWSCGGYHCSAYDTWGAGGSSDCGTCTYTYTYEWYVD

SEQ ID NO: 528 - protein, 60 aa, Artificial Sequence
  Anti-C5 ultralong CDR-H3
TTVHQRTKKTCPLGYDLNDRCDHFNTCRVEECCKNGVVNAYGICEYAGGSATYTYKWYVD

SEQ ID NO: 529 - protein, 60 aa, Artificial Sequence
  Anti-C5 ultralong CDR-H3
TTVHQRTVKSGRPPGTVAGVHCSPGSDCSWGCYDRDDRRVDGAGADSGLGSTSTYEFYVN

SEQ ID NO: 530 - protein, 56 aa, Artificial Sequence
  Anti-C5 ultralong CDR-H3
TTVHQQTNLRQRSCPDGYKDNRFCSPDGGCSAVSHWGWDSSCVSYTYTDTYEWYVD

SEQ ID NO: 531 - protein, 60 aa, Artificial Sequence
  Anti-C5 ultralong CDR-H3
TTVHQRTVKSGCPTGTVAGVHCSPGSDCSWGCYDKDDRRVDGAGVASGLGSTYTYEFYVN

SEQ ID NO: 532 - protein, 60 aa, Artificial Sequence
  Anti-C5 ultralong CDR-H3
TTVHQRTIKSGCPPGYKSGVDCSPGSECKWGCYAVDGRRYGGYGADSGVGSTYTHEFYVN

SEQ ID NO: 533 - protein, 61 aa, Artificial Sequence
  Anti-C5 ultralong CDR-H3
TTVVQRTHKTTSCPDGYHFIEPCHSGLCWREGACNGDGICANGLGRCRTVSETSTYELYVE

SEQ ID NO: 534 - protein, 60 aa, Artificial Sequence
  Anti-C5 ultralong CDR-H3
TTVHQRTVKSGCPTGTVAGVHCSPGSDCSWGCYDKDDRRVDGAGAASGLGSTYTYEYHVE

SEQ ID NO: 535 - protein, 61 aa, Artificial Sequence
  Anti-C5 ultralong CDR-H3
TTVHQKTRSRCPDGCFLSSYCPVGYACSGFACCDCGGYDYAGGVRGGRCSVRSTTYEWYVD

SEQ ID NO: 536 - protein, 56 aa, Artificial Sequence
  Anti-C5 ultralong CDR-H3
TTVHQQTNKRRQNCPDGYKYNGFCTPDGGCSRVSSWGWDRSCISPTYTYTYEWYVD

SEQ ID NO: 537 - protein, 60 aa, Artificial Sequence
  Anti-C5 ultralong CDR-H3
TTVHQRTVKSGCPPGTVAGVHCSPGSDCSWGCYDRDDRRVDGAGADSGLGSTSTYEFYVN

SEQ ID NO: 538 - protein, 61 aa, Artificial Sequence
  Anti-C5 ultralong CDR-H3
TTVVQRTHKKTSCPDGYHFIEPCHSGLCWREGACNGDGICANGLGRCRTVSETSTYEFYVN

SEQ ID NO: 539 - protein, 61 aa, Artificial Sequence
  Anti-C5 ultralong CDR-H3
TTVVQRTHKTTSCPDGYHFIEPCHSGLCWREGACNGDGICANGLGRCRTVSETSTYEWYVD

SEQ ID NO: 540 - protein, 60 aa, Artificial Sequence
  Anti-C5 ultralong CDR-H3
TTVHQRTVKSGCPTGTVAGVHCSPGSDCSWGCYDADDRRVDGYGADSGVGSPYTYEYHVE

SEQ ID NO: 541 - protein, 60 aa, Artificial Sequence
  Anti-C5 ultralong CDR-H3
TTVHQRTIKSGCPPGYKSGVDCSPGSECKWGCYAVDGRRYGGYGADSGVGSTYTHEYHVE

SEQ ID NO: 542 - protein, 61 aa, Artificial Sequence
  Anti-C5 ultralong CDR-H3
TTVVQRTHKTTSCPDGYHFIEPCHSGLCWREGACNGDGICANGLGRCRTVSETSTYEFYVN

SEQ ID NO: 543 - protein, 54 aa, Artificial Sequence
  Anti-C5 ultralong CDR-H3
TTVHQRTLKNRNCPDGYGYQRHCTVGEDCTDHCCDAYGLCTSYTYTYTYEWYVD

SEQ ID NO: 544 - protein, 54 aa, Artificial Sequence
  Anti-C5 ultralong CDR-H3
TTVHQRTLKNRNCPDGYGYQRHCTVGEDCTDSCCDRYGLCTTSTETYTYELYVE

SEQ ID NO: 545 - protein, 54 aa, Artificial Sequence
  Anti-C5 ultralong CDR-H3
TTVHQRTLKNRNRPEGYGYQRHCTVGEECTDSCCDRYGLCTTSTETYTYEWYVD

SEQ ID NO: 546 - protein, 60 aa, Artificial Sequence
  Anti-C5 ultralong CDR-H3
TTVHQRTKKTCPLGYDLNDRCDHFNTCRVEECCKNGVVNAYGICEYAGGSATYTYEWYVE

SEQ ID NO: 547 - protein, 54 aa, Artificial Sequence
  Anti-C5 ultralong CDR-H3
TTVHQRTLKNRNCPDGYGYQRHCTVGEDCTDSCCDRYGLCTTSTETYTYEFYVN

SEQ ID NO: 548 - protein, 54 aa, Artificial Sequence
  Anti-C5 ultralong CDR-H3
TTVHQRTLKNRNCPDGYGYQRHCTVGEDCTDSCCDRYGLCTTSTETYTYEWYVD

SEQ ID NO: 549 - protein, 54 aa, Artificial Sequence; identical to SEQ ID NO: 548
  Anti-C5 ultralong CDR-H3

SEQ ID NO: 550 - protein, 54 aa, Artificial Sequence
  Anti-C5 ultralong CDR-H3
TTVHQRTLHNRNCPDGYGYQRHCTVGEDCTERCCDNYGLCTSYTDTYTYEFYVN

SEQ ID NO: 551 - protein, 60 aa, Artificial Sequence
  Anti-C5 ultralong CDR-H3
TTVHQRTKKTCPLGYDLNYRCDHFNTCRVEECCKNGVVNAYGICEYAGGSATYTYEWYVD

SEQ ID NO: 552 - protein, 56 aa, Artificial Sequence
  Anti-C5 ultralong CDR-H3
TTVHQRTKEERTCPSGCSWFSGCWDTYRCGPSVCCRDGRYGCAAIICRDTYEWYVD

SEQ ID NO: 553 - protein, 60 aa, Artificial Sequence
  Anti-C5 ultralong CDR-H3
TTVHQKTKKTCPRGYHYNDRCEFFNTCRVEECCLNGVVNTYGICEYEGGSATYTYEWYVD

SEQ ID NO: 554 - protein, 60 aa, Artificial Sequence
  Anti-C5 ultralong CDR-H3
TTVHQKTKKTCPRGYHYNDRCDFFNTCRVEECCLNGVVNTYGICEYEGGSATYTYEWYVD

SEQ ID NO: 555 - protein, 60 aa, Artificial Sequence; identical to SEQ ID NO: 553
  Anti-C5 ultralong CDR-H3

SEQ ID NO: 556 - protein, 62 aa, Artificial Sequence
  Anti-C5 ultralong CDR-H3
TTVHQKTTRVNSCPDGYGYGDGYCYDSGCSASDCYGVDALYSYGHCGCSIYTERPRYEWYVD

SEQ ID NO: 557 - protein, 60 aa, Artificial Sequence
  Anti-C5 ultralong CDR-H3
TTVHLKTKKSCPLGYAINDRCDDLKTCGPDECCLNGVVNAYGICEYEGESATHTYEWYVD

SEQ ID NO: 558 - protein, 60 aa, Artificial Sequence
  Anti-C5 ultralong CDR-H3
TTVHQKTKKSCPLGYAINDRCDDLKTCGPDECCLNGVVNAYGICEYEGESATHTYELYVE

SEQ ID NO: 559 - protein, 60 aa, Artificial Sequence
  Anti-C5 ultralong CDR-H3
TTVHQRTAKRCPSNNEDATACRYSSVCGDYVCEGLSESYAQGWGACRRYACRDSYEWYVD

SEQ ID NO: 560 - protein, 60 aa, Artificial Sequence; identical to SEQ ID NO: 559
  Anti-C5 ultralong CDR-H3

SEQ ID NO: 561 - protein, 56 aa, Artificial Sequence
  Anti-C5 ultralong CDR-H3
TTVHQQTNKRRQNCPDGYEYNGFCTPDGGCSRVSNWGWDRSCISPTYTYTYEWYVE

SEQ ID NO: 562 - protein, 60 aa, Artificial Sequence
  Anti-C5 ultralong CDR-H3
TTVHQQTTKKSSCPDGYCDCNGCGYGNGCSRGGCFDFRLYSGYSADIVVSTTYTHDFYID

SEQ ID NO: 563 - protein, 61 aa, Artificial Sequence
  Anti-C5 ultralong CDR-H3
TTVHQQTKKQKSCPDGWGHSDDCNCACSANAYACCKRDWLLPGPSCECSTYCVSHTYQWYV

SEQ ID NO: 564 - protein, 61 aa, Artificial Sequence
  Anti-C5 ultralong CDR-H3
TTVHQHTRKSGSCPDGWSDCHGSCDGVGCTGSDCVRYNARGYGRHACSGYAYTYSYEFYVN

SEQ ID NO: 565 - protein, 60 aa, Artificial Sequence
  Anti-C5 ultralong CDR-H3
TTVHQRTIKSGCPPGYKSGVDCSPGSECKWGCYAVDGRRYGGYGADSGVGSTYTHELYVE

SEQ ID NO: 566 - protein, 52 aa, Artificial Sequence
  Anti-C5 ultralong CDR-H3
TTVVQRTKRTCPEGLVYNSDQSRCCAADSGVCWEYWRGERVTRGFTYEWYVD

SEQ ID NO: 567 - protein, 60 aa, Artificial Sequence
  Anti-C5 ultralong CDR-H3
TTVHQRTTKRCPSNNEDATACRYSSVCGDYVCEGLSESYAQGWGACRRYACRDSYEWYVD

SEQ ID NO: 568 - protein, 62 aa, Artificial Sequence
  Anti-C5 ultralong CDR-H3
TTVVQRTRKIVTCPDGYSYSEGCGKGDDCGGVHCCANGGVTCWYRHCCSTGTDTYSYEWYVD

SEQ ID NO: 569 - protein, 60 aa, Artificial Sequence
  Anti-C5 ultralong CDR-H3
TTVHQRTAKRCPSNNEEATACRYSSVCGDYVCEGLSESYAQGWGACRRYACRDSYEWYVD

SEQ ID NO: 570 - protein, 60 aa, Artificial Sequence
  Anti-C5 ultralong CDR-H3
TTVHQRTIKSGCPPGYKSGVDCSPGSEYKWGCYAVDGRRYGGYGADSGVGSTYTHEFYVN

SEQ ID NO: 571 - protein, 60 aa, Artificial Sequence
  Anti-C5 ultralong CDR-H3
TTVHQRTIKSGCPPGYKSGVDCSPGSECKWGCYAVDGRRYGGYGADSGVGSTYTHEWYVD

SEQ ID NO: 572 - protein, 43 aa, Artificial Sequence
  Anti-C5 ultralong CDR-H3 knob domain
TCPLGYNLNDRCDHFNTCRVEKCCQNGVVNAYGICEYAGGNAT

SEQ ID NO: 573 - protein, 43 aa, Artificial Sequence
  Anti-C5 ultralong CDR-H3 knob domain
TCPLGYAINDRCDDLKTCGPDECCLNGVVNAYGICEYEGESAT

SEQ ID NO: 574 - protein, 38 aa, Artificial Sequence
  Anti-C5 ultralong CDR-H3 knob domain
SCPYGYIYTGGCHTTYGCGNYVCYPGSGPPRVGDVSVS

SEQ ID NO: 575 - protein, 43 aa, Artificial Sequence
  Anti-C5 ultralong CDR-H3 knob domain
TCPLGYDLNDRCDHFNTCRVEECCKNGVVNAYGICEYAGGSAT

SEQ ID NO: 576 - protein, 40 aa, Artificial Sequence
  Anti-C5 ultralong CDR-H3 knob domain
LCPDGYTRSGRPGCYYGCPDSTCCSRTRTLHVSEHCIAPA

SEQ ID NO: 577 - protein, 34 aa, Artificial Sequence
  Anti-C5 ultralong CDR-H3 knob domain
NCPDGYGYQRHCTVGEDCTEGCCDNYGRCTTYTD

SEQ ID NO: 578 - protein, 41 aa, Artificial Sequence
  Anti-C5 ultralong CDR-H3 knob domain
SCPGSSGDRTICERSWSCGGYHCSAYDTWGAGGSSDCGTCT

SEQ ID NO: 579 - protein, 34 aa, Artificial Sequence
  Anti-C5 ultralong CDR-H3 knob domain
HCSPGSDCSWGCYDRDDRRVDGAGADSGLGSTST

SEQ ID NO: 580 - protein, 32 aa, Artificial Sequence
  Anti-C5 ultralong CDR-H3 knob domain
SCPDGYKDNRFCSPDGGCSAVSHWGWDSSCVS

SEQ ID NO: 581 - protein, 32 aa, Artificial Sequence
  Anti-C5 ultralong CDR-H3 knob domain
HCSPGSDCSWGCYDKDDRRVDGAGVASGLGST

SEQ ID NO: 582 - protein, 42 aa, Artificial Sequence
  Anti-C5 ultralong CDR-H3 knob domain
GCPPGYKSGVDCSPGSECKWGCYAVDGRRYGGYGADSGVGST

SEQ ID NO: 583 - protein, 44 aa, Artificial Sequence
  Anti-C5 ultralong CDR-H3 knob domain
SCPDGYHFIEPCHSGLCWREGACNGDGICANGLGRCRTVSETST

SEQ ID NO: 584 - protein, 42 aa, Artificial Sequence
  Anti-C5 ultralong CDR-H3 knob domain
GCPTGTVAGVHCSPGSDCSWGCYDKDDRRVDGAGAASGLGST

SEQ ID NO: 585 - protein, 46 aa, Artificial Sequence
  Anti-C5 ultralong CDR-H3 knob domain
RCPDGCFLSSYCPVGYACSGFACCDCGGYDYAGGVRGGRCSVRSTT

SEQ ID NO: 586 - protein, 34 aa, Artificial Sequence
  Anti-C5 ultralong CDR-H3 knob domain
NCPDGYKYNGFCTPDGGCSRVSSWGWDRSCISPT

SEQ ID NO: 587 - protein, 44 aa, Artificial Sequence
  Anti-C5 ultralong CDR-H3 knob domain
GCPPGTVAGVHCSPGSDCSWGCYDRDDRRVDGAGADSGLGSTST

SEQ ID NO: 588 - protein, 44 aa, Artificial Sequence; identical to SEQ ID NO: 583
  Anti-C5 ultralong CDR-H3 knob domain

SEQ ID NO: 589 - protein, 42 aa, Artificial Sequence
  Anti-C5 ultralong CDR-H3 knob domain
GCPTGTVAGVHCSPGSDCSWGCYDADDRRVDGYGADSGVGSP

SEQ ID NO: 590 - protein, 31 aa, Artificial Sequence
  Anti-C5 ultralong CDR-H3 knob domain
NCPDGYGYQRHCTVGEDCTDHCCDAYGLCTS

SEQ ID NO: 591 - protein, 35 aa, Artificial Sequence
  Anti-C5 ultralong CDR-H3 knob domain
NCPDGYGYQRHCTVGEDCTDSCCDRYGLCTTSTET

SEQ ID NO: 592 - protein, 25 aa, Artificial Sequence
  Anti-C5 ultralong CDR-H3 knob domain
HCTVGEECTDSCCDRYGLCTTSTET

SEQ ID NO: 593 - protein, 35 aa, Artificial Sequence
  Anti-C5 ultralong CDR-H3 knob domain
NCPDGYGYQRHCTVGEDCTERCCDNYGLCTSYTDT

SEQ ID NO: 594 - protein, 43 aa, Artificial Sequence
  Anti-C5 ultralong CDR-H3 knob domain
TCPLGYDLNYRCDHFNTCRVEECCKNGVVNAYGICEYAGGSAT

SEQ ID NO: 595 - protein, 39 aa, Artificial Sequence
  Anti-C5 ultralong CDR-H3 knob domain
TCPSGCSWFSGCWDTYRCGPSVCCRDGRYGCAAIICRDT

SEQ ID NO: 596 - protein, 43 aa, Artificial Sequence
  Anti-C5 ultralong CDR-H3 knob domain
TCPRGYHYNDRCEFFNTCRVEECCLNGVVNTYGICEYEGGSAT

SEQ ID NO: 597 - protein, 43 aa, Artificial Sequence
  Anti-C5 ultralong CDR-H3 knob domain
TCPRGYHYNDRCDFFNTCRVEECCLNGVVNTYGICEYEGGSAT

SEQ ID NO: 598 - protein, 45 aa, Artificial Sequence
  Anti-C5 ultralong CDR-H3 knob domain
SCPDGYGYGDGYCYDSGCSASDCYGVDALYSYGHCGCSIYTERPR

SEQ ID NO: 599 - protein, 43 aa, Artificial Sequence
  Anti-C5 ultralong CDR-H3 knob domain
SCPLGYAINDRCDDLKTCGPDECCLNGVVNAYGICEYEGESAT

SEQ ID NO: 600 - protein, 45 aa, Artificial Sequence
  Anti-C5 ultralong CDR-H3 knob domain
RCPSNNEDATACRYSSVCGDYVCEGLSESYAQGWGACRRYACRDS

SEQ ID NO: 601 - protein, 34 aa, Artificial Sequence
  Anti-C5 ultralong CDR-H3 knob domain
NCPDGYEYNGFCTPDGGCSRVSNWGWDRSCISPT

SEQ ID NO: 602 - protein, 41 aa, Artificial Sequence
  Anti-C5 ultralong CDR-H3 knob domain
SCPDGYCDCNGCGYGNGCSRGGCFDFRLYSGYSADIVVSTT

SEQ ID NO: 603 - protein, 43 aa, Artificial Sequence
  Anti-C5 ultralong CDR-H3 knob domain
SCPDGWGHSDDCNCACSANAYACCKRDWLLPGPSCECSTYCVS

SEQ ID NO: 604 - protein, 38 aa, Artificial Sequence
  Anti-C5 ultralong CDR-H3 knob domain
SCPDGWSDCHGSCDGVGCTGSDCVRYNARGYGRHACSG

SEQ ID NO: 605 - protein, 42 aa, Artificial Sequence; identical to SEQ ID NO: 582
  Anti-C5 ultralong CDR-H3 knob domain

SEQ ID NO: 606 - protein, 37 aa, Artificial Sequence
  Anti-C5 ultralong CDR-H3 knob domain
TCPEGLVYNSDQSRCCAADSGVCWEYWRGERVTRGFT

SEQ ID NO: 607 - protein, 43 aa, Artificial Sequence
  Anti-C5 ultralong CDR-H3 knob domain
TCPDGYSYSEGCGKGDDCGGVHCCANGGVTCWYRHCCSTGTDT

SEQ ID NO: 608 - protein, 45 aa, Artificial Sequence
  Anti-C5 ultralong CDR-H3 knob domain
RCPSNNEEATACRYSSVCGDYVCEGLSESYAQGWGACRRYACRDS

SEQ ID NO: 609 - protein, 43 aa, Artificial Sequence
  Anti-C5 ultralong CDR-H3 knob domain
SGCPPGYKSGVDCSPGSEYKWGCYAVDGRRYGGYGADSGVGST